SEQUENCE LISTING (52 sequences)

SEQ ID NO: 1 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 2 - DNA, 2057 bp, Homo sapiens
  source 1..2057: mol_type = genomic DNA
atattgtgcttccaccactgccaataacaaaataactagcaaccatgaagtgggtggaatcaatttttttaattttcctactaaattttactgaatccagaacactgcatagaaatgaatatggaatagcttccatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaaaaaaatatcttcttggcaagttttgttcatgaatattcaagaagacatcctcagcttgctgtctcagtaattctaagagttgctaaaggataccaggagttattggagaagtgtttccagactgaaaaccctcttgaatgccaagataaaggagaagaagaattacagaaatacatccaggagagccaagcattggcaaagcgaagctgcggcctcttccagaaactaggagaatattacttacaaaatgcgtttctcgttgcttacacaaagaaagccccccagctgacctcgtcggagctgatggccatcaccagaaaaatggcagccacagcagccacttgttgccaactcagtgaggacaaactattggcctgtggcgagggagcggctgacattattatcggacacttatgtatcagacatgaaatgactccagtaaaccctggtgttggccagtgctgcacttcttcatatgccaacaggaggccatgcttcagcagcttggtggtggatgaaacatatgtccctcctgcattctctgatgacaagttcattttccataaggatctgtgccaagctcagggtgtagcgctgcaaacgatgaagcaagagtttctcattaaccttgtgaagcaaaagccacaaataacagaggaacaacttgaggctgtcattgcagatttctcaggcctgttggagaaatgctgccaaggccaggaacaggaagtctgctttgctgaagagggacaaaaactgatttcaaaaactcgtgctgctttgggagtttaaattacttcaggggaagagaagacaaaacgagtctttcattcggtgtgaacttttctctttaattttaactgatttaacactttttgtgaattaatgaaatgataaagacttttatgtgagatttccttatcacagaaataaaatatctccaaatgtttccttttcaaaaaaaaaaaaaaaaaa

SEQ ID NO: 3 - protein, 609 aa, Homo sapiens [sequence omitted]

SEQ ID NO: 4 - DNA, 2264 bp, Homo sapiens
  source 1..2264: mol_type = genomic DNA
agtatattagtgctaatttccctccgtttgtcctagcttttctcttctgtcaaccccacacgcctttggcacaatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataacatcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaaaaaaaaaaaaaaaaaaa

SEQ ID NO: 5 - protein, 313 aa, Homo sapiens
MYVSYLLDKDVSMYPSSVRHSGGLNLAPQNFVSPPQYPDYGGYHVAAAAAAAANLDSAQSPGPSWPAAYGAPLREDWNGYAPGGAAAAANAVAHGLNGGSPAAAMGYSSPADYHPHHHPHHHPHHPAAAPSCASGLLQTLNPGPPGPAATAAAEQLSPGGQRRNLCEWMRKPAQQSLGSQVKTRTKDKYRVVYTDHQRLELEKEFHYSRYITIRRKAELAATLGLSERQVKIWFQNRRAKERKINKKKLQQQQQQQPPQPPPPPPQPPQPQPGPLRSVPEPLSPVSSLQASVSGSVPGVLGPTGGVLNPTVTQ

SEQ ID NO: 6 - DNA, 2370 bp, Homo sapiens
  source 1..2370: mol_type = genomic DNA
ctccaaccattggtgtctgtgtcattactaatagagtcttgtaaacactcgttaatcacggaaggccgccggcctggggctccgcacgccagcctgtggcgggtcttccccgcctctgcagcctagtgggaaggaggtgggaggaaagaaggaagaaagggagggagggaggaggcaggccagagggagggaccgcctcggaggcagaagagccgcgaggagccagcggagcaccgcgggctggggcgcagccacccgccgctcctcgagtcccctcgcccctttcccttcgtgccccccggcagcctccagcgtcggtccccaggcagcatggtgaggtctgctcccggaccctcgccaccatgtacgtgagctacctcctggacaaggacgtgagcatgtaccctagctccgtgcgccactctggcggcctcaacctggcgccgcagaacttcgtcagccccccgcagtacccggactacggcggttaccacgtggcggccgcagctgcagcggcagcgaacttggacagcgcgcagtccccggggccatcctggccggcagcgtatggcgccccactccgggaggactggaatggctacgcgcccggaggcgccgcggccgccgccaacgccgtggctcacggcctcaacggtggctccccggccgcagccatgggctacagcagccccgcagactaccatccgcaccaccacccgcatcaccacccgcaccacccggccgccgcgccttcctgcgcttctgggctgctgcaaacgctcaaccccggccctcctgggcccgccgccaccgctgccgccgagcagctgtctcccggcggccagcggcggaacctgtgcgagtggatgcggaagccggcgcagcagtccctcggcagccaagtgaaaaccaggacgaaagacaaatatcgagtggtgtacacggaccaccagcggctggagctggagaaggagtttcactacagtcgctacatcaccatccggaggaaagccgagctagccgccacgctggggctctctgagaggcaggttaaaatctggtttcagaaccgcagagcaaaggagaggaaaatcaacaagaagaagttgcagcagcaacagcagcagcagccaccacagccgcctccgccgccaccacagcctccccagcctcagccaggtcctctgagaagtgtcccagagcccttgagtccggtgtcttccctgcaagcctcagtgtctggctctgtccctggggttctggggccaactgggggggtgctaaaccccaccgtcacccagtgacccaccgggttctgcagcggcagagcaattccaggctgagccatgaggagcgtggactctgctagactcctcaggagagacccctcccctcccacccacagccatagacctacagacctggctctcagaggaaaaatgggagccaggagtaagacaagtgggatttggggcctcaagaaatatactctcccagatttttactttttcccatctggctttttctgccactgaggagacagaaagcctccgctgggcttcattccggactggcagaagcattgcctggactgaccacaccaaccaggccttcatcctcctccccagctcttctcttcctagatctgcaggctgcacctctggctagagccgaggggagagagggactcaagggaaaggcaagcttgaggccaagatggctgctgcctgctcatggccctcggaggtccagctgggcctcctgcctccgggcaggcaaggtttacactgcggaagccaaaggcagctaagatagaaagctggactgaccaaagactgcagaacccccaggtggcctgcgtcttttttctcttcccttcccagaccaggaaaggcttggctggtgtatgcacagggtgtggtatgagggggtggttattggactccaggcctgaccagggggcccgaacagggacttgtttagagagcctgtcaccagagcttctctgggctgaatgtatgtcagtgctataaatgccagagccaacctggacttcctgtcattttcacaatcttggggctgatgaagaagggggtggggggagtttgtgttgttgttgctgctgtttgggttgttggtctgtgtaacatccaagccagagtttttaaagccttctggatccatggggggagaagtgatatggtgaagggaagtggggagtatttgaacacagttgaattttttctaaaaagaaaaagagataaatgagctttccagatttcagattctgtatttatcttcagattttgtctgcaactattttttattttttaaagaaatgaaatatcttcaaaaaaaaaaaaaaaaaaa

SEQ ID NO: 7 - protein, 503 aa, Homo sapiens
MDLIPNLAVETWLLLAVSLILLYLYGTRTHGLFKKLGIPGPTPLPFLGNALSFRKGYWTFDMECYKKYRKVWGIYDCQQPMLAITDPDMIKTVLVKECYSVFTNRRPFGPVGFMKNAISIAEDEEWKRIRSLLSPTFTSGKLKEMVPIIAQYGDVLVRNLRREAETGKPVTLKHVFGAYSMDVITSTSFGVSIDSLNNPQDPFVENTKKLLRFNPLDPFVLSIKVFPFLTPILEALNITVFPRKVISFLTKSVKQIKEGRLKETQKHRVDFLQLMIDSQNSKDSETHKALSDLELMAQSIIFIFAGYETTSSVLSFIIYELATHPDVQQKVQKEIDTVLPNKAPPTYDTVLQLEYLDMVVNETLRLFPVAMRLERVCKKDVEINGMFIPKGVVVMIPSYVLHHDPKYWTEPEKFLPERFSKKNKDNIDPYIYTPFGSGPRNCIGMRFALVNMKLALVRVLQNFSFKPCKETQIPLKLRFGGLLLTEKPIVLKAESRDETVSGA

SEQ ID NO: 8 - DNA, 2099 bp, Homo sapiens
  source 1..2099: mol_type = genomic DNA
aatcactgctgtgcagggcaggaaagctccacacacacagcccagcaaacagcagcacgctgctgaaaaaaagactcagaggagagagataaggaaggaaagtagtgatggatctcatcccaaacttggccgtggaaacctggcttctcctggctgtcagcctgatactcctctatctatatggaacccgtacacatggactttttaagaagcttggaattccagggcccacacctctgccttttttgggaaatgctttgtccttccgtaagggctattggacgtttgacatggaatgttataaaaagtatagaaaagtctggggtatttatgactgtcaacagcctatgctggctatcacagatcccgacatgatcaaaacagtgctagtgaaagaatgttattctgtcttcacaaaccggaggcctttcgggccagtgggatttatgaaaaatgccatctctatagctgaggatgaagaatggaagagaatacgatcattgctgtctccaacattcaccagcggaaaactcaaggagatggtccctatcattgcccagtatggagatgtgttggtgagaaatctgaggcgggaagcagagacaggcaagcctgtcaccttgaaacacgtctttggggcctacagcatggatgtgatcactagcacatcatttggagtgagcatcgactctctcaacaatccacaagacccctttgtggaaaacaccaagaagcttttaagatttaatccattagatccattcgttctctcaataaaagtctttccattccttaccccaattcttgaagcattaaatatcactgtgtttccaagaaaagttataagttttctaacaaaatctgtaaaacagataaaagaaggtcgcctcaaagagacacaaaagcaccgagtggatttccttcagctgatgattgactctcagaattcaaaagactctgagacccacaaagctctgtctgatctggagctcatggcccaatcaattatctttatttttgctggctatgaaaccacgagcagtgttctctccttcattatatatgaactggccactcaccctgatgtccagcagaaagtgcagaaggaaattgatacagttttacccaataaggcaccacccacctatgatactgtgctacagttggagtatcttgacatggtggtgaatgaaacactcagattattcccagttgctatgagacttgagagggtctgcaaaaaagatgttgaaatcaatgggatgtttattcccaaaggggtggtggtgatgattccaagctatgttcttcatcatgacccaaagtactggacagagcctgagaagttcctccctgaaaggttcagtaaaaagaacaaggacaacatagatccttacatatacacaccctttggaagtggacccagaaactgcattggcatgaggtttgctctcgtgaacatgaaacttgctctagtcagagtccttcagaacttctccttcaaaccttgtaaagaaacacagatccccctgaaattacgctttggaggacttcttctaacagaaaaacccattgttctaaaggctgagtcaagggatgagaccgtaagtggagcctgatttccctaaggacttctggtttgctctttaagaaagctgtgccccagaacaccagagacctcaaattactttacaaatagaaccctgaaatgaagacgggcttcatccaatgtgctgcataaataatcagggattctgtacgtgcattgtgctctctcatggtctgtatagagtgttatacttggtaatatagaggagatgaccaaatcagtgctggggaagtagatttggcttctctgcttctcataggactatctccaccacccccagttagcaccattaactcctcctgagctctgataacataattaacatttctcaataatttcaaccacaatcattaataaaaataggaattattttgatggctctaacagtgacatttatatcatgtgttatatctgtagtattctatagtaagctttatattaagcaaatcaataaaaacctctttacaaaagtaaaaaaaaaaaaaaaaaa

SEQ ID NO: 9 - protein, 435 aa, Homo sapiens
MSNKDRHIDSSCSSFIKTEPSSPASLTDSVNHHSPGGSSDASGSYSSTMNGHQNGLDSPPLYPSAPILGGSGPVRKLYDDCSSTIVEDPQTKCEYMLNSMPKRLCLVCGDIASGYHYGVASCEACKAFFKRTIQGNIEYSCPATNECEITKRRRKSCQACRFMKCLKVGMLKEGVRLDRVRGGRQKYKRRIDAENSPYLNPQLVQPAKKPYNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEDPRRAGKMLMTLPLLRQTSTKAVQHFYNIKLEGKVPMHKLFLEMLEAKV

SEQ ID NO: 10 - DNA, 5559 bp, Homo sapiens
  source 1..5559: mol_type = genomic DNA
aagctccaatcggggctttaagtccttgattaggagagtgtgagagctttggtcccaactggctgtgcctataggcttgtcactaggagaacatttgtgttaattgcactgtgctctgtcaaggaaactttgatttatagctggggtgcacaaataatggttgccggtcgcacatggattcggtagaactttgccttcctgaatctttttccctgcactacgaggaagagtagacttgaatgagacctgcctcatcagtcatgggatcatagtgtcacagatggaaaagcaactatcagctgaattgtactgaactacacacttggctaattcatcttattgctctacacatctaaaggaaggctcattctgttcttggagtctagacagcatcaggagttgggctcagtgaacaaaactttaatgtctagagcatttatgagggttttaatgattggaaaatctatcctgagaatgtggtcaccatatgtgacagccttgctttctatcttgtcttcagtttctggggcttctctgcagaatgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccatataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcgtttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtctgactaaaagctccctgggccttcccatccttcatgttgaaaaagggaaaataaacccaagagtgatgtcgaagaaacttagagtttagttaacaacatcaaaaatcaacagactgcactgataatttagcagcaagactatgaagcagctttcagattcctccataggttcctgatgagtttctttctactttctccatcatcttctttcctctttcttcccacatttctctttctctttattttttctccttttcttctttcacctcccttatttctttgcttctttcattcctagttcccattctcctttattttcttcccgtctgcctgccttctttcttttctttacctactctcattcctctcttttctcatccttccccttttttctaaatttgaaatagctttagtttaaaaaaaaatcctcccttccccctttcctttccctttctttcctttttccctttccttttccctttcctttcctttcctcttgaccttctttccatctttctttttcttccttctgctgctgaacttttaaaagaggtctctaactgaagagagatggaagccagccctgccaaaggatggagatccataatatggatgccagtgaacttattgtgaaccatactgtccccaatgactaaggaatcaaagagagagaaccaacgttcctaaaagtacagtgcaacatatacaaattgactgagtgcagtattagatttcatgggagcagcctctaattagacaacttaagcaacgttgcatcggctgcttcttatcattgcttttccatctagatcagttacagccatttgattccttaattgttttttcaagtcttccaggtatttgttagtttagctactatgtaactttttcagggaatagtttaagctttattcattcatgcaatactaaagagaaataagaatactgcaattttgtgctggctttgaacaattacgaacaataatgaaggacaaatgaatcctgaaggaagatttttaaaaatgttttgtttcttcttacaaatggagatttttttgtaccagctttaccacttttcagccatttattaatatgggaatttaacttactcaagcaatagttgaagggaaggtgcatattatcacggatgcaatttatgttgtgtgccagtctggtcccaaacatcaatttcttaacatgagctccagtttacctaaatgttcactgacacaaaggatgagattacacctacagtgactctgagtagtcacatatataagcactgcacatgagatatagatccgtagaattgtcaggagtgcacctctctacttgggaggtacaattgccatatgatttctagctgccatggtggttaggaatgtgatactgcctgtttgcaaagtcacagaccttgcctcagaaggagctgtgagccagtattcatttaagaggcaataaggcaaatgccagaattaaaaaaaaaaatcatcaaagacagaaaatgcctgaccaaattctaaaacctaatccatataagtttattcatttaggaatgttcgtttaaattaatctgcagtttttaccaagagctaagccaatatatgtgcttttcaaccagtattgtcacagcatgaaagtcaagtcaggttccagactgttaagaggtgtaatctaatgaagaaatcaattagatgccccgaaatctacagtcgctgaataaccaataaacagtaacctccatcaaatgctataccaatggaccagtgttagtagctgctccctgtattatgtgaacagtcttattctatgtacacagatgtaattaaaattgtaatcctaacaaacaaaagaaatgtagttcagcttttcaatgtttcatgtttgctgtgcttttctgaattttatgttgcattcaaagactgttgtcttgttcttgtggtgtttggattcttgtggtgtgtgcttttagacacagggtagaattagagacaatattggatgtacaattcctcaggagactacagtagtatattctattccttaccagtaataaggttcttcctaataataattaagagattgaaactccaaacaagtattcattatgaacagatacacatcaaaatcataataatattttcaaaacaaggaataatttctctaatggtttattatagaataccaatgtatagcttagaaataaaactttgaatatttcaagaatatagataagtctaatttttaaatgctgtatatatggctttcactcaatcatctctcagatgttgttattaactcgctctgtgttgttgcaaaactttttggtgcagattcgtttccaaaactattgctactttgtgtgctttaaacaaaataccttgggttgatgaaacatcaacccagtgctaggaatactgtgtatctatcattagctatatgggactatattgtagattgtggtttctcagtagagaagtgactgtagtgtgattctagataaatcatcattagcaattcattcagatggtcaataacttgaaatttatagctgtgataggagttcagaaattggcacatccctttaaaaataacaacagaaaatacaactcctgggaaaaaaggtgctgattctataagattatttatatatgtaagtgtttaaaaagattattttccagaaagtttgtgcagggtttaagttgctactattcaactacactatatataaataaaatatatacaatatatacattgttttcactgtatcacattaaagtacttgggcttcagaagtaagagccaaccaactgaaaacctgagatggagatatgttcaaagaatgagatacaattttttagttttcagtttaagtaactctcagcattacaaaagagtaagtatctcacaaataggaaataaaactaaaacgtggatttaaaaagaactgcacgggctttagggtaaatgctcatcttaaacctcactagagggaagtcttctcaagtttcaagcaagaccatttacttaatgtgaagttttggaaagttataaaggtgtatgttttagccatatgattttaattttaattttgcttcttttaggttcgttcttatttaaagcaatatgattgtgtgactccttgtagttacacttgtgtttcaatcagatcagattgttgtatttattccactattttgcatttaaatgataacataaaagatataaaaaatttaaaactgctatttttcttatagaagagaaaatgggtgttggtgattgtattttaattatttaagcgtctctgtttacctgcctaggaaaacattttatggcagtcttatgtgcaaagatcgtaaaaggacaaaaaatttaaactgcttataataatccaggagttgcattatagccagtagtaaaaataataataataataataaaaccatgtctatagctgtagatgggcttcacatctgtaaagcaatcaattgtatatttttgtgatgtgtaccatactgtgtgctccagcaaatgtccatttgtgtaaatgtatttattttatattgtatatattgttaaatgcaaaaaggagatatgattctgtaactccaatcagttcagatgtgtaactcaaattattatgcctttcaggatgatggtagagcaatattaaacaagcttccacttttgactgctaaaaaaaaaaaaaaaaa

SEQ ID NO: 11 - protein, 463 aa, Homo sapiens
MHSASSMLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 12 - DNA, 2428 bp, Homo sapiens
  source 1..2428: mol_type = genomic DNA
cccgcccacttccaactaccgcctccggcctgcccagggagagagagggagtggagcccagggagagggagcgcgagagagggagggaggaggggacggtgctttggctgacttttttttaaaagagggtgggggtggggggtgattgctggtcgtttgttgtggctgttaaattttaaactgccatgcactcggcttccagtatgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaagaagacgacggcttcaggcccggctaactctggcaccccggatcgaggacaagtgagagagcaagtgggggtcgagactttggggagacggtgttgcagagacgcaagggagaagaaatccataacacccccaccccaacacccccaagacagcagtcttcttcacccgctgcagccgttccgtcccaaacagagggccacacagataccccacgttctatataaggaggaaaacgggaaagaatataaagttaaaaaaaagcctccggtttccactactgtgtagactcctgcttcttcaagcacctgcagattctgatttttttgttgttgttgttctcctccattgctgttgttgcagggaagtcttacttaaaaaaaaaaaaaaattttgtgagtgactcggtgtaaaaccatgtagttttaacagaaccagagggttgtactattgtttaaaaacaggaaaaaaaataatgtaagggtctgttgtaaatgaccaagaaaaagaaaaaaaaagcattcccaatcttgacacggtgaaatccaggtctcgggtccgattaatttatggtttctgcgtgctttatttatggcttataaatgtgtattctggctgcaagggccagagttccacaaatctatattaaagtgttatacccggttttatcccttgaatcttttcttccagatttttcttttctttacttggcttacaaaatatacaggcttggaaattatttcaagaaggagggagggataccctgtctggttgcaggttgtattttattttggcccagggagtgttgctgttttcccaacattttattaataaaattttcagacataaaaaa

SEQ ID NO: 13 - protein, 595 aa, Homo sapiens
MALTDGGWCLPKRFGAAGADASDSRAFPAREPSTPPSPISSSSSSCSRGGERGPGGASNCGTPQLDTEAAAGPPARSLLLSSYASHPFGAPHGPSAPGVAGPGGNLSSWEDLLLFTDLDQAATASKLLWSSRGAKLSPFAPEQPEEMYQTLAALSSQGPAAYDGAPGGFVHSAAAAAAAAAAASSPVYVPTTRVGSMLPGLPYHLQGSGSGPANHAGGAGAHPGWPQASADSPPYGSGGGAAGGGAAGPGGAGSAAAHVSARFPYSPSPPMANGAAREPGGYAAAGSGGAGGVSGGGSSLAAMGGREPQYSSLSAARPLNGTYHHHHHHHHHHPSPYSPYVGAPLTPAWPAGPFETPVLHSLQSRAGAPLPVPRGPSADLLEDLSESRECVNCGSIQTPLWRRDGTGHYLCNACGLYSKMNGLSRPLIKPQKRVPSSRRLGLSCANCHTTTTTLWRRNAEGEPVCNACGLYMKLHGVPRPLAMKKEGIQTRKRKPKNINKSKTCSGNSNNSIPMTPTSTSSNSDDCSKNTSPTTQPTASGAGAPVMTGAGESTNPENSELKYSGQDGLYIGVSLASPAEVTSSVRPDSWCALALA

SEQ ID NO: 14 - DNA, 3785 bp, Homo sapiens
  source 1..3785: mol_type = genomic DNA
agttccgacccacagcctggcacccttcggcgagcgctgtttgtttagggctcggtgagtccaatcaggagcccaggctgcagttttccggcagagcagtaagaggcgcctcctctctcctttttattcaccagcagcgcggcgcagaccccggactcgcgctcgcccgctggcgccctcggcttctctccgcgcctgggagcaccctccgccgcggccgttctccatgcgcagcgcccgcccgaggagctagacgtcagcttggagcggcgccggaccgtggatggccttgactgacggcggctggtgcttgccgaagcgcttcggggccgcgggtgcggacgccagcgactccagagcctttccagcgcgggagccctccacgccgccttcccccatctcttcctcgtcctcctcctgctcccggggcggagagcggggccccggcggcgccagcaactgcgggacgcctcagctcgacacggaggcggcggccggacccccggcccgctcgctgctgctcagttcctacgcttcgcatcccttcggggctccccacggaccttcggcgcctggggtcgcgggccccgggggcaacctgtcgagctgggaggacttgctgctgttcactgacctcgaccaagccgcgaccgccagcaagctgctgtggtccagccgcggcgccaagctgagccccttcgcacccgagcagccggaggagatgtaccagaccctcgccgctctctccagccagggtccggccgcctacgacggcgcgcccggcggcttcgtgcactctgcggccgcggcggcagcagccgcggcggcggccagctccccggtctacgtgcccaccacccgcgtgggttccatgctgcccggcctaccgtaccacctgcaggggtcgggcagtgggccagccaaccacgcgggcggcgcgggcgcgcaccccggctggcctcaggcctcggccgacagccctccatacggcagcggaggcggcgcggctggcggcggggccgcggggcctggcggcgctggctcagccgcggcgcacgtctcggcgcgcttcccctactctcccagcccgcccatggccaacggcgccgcgcgggagccgggaggctacgcggcggcgggcagtgggggcgcgggaggcgtgagcggcggcggcagtagcctggcggccatgggcggccgcgagccccagtacagctcgctgtcggccgcgcggccgctgaacgggacgtaccaccaccaccaccaccaccaccaccaccatccgagcccctactcgccctacgtgggggcgccactgacgcctgcctggcccgccggacccttcgagaccccggtgctgcacagcctgcagagccgcgccggagccccgctcccggtgccccggggtcccagtgcagacctgctggaggacctgtccgagagccgcgagtgcgtgaactgcggctccatccagacgccgctgtggcggcgggacggcaccggccactacctgtgcaacgcctgcgggctctacagcaagatgaacggcctcagccggcccctcatcaagccgcagaagcgcgtgccttcatcacggcggcttggattgtcctgtgccaactgtcacaccacaactaccaccttatggcgcagaaacgccgagggtgaacccgtgtgcaatgcttgtggactctacatgaaactccatggggtgcccagaccacttgctatgaaaaaagagggaattcaaaccaggaaacgaaaacctaagaacataaataaatcaaagacttgctctggtaatagcaataattccattcccatgactccaacttccacctcttctaactcagatgattgcagcaaaaatacttcccccacaacacaacctacagcctcaggggcgggtgccccggtgatgactggtgcgggagagagcaccaatcccgagaacagcgagctcaagtattcgggtcaagatgggctctacataggcgtcagtctcgcctcgccggccgaagtcacgtcctccgtgcgaccggattcctggtgcgccctggccctggcctgagcccacgccgccaggaggcagggagggctccgccgcgggcctcactccactcgtgtctgcttttgtgcagcggtccagacagtggcgactgcgctgacagaacgtgattctcgtgcctttattttgaaagagatgtttttcccaagaggcttgctgaaagagtgagagaagatggaagggaagggccagtgcaactgggcgcttgggccactccagccagcccgcctccggggcggaccctgctccacttccagaagccaggactaggacctgggccttgcctgctatggaatattgagagagattttttaaaaaagattttgcattttgtccaaaatcatgtgcttcttctgatcaattttggttgttccagaatttcttcataccttttccacatccagatttcatgtgcgttcatggagaagatcacttgaggccatttggtacacatctctggaggctgagtcggttcatgaggtctcttatcaaaaatattactcagtttgcaagactgcattgtaactttaacatacactgtgactgacgtttctcaaagttcatattgtgtggctgatctgaagtcagtcggaatttgtaaacagggtagcaaacaagatatttttcttccatgtatacaataatttttttaaaaagtgcaatttgcgttgcagcaatcagtgttaaatcatttgcataagatttaacagcattttttataatgaatgtaaacattttaacttaatggtacttaaaataatttaaaagaaaaatgttaacttagacattcttatgcttcttttacaactacatcccattttatatttccaattgttaaagaaaaatatttcaagaacaaatcttctctcaggaaaattgcctttctctatttgttaagaatttttatacaagaacaccaatataccccctttattttactgtggaatatgtgctggaaaaattgcaacaacactttactacctaacggatagcatttgtaaatactctaggtatctgtaaacactctgatgaagtctgtatagtgtgactaacccacaggcaggttggtttacattaatttttttttttgaatgggatgtcctatggaaacctatttcaccagagttttaaaaataaaaagggtattgttttgtcttctgtacagtgagttccttcccttttcaaagctttctttttatgctgtatgtgactatagatattcatataaaacaagtgcacgtgaagtttgcaaaatgctttaaggccttcctttcaaagcatagtccttttggagccgttttgtaccttttataccttggcttatttgaagttgacacatggggttagttactactctccatgtgcattggggacagtttttataagtgggaaggactcagtattattatatttgagatgataagcattttgtttgggaacaatgcttaaaaatattccagaaagttcagattttttttctttgtgaatgaaatatattctggcccacgaacagggcgatttcctttcagttttttccttttgcaacgtgccttgaagtctcaaagctcacctgaggttgcagacgttacccccaacagaagataggtagaaatgattccagtggcctctttgtattttcttcattgttgagtagatttcaggaaatcaggaggtgtttcacaatacagaatgatggcctttaactgtgaaaaaaaaa

SEQ ID NO: 15 - protein, 394 aa, Homo sapiens
MKRQNVRTLALIVCTFTYLLVGAAVFDALESEPELIERQRLELRQQELRARYNLSQGGYEELERVVLRLKPHKAGVQWRFAGSFYFAITVITTIGYGHAAPSTDGGKVFCMFYALLGIPLTLVMFQSLGERINTLVRYLLHRAKKGLGMRRADVSMANMVLIGFFSCISTLCIGAAAFSHYEHWTFFQAYYYCFITLTTIGFGDYVALQKDQALQTQPQYVAFSFVYILTGLTVIGAFLNLVVLRFMTMNAEDEKRDAEHRALLTRNGQAGGGGGGGSAHTTDTASSTAAAGGGGFRNVYAEVLHFQSMCSCLWYKSREKLQYSIPMIIPRDLSTSDTCVEQSHSSPGGGGRYSDTPSRRCLCSGAPRSAISSVSTGLHSLSTFRGLMKRRSSV

SEQ ID NO: 16 - DNA, 3978 bp, Homo sapiens
  source 1..3978: mol_type = genomic DNA
ggcggcggcggcggcggcggccccgggcgctgagcgggtgcccggcgcggagagcggcgagcgcagccatgccccaggccgcctccggggcagcagcagcggcggccggggccgaggcgcgggccgggggcgccggggggccggcggcggcccgggcgggacgatgaagcggcagaacgtgcgcacgctggcgctcatcgtgtgcaccttcacctacctgctggtgggcgccgcggtcttcgacgcgctggagtcggagcccgagctgatcgagcggcagcggctggagctgcggcagcaggagctgcgggcgcgctacaacctcagccagggcggctacgaggagctggagcgcgtcgtgctgcgcctcaagccgcacaaggccggcgtgcagtggcgcttcgccggctccttctacttcgccatcaccgtcatcaccaccatcggctacgggcacgcggcacccagcacggatggcggcaaggtgttctgcatgttctacgcgctgctgggcatcccgctcacgctcgtcatgttccagagcctgggcgagcgcatcaacaccttggtgaggtacctgctgcaccgcgccaagaaggggctgggcatgcggcgcgccgacgtgtccatggccaacatggtgctcatcggcttcttctcgtgcatcagcacgctgtgcatcggcgccgccgccttctcccactacgagcactggaccttcttccaggcctactactactgcttcatcaccctcaccaccatcggcttcggcgactacgtggcgctgcagaaggaccaggccctgcagacgcagccgcagtacgtggccttcagcttcgtctacatccttacgggcctcacggtcatcggcgccttcctcaacctcgtggtgctgcgcttcatgaccatgaacgccgaggacgagaagcgcgacgccgagcaccgcgcgctgctcacgcgcaacgggcaggcgggcggcggcggagggggtggcagcgcgcacactacggacaccgcctcatccacggcggcagcgggcggcggcggcttccgcaacgtctacgcggaggtgctgcacttccagtccatgtgctcgtgcctgtggtacaagagccgcgagaagctgcagtactccatccccatgatcatcccgcgggacctctccacgtccgacacgtgcgtggagcagagccactcgtcgccgggagggggcggccgctacagcgacacgccctcgcgacgctgcctgtgcagcggggcgccacgctccgccatcagctcggtgtccacgggtctgcacagcctgtccaccttccgcggcctcatgaagcgcaggagctccgtgtgactgccccgaggggcctggagcacctgggggcgcgggcgggggacccctgctgggaggccaggagactgcccctgctgccttctgcccagtgggaccccgcacaacatccctcaccactctcccccagcacccccatctccgactgtgcctgcttgcaccagccggcaggaggccgggctctgaggacccctggggcccccatcggagccctgcaaattccgagaaatgtgaaacttggtggggtcagggaggaaaggcagaagctgggagcctcccttccctttgaaaatctaagaagctcccagtcctcagagaccctgctggtacccagacccccaccttcggaggggacttcatgttccgtgtacgtttgcatctctatttatacctctgtcctgctaggtctcccaccttcccttggttccaaaagccagggtgtctatgtccaagtcacccctactcagccccactccccttcctcatccccagctgtgtctcccaacctcccttcgtgttgttttgcatggctttgcagttatggagaaagtggaaacccagcagtccctaaagctggtccccagaaagcaggacagaaagaaggagggacaggcaggcagcaggaggggcgagctgggaggcaggaggcagcggcctgtcagtctgcagaatggtcgcactggaggttcaagctaactggcctccagccacattctcatagcaggtaggacttcagccttccagacactgcccttagaatctggaacagaagacttcagactcaccataattgctgataattacccactcttaaatttgtcgagtgatttttagcctctgaaaactctatgctggccactgattcctttgagtctcacaaaaccctacttaggtcatcagggcaggagttctcactcccattttacagatgagaatactgaggcctggacaggtgaagtgaccagagagcaaaaggcaaaggggtgggggctgggtgcagtggctcacacctgtattcccaacacttttggaggctgaggttggaggattgcttgagcccaggaatttgagaccagcctaggtgacatagtgagaccccatctctacaaaaaataaaaaattaaccaggtgtggtggcacgtgcctgggagtcccagcgacttgggaggctgaggtgggaggattgtttgagcctgggaggtcgaggctgtagtgagccctgattgcaccactgtactccagcctgggtgacagggcaagaccctgtctcaaaaaaaaaaaaaaaaatggcaaagggagacaagagcccagcctgcttgttgctagccaaagtgttctttccttccagcttggcctgctcttaaaagcaaagctcctgcagtgtacatcctggcattgtgtggctacctgggttttaaaccagaatcagaagtcccggatcagagggcactgctgaggttcagcctcttctcttcttggccaggaggcagcagctctgaatgggcccctgaggctgcacaggggcctttgtcactggggcgcatgcttacaaacagtgcagttcttgggaccgaggtaagcagggctgggtctcatggcagaaaggccaggatctggggctctaggaatttgggaattgggcagagtggccaagaaagctggcaggcatatcctatgggacatcacacctggcaccattgtcattgttggtgcctgtgtcccaagtagctagtgataagctgaggctgcagcaagaaacacccttcccaggtgggggagtttggaccagaggtgccctctgcccaccacacctgcaacccagaagcccagatggaacgcagctgacgaaggtgatgcttgaggctcacttttggggccccacagctggagccggtataatgactgggacaacatcaaggggtggatgaggggcctctcctcccgcaacactgccttcccatgctgttcccctgccagctccttaacactgccgaccaaggccagccctggcattcagggaaattggagggcagcacccgtagggtggccagcctcaggccccaccccagctgtgtcctctagtctctggggacccctggggggaagaagtctaccctgcttgtgagtcccgtctcagtgtggaggaactggctgcacgtgggacctgaaggtgccctctgtgtttatgttgggggtgggggggcagtgctggctgcctctgtcctgtgtgtgaccctgccctcgaagggtcctgtcctgtcagtcccgagggagccacaaccaaagctgcggagagaaggtggggaagggtgcagaatggccgtggggcacagcgtggcagactgttcagtctctgctgggtctttcctagggacctggaaggccagtgttgcttccccctcactccctttcactgcaggcagcctctctgcttccccaatgccttatgcctgggcacactgccacagaatatgcaatatgtgtgggtgaccatgccctcacgaccacacccccaccccgggcagcccccggactccaaaggtcgtggctgccacagcctccctcagctcttcctgcctatctgtcttcacactgagaatggcgcccaataaatgctatccacggagaccagg

SEQ ID NO: 17 - protein, 676 aa, Homo sapiens [sequence omitted]

SEQ ID NO: 18 - DNA, 3262 bp, Homo sapiens
  source 1..3262: mol_type = genomic DNA
gcggcggggctggcagcagtggctgcccgcactgcgcccgggcgctcgccttcgctgcagctcccggtgccgccgctcgggccggccccccggcaggccctcctcgttatggccgcggcctcctccccgcccagggccgagaggaagcgctggggttggggccgcctgccaggcgcccggcggggcagcgcgggcctggccaagaagtgccccttctcgctggagctggcggagggcggcccggcgggcggcgcgctctacgcgcccatcgcgcccggcgccccaggtcccgcgccccctgcgtccccggccgcgcccgccgcgcccccagttgcctccgaccttggcccgcggccgccggtgagcctagacccgcgcgtctccatctacagcacgcgccgcccggtgttggcgcgcacccacgtccagggccgcgtctacaacttcctcgagcgtcccaccggctggaaatgcttcgtttaccacttcgccgtcttcctcatcgtcctggtctgcctcatcttcagcgtgctgtccaccatcgagcagtatgccgccctggccacggggactctcttctggatggagatcgtgctggtggtgttcttcgggacggagtacgtggtccgcctctggtccgccggctgccgcagcaagtacgtgggcctctgggggcggctgcgctttgcccggaagcccatttccatcatcgacctcatcgtggtcgtggcctccatggtggtcctctgcgtgggctccaaggggcaggtgtttgccacgtcggccatcaggggcatccgcttcctgcagatcctgaggatgctacacgtcgaccgccagggaggcacctggaggctcctgggctccgtggtcttcatccaccgccaggagctgataaccaccctgtacatcggcttcctgggcctcatcttctcctcgtactttgtgtacctggctgagaaggacgcggtgaacgagtcaggccgcgtggagttcggcagctacgcagatgcgctgtggtggggggtggtcacagtcaccaccatcggctatggggacaaggtgccccagacgtgggtcgggaagaccatcgcctcctgcttctctgtctttgccatctccttctttgcgctcccagcggggattcttggctcggggtttgccctgaaggtgcagcagaagcagaggcagaagcacttcaaccggcagatcccggcggcagcctcactcattcagaccgcatggaggtgctatgctgccgagaaccccgactcctccacctggaagatctacatccggaaggccccccggagccacactctgctgtcacccagccccaaacccaagaagtctgtggtggtaaagaaaaaaaagttcaagctggacaaagacaatggggtgactcctggagagaagatgctcacagtcccccatatcacgtgcgaccccccagaagagcggcggctggaccacttctctgtcgacggctatgacagttctgtaaggaagagcccaacactgctggaagtgagcatgccccatttcatgagaaccaacagcttcgccgaggacctggacctggaaggggagactctgctgacacccatcacccacatctcacagctgcgggaacaccatcgggccaccattaaggtcattcgacgcatgcagtactttgtggccaagaagaaattccagcaagcgcggaagccttacgatgtgcgggacgtcattgagcagtactcgcagggccacctcaacctcatggtgcgcatcaaggagctgcagaggaggctggaccagtccattgggaagccctcactgttcatctccgtctcagaaaagagcaaggatcgcggcagcaacacgatcggcgcccgcctgaaccgagtagaagacaaggtgacgcagctggaccagaggctggcactcatcaccgacatgcttcaccagctgctctccttgcacggtggcagcacccccggcagcggcggcccccccagagagggcggggcccacatcacccagccctgcggcagtggcggctccgtcgaccctgagctcttcctgcccagcaacaccctgcccacctacgagcagctgaccgtgcccaggaggggccccgatgaggggtcctgaggaggggatggggctgggggatgggcctgagtgagaggggaggccaagagtggccccacctggccctctctgaaggaggccacctcctaaaaggcccagagagaagagccccactctcagaggccccaataccccatggaccatgctgtctggcacagcctgcacttgggggctcagcaaggccacctcttcctggccggtgtgggggccccgtctcaggtctgagttgttaccccaagcgccctggcccccacatggtgatgttgacatcactggcatggtggttgggacccagtggcagggcacagggcctggcccatgtatggccaggaagtagcacaggctgagtgcaggcccaccctgcttggcccagggggcttcctgaggggagacagagcaacccctggaccccagcctcaaatccaggaccctgccaggcacaggcagggcaggaccagcccacgctgactacagggccgccggcaataaaagcccaggagcccatttggagggcctgggcctggctccctcactctcaggaaatgctgacccatgggcaggagactgtggagactgctcctgagcccccagcttccagcaggagggacagtctcaccatttccccagggcacgtggttgagtggggggaacgcccacttccctgggttagactgccagctcttcctagctggagaggagccctgcctctccgcccctgagcccactgtgcgtggggctcccgcctccaacccctcgcccagtcccagcagccagccaaacacacagaaggggactgccacctccccttgccagctgctgagccgcagagaagtgacggttcctacacaggacaggggttccttctgggcattacatcgcatagaaatcaataatttgtggtgatttggatctgtgttttaatgagtttcacagtgtgattttgattattaattgtgcaagcttttcctaataaacgtggagaatcacaggctgggctgggcactgctctcaccttggttcctggggcatccatggggtctctcacagacaggacccctgcagttcccctggaagcagtgcccaggtggctgtggaataggaacgctaaaaaaaaaaaaaaaaa

SEQ ID NO: 19 - protein, 907 aa, Homo sapiens
MDTSRLGVLLSLPVLLQLATGGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRHVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSLISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIRIGVWTIAVLALTCNALVTSTVFRSPLYISPIKLLIGVIAAVNMLTGVSSAVLAGVDAFTFGSFARHGAWWENGVGCHVIGFLSIFASESSVFLLTLAALERGFSVKYSAKFETKAPFSSLKVIILLCALLALTMAAVPLLGGSKYGASPLCLPLPFGEPSTMGYMVALILLNSLCFLMMTIAYTKLYCNLDKGDLENIWDCSMVKHIALLLFTNCILNCPVAFLSFSSLINLTFISPEVIKFILLVVVPLPACLNPLLYILFNPHFKEDLVSLRKQTYVWTRSKHPSLMSINSDDVEKQSCDSTQALVTFTSSSITYDLPPSSVPSPAYPVTESCHLSSVAFVPCL

SEQ ID NO: 20 - DNA, 4625 bp, Homo sapiens
  source 1..4625: mol_type = genomic DNA
aaaaaacgagcgtgcaagcagagatgctgctccacaccgctcaggccgcgagcagcagcaaggcgcaccgccactgtcgccgctgcagccagggctgctccgaaggccggcgtggcggcaaccggcacctctgtccccgccgcgcttctcctcgccgcccacgccgtggggtcaggaacgcggcgtctggcgctgcagacgcccgctgagttgcagaagcccacggagcggcgcccggcgcgccacggcccgtagcagtccggtgctgctctccgcccgcgtccggctcgtggccccctacttcgggcaccatggacacctcccggctcggtgtgctcctgtccttgcctgtgctgctgcagctggcgaccgggggcagctctcccaggtctggtgtgttgctgaggggctgccccacacactgtcattgcgagcccgacggcaggatgttgctcagggtggactgctccgacctggggctctcggagctgccttccaacctcagcgtcttcacctcctacctagacctcagtatgaacaacatcagtcagctgctcccgaatcccctgcccagtctccgcttcctggaggagttacgtcttgcgggaaacgctctgacatacattcccaagggagcattcactggcctttacagtcttaaagttcttatgctgcagaataatcagctaagacacgtacccacagaagctctgcagaatttgcgaagccttcaatccctgcgtctggatgctaaccacatcagctatgtgcccccaagctgtttcagtggcctgcattccctgaggcacctgtggctggatgacaatgcgttaacagaaatccccgtccaggcttttagaagtttatcggcattgcaagccatgaccttggccctgaacaaaatacaccacataccagactatgcctttggaaacctctccagcttggtagttctacatctccataacaatagaatccactccctgggaaagaaatgctttgatgggctccacagcctagagactttagatttaaattacaataaccttgatgaattccccactgcaattaggacactctccaaccttaaagaactaggatttcatagcaacaatatcaggtcgatacctgagaaagcatttgtaggcaacccttctcttattacaatacatttctatgacaatcccatccagtttgttgggagatctgcttttcaacatttacctgaactaagaacactgactctgaatggtgcctcacaaataactgaatttcctgatttaactggaactgcaaacctggagagtctgactttaactggagcacagatctcatctcttcctcaaaccgtctgcaatcagttacctaatctccaagtgctagatctgtcttacaacctattagaagatttacccagtttttcagtctgccaaaagcttcagaaaattgacctaagacataatgaaatctacgaaattaaagttgacactttccagcagttgcttagcctccgatcgctgaatttggcttggaacaaaattgctattattcaccccaatgcattttccactttgccatccctaataaagctggacctatcgtccaacctcctgtcgtcttttcctataactgggttacatggtttaactcacttaaaattaacaggaaatcatgccttacagagcttgatatcatctgaaaactttccagaactcaaggttatagaaatgccttatgcttaccagtgctgtgcatttggagtgtgtgagaatgcctataagatttctaatcaatggaataaaggtgacaacagcagtatggacgaccttcataagaaagatgctggaatgtttcaggctcaagatgaacgtgaccttgaagatttcctgcttgactttgaggaagacctgaaagcccttcattcagtgcagtgttcaccttccccaggccccttcaaaccctgtgaacacctgcttgatggctggctgatcagaattggagtgtggaccatagcagttctggcacttacttgtaatgctttggtgacttcaacagttttcagatcccctctgtacatttcccccattaaactgttaattggggtcatcgcagcagtgaacatgctcacgggagtctccagtgccgtgctggctggtgtggatgcgttcacttttggcagctttgcacgacatggtgcctggtgggagaatggggttggttgccatgtcattggttttttgtccatttttgcttcagaatcatctgttttcctgcttactctggcagccctggagcgtgggttctctgtgaaatattctgcaaaatttgaaacgaaagctccattttctagcctgaaagtaatcattttgctctgtgccctgctggccttgaccatggccgcagttcccctgctgggtggcagcaagtatggcgcctcccctctctgcctgcctttgccttttggggagcccagcaccatgggctacatggtcgctctcatcttgctcaattccctttgcttcctcatgatgaccattgcctacaccaagctctactgcaatttggacaagggagacctggagaatatttgggactgctctatggtaaaacacattgccctgttgctcttcaccaactgcatcctaaactgccctgtggctttcttgtccttctcctctttaataaaccttacatttatcagtcctgaagtaattaagtttatccttctggtggtagtcccacttcctgcatgtctcaatccccttctctacatcttgttcaatcctcactttaaggaggatctggtgagcctgagaaagcaaacctacgtctggacaagatcaaaacacccaagcttgatgtcaattaactctgatgatgtcgaaaaacagtcctgtgactcaactcaagccttggtaacctttaccagctccagcatcacttatgacctgcctcccagttccgtgccatcaccagcttatccagtgactgagagctgccatctttcctctgtggcatttgtcccatgtctctaattaatatgtgaaggaaaatgttttcaaaggttgagaacctgaaaatgtgagattgagtatatcagagcagtaattaataagaagagctgaggtgaaactcggtttaaaaaccaaaaaagaatctctcagttagtaagaaaaggctgaaaacctcttgatacttgagagtgaatataagtctaaatgctgctttgtataatttgttcagctaagggatagatcgatcacactatttaagtgagcccagatcaaaaaagcagattgaaattttctttagaaaagattctccatgatttgaattgcattctctttaaactcaccaatgtaatcattttgggaggagggagaacccacttgctttccaaatgggtttatttaaacccacaaactcaagaggttgttgggggaattaggaaaataagggttttcaatgacctacattgctaggtagaggctgtgatccatgggatttcattctaatgaccatgtgaagatgtttgagtcctcctttgcctttcctcagaaagaatccttctaaggcacaaatcccttagatggataatgtaaggtattgttaactcactcatattgagatcatttttagagataccaggttttatgtatcagcactagatggttccaccctcatgggataaaactgcttacaagtattttgaaagaaaaactgaccaaaattcttaaattgttactaaggcaatcatgcacaggtgacgtatgtcttatctgatttgtttttaactccttggtgcccaaagctcagaagggaattccactgccagcaatgaacatacctggaaaagaaagtaagcaatctgggattttttttctgggttagtaaagaatttttgcaataagttttatcagttgattcaaactgatgtgcatcttaatgatcaaatgtgcacattacataaattaagtccactgatacaacttcttacacatgtatctctagtagctctggcaaacccaatatctgacaccactttggactcaagagactcagtaacgtattatcctgtttatttagcttggttttagctgtgttctctctggataacccacttgatgttaggaacattacttctctgcttattccatattaatactgtgttaggtattttaagaagcaagttattaaataagaaaagtcaaagtattaattcttaccttctattatcctatattagcttcaatacatccaaaccaaatggctgttaggtagatttatttttatataagcatgtttattttgatcagatgttttaacttggatttgaaaaaatacatttatgagatgttttataagatgtgtaaatatagaactgtatttattactatagtaaaggttcagtaacattaaggaccatgataatgataataaaccttgtacagtggcatattctttgatttatattgtgtttctctgcccattttctttaaattcattaactgtatatatgtaaatatatagtacttgtaaatagattccaaatttgcttttctattgggtaaaaaataaatttgtaataaaatgtgtgactatgaaacaaaaaaaaaaaaaaaaaa

SEQ ID NO: 21 - protein, 332 aa, Homo sapiens
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 22 - DNA, 2226 bp, Homo sapiens
  source 1..2226: mol_type = genomic DNA
gtctgccggtcggttgtctggctgcgcgcgccacccgggcctctccagtgccccgcctggctcggcatccacccccagcccgactcacacgtgggttcccgcacgtccgccggccccccccgctgacgtcagcatagctgttccacttaaggcccctcccgcgcccagctcagagtgctgcagccgctgccgccgattccggatctcattgccacgcgcccccgacgaccgcccgacgtgcattcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatctttaaattcatcattcctaatgttgtaaaatacagcccgaactgcaagttgcttattgtttcaaatccagtggatatcttgacctacgtggcttggaagataagtggttttcccaaaaaccgtgttattggaagcggttgcaatctggattcagcccgattccgttacctaatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtgactactttggttaattcattatattaagatataaagtcataaagctgctagttattatattaatttggaaatattaggctattcttgggcaaccctgcaacgattttttctaacagggatattattgactaatagcagaggatgtaatagtcaactgagttgtattggtaccacttccattgtaagtcccaaagtattatatatttgataataatgctaatcataattggaaagtaacattctatatgtaaatgtaaaatttatttgccaactgaatataggcaatgatagtgtgtcactatagggaacacagatttttgagatcttgtcctctggaagctggtaacaattaaaaacaatcttaaggcagggaaaaaaaaaaaaaaaaaa

SEQ ID NO: 23 - protein, 353 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 24 - DNA, 2373 bp, Homo sapiens
  source 1..2373: mol_type = genomic DNA
gcgcggccgggcgcgggccccgggcgatggccgcggagctggcgatgggcgccgagctgcccagcagcccgctggccatcgagtacgtcaacgacttcgacctgatgaagttcgaggtgaagaaggagcctcccgaggccgagcgcttctgccaccgcctgccgccaggctcgctgtcctcgacgccgctcagcacgccctgctcctccgtgccctcctcgcccagcttctgcgcgcccagcccgggcaccggcggcggcggcggcgcggggggcggcggcggctcgtctcaggccgggggcgcccccgggccgccgagcgggggccccggcgccgtcgggggcacctcggggaagccggcgctggaggatctgtactggatgagcggctaccagcatcacctcaaccccgaggcgctcaacctgacgcccgaggacgcggtggaggcgctcatcggcagcggccaccacggcgcgcaccacggcgcgcaccacccggcggccgccgcagcctacgaggctttccgcggcccgggcttcgcgggcggcggcggagcggacgacatgggcgccggccaccaccacggcgcgcaccacgccgcccaccatcaccacgccgcccaccaccaccaccaccaccaccaccaccatggcggcgcgggacacggcggtggcgcgggccaccacgtgcgcctggaggagcgcttctccgacgaccagctggtgtccatgtcggtgcgcgagctgaaccggcagctccgcggcttcagcaaggaggaggtcatccggctcaagcagaagcggcgcacgctcaagaaccgcggctacgcgcagtcctgccgcttcaagcgggtgcagcagcggcacattctggagagcgagaagtgccaactccagagccaggtggagcagctgaagctggaggtggggcgcctggccaaagagcgggacctgtacaaggagaaatacgagaagctggcgggccggggcggccccgggagcgcgggcggggccggtttcccgcgggagccttcgccgccgcaggccggtcccggcggggccaagggcacggccgacttcttcctgtaggcgccggaccccgagcccgcgccgccgtcgccggggacaagttcgcgcaggcctctcggggcctcggctcggactccgcggtacaggacgtggacaccaggcccggcccggccgtgctggccccggtgccaagtctgcgggcgcggggctggaggccccttcgctcccggtccccgttcgcgcgcgtcggcccgggtcgccgtcctgaggttgagcggagaacggtgatttctaaggaaacttgagccaggtctaacttctttccaagcgtccgcttgtacatacgttgaacgtggttctccgttcccaccttcgccctgccagcctagagggaccgcgctgccgtcccttcccgggtggcccctgcctgcccccgccctccttcgttctcttctcagcctccctttccttgccttttttaacttcccctccccgttttaaaatcggtcttattttcgaagtatttataattattatgcttggtgattagaaaagaaaaccttggaggaagccccttctttccccagccggggtccgccctcagtcgcgagtcacagcatgagtcgctcgccaggaggggcccggcccctgcctgccccctccccgcttgcccccgaccctgctaccggcgttccttggaggtcgaagccagggacgtcacccgtgctgtgtccaggcctgctgtcctactatgctcaaccgggggtggggggaggggggtgagtcctgtgctcagtcgggtgggggctggcccggatcccgagctgctgtctctctatgcaccagaacatatctgtaactcctggggaaatacatcttgttttaaccttcaagagaagtgaaagaaaaaagtaatgcacagtatttctagcagaaaatttttttttttaagaggaggcttgggccagagccttctggcatggggcgggtggagaaagtgtttttattttaatttaaattgtgtttcgttttgtttgtggaatctttctttaatgcttcgtcgctctttggactagccgggagagagggcgaggaggcgggtgctccaggccctgtaggctgggccaggcgcctgggggatctgcccgttttcggaggccctcaggggccatcagtgggattccagccgctccacacccctcccctgagcactcggagtggaaggcgcgccgactcgttgaaagttttgttgtgtagttggttttcgttgagttcttttttcatttgctacgaaactgagaaaaagaaaaaaatacacaaaataaatctgttcagatccaagtca

SEQ ID NO: 25 - protein, 1939 aa, Homo sapiens
MTDAQMADFGAAAQYLRKSEKERLEAQTRPFDIRTECFVPDDKEEFVKAKILSREGGKVIAETENGKTVTVKEDQVLQQNPPKFDKIEDMAMLTFLHEPAVLFNLKERYAAWMIYTYSGLFCVTVNPYKWLPVYNAEVVAAYRGKKRSEAPPHIFSISDNAYQYMLTDRENQSILITGESGAGKTVNTKRVIQYFASIAAIGDRGKKDNANANKGTLEDQIIQANPALEAFGNAKTVRNDNSSRFGKFIRIHFGATGKLASADIETYLLEKSRVIFQLKAERNYHIFYQILSNKKPELLDMLLVTNNPYDYAFVSQGEVSVASIDDSEELMATDSAFDVLGFTSEEKAGVYKLTGAIMHYGNMKFKQKQREEQAEPDGTEDADKSAYLMGLNSADLLKGLCHPRVKVGNEYVTKGQSVQQVYYSIGALAKAVYEKMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLYDNHLGKSNNFQKPRNIKGKQEAHFSLIHYAGTVDYNILGWLEKNKDPLNETVVALYQKSSLKLMATLFSSYATADTGDSGKSKGGKKKGSSFQTVSALHRENLNKLMTNLRTTHPHFVRCIIPNERKAPGVMDNPLVMHQLRCNGVLEGIRICRKGFPNRILYGDFRQRYRILNPVAIPEGQFIDSRKGTEKLLSSLDIDHNQYKFGHTKVFFKAGLLGLLEEMRDERLSRIITRMQAQARGQLMRIEFKKIVERRDALLVIQWNIRAFMGVKNWPWMKLYFKIKPLLKSAETEKEMATMKEEFGRIKETLEKSEARRKELEEKMVSLLQEKNDLQLQVQAEQDNLNDAEERCDQLIKNKIQLEAKVKEMNERLEDEEEMNAELTAKKRKLEDECSELKKDIDDLELTLAKVEKEKHATENKVKNLTEEMAGLDEIIAKLTKEKKALQEAHQQALDDLQVEEDKVNSLSKSKVKLEQQVDDLEGSLEQEKKVRMDLERAKRKLEGDLKLTQESIMDLENDKLQLEEKLKKKEFDINQQNSKIEDEQVLALQLQKKLKENQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKVSRTLEDQANEYRVKLEEAQRSLNDFTTQRAKLQTENGELARQLEEKEALISQLTRGKLSYTQQMEDLKRQLEEEGKAKNALAHALQSARHDCDLLREQYEEETEAKAELQRVLSKANSEVAQWRTKYETDAIQRTEELEEAKKKLAQRLQDAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGEGGKNVHELEKVRKQLEVEKLELQSALEEAEASLEHEEGKILRAQLEFNQIKAEIERKLAEKDEEMEQAKRNHQRVVDSLQTSLDAETRSRNEVLRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMESDLTQLQSEVEEAVQECRNAEEKAKKAITDAAMMAEELKKEQDTSAHLERMKKNMEQTIKDLQHRLDEAEQIALKGGKKQLQKLEARVRELEGELEAEQKRNAESVKGMRKSERRIKELTYQTEEDKKNLLRLQDLVDKLQLKVKAYKRQAEEAEEQANTNLSKFRKVQHELDEAEERADIAESQVNKLRAKSRDIGAKQKMHDEE

SEQ ID NO: 26 - DNA, 5941 bp, Homo sapiens
  source 1..5941: mol_type = genomic DNA
agatagagagactcctgcggcccagattcttcaggattctccgtgaagggataaccaggggaagcaccaagatgaccgatgcccagatggctgactttggggcagcggcccagtacctccgcaagtcagagaaggagcgtctagaggcccagacccggccctttgacattcgcactgagtgcttcgtgcccgatgacaaggaagagtttgtcaaagccaagattttgtcccgggagggaggcaaggtcattgctgaaaccgagaatgggaagacggtgactgtgaaggaggaccaggtgttgcagcagaacccacccaagttcgacaagattgaggacatggccatgctgaccttcctgcacgagcccgcggtgcttttcaacctcaaggagcgctacgcggcctggatgatatatacctactcgggcctcttctgtgtcactgtcaacccctacaagtggctgccggtgtacaatgccgaggtggtggccgcctaccggggcaagaagaggagtgaggccccgccccacatcttctccatctccgacaacgcctatcagtacatgctgacagatcgggagaaccagtccatcctcatcacgggagaatccggggcggggaagactgtgaacaccaagcgtgtcatccagtactttgccagcattgcagccataggtgaccgtggcaagaaggacaatgccaatgcgaacaagggcaccctggaggaccagatcatccaggccaaccccgctctggaggccttcggcaatgccaagactgtccggaacgacaactcctcccgctttgggaaattcattaggatccactttggggccactggaaagctggcttctgcagacatagagacctacctgctggagaagtcccgggtgatcttccagctgaaagctgagagaaactaccacatcttctaccagattctgtccaacaagaagccggagttgctggacatgctgctggtcaccaacaatccctacgactacgccttcgtgtctcagggagaggtgtccgtggcctccattgatgactccgaggagctcatggccaccgatagtgcctttgacgtgctgggcttcacttcagaggagaaagctggcgtctacaagctgacgggagccatcatgcactacgggaacatgaagttcaagcagaagcagcgggaggagcaggcggagccagacggcaccgaagatgctgacaagtcggcctacctcatggggctgaactcagctgacctgctcaaggggctgtgccaccctcgggtgaaagtgggcaacgagtatgtcaccaaggggcagagcgtgcagcaggtgtactactccatcggggctctggccaaggcagtgtatgagaagatgttcaactggatggtgacgcgcatcaacgccaccctggagaccaagcagccacgccagtacttcataggagtcctggacatcgctggcttcgagatcttcgacttcaacagctttgagcagctctgcatcaacttcaccaacgagaagctgcagcagttcttcaaccaccacatgttcgtgctggagcaggaggagtacaagaaggagggcattgagtggacattcattgactttggcatggacctgcaggcctgcattgacctcatcgagaagcccatgggcatcatgtccatcctggaggaggagtgcatgttccccaaggccactgacatgaccttcaaggccaagctgtacgacaaccacctgggcaagtccaacaatttccagaagccacgcaacatcaaggggaagcaggaagcccacttctccctgatccactacgccggcactgtggactacaacatcctgggctggctggaaaaaaacaaggatcctctcaacgagactgttgtggccctgtaccagaagtcctccctcaagctcatggccactctcttctcctcctacgcaactgccgatactggggacagtggtaaaagcaaaggaggcaagaaaaagggctcatccttccagacggtgtcggctctccaccgggaaaatctcaacaagctaatgaccaacctgaggaccacccatcctcactttgtgcgttgcatcatccccaatgagcggaaggctccaggggtgatggacaaccccctggtcatgcaccagctgcgctgcaatggcgtgctggagggcatccgcatctgcaggaagggcttccccaaccgcatcctctacggggacttccggcagaggtatcgcatcctgaacccagtggccatccctgagggacagttcattgatagcaggaaggggacagagaagctgctcagctctctggacattgatcacaaccagtacaagtttggccacaccaaggtgttcttcaaggcagggctgcttgggctgctggaggagatgcgggatgagaggctgagccgcatcatcacgcgcatgcaggcccaagcccggggccagctcatgcgcattgagttcaagaagatagtggaacgcagggatgccctgctggtaatccagtggaacattcgggccttcatgggggtcaagaattggccctggatgaagctctacttcaagatcaagccgctgctgaagagcgcagagacggagaaggagatggccaccatgaaggaagagttcgggcgcatcaaagagacgctggagaagtccgaggctcgccgcaaggagctggaggagaagatggtgtccctgctgcaggagaagaatgacctgcagctccaagtgcaggcggaacaagacaacctcaatgatgctgaggagcgctgcgaccagctgatcaaaaacaagattcagctggaggccaaagtaaaggagatgaatgagaggctggaggatgaggaggagatgaacgcggagctcactgccaagaagcgcaagctggaagacgagtgctcagagctcaagaaggacattgatgacctggagctgacactggccaaggtggagaaggagaagcatgcaacagagaacaaggtgaagaacctaacagaggagatggctgggctggatgaaatcatcgctaagctgaccaaggagaagaaagctctacaagaggcccatcagcaggccctggatgaccttcaggttgaggaagacaaggtcaacagcctgtccaagtctaaggtcaagctggagcagcaggtggatgatctggagggatccctagagcaagagaagaaggtgcgcatggacctggagcgagcaaagcggaaactggagggcgacctgaagctgacccaggagagcatcatggacctggaaaatgataaactgcagctggaagaaaagcttaagaagaaggagtttgacattaatcagcagaacagtaagattgaggatgagcaggtgctggcccttcaactacagaagaaactgaaggaaaaccaggcacgcatcgaggagctggaggaggagctggaggccgagcgcaccgccagggctaaggtggagaagctgcgctcagacctgtctcgggagctggaggagatcagcgagcggctggaagaggccggcggggccacgtccgtgcagatcgagatgaacaagaagcgcgaggccgagttccagaagatgcggcgggacctggaggaggccacgctgcagcacgaggccactgccgcggccctgcgcaagaagcacgccgacagcgtggccgagctgggcgagcagatcgacaacctgcagcgggtgaagcagaagctggagaaggagaagagcgagttcaagctggagctggatgacgtcacctccaacatggagcagatcatcaaggccaaggcaaacctggagaaagtgtctcggacgctggaggaccaggccaatgagtaccgcgtgaagctagaagaggcccaacgctccctcaatgatttcaccacccagcgagccaagctgcagaccgagaatggagagttggcccggcagctagaggaaaaggaggcgctaatctcgcagctgacccgggggaagctctcttatacccagcaaatggaggacctcaaaaggcagctggaggaggagggcaaggcgaagaacgccctggcccatgcactgcagtcggcccggcatgactgcgacctgctgcgggagcagtacgaggaggagacagaggccaaggccgagctgcagcgcgtcctgtccaaggccaactcggaggtggcccagtggaggaccaagtatgagacggacgccattcagcggactgaggagctcgaagaggccaaaaagaagctggcccagcggctgcaggatgccgaggaggccgtggaggctgttaatgccaagtgctcctcactggagaagaccaagcaccggctacagaatgagatagaggacttgatggtggacgtagagcgctccaatgctgctgctgcagccctggacaagaagcagagaaactttgacaagatcctggccgagtggaagcagaagtatgaggagtcgcagtctgagctggagtcctcacagaaggaggctcgctccctcagcacagagctcttcaagctcaagaacgcctacgaggagtccctggagcacctagagaccttcaagcgggagaacaagaaccttcaggaggaaatctcggaccttactgagcagctaggagaaggaggaaagaatgtgcatgagctggagaaggtccgcaaacagctggaggtggagaagctggagctgcagtcagccctggaggaggcagaggcctccctggagcacgaggagggcaagatcctccgggcccagctagagttcaaccagatcaaggcagagatcgagcggaagctggcagagaaggacgaggagatggaacaggccaagcgcaaccaccagcgggtggtggactcgctgcagacctccctggatgcagagacacgcagccgcaacgaggtcctgagggtgaagaagaagatggaaggagacctcaatgagatggagatccagctcagccacgccaaccgcatggctgccgaggcccagaagcaagtcaagagcctccagagcttgctgaaggacacccagatccagctggacgatgcggtccgtgccaacgacgacctgaaggagaacatcgccatcgtggagcggcgcaacaacctgctgcaggctgagctggaggagctgcgtgccgtggtggagcagacagagcggtcccggaagctggcggagcaggagctgattgagaccagcgagcgggtgcagctgctgcattcccagaacaccagcctcatcaaccagaagaagaagatggagtcggatctgacccagctccagtcggaagtggaggaggcagtgcaggagtgcagaaacgccgaggagaaggccaagaaggccatcacggatgccgccatgatggcagaggagctgaagaaggagcaggacaccagcgcccacctggagcgcatgaagaagaacatggagcagaccattaaggacctgcagcaccggctggacgaggccgagcagatcgccctcaagggaggcaagaagcagctgcagaagctggaagcgcgggtgcgggagctggagggtgagctggaggccgagcagaagcgcaacgcagagtcggtgaagggcatgaggaagagcgagcggcgcatcaaggagctcacctaccagacagaggaagacaaaaagaacctgctgcggctacaggacctggtggacaagctgcaactgaaggtcaaggcctacaagcgccaggccgaggaggcggaggagcaagccaacaccaacctgtccaagttccgcaaggtgcagcatgagctggatgaggcagaggagcgggcggacatcgctgagtcccaggtcaacaagcttcgagccaagagccgtgacattggtgccaagcaaaaaatgcacgatgaggagtgacactgcctcgggaacctcactcttgccaacctgtaataaatatgagtgcc

SEQ ID NO: 27 - protein, 175 aa, Homo sapiens
MASRKAGTRGKVAATKQAQRGSSNVFSMFEQAQIQEFKEAFSCIDQNRDGIICKADLRETYSQLGKVSVPEEELDAMLQEGKGPINFTVFLTLFGEKLNGTDPEEAILSAFRMFDPSGKGVVNKDEFKQLLLTQADKFSPAEVEQMFALTPMDLAGNIDYKSLCYIITHGDEKEE

SEQ ID NO: 28 - DNA, 619 bp, Homo sapiens
  source 1..619: mol_type = genomic DNA
tctgcagagagaatggccagcaggaaggcggggacccggggcaaggtggcagccaccaagcaggcccaacgtggttcttccaacgtcttttccatgtttgaacaagcccagatacaggagttcaaagaagccttcagctgtatcgaccagaatcgtgatggcatcatctgcaaggcagacctgagggagacctactcccagctggggaaggtgagtgtcccagaggaggagctggacgccatgctgcaagagggcaagggccccatcaacttcaccgtcttcctcacgctctttggggagaagctcaatgggacagaccccgaggaagccatcctgagtgccttccgcatgtttgaccccagcggcaaaggggtggtgaacaaggatgagttcaagcagcttctcctgacccaggcagacaagttctctccagctgaggtggagcagatgttcgccctgacacccatggacctggcggggaacatcgactacaagtcactgtgctacatcatcacccatggagacgagaaagaggaatgaggggcagggccaggcccacgggggggcacctcaataaactctgttgcaaaattggaaaaaaaaaaaaaaaaaaaaaaaa

SEQ ID NO: 29 - protein, 5179 aa, Homo sapiens
MGLPLARLAAVCLALSLAGGSELQTEGRTRNHGHNVCSTWGNFHYKTFDGDVFRFPGPCDYNFASDCRGSYKEFAVHLKRGPGQAEAPAGVESILLTIKDDTIYLTRHLAVLNGAVVSTPHYSPGLLIEKSDAYTKVYSRAGLTLMWNREDALMLELDTKFRNHTCGLCGDYNGLQSYSEFLSDGVLFSPLEFGNMQKINQPDVVCEDPEEEVAPASCSEHRAECERLLTAEAFADCQDLVPLEPYLRACQQDRCRCPGGDTCVCSTVAEFSRQCSHAGGRPGNWRTATLCPKTCPGNLVYLESGSPCMDTCSHLEVSSLCEEHRMDGCFCPEGTVYDDIGDSGCVPVSQCHCRLHGHLYTPGQEITNDCEQCVCNAGRWVCKDLPCPGTCALEGGSHITTFDGKTYTFHGDCYYVLAKGDHNDSYALLGELAPCGSTDKQTCLKTVVLLADKKKNVVVFKSDGSVLLNELQVNLPHVTASFSVFRPSSYHIMVSMAIGVRLQVQLAPVMQLFVTLDQASQGQVQGLCGNFNGLEGDDFKTASGLVEATGAGFANTWKAQSTCHDKLDWLDDPCSLNIESANYAEHWCSLLKKTETPFGRCHSAVDPAEYYKRCKYDTCNCQNNEDCLCAALSSYARACTAKGVMLWGWREHVCNKDVGSCPNSQVFLYNLTTCQQTCRSLSEADSHCLEGFAPVDGCGCPDHTFLDEKGRCVPLAKCSCYHRGLYLEAGDVVVRQEERCVCRDGRLHCRQIRLIGQSCTAPKIHMDCSNLTALATSKPRALSCQTLAAGYYHTECVSGCVCPDGLMDDGRGGCVVEKECPCVHNNDLYSSGAKIKVDCNTCTCKRGRWVCTQAVCHGTCSIYGSGHYITFDGKYYDFDGHCSYVAVQDYCGQNSSLGSFSIITENVPCGTTGVTCSKAIKIFMGRTELKLEDKHRVVIQRDEGHHVAYTTREVGQYLVVESSTGIIVIWDKRTTVFIKLAPSYKGTVCGLCGNFDHRSNNDFTTRDHMVVSSELDFGNSWKEAPTCPDVSTNPEPCSLNPHRRSWAEKQCSILKSSVFSICHSKVDPKPFYEACVHDSCSCDTGGDCECFCSAVASYAQECTKEGACVFWRTPDLCPIFCDYYNPPHECEWHYEPCGNRSFETCRTINGIHSNISVSYLEGCYPRCPKDRPIYEEDLKKCVTADKCGCYVEDTHYPPGASVPTEETCKSCVCTNSSQVVCRPEEGKILNQTQDGAFCYWEICGPNGTVEKHFNICSITTRPSTLTTFTTITLPTTPTTFTTTTTTTTPTSSTVLSTTPKLCCLWSDWINEDHPSSGSDDGDRETFDGVCGAPEDIECRSVKDPHLSLEQLGQKVQCDVSVGFICKNEDQFGNGPFGLCYDYKIRVNCCWPMDKCITTPSPPTTTPSPPPTSTTTLPPTTTPSPPTTTTTTPPPTTTPSPPITTTTTPPPTTTPSPPISTTTTPPPTTTPSPPTTTPSPPTTTPSPPTTTTTTPPPTTTPSPPTTTPITPPASTTTLPPTTTPSPPTTTTTTPPPTTTPSPPTTTPITPPTSTTTLPPTTTPSPPPTTTTTPPPTTTPSPPTTTTPSPPTITTTTPPPTTTPSPPTTTTTTPPPTTTPSPPTTTPITPPTSTTTLPPTTTPSPPPTTTTTPPPTTTPSPPTTTTPSPPITTTTTPPPTTTPSSPITTTPSPPTTTMTTPSPTTTPSSPITTTTTPSSTTTPSPPPTTMTTPSPTTTPSPPTTTMTTLPPTTTSSPLTTTPLPPSITPPTFSPFSTTTPTTPCVPLCNWTGWLDSGKPNFHKPGGDTELIGDVCGPGWAANISCRATMYPDVPIGQLGQTVVCDVSVGLICKNEDQKPGGVIPMAFCLNYEINVQCCECVTQPTTMTTTTTENPTPPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTGPPTHTSTAPIAELTTSNPPPESSTPQTSRSTSSPLTESTTLLSTLPPAIEMTSTAPPSTPTAPTTTSGGHTLSPPPSTTTSPPGTPTRGTTTGSSSAPTPSTVQTTTTSAWTPTPTPLSTPSIIRTTGLRPYPSSVLICCVLNDTYYAPGEEVYNGTYGDTCYFVNCSLSCTLEFYNWSCPSTPSPTPTPSKSTPTPSKPSSTPSKPTPGTKPPECPDFDPPRQENETWWLCDCFMATCKYNNTVEIVKVECEPPPMPTCSNGLQPVRVEDPDGCCWHWECDCYCTGWGDPHYVTFDGLYYSYQGNCTYVLVEEISPSVDNFGVYIDNYHCDPNDKVSCPRTLIVRHETQEVLIKTVHMMPMQVQVQVNRQAVALPYKKYGLEVYQSGINYVVDIPELGVLVSYNGLSFSVRLPYHRFGNNTKGQCGTCTNTTSDDCILPSGEIVSNCEAAADQWLVNDPSKPHCPHSSSTTKRPAVTVPGGGKTTPHKDCTPSPLCQLIKDSLFAQCHALVPPQHYYDACVFDSCFMPGSSLECASLQAYAALCAQQNICLDWRNHTHGACLVECPSHREYQACGPAEEPTCKSSSSQQNNTVLVEGCFCPEGTMNYAPGFDVCVKTCGCVGPDNVPREFGEHFEFDCKNCVCLEGGSGIICQPKRCSQKPVTHCVEDGTYLATEVNPADTCCNITVCKCNTSLCKEKPSVCPLGFEVKSKMVPGRCCPFYWCESKGVCVHGNAEYQPGSPVYSSKCQDCVCTDKVDNNTLLNVIACTHVPCNTSCSPGFELMEAPGECCKKCEQTHCIIKRPDNQHVILKPGDFKSDPKNNCTFFSCVKIHNQLISSVSNITCPNFDASICIPGSITFMPNGCCKTCTPRNETRVPCSTVPVTTEVSYAGCTKTVLMNHCSGSCGTFVMYSAKAQALDHSCSCCKEEKTSQREVVLSCPNGGSLTHTYTHIESCQCQDTVCGLPTGTSRRARRSPRHLGSG

SEQ ID NO: 30 - DNA, 15720 bp, Homo sapiens
  source 1..15720: mol_type = genomic DNA
caacccacaccgcccctgccagccaccatggggctgccactagcccgcctggcggctgtgtgcctggccctgtctttggcagggggctcggagctccagacagagggcagaacccgaaaccacggccacaacgtctgcagcacctggggcaacttccactacaagaccttcgacggggacgtcttccgcttccccggcccctgcgactacaacttcgcctccgactgccgaggctcctacaaggaatttgctgtgcacctgaagcggggtccgggccaggctgaggcccccgccggggtggagtccatcctgctgaccatcaaggatgacaccatctacctcacccgccacctggctgtgcttaacggggccgtggtcagcaccccgcactacagccccgggctgctcattgagaagagcgatgcctacaccaaagtctactcccgcgccggcctcaccctcatgtggaaccgggaggatgcactcatgctggagctggacactaagttccggaaccacacctgtggcctctgcggggactacaacggcctgcagagctattcagaattcctctctgacggcgtgctcttcagtcccctggagtttgggaacatgcagaagatcaaccagcccgatgtggtgtgtgaggatcccgaggaggaggtggcccccgcatcctgctccgagcaccgcgccgagtgtgagaggctgctgaccgccgaggccttcgcggactgtcaggacctggtgccgctggagccgtatctgcgcgcctgccagcaggaccgctgccggtgcccgggcggtgacacctgcgtctgcagcaccgtggccgagttctcccgccagtgctcccacgccggcggccggcccgggaactggaggaccgccacgctctgccccaagacctgccccgggaacctggtgtacctggagagcggctcgccctgcatggacacctgctcacacctggaggtgagcagcctgtgcgaggagcaccgcatggacggctgtttctgcccagaaggcaccgtatatgacgacatcggggacagtggctgcgttcctgtgagccagtgccactgcaggctgcacggacacctgtacacaccgggccaggagatcaccaatgactgcgagcagtgtgtctgtaacgctggccgctgggtgtgcaaagacctgccctgccccggcacctgtgccctggaaggcggctcccacatcaccaccttcgatgggaagacgtacaccttccacggggactgctactatgtcctggccaagggtgaccacaacgattcctacgctctcctgggcgagctggccccctgtggctccacagacaagcagacctgcctgaagacggtggtgctgctggctgacaagaagaagaatgtggtggtcttcaagtccgatggcagtgtactgctcaacgagctgcaggtgaacctgccccacgtgaccgcgagcttctctgtcttccgcccgtcttcctaccacatcatggtgagcatggccattggcgtccggctgcaggtgcagctggccccagtcatgcaactctttgtgacactggaccaggcctcccaggggcaggtgcagggcctctgcgggaacttcaacggcctggaaggtgacgacttcaagacggccagcgggctggtggaggccacgggggccggctttgccaacacctggaaggcacagtcaacctgccatgacaagctggactggttggacgatccctgctccctgaacatcgagagcgccaactacgccgagcactggtgctccctcctgaagaagacagagaccccctttggcaggtgccactcggctgtggaccctgctgagtattacaagaggtgcaaatatgacacgtgtaactgtcagaacaatgaggactgcctgtgcgccgccctgtcctcctacgcgcgcgcctgcaccgccaagggcgtcatgctgtggggctggcgggagcatgtctgcaacaaggatgtgggctcctgccccaactcgcaggtcttcctgtacaacctgaccacctgccagcagacctgccgctccctctccgaggccgacagccactgtctcgagggctttgcgcctgtggacggctgcggctgccctgaccacaccttcctggacgagaagggccgctgcgtacccctggccaagtgctcctgttaccaccgcggtctctacctggaggcgggggacgtggtcgtcaggcaggaagaacgatgtgtgtgccgggatgggcggctgcactgtaggcagatccggctgatcggccagagctgcacggccccaaagatccacatggactgcagcaacctgactgcactggccacctcgaagccccgagccctcagctgccagacgctggccgccggctattaccacacagagtgtgtcagtggctgtgtgtgccccgacgggctgatggatgacggccggggtggctgcgtggtggagaaggaatgcccttgcgtccataacaacgacctgtattcttccggcgccaagatcaaggtggactgcaatacctgcacctgcaagagaggacgctgggtgtgcacccaggctgtgtgccatggcacctgctccatttacgggagtggccactacatcacctttgacgggaagtactacgactttgacggacactgctcctacgtggctgttcaggactactgcggccagaactcctcactgggctcattcagcatcatcaccgagaacgtcccctgtggcactacgggcgtcacctgctccaaggccatcaagatcttcatggggaggacggagctgaagttggaagacaagcaccgtgtggtgatccagcgtgatgagggtcaccacgtggcctacaccacgcgggaggtgggccagtacctggtggtggagtccagcacgggcatcatcgtcatctgggacaagaggaccaccgtgttcatcaagctggctccctcctacaagggcaccgtgtgtggcctgtgtgggaactttgaccaccgctccaacaacgacttcaccacgcgggaccacatggtggtgagcagcgagctggacttcgggaacagctggaaggaggcccccacctgcccagatgtgagcaccaaccccgagccctgcagcctgaacccgcaccgccgctcctgggccgagaagcagtgcagcatcctcaaaagcagcgtgttcagcatctgccacagcaaggtggaccccaagcccttctacgaggcctgtgtgcacgactcgtgctcctgtgacacgggtggggactgtgagtgcttctgctctgccgtggcctcctacgcccaggagtgtaccaaagagggggcctgcgtgttctggaggacgccggacctgtgccccatattctgcgactactacaaccctccgcatgagtgtgagtggcactatgagccatgtgggaaccggagcttcgagacctgcaggaccatcaatggcatccactccaacatctccgtgtcctacctggagggctgctacccccggtgccccaaggacaggcccatctatgaggaggatctgaagaagtgtgtcactgcagacaagtgtggctgctatgtcgaggacacccactacccacctggagcatcggttcccaccgaggagacctgcaagtcctgcgtgtgtaccaactcctcccaagtcgtctgcaggccggaggaaggaaagattcttaaccagacccaggatggcgccttctgctactgggagatctgtggccccaacgggacggtggagaagcacttcaacatctgttccattacgacacgcccgtccaccctgaccaccttcaccaccatcaccctccccaccacccccaccaccttcaccactaccaccaccaccaccaccccgacctccagcacagttttatcaacaactccgaagctgtgctgcctctggtctgactggatcaatgaggaccaccccagcagtggcagcgacgacggtgaccgagaaacatttgatggggtctgcggggcccctgaggacatcgagtgcaggtcggtcaaggatccccacctcagcttggagcagctaggccagaaggtgcagtgtgatgtctctgttgggttcatttgcaagaatgaagaccagtttggaaatggaccatttggactgtgttacgactacaagatacgtgtcaattgttgctggcccatggataagtgtatcaccactcccagccctccaactaccactcccagccctccaccaaccagcacgaccacccttccaccaaccaccacccccagccctccaaccaccaccacaaccacccctccaccaaccaccacccccagccctccaataaccaccacgaccacccctccaccaaccaccactcccagccctccaataagcaccacaaccacccctccaccaaccaccactcccagccctccaaccaccactcccagccctccaaccaccactcccagccctccaacaaccaccacaaccacccctccaccaaccaccactcccagccctccaacgactacgcccatcactccaccagccagcactaccacccttccaccaaccaccactcccagccctccaacaaccaccacaaccacccctccaccaaccaccactcccagtcctccaacgactacgcccatcactccaccaaccagcactactacccttccaccaaccaccactcccagccctccaccaaccaccacaaccacccctccaccaaccaccactcccagccctccaacaaccaccactcccagtcctccaacaatcaccacaaccacccctccaccaaccaccactcccagccctccaacaacgaccacaaccacccctccaccaaccaccactcccagccctccaacgactacacccatcactccaccaaccagcactaccacccttccaccaaccaccactcccagccctccaccaaccaccacaaccacccctccaccaaccaccactcccagccctccaacaaccaccactcccagccctccaataaccaccacaaccacccctccaccaaccaccactcccagctctccaataaccaccactcccagccctccaacaaccaccatgaccaccccttcaccaaccaccacccccagctctccaataaccaccacaaccaccccttcctcaactaccactcccagccctccaccaaccaccatgaccaccccttcaccaaccaccactcccagccctccaacaaccaccatgaccacccttccaccaaccaccacttccagccctctaacaactactcctctacctccatcaataactcctcctacattttcaccattctcaacgacaacccctactaccccatgcgtgcctctctgcaattggactggctggctggattctggaaaacccaactttcacaaaccaggtggagacacagaattgattggagacgtctgtggaccaggctgggcagctaacatctcttgcagagccaccatgtatcctgatgttcccattggacagcttggacaaacagtggtgtgtgatgtctctgtggggctgatatgcaaaaatgaagaccaaaagccaggtggggtcatccctatggccttctgcctcaactacgagatcaacgttcagtgctgtgagtgtgtcacccaacccaccaccatgacaaccaccaccacagagaacccaactccgccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccgggccccccacccacacaagcacagcaccgattgctgagttgaccacatccaatcctccgcctgagtcctcaacccctcagacctctcggtccacctcttcccctctcacggagtcaaccacccttctgagtaccctaccacctgccattgagatgaccagcacggccccaccctccacacccacggcacccacgaccacgagcggaggccacacactgtctccaccgcccagcaccaccacgtcccctccaggcacccccactcgcggtaccacgactgggtcatcttcagcccccacccccagcactgtgcagacgaccaccaccagtgcctggacccccacgccgaccccactctccacacccagcatcatcaggaccacaggcctgaggccctacccttcctctgtgcttatctgctgtgtcctgaacgacacctactacgcaccaggtgaggaggtgtacaacggcacatacggagacacctgttatttcgtcaactgctcactgagctgtacgttggagttctataactggtcctgcccatccacgccctccccaacacccacgccctccaagtcgacgcccacgccttccaagccatcgtccacgccctccaagccgacgcccggcaccaagccccccgagtgcccagactttgatcctcccagacaggagaacgagacttggtggctgtgcgactgcttcatggccacgtgcaagtacaacaacacggtggagatcgtgaaggtggagtgtgagccgccgcccatgcccacctgctccaacggcctccaacccgtgcgcgtcgaggaccccgacggctgctgctggcactgggagtgcgactgctactgcacgggctggggcgacccgcactatgtcaccttcgacggactctactacagctaccagggcaactgcacctacgtgctggtggaggagatcagcccctccgtggacaacttcggagtttacatcgacaactaccactgcgatcccaacgacaaggtgtcctgcccccgcaccctcatcgtgcgccacgagacccaggaggtgctgatcaagaccgtgcatatgatgcccatgcaggtgcaggtgcaggtgaacaggcaggcggtggcactgccctacaagaagtacgggctggaggtgtaccagtctggcatcaactacgtggtggacatccccgagctgggtgtcctcgtctcctacaatggcctgtccttctccgtcaggctgccctaccaccggtttggcaacaacaccaagggccagtgtggcacctgcaccaacaccacctccgacgactgcattctgcccagcggggagatcgtctccaactgtgaggctgcggctgaccagtggctggtgaacgacccctccaagccacactgcccccacagcagctccacgaccaagcgcccggccgtcactgtgcccgggggcggtaaaacgaccccacacaaggactgcaccccatctcccctctgccagctcatcaaggacagcctgtttgcccagtgccacgcactggtgcccccgcagcactactacgatgcctgcgtgttcgacagctgcttcatgccgggctcgagcctggagtgcgccagtctgcaggcctacgcagccctctgtgcccagcagaacatctgcctcgactggcggaaccacacgcatggggcctgcttggtggagtgcccatctcacagggagtaccaggcctgtggccctgcagaagagcccacgtgcaaatccagctcctcccagcagaacaacacagtcctggtggaaggctgcttctgtcctgagggcaccatgaactacgctcctggctttgatgtctgcgtgaagacctgcggctgtgtgggacctgacaatgtgcccagagagtttggggagcacttcgagttcgactgcaagaactgtgtctgcctggagggtggaagtggcatcatctgccaacccaagaggtgcagccagaagcccgttacccactgcgtggaagacggcacctacctcgccacggaggtcaaccctgccgacacctgctgcaacattaccgtctgcaagtgcaacaccagcctgtgcaaagagaagccctccgtgtgcccgctgggattcgaagtgaagagcaagatggtgcctggaaggtgctgtcctttctactggtgtgagtccaagggggtgtgtgttcacgggaatgctgagtaccagcccggttctccagtttattcctccaagtgccaggactgcgtgtgcacggacaaggtggacaacaacaccctgctcaacgtcatcgcctgcacccacgtgccctgcaacacctcctgcagccctggcttcgaactcatggaggcccccggggagtgctgtaagaagtgtgaacagacgcactgtatcatcaaacggcccgacaaccagcacgtcatcctgaagcccggggacttcaagagcgacccgaagaacaactgcacattcttcagctgcgtgaagatccacaaccagctcatctcgtccgtctccaacatcacctgccccaactttgatgccagcatttgcatcccgggctccatcacattcatgcccaatggatgctgcaagacctgcacccctcgcaatgagaccagggtgccctgctccaccgtccccgtcaccacggaggtttcgtacgccggctgcaccaagaccgtcctcatgaatcattgctccgggtcctgcgggacatttgtcatgtactcggccaaggcccaggccctggaccacagctgctcctgctgcaaagaggagaaaaccagccagcgtgaggtggtcctgagctgccccaatggcggctcgctgacacacacctacacccacatcgagagctgccagtgccaggacaccgtctgcgggctccccaccggcacctcccgccgggcccggcgctcccctaggcatctggggagcgggtgagcggggtgggcacagcccccttcactgccctcgacagctttacctcccccggaccctctgagcctcctaagctcggcttcctctcttcagatatttattgtctgagtctttgttcagtccttgctttccaataataaactcagggggacatgc

SEQ ID NO: 31 - protein, 324 aa, Homo sapiens
MFPSPALTPTPFSVKDILNLEQQQRSLAAAGELSARLEATLAPSSCMLAAFKPEAYAGPEAAAPGLPELRAELGRAPSPAKCASAFPAAPAFYPRAYSDPDPAKDPRAEKKELCALQKAVELEKTEADNAERPRARRRRKPRVLFSQAQVYELERRFKQQRYLSAPERDQLASVLKLTSTQVKIWFQNRRYKCKRQRQDQTLELVGLPPPPPPPARRIAVPVLVRDGKPCLGDSAPYAPAYGVGLNPYGYNAYPAYPGYGGAACSPGYSCTAAYPAGPSPAQPATAAANNNFVNFGVGDLNAVQSPGIPQSNSGVSTLHGIRAW

SEQ ID NO: 32 - DNA, 1669 bp, Homo sapiens
  source 1..1669: mol_type = genomic DNA
gctcctgtcatcgaggcccctggcccaatggcaggctgagtccccctcctctggcctggtcccgcctctcctgccccttgtgctcagcgctacctgctgcccggacacatccagagctggccgacgggtgcgcgggcgggcggcggcaccatgcagggaagctgccaggggccgtgggcagcgccgctttctgccgcccacctggcgctgtgagactggcgctgccaccatgttccccagccctgctctcacgcccacgcccttctcagtcaaagacatcctaaacctggaacagcagcagcgcagcctggctgccgccggagagctctctgcccgcctggaggcgaccctggcgccctcctcctgcatgctggccgccttcaagccagaggcctacgctgggcccgaggcggctgcgccgggcctcccagagctgcgcgcagagctgggccgcgcgccttcaccggccaagtgtgcgtctgcctttcccgccgcccccgccttctatccacgtgcctacagcgaccccgacccagccaaggaccctagagccgaaaagaaagagctgtgcgcgctgcagaaggcggtggagctggagaagacagaggcggacaacgcggagcggccccgggcgcgacggcggaggaagccgcgcgtgctcttctcgcaggcgcaggtctatgagctggagcggcgcttcaagcagcagcggtacctgtcggcccccgaacgcgaccagctggccagcgtgctgaaactcacgtccacgcaggtcaagatctggttccagaaccggcgctacaagtgcaagcggcagcggcaggaccagactctggagctggtggggctgcccccgccgccgccgccgcctgcccgcaggatcgcggtgccagtgctggtgcgcgatggcaagccatgcctaggggactcggcgccctacgcgcctgcctacggcgtgggcctcaatccctacggttataacgcctaccccgcctatccgggttacggcggcgcggcctgcagccctggctacagctgcactgccgcttaccccgccgggccttccccagcgcagccggccactgccgccgccaacaacaacttcgtgaacttcggcgtcggggacttgaatgcggttcagagccccgggattccgcagagcaactcgggagtgtccacgctgcatggtatccgagcctggtagggaagggacccgcgtggcgcgaccctgaccgatcccacctcaacagctccctgactctcggggggagaaggggctcccaacatgaccctgagtcccctggattttgcattcactcctgcggagacctaggaactttttctgtcccacgcgcgtttgttcttgcgcacgggagagtttgtggcggcgattatgcagcgtgcaatgagtgatcctgcagcctggtgtcttagctgtccccccaggagtgccctccgagagtccatgggcacccccggttggaactgggactgagctcgggcacgcagggcctgagatctggccgcccattccgcgagccagggccgggcgcccgggcctttgctatctcgccgtcgcccgcccacgcacccacccgtatttatgtttttacctattgctgtaagaaatgacgatccccttcccattaaagagagtgcgttgaccccg

SEQ ID NO: 33 - protein, 356 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 34 - DNA, 3002 bp, Homo sapiens
  source 1..3002: mol_type = genomic DNA
ggggaggaggggagaacggggagcgcacagcctggacgcgtgcgcaggcgtcaggcgcatagacctgctagcccctcagctagcggccccgcccgcgcttagcatcactaactgggctatataacctgagcgcccgcgcggccacgacacgaggaattcgcccacgcaggaggcgcggcgtccggaggccccagggttatgagactatcactgctcaggacctactaacaacaaaggaaatcgaaacatgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattagaggcacgccagtttcaccatttccgggaaacgaacccactgtgcttacagtgactgtcgtgtttacaaaaggcagccctttgggtactactgctgcaaagtgcaaatactccaagcttcaagtgatatatgtatttattgtcattactgcctttggaagaaacaggggatcaaagttcctgttcaccttatgtattattttctatagctcttctatttaaaaaataaaaaaatacagtaaagtttaaaaaatacaccacgaatttggtgtggctgtattcagatcgtattaattatctgatcgggataacaaaatcacaagcaataattaggatctatgcaatttttaaactagtaatgggccaattaaaatatatataaatatatatttttcaaccagcattttactacttgttacctttcccatgctgaattattttgttgtgattttgtacagaatttttaatgactttttataatgtggatttcctattttaaaaccatgcagcttcatcaatttttatacatatcagaaaagtagaattatatctaatttatacaaaataatttaactaatttaaaccagcagaaaagtgcttagaaagttattgtgttgccttagcacttctttcctctccaattgtaaaaaaaaaaaaaaaaaaaaaaaaaaaaaaaaaattgcacaatttgagcaattcatttcactttaaagtctttccgtctccctaaaataaaaaccagaatcataattttcaagagaagaaaaaattaagagatacattccctatcaaaacatatcaattcaacacattacttgcacaagcttgtatatacatattataaataaatgccaacatacccttctttaaatcaaaagctgcttgactatcacatacaatttgcactgttactttttagtcttttactcctttgcattccatgattttacagagaatctgaagctattgatgtttccagaaaatataaatgcatgattttatacatagtcacaaaaatggtggtttgtcatatattcatgtaataaatctgagcctaaatctaatcaggttgttaatgttgggatttatatctatagtagtcaattagtacagtagcttaaataaattcaaaccatttaattcataattagaacaatagctattgcatgtaaaatgcagtccagaataagtgctgtttgagatgtgatgctggtaccactggaatcgatctgtactgtaattttgtttgtaatcctgtatattatggtgtaatgcacaatttagaaaacattcatccagttgcaataaaatagtattgaaagtgagagcaattgttgcatttcttcttaaagggattctgtttttatttttggggaaagtagttgcttttttgctgagttaaaaaatactaaacactatatgtagaataaaagaaaagaaaaaagtttaccttggcatatgctcttgtctgtttatcttgcacagggagtcaccagttctatgtagataatgaaaagacctaactgatatttcattatttggaatatgggactggacggcagtacaaacagtgtgtttttttctttgttttaagtggcttagcctttaggttttttatttccatttttaaaaatgattgttacatgttttcttctatttctttttttaaaaggtggattttaataa

SEQ ID NO: 35 - protein, 367 aa, Homo sapiens
MLAVGAMEGTRQSAFLLSSPPLAALHSMAEMKTPLYPAAYPPLPAGPPSSSSSSSSSSSPSPPLGTHNPGGLKPPATGGLSSLGSPPQQLSAATPHGINDILSRPSMPVASGAALPSASPSGSSSSSSSSASASSASAAAAAAAAAAAAASSPAGLLAGLPRFSSLSPPPPPPGLYFSPSAAAVAAVGRYPKPLAELPGRTPIFWPGVMQSPPWRDARLACTPHQGSILLDKDGKRKHTRPTFSGQQIFALEKTFEQTKYLAGPERARLAYSLGMTESQVKVWFQNRRTKWRKKHAAEMATAKKKQDSETERLKGASENEEEDDDYNKPLDPNSDDEKITQLLKKHKSSSGGGGGLLLHASEPESSS

SEQ ID NO: 36 - DNA, 1116 bp, Homo sapiens
  source 1..1116: mol_type = genomic DNA
cgtgggatgttagcggtgggggcaatggagggcacccggcagagcgcattcctgctcagcagccctcccctggccgccctgcacagcatggccgagatgaagaccccgctgtaccctgccgcgtatcccccgctgcctgccggccccccctcctcctcgtcctcgtcgtcgtcctcctcgtcgccctccccgcctctgggcacccacaacccaggcggcctgaagcccccggccacgggggggctctcatccctcggcagccccccgcagcagctctcggccgccaccccacacggcatcaacgatatcctgagccggccctccatgcccgtggcctcgggggccgccctgccctccgcctcgccctccggttcctcctcctcctcttcctcgtccgcctctgcctcctccgcctctgccgccgccgcggctgctgccgcggccgcagccgccgcctcatccccggcggggctgctggccggactgccacgctttagcagcctgagcccgccgccgccgccgcccgggctctacttcagccccagcgccgcggccgtggccgccgtgggccggtaccccaagccgctggctgagctgcctggccggacgcccatcttctggcccggagtgatgcagagcccgccctggagggacgcacgcctggcctgtacccctcatcaaggatccattttgttggacaaagacgggaagagaaaacacacgagacccactttttccggacagcagatcttcgccctggagaagactttcgaacaaacaaaatacttggcggggcccgagagggctcgtttggcctattcgttggggatgacagagagtcaggtcaaggtctggttccagaaccgccggaccaagtggaggaagaagcacgctgccgagatggccacggccaagaagaagcaggactcggagacagagcgcctcaagggggcctcggagaacgaggaagaggacgacgactacaataagcctctggatcccaactcggacgacgagaaaatcacgcagctgttgaagaagcacaagtccagcagcggcggcggcggcggcctcctactgcacgcgtccgagccggagagctcatcctgaacgccg

SEQ ID NO: 37 - protein, 353 aa, Homo sapiens; identical to SEQ ID NO: 23

SEQ ID NO: 38 - DNA, 2058 bp, Homo sapiens
  source 1..2058: mol_type = genomic DNA
gggtccttcaggtaggaggtcctgggtgactttggaagtccgtagtgtctcattgcagataatttttagcttagggcctggtggctaggtcggttctctcctttccagtcggagacctctgccgcaaacatgctccgccagatcatcggtcaggccaagaagcatccgagcttgatccccctctttgtatttattggaactggagctactggagcaacactgtatctcttgcgtctggcattgttcaatccagatgtttgttgggacagaaataacccagagccctggaacaaactgggtcccaatgatcaatacaagttctactcagtgaatgtggattacagcaagctgaagaaggaacgtccagatttctaaatgaaatgtttcactataacgctgctttagaatgaaggtcttccagaagccacatccgcacaattttccacttaaccaggaaatatttctcctctaaatgcatgaaatcatgttggagatctctattgtaatctctattggagattacaatgattaaatcaataaataactgaaacttgatatgtgtcacttttttatgctgaaagtatgctctgaactttagagtataggaaattaactattagaatttaaagaatttcttgaatttctgtagtttgaaaatacgactttaagctgctttagtaaaacacttccattttgtgtatagactgttggtaacttcactagagcatacataacaactggaactggaaattatacaaaagtaaattgggaaggatactccagcatctgacactggcaaaatggaaacctttgagtttctcttactggctgttgaagtgtgtgcagtttttaacaatggtttttacttggcatctctttgttgtgattttcaaggttataagttgctttggtcctaggattgaagttgaaatctgagtttatcagtgctaaccatggtgctagtagtcaagagatcttgagaattttggctgctgagtcttggtgcagggtgcaggttttcttttcttttttcttttttttttttttgagatagtctctgtcacccaggctggagtgcagtggtacaaacatggatcactgcagcctctacctcccgggcttaagtgatcctcctgcctcagcccctaagtagccgggactacaggtatgtgccaccatgcccagttaatttttgtaattttttttagagacagggttttgccatgttgcccaggctggtctcaaactcttgagctcaagcgatccattctcctcagcctcccagggtgctgggattacaggcgtgagccattgcgcttagccatggtgcaggttttcaaaggccaggaagtatattcataattttaagatggggaatatagcaagttttcacataggtgtgtgtaagtcatcacatcatagaaacttgaggaattcagtgacattaattttggattttcatacgtaagtatacaattaaatgtttacagggtagtagaagcacattttaaatgtcaggaactgaactaagtatttgaattacgtggattatctcaaaaattttgaaattgttaaacgagttgaattacttgaattcattctgttagtcaaatggtggatatttacacccatgtagttttgaatttagagtgtgtagagtgttttcagttaccagactccatgcttttacctcctatgtgtcaggtataatttgaacctctaagaacagggtttctcaaccttgccactgttgactatttctgaaagacagtttggtttagcagaccatcccatgcgctttagcttgtttagtagctaacttgggctctgccactacagacaaaaagcactctttccctccaattcccacaggctatgagaagaatggagacattaccaaatgtccattggtgggcaaaattgcttcattcctacctctgttgagaattactctagatcctttggcacaaattacctcaaagtttaaaattgtgtaaacaaacagtgtgtcatgtaattgaaaaacattaagcaactccaaataaatgctacattaag

SEQ ID NO: 39 - protein, 343 aa, Homo sapiens
MNQLGGLFVNGRPLPLDTRQQIVRLAVSGMRPCDISRILKVSNGCVSKILGRYYRTGVLEPKGIGGSKPRLATPPVVARIAQLKGECPALFAWEIQRQLCAEGLCTQDKTPSVSSINRVLRALQEDQGLPCTRLRSPAVLAPAVLTPHSGSETPRGTHPGTGHRNRTIFSPSQAEALEKEFQRGQYPDSVARGKLATATSLPEDTVRVWFSNRRAKWRRQEKLKWEMQLPGASQGLTVPRVAPGIISAQQSPGSVPTAALPALEPLGPSCYQLCWATAPERCLSDTPPKACLKPCWGHLPPQPNSLDSGLLCLPCPSSHCHLASLSGSQALLWPGCPLLYGLE

SEQ ID NO: 40 - DNA, 2010 bp, Homo sapiens
  source 1..2010: mol_type = genomic DNA
caaagactcacccgtgagccagctctcaaagaaagcagcttgcgttgacagcctgggggcagcaaggatgcagtctcccaggagaggatgcactcggtggtgggaagccaggctggaggggcctgagtgaccctctccacaggcgggcagggcagtgggagaggtggtgtgtggatacctctgtctcacgcccagggatcagcagcatgaaccagcttggggggctctttgtgaatggccggcccctgcctctggatacccggcagcagattgtgcggctagcagtcagtggaatgcggccctgtgacatctcacggatccttaaggtatctaatggctgtgtgagcaagatcctagggcgttactaccgcacaggtgtcttggagccaaagggcattgggggaagcaagccacggctggctacaccccctgtggtggctcgaattgcccagctgaagggtgagtgtccagccctctttgcctgggaaatccaacgccagctttgtgctgaagggctttgcacccaggacaagactcccagtgtctcctccatcaaccgagtcctgcgggcattacaggaggaccagggactaccgtgcacacggctcaggtcaccagctgttttggctccagctgtcctcactccccatagtggctctgagactccccggggtacccacccagggaccggccaccggaatcggactatcttctccccaagccaagcagaggcactggagaaagagttccagcgtgggcagtatcctgattcagtggcccgtggaaagctggctactgccacctctctgcctgaggacacggtgagggtctggttttccaacagaagagccaaatggcgtcggcaagagaagctcaagtgggaaatgcagctgccaggtgcttcccaggggctgactgtaccaagggttgccccaggaatcatctctgcacagcagtcccctggcagtgtgcccacagcagccctgcctgccctggaaccactgggtccctcctgctatcagctgtgctgggcaacagcaccagaaaggtgtctgagtgacaccccacctaaagcctgtctcaagccctgctggggccacttgcccccacagccgaattccctggactcaggactgctttgccttccttgcccttcctcccactgtcacctggccagtcttagtggctctcaggccctgctctggcctggctgcccactactgtatggcttggaatgaggcaggagtgggaaggagatggcatagagaagatctaataccatcctgcccattgtccttaccgtcctgcccatacagactgtggctccttcctccttcctgtgattgctccctcctgtgtggacgttgcctggccctgcctcgatgcctctctggcgcatcacctgattggaggggctggtaaagcaacacccacccacttctcacactagccttaagaggcctccactcagcagtaataaaagctgtttttattagcagtagttctgttgtccatcatgttttccctatgagcacccctatgcccactctaatattcaacaattatagacaatttgccctatcatttatttacatctatgtatctaccatctaatctatgcatgtatgtaggcaatacatgtatctaaacaatgtatttgtcaatgcatcaatttacctactctatgtatgcatctatatgtgtattatgtatgcgtgcatgcgtgcgcgcacacacacacacacacacacacactgacattatatcatggcattttattcctaaatcttccagcatgcatccccaaaaaacaagaaacttgtcttacataatcacaataatatatccacatctaagaaaatttactgtaacttcttaatctaagaaaattatgtatttttgtcatatgtattttgtcatatgtattttgtatttgcatatgtattttgtatttgcatatgtatttttgtcatagcagcaaacagagtgaaatgccatttttcatattct

SEQ ID NO: 41 - protein, 286 aa, Homo sapiens
MGADGMYDKLRMLNGQTGSWGTRPGWYPGTSVPGQPTQDGCQQQEGGGENTNSISSNGEDSDEAQMRLQLKRKLQRNRTSFTQEQIEALEKEFERTHYPDVFARERLAAKIDLPEARIQVWFSNRRAKWRREEKLRNQRRQASNTPSHIPISSSFSTSVYQPIPQPTTPVSSFTSGSMLGRTDTALTNTYSALPPMPSFTMANNLPMQPPVPSQTSSYSCMLPTSPSVNGRSYDTYTPPHMQTHMNSQPMGTSGTTSTGLISPGVSVPVQVPGSEPDMSQYWPRLQ

SEQ ID NO: 42 - DNA, 6732 bp, Homo sapiens
  source 1..6732: mol_type = genomic DNA
cttttcaattagccttccatgcatgatccggagcgacttccgcctatttccagaaattaagctcaaacttgacgtgcagctagttttattttaaagacaaatgtcagagaggctcatcatattttcccccctcttctatatttggagcttatttattgctaagaagctcaggctcctggcgtcaatttatcagtaggctccaaggagaagagaggagaggagaggagagctgaacagggagccacgtcttttcctgggagggctgctatctaagtcggggctgcaggtcacagcggagtgaatcagctcggtggtgtctttgtcaacgggcggccactgccggactccacccggcagaagattgtagagctagctcacagcggggcccggccgtgcgacatttcccgaattctgcagacccatgcagatgcaaaagtccaagtgctggacaatcaaaacgtgtccaacggatgtgtgagtaaaattctgggcaggtattacgagactggctccatcagacccagggcaatcggtggtagtaaaccgagagtagcgactccagaagttgtaagcaaaatagcccagtataagcgggagtgcccgtccatctttgcttgggaaatccgagacagattactgtccgagggggtctgtaccaacgataacataccaagcgtgtcatcaataaacagagttcttcgcaacctggctagcgaaaagcaacagatgggcgcagacggcatgtatgataaactaaggatgttgaacgggcagaccggaagctggggcacccgccctggttggtatccggggacttcggtgccagggcaacctacgcaagatggctgccagcaacaggaaggagggggagagaataccaactccatcagttccaacggagaagattcagatgaggctcaaatgcgacttcagctgaagcggaagctgcaaagaaatagaacatcctttacccaagagcaaattgaggccctggagaaagagtttgagagaacccattatccagatgtgtttgcccgagaaagactagcagccaaaatagatctacctgaagcaagaatacaggtatggttttctaatcgaagggccaaatggagaagagaagaaaaactgaggaatcagagaagacaggccagcaacacacctagtcatattcctatcagcagtagtttcagcaccagtgtctaccaaccaattccacaacccaccacaccggtttcctccttcacatctggctccatgttgggccgaacagacacagccctcacaaacacctacagcgctctgccgcctatgcccagcttcaccatggcaaataacctgcctatgcaacccccagtccccagccagacctcctcatactcctgcatgctgcccaccagcccttcggtgaatgggcggagttatgatacctacacccccccacatatgcagacacacatgaacagtcagccaatgggcacctcgggcaccacttcaacaggactcatttcccctggtgtgtcagttccagttcaagttcccggaagtgaacctgatatgtctcaatactggccaagattacagtaaaaaaaaaaaaaaaaaaaaaaaggaaaggaaatattgtgttaattcagtcagtgactatggggacacaacagttgagctttcaggaaagaaagaaaaatggctgttagagccgcttcagttctacaattgtgtcctgtattgtaccactggggaaggaatggacttgaaacaaggacctttgtatacagaaggcacgatatcagttggaacaaatcttcattttggtatccaaacttttattcattttggtgtattatttgtaaatgggcatttgtatgttataatgaaaaaaagaacaatgtagactggatggatgtttgatctgtgttggtcatgaagttgttttttttttttttaaaaagaaaaccatgatcaacaagctttgccacgaatttaagagttttatcaagatatatcgaatacttctacccatctgttcatagtttatggactgatgttccaagtttgtatcattcctttgcatataattaaacctggaacaacatgcactagatttatgtcagaaatatctgttggttttccaaaggttgttaacagatgaagtttatgtgcaaaaaagggtaagatataaattcaaggaagaaaaaaagttgatagctaaaaggtagagtgtgtcttcgatataatccaatttgttttatgtcaaaatgtaagtatttgtcttccctagaaatcctcagaatgatttctataataaagttaatttcatttatatttgacaagaatatagatgttttatacacattttcatgcaatcatacgtttcttttttggccagcaaaagttaattgttcttagatatagttgtattactgttcacggtccaatcattttgtgcatctagagttcattcctaatcaattaaaagtgcttgcaagagttttaaacttaagtgttttgaagttgttcacaactacatatcaaaattaaccattgttgattgtaaaaaaccatgccaaagcctttgtatttcctttattatacagttttctttttaaccttatagtgtggtgttacaaattttatttccatgttagatcaacattctaaaccaatggttactttcacacacactctgttttacatcctgatgatccttaaaaaataatccttatagataccataaatcaaaaacgtgttagaaaaaaattccacttacagcagggtgtagatctgtgcccatttatacccacaacatatatacaaaatggtaacatttcccagttagccatttaattctaaagctcaaagtctagaaataatttaaaaatgcaacaagcgattagctaggaattgttttttgaattaggactggcattttcaatctgggcagatttccattgtcagcctatttcaacaatgatttcactgaagtatattcaaaagtagatttcttaaaggagactttctgaaagctgttgcctttttcaaataggccctctcccttttctgtctccctcccctttgcacaagaggcatcatttcccattgaaccactacagctgttcccatttgaatcttgctttctgtgcggttgtggatggttggagggtggaggggggatgttgcatgtcaaggaataatgagcacagacacatcaacagacaacaacaaagcagactgtgactggccggtgggaattaaaggccttcagtcattggcagcttaagccaaacattcccaaatctatgaagcagggcccattgttggtcagttgttatttgcaatgaagcacagttctgatcatgtttaaagtggaggcacgcagggcaggagtgcttgagcccaagcaaaggatggaaaaaaataagcctttgttgggtaaaaaaggactgtctgagactttcatttgttctgtgcaacatataagtcaatacagataagtcttcctctgcaaacttcactaaaaagcctgggggttctggcagtctagattaaaatgcttgcacatgcagaaacctctggggacaaagacacacttccactgaattatactctgctttaaaaaaatccccaaaagcaaatgatcagaaatgtagaaattaatggaaggatttaaacatgaccttctcgttcaatatctactgttttttagttaaggaattacttgtgaacagataattgagattcattgctccggcatgaaatatactaataattttattccaccagagttgctgcacatttggagacaccttcctaagttgcagtttttgtatgtgtgcatgtagttttgttcagtgtcagcctgcactgcacagcagcacatttctgcaggggagtgagcacacatacgcactgttggtacaattgccggtgcagacatttctacctcctgacattttgcagcctacattccctgagggctgtgtgctgagggaactgtcagagaagggctatgtgggagtgcatgccacagctgctggctggcttacttcttccttctcgctggctgtaatttccaccacggtcaggcagccagttccggcccacggttctgttgtgtagacagcagagactttggagacccggatgtcgcacgccaggtgcaagaggtgggaatgggagaaaaggagtgacgtgggagcggagggtctgtatgtgtgcacttgggcacgtatatgtgtgctctgaaggtcaggattgccagggcaaagtagcacagtctggtatagtctgaagaagcggctgctcagctgcagaagccctctggtccggcaggatgggaacggctgccttgccttctgcccacaccctagggacatgagctgtccttccaaacagagctccaggcactctcttggggacagcatggcaggctctgtgtggtagcagtgcctgggagttggccttttactcattgttgaaataatttttgtttattatttatttaacgatacatatatttatatatttatcaatggggtatctgcagggatgttttgacaccatcttccaggatggagattatttgtgaagacttcagtagaatcccaggactaaacgtctaaattttttctccaaacttgactgacttgggaaaaccaggtgaatagaataagagctgaatgttttaagtaataaacgttcaaactgctctaagtaaaaaaatgcattttactgcaatgaatttctagaatatttttcccccaaagctatgcctcctaacccttaaatggtgaacaactggtttcttgctacagctcactgccatttcttcttactatcatcactaggtttcctaagattcactcatacagtattatttgaagattcagctttgttctgtgaatgtcatcttaggattgtgtctatattcttttgcttatttctttttactctgggcctctcatactagtaagattttaaaaagccttttcttctctgtatgtttggctcaccaaggcgaaatatatattcttctctttttcatttctcaagaataaacctcatctgcttttttgtttttctgtgttttggcttggtactgaatgactcaactgctcggttttaaagttcaaagtgtaagtacttagggttagtactgcttatttcaataatgttgacggtgactatctttggaaagcagtaacatgctgtcttagaaatgacattaataatgggcttaaacaaatgaataggggggtccccccactctccttttgtatgcctatgtgtgtctgatttgttaaaagatggacagggaattgattgcagagtgtcgcttccttctaaagtagttttattttgtctactgttagtatttaaagatcctggaggtggacataaggaataaatggaagagaaaagtagatattgtatggtggctactaaaaggaaattcaaaaagtcttagaacccgagcacctgagcaaactgcagtagtcaaaatatttatctcatgttaaagaaaggcaaatctagtgtaagaaatgagtaccatatagggttttgaagttcatatactagaaacacttaaaagatatcatttcagatattacgtttggcattgttcttaagtatttatatctttgagtcaagctgataattaaaaaaaatctgttaatggagtgtatatttcataatgtatcaaaatggtgtctatacctaaggtagcattattgaagagagatatgtttatgtagtaagttattaacataatgagtaacaaataatgtttccagaagaaaggaaaacacattttcagagtgcgtttttatcagaggaagacaaaaatacacacccctctccagtagcttatttttacaaagccggcccagtgaattagaaaaacaaagcacttggatatgatttttggaaagcccaggtacacttattattcaaaatgcacttttactgagtttgaaaagtttcttttatatttaaaataagggttcaaatatgcatattcaatttttatagtagttatctatttgcaaagcatatattaactagtaattggctgttaattttatagacatggtagccagggaagtatatcaatgacctattaagtattttgacaagcaatttacatatctgatgacctcgtatctctttttcagcaagtcaaatgctatgtaattgttccattgtgtgttgtataaaatgaatcaacacggtaagaaaaaggttagagttattaaaataataaactgactaaaatactcatttgaatttattcagaatgttcataatgctttcaaaggacatagcagagcttttgtggagtatccgcacaacattatttattatctatggactaaatcaattttttgaagttgctttaaaatttaaaagcacctttgcttaatataaagccctttaattttaactgacagatcaattctgaaactttattttgaaaagaaaatggggaagaatctgtgtctttagaattaaaagaaatgaaaaaaataaacccgacattctaaaaaaatagaataagaaacctgatttttagtactaatgaaatagcgggtgacaaaatagttgtctttttgattttgatcacaaaaaataaactggtagtgacaggatatgatggagagatttgacatcctggcaaatcactgtcattgattcaattattctaattctgaataaaagctgtatacagtaaaa

SEQ ID NO: 43 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 44 - DNA, 2573 bp, Homo sapiens
  source 1..2573: mol_type = genomic DNA
gggtggcgccgggagtgggaacgccacacagtgccaaatccccggctccagctcccgactcccggctcccggctcccggctcccggtgcccaatcccgggccgcagccatgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgagaggcaggagctgctcctggctgaggggcttcaaccactcgccgaggaggagcagagggcctaggaggaccccgggcgtggaccacccgccctggcagttgaatggggcggcaattgcggggcccaccttagaccgaaggggaaaacccgctctctcaggcgcatgtgccagttggggccccgcgggtagatgccggcaggccttccggaagaaaaagagccattggtttttgtagtattggggccctcttttagtgatactggattggcgttgtttgtggctgttgcgcacatccctgccctcctacagcactccaccttgggacctgtttagagaagccggctcttcaaagacaatggaaactgtaccatacacattggaaggctccctaacacacacagcggggaagctgggccgagtaccttaatctgccataaagccattcttactcgggcgacccctttaagtttagaaataattgaaaggaaatgtttgagttttcaaagatcccgtgaaattgatgccagtggaatacagtgagtcctcctcttcctcctcctcctcttccccctccccttcctcctcctcctcttcttttccctcctcttcctcttcctcctgctctcctttcctccccctcctcttttccctcctcttcctcttcctcctgctctcctttcctccccctcctctttctcctcctcctcctcttcttccccctcctctccctcctcctcttcttccccctcctctccctcctcctcttcttctccctcctcttcctcttcctcctcttccacgtgctctcctttcctccccctcctcttgctccccttcttccccgtcctcttcctcctcctcctcttcttctccctcctcttcctcctcctctttcttcctgacctctttctttctcctcctcctccttctacctccccttctcatccctcctcttcctcttctctagctgcacacttcactactgcacatcttataacttgcacccctttcttctgaggaagagaacatcttgcaaggcagggcgagcagcggcagggctggcttaggagcagtgcaagagtccctgtgctccagttccacactgctggcagggaaggcaaggggggacgggcctggatctgggggtgagggagaaagatggacccctgggtgaccactaaaccaaagatattcggaactttctatttaggatgtggacgtaattcctgttccgaggtagaggctgtgctgaagacaagcacagtggcctggtgcgccttggaaaccaacaactattcacgagccagtatgaccttcacatctttagaaattatgaaaacgtatgtgattggagggtttggaaaaccagttatcttatttaacattttaaaaattacctaacagttatttacaaacaggtctgtgcatcccaggtctgtcttcttttcaaggtctgggccttgtgctcgggttatgtttgtgggaaatgcttaataaatactgataatatgggaagagatgaaaactgattctcctcactttgtttcaaacctttctggcagtgggatgattcgaattcacttttaaaattaaattagcgtgttttgttttg

SEQ ID NO: 45 - protein, 328 aa, Homo sapiens
MDAVLLEHFPGGLDAFPSSYFDEDDFFTDQSSRDPLEDGDELLADEQAEVEFLSHQLHEYCYRDGACLLLQPAPPAAPLALAPPSSGGLGEPDDGGGGGYCCETGAPPGGFPYSPGSPPSCLAYPCAGAAVLSPGARLRGLSGAAAAAARRRRRVRSEAELQQLRQAANVRERRRMQSINDAFEGLRSHIPTLPYEKRLSKVDTLRLAIGYINFLSELVQADLPLRGGGAGGCGGPGGGGRLGGDSPGSQAQKVIICHRGTRSPSPSDPDYGLPPLAGHSLSWTDEKQLKEQNIIRTAKVWTPEDPRKLNSKSSFNNIENEPPFEFVS

SEQ ID NO: 46 - DNA, 1339 bp, Homo sapiens
  source 1..1339: mol_type = genomic DNA
atggacgcggtgttgctggagcacttccccgggggcctagacgcctttccttcttcgtacttcgacgaggacgacttcttcaccgaccagtcttcacgggaccccctggaggacggcgatgagctgctggcggacgagcaggccgaggtggagttccttagccaccagctccacgagtactgctaccgcgacggggcgtgcctgctgctgcagcccgcgcccccggccgccccgctagcgctcgccccgccgtcctcggggggcctcggtgagccagacgacggcggcggcggcggctactgctgcgagacgggggcgcccccaggcggcttcccctactcgcccggctcgccgccctcgtgcctggcctacccgtgcgccggggcggcagtactgtctcccggggcgcggctgcgcggcctgagcggagcggcggctgcggcggcgcggcgccggcggcgggtgcgctccgaggcggagctgcagcagctgcggcaggcggccaacgtgcgcgagcggcggcgcatgcagtccatcaacgacgccttcgaggggctgcgctcgcacatccccacgctgccctacgagaagcgcctctccaaggtggacacgctgcgcctggccatcggctacatcaacttcctcagcgagctcgtgcaggccgacctgcccttgcgcggcggtggcgcgggcggctgcggggggccgggcggcggcgggcgcctgggcggggacagcccgggcagccaggcccagaaggtcatcatctgccatcggggcacccggtccccctcccccagcgaccctgattatggcctccctcccctagcaggacactctctctcatggactgatgaaaaacaactcaaggaacaaaatattatccgaacagccaaagtctggaccccagaggaccccagaaaactcaacagcaaatcttccttcaacaacatagaaaacgaaccaccatttgagtttgtgtcctgagaagtcccagactcggctgaagatctgattatgtctctgtgcatattgtacatgtaaatatctataatgtaaatgtaatttaagaatcaaatttttcgaatggcaatcaactgtttattatttatctatttattatcctgttgagttgatgaaatagatgatttctttttaaatatataatttatataacttatcctgattttctgaaaatatgcaatagcctatgattttcctgaactctgtgttgttgggagaactctggccagaaaacgtcctgcttatttattgccagatatggtttatttctaagcgttgtcaataaatgctatttacaccttttcctgaaaaaaaa

SEQ ID NO: 47 - DNA, 1555 bp, Homo sapiens
  source 1..1555: mol_type = genomic DNA
cggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctccctgggggtggggctcctacctgggggcagaactcctacctgaaggcagggctcctccctggagctagtgtctcctctctggtggctgggctgctcctgaatgaggcggagctccaggatggggaggggctctgcgttggcttctccctggggacggggctcccctggacagaggcggggctacagattgggcggggcttctcttgggtgggacagggcttctcctgcgggggcgaggcccctcccagtaagggcgtggctctgggtgggcggggcactaggtaggcttctacctgcaggcggggctcctcctgaaggaggcggggctctaggatggggcacggctctggggtaggctgctccctgagggcg

SEQ ID NO: 48 - protein, 385 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCKNPGSRAGNSAHQPPHPQPPVRFHPPLRRAGKVP

SEQ ID NO: 49 - DNA, 1056 bp, Homo sapiens
  source 1..1056: mol_type = genomic DNA
atgagtccccgctcgtgcctgcgttcgctgcgcctcctcgtcttcgccgtcttctcagccgccgcgagcaactggctgtacctggccaagctgtcgtcggtggggagcatctcagaggaggagacgtgcgagaaactcaagggcctgatccagaggcaggtgcagatgtgcaagcggaacctggaagtcatggactcggtgcgccgcggtgcccagctggccattgaggagtgccagtaccagttccggaaccggcgctggaactgctccacactcgactccttgcccgtcttcggcaaggtggtgacgcaagggattcgggaggcggccttggtgtacgccatctcttcggcaggtgtggcctttgcagtgacgcgggcgtgcagcagtggggagctggagaagtgcggctgtgacaggacagtgcatggggtcagcccacagggcttccagtggtcaggatgctctgacaacatcgcctacggtgtggccttctcacagtcgtttgtggatgtgcgggagagaagcaagggggcctcgtccagcagagccctcatgaacctccacaacaatgaggccggcaggaaggccatcctgacacacatgcgggtggaatgcaagtgccacggggtgtcaggctcctgtgaggtaaagacgtgctggcgagccgtgccgcccttccgccaggtgggtcacgcactgaaggagaagtttgatggtgccactgaggtggagccacgccgcgtgggctcctccagggcactggtgccacgcaacgcacagttcaagccgcacacagatgaggacttggtgtacttggagcctagccccgacttctgtgagcaggacatgcgcagcggcgtgctgggcacgaggggccgcacatgcaacaagacgtccaaggccatcgacggctgtgagctgctgtgctgtggccgcggcttccacacggcgcaggtggagctggctgaacgctgcagctgcaaattccactggtgctgcttcgtcaagtgccggcagtgccagcggctcgtggagttgcacacgtgccgatga

SEQ ID NO: 50 - protein, 351 aa, Homo sapiens
MSPRSCLRSLRLLVFAVFSAAASNWLYLAKLSSVGSISEEETCEKLKGLIQRQVQMCKRNLEVMDSVRRGAQLAIEECQYQFRNRRWNCSTLDSLPVFGKVVTQGIREAALVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDMRSGVLGTRGRTCNKTSKAIDGCELLCCGRGFHTAQVELAERCSCKFHWCCFVKCRQCQRLVELHTCR

SEQ ID NO: 51 - DNA, 1143 bp, Homo sapiens
  source 1..1143: mol_type = genomic DNA
atgaagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgaaggtggaactgcagcactgtggataacacctctgtttttggcagggtgatgcagataggcagccgcgagacggccttcacatacgcggtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtggctacgaccagttcaagaccgtgcagacggagcgctgccactgcaagttccactggtgctgctacgtcaagtgcaagaagtgcacggagatcgtggaccagtttgtgtgcaagtag

SEQ ID NO: 52 - protein, 380 aa, Homo sapiens
MKKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

(19) United States
(12) Patent Application Publication
(10) Pub. No.: US 2024/0101962 A1
(43) Pub. Date: Mar. 28, 2024

(54) COMPOSITIONS AND METHODS FOR ORGANOID GENERATION AND DISEASE MODELING

(71) Applicant: Salk Institute for Biological Studies, La Jolla, CA (US)

(72) Inventors: Ronald EVANS, La Jolla, CA (US); Michael DOWNES, La Jolla, CA (US); Annette ATKINS, La Jolla, CA (US); Eiji YOSHIHARA, La Jolla, CA (US); Ruth YU, La Jolla, CA (US)

(73) Assignee: Salk Institute for Biological Studies, La Jolla, CA (US)

(21) Appl. No.: 18/340,537

(22) Filed: Jun. 23, 2023

Related U.S. Application Data
(63) Continuation of application No. 17/706,272, filed on Mar. 28, 2022, now Pat. No. 11,685,901.
Application No. 17/706,272 is a division of application No. 16/301,671, filed on Nov. 14, 2018, now Pat. No. 11,760,977.
(60) Provisional application No. 62/341,461, filed on May 25, 2016.

(51) Int. Cl. C12N 5/071; A61K 35/39; G01N 33/50

(52) U.S. Cl. CPC C12N 5/0677; A61K 35/39; G01N 33/5088; C12N 2501/117; C12N 2501/16; C12N 2501/385; C12N 2501/395; C12N 2501/415; C12N 2501/727; C12N 2501/999; C12N 2502/1382; C12N 2502/28; C12N 2503/02; C12N 2506/02; C12N 2506/45; C12N 2513/00; C12N 2533/52; C12N 2533/54; C12N 2533/90; G01N 2800/04; G01N 2800/7028

(57) ABSTRACT

The invention features pancreatic islet and pancreatic organoids, and cell cultures and methods that are useful for the rapid and reliable generation of pancreatic islet and pancreatic islet organoids. The invention also features methods of treating pancreatic diseases and methods of identifying agents that are useful for treatment of pancreatic diseases, such as type 2 diabetes and pancreatic cancer, using the pancreatic islet and pancreatic organoids of the invention.

### CROSS REFERENCE TO RELATED APPLICATIONS

This application is a continuation of U.S. application Ser. No. 17/706,272, filed Mar. 28, 2022, which is a divisional of U.S. application Ser. No. 16/301,671, filed Nov. 14, 2018, which is a U.S. National Stage application under 35 U.S.C. § 371 of International PCT Application No. PCT/US2017/034278, filed May 24, 2017, which claims priority to and benefit of U.S. Provisional Patent Application No. 62/341,461, filed May 25, 2016, the entire contents of each of which are incorporated by reference herein.

### STATEMENT OF RIGHTS TO INVENTIONS MADE UNDER FEDERALLY SPONSORED RESEARCH

This invention was made with government support under Grant Nos. DK057978 and DK0909962 awarded by the National Institutes of Health. The government has certain rights in the invention.

### SEQUENCE LISTING

The present application contains a Sequence Listing which has been submitted electronically in XML format following conversion from the originally filed TXT format.

The contents of the electronic XML Sequence Listing, (Date of creation: Jun. 23, 2023; Size: 146,117 bytes; Name: 167776-010910USCON-SL.xml), and the original TXT format, is herein incorporated by reference in its entirety.

### BACKGROUND OF THE INVENTION

Although animal disease models can yield insight into the pathogenesis of diseases, drugs screened and selected using animal models often fail to be adopted in human patients. Because evolutionary biology, molecular biology, and genetic studies show animals and humans can profoundly differ, recapitulating human disease using human cells and generation of functional human organs is urgently needed.

### SUMMARY OF THE INVENTION

As described below, the present invention features compositions and methods for generating an organoid, including a pancreatic islet organoid or a pancreatic organoid.

In one aspect, the invention provides a method of generating a pancreatic islet organoid, the method involving culturing an induced pluripotent stem cell (iPSC)-derived beta-like cell in a 3-dimensional matrix containing gellan gum, thereby generating a pancreatic islet organoid.

In another aspect, the invention provides a cell culture including an iPSC-derived beta-like cell in a three-dimensional matrix containing gellan gum.

In another aspect, the invention provides a cell culture including a human iPSC-derived beta-like cell, a human adipose-derived stem cell (hADSC), and a human umbilical vein endothelial cell (HUVEC) in a three-dimensional matrix containing gellan gum.

In various embodiments of any aspect delineated herein, the cell culture includes an adipose-derived stem cell and/or an endothelial cell.

In another aspect, the invention provides a pancreatic islet organoid containing an iPSC-derived beta-like cell, where the organoid is vascularized and exhibits glucose-stimulated insulin secretion (GSIS).

In another aspect, the invention provides a pancreatic islet organoid containing an iPSC-derived beta-like cell, an iPSC-derived alpha cell, an iPSC-derived delta cell, an iPSC-derived duct cell, an adipose-derived stem cell (hADSC), and an endothelial cell where the organoid is vascularized and exhibits glucose-stimulated insulin secretion (GSIS), KCl-stimulated insulin secretion, GLP-1 stimulated insulin secretion, somatostatin secretion, and glucagon secretion.

In a related aspect, the invention provides a non-human organism transplanted with the organoid of any aspect delineated herein.

In another aspect, the invention provides a method of identifying an agent that modulates pancreatic activity and/or treats a pancreatic disease involving contacting a candidate agent with a pancreatic islet organoid or a pancreatic organoid; and measuring an activity of the organoid contacted with the candidate agent, where the candidate agent is identified as an agent that modulates pancreatic activity and/or treats a pancreatic disease if the activity of the organoid is altered relative to a reference.

In another aspect, the invention provides a method of identifying an agent that modulates pancreatic activity and/or treats a pancreatic disease involving administering a candidate agent to a non-human subject transplanted with a pancreatic islet organoid or a pancreatic organoid; and measuring a pancreatic activity of the non-human subject, where the candidate agent is identified as an agent that modulates pancreatic activity and/or treats a pancreatic disease if the pancreatic activity of the non-human subject is altered relative to a reference.

In another aspect, the invention provides a method of treating a pancreatic disease in a subject involving transplanting a pancreatic islet organoid into the subject, where the pancreatic islet organoid contains an iPSC-derived beta-like cell, is vascularized, and exhibits glucose-stimulated insulin secretion (GSIS).

In another aspect, the invention provides a method of treating type 1 diabetes in a subject, involving transplanting a pancreatic islet organoid into the subject, where the pancreatic islet organoid contains an iPSC-derived beta-like cell, is vascularized, and exhibits glucose-stimulated insulin secretion (GSIS).

In another aspect, the invention provides a pancreatic islet organoid generated by culturing an induced pluripotent stem cell (iPSC)-derived beta-like cell in a 3-dimensional matrix containing gellan gum.

In another aspect, the invention provides a pancreatic organoid generated by culturing an induced pluripotent stem cell (iPSC)-derived beta-like cell and an iPSC-derived exocrine component cell in a 3-dimensional matrix containing gellan gum.

In another aspect, the invention provides a liver organoid generated by culturing an induced pluripotent stem cell (iPSC)-derived hepatocyte in a 3-dimensional matrix containing gellan gum.

In another aspect, the invention provides a heart organoid generated by culturing an induced pluripotent stem cell (iPSC)-derived cardiomyocyte in a 3-dimensional matrix containing gellan gum.

In another aspect, the invention provides an intestinal organoid generated by culturing an induced pluripotent stem cell (iPSC)-derived intestinal cell in a 3-dimensional matrix containing gellan gum.

In various embodiments of any aspect delineated herein, the method involves culturing the iPSC-derived beta-like cell with an adipose-derived stem cell and/or an endothelial cell. In various embodiments of any aspect delineated herein, the method involves culturing the iPSC-derived beta-like cell with an iPSC-derived alpha-like cell, an iPSC-derived delta-like cell, and/or an iPSC-derived duct-like cell.

In various embodiments of any aspect delineated herein, the pancreatic islet organoid contains an iPSC-derived alpha-like cell, an iPSC-derived delta-like cell, and/or an iPSC-derived duct-like cell. In various embodiments of any aspect delineated herein, the pancreatic islet organoid includes an adipose-derived stem cell and/or an endothelial cell. In various embodiments of any aspect delineated herein, the pancreatic islet organoid exhibits KCl-stimulated insulin secretion, GLP-1 stimulated insulin secretion, somatostatin secretion, and/or glucagon secretion. In various embodiments of any aspect delineated herein, the pancreatic islet organoid expresses one or more of the beta cell transcription factors Pdx1, MafA, Pax4, Pax6, NeuroD1, Nkx6-1,Gata6, and Foxa2. In certain embodiments, the pancreatic islet organoid contains an iPSC-derived beta-like cell, an iPSC-derived alpha cell, an iPSC-derived delta cell, an iPSC-derived duct cell, an adipose-derived stem cell (hADSC), and an endothelial cell, where the organoid is vascularized and exhibits glucose-stimulated insulin secretion (GSIS), KCl-stimulated insulin secretion, GLP-1 stimulated insulin secretion, somatostatin secretion, and glucagon secretion. In various embodiments of any aspect delineated herein, the pancreatic islet organoid is surrounded by an iPSC-derived exocrine component. In various embodiments, the iPSC-derived exocrine component expresses one or more of the markers PDX1, Nkx6-1, and Ptf1.

In various embodiments of any aspect delineated herein, the method involves inducing or mimicking a pancreatic disease in the organoid or non-human subject. In various embodiments of any aspect delineated herein, the disease is induced by contacting the organoid with or administering to the non-human subject one or more of the following agents: a free fatty acid (FFA), glucose, and cytokine. In various embodiments of any aspect delineated herein, the disease is mimicked by culturing the organoid with pancreatic cancer cells, stellate cells, and immune cells to create human pancreatic cancer microenvironment.

In various embodiments of any aspect delineated herein, the pancreatic activity is one or more of insulin secretion, beta cell apoptosis, expression or activity of a NDUFA4, ESRRG, G6PC2, MDH1, LDHA, KCNK3, or MAFA polypeptide or polynucleotide, amylase secretion, apoptosis of an exocrine component, collagen synthesis, and stellate cell activation. In various embodiments, the non-human subject is also transplanted with a liver organoid.

In various embodiments of any aspect delineated herein, the candidate agent increases insulin secretion. In various embodiments of any aspect delineated herein, the candidate agent identified as an agent that modulates pancreatic activity is tested for the ability to treat a pancreatic disease. In various embodiments, the pancreatic disease is type 2 diabetes or pancreatic cancer.

In various embodiments of any aspect delineated herein, the organoid is an organoid according to any aspect delineated herein.

In various embodiments, the non-human organism is a mammal (e.g., a mouse).

In various embodiments of any aspect delineated herein, the pancreatic islet organoid contains an iPSC-derived alpha-like cell, an iPSC-derived delta-like cell, and/or an iPSC-derived duct-like cell.

In various embodiments of any aspect delineated herein, the pancreatic islet organoid contains an adipose-derived stem cell and/or an endothelial cell.

In various embodiments of any aspect delineated herein, a pancreatic disease is induced or mimicked in the subject. In particular embodiments, the pancreatic disease is type 1 diabetes or type 2 diabetes. In certain embodiments, the subject is a mammal (e.g., human). In various embodiments of any aspect delineated herein, the subject is administered an immunosuppressive agent.

In various embodiments of any aspect delineated herein, the liver organoid expresses one or more of the markers AFP, ALB, and Cyp3a7. In various embodiments of any aspect delineated herein, the liver organoid exhibits insulin signaling, insulin resistance by palmitic acids, and lipid accumulation.

In various embodiments of any aspect delineated herein, the heart organoid expresses one or more of the markers hMlc2a, hNkx2-5, alphaMHC and KCNQ1. In various embodiments of any aspect delineated herein, the heart organoid exhibits cardiac beating.

In various embodiments of any aspect delineated herein, the intestinal organoid expresses one or more of the markers CDX2, Muc2, and Lgr5. In various embodiments of any aspect delineated herein, the intestinal organoid exhibits budding in response to R-spondin.

In various embodiments of any aspect delineated herein, the iPSC-derived beta-like cell, iPSC-derived alpha-like cell, iPSC-derived delta-like cell, and/or iPSC-derived duct-like cell is human. In various embodiments of any aspect delineated herein, the iPSC-derived beta-like cell, iPSC-derived exocrine component cell, iPSC-derived hepatocyte, iPSC-derived cardiomyocyte, or iPSC-derived intestinal cell is human. In various embodiments, the adipose-derived stem cell is a human adipose-derived stem cell (hADSC). In various embodiments of any aspect delineated herein, the endothelial cell is a human umbilical vein endothelial cell (HUVEC).

In various embodiments of any aspect delineated herein, the pancreatic islet organoid, pancreatic organoid, liver organoid, heart organoid, or intestinal organoid, contains an adipose-derived stem cell and/or an endothelial cell. In various embodiments of any aspect delineated herein, the pancreatic islet organoid, pancreatic organoid, liver organoid, heart organoid, or intestinal organoid is vascularized.

In another aspect, the invention provides a method of generating a pancreatic islet organoid, the method comprising culturing an induced pluripotent stem cell (iPSC)-derived beta-like cell in a medium comprising Wnt4 or Wnt5a protein. In an embodiment, the induced pluripotent stem cell (iPSC)-derived beta-like cell is cultured in a 3-dimensional matrix. In an embodiment of the foregoing aspect, the Wnt4 or Wnt5a protein is a recombinant human Wnt4 or Wnt5a protein. In a particular embodiment, the medium comprises recombinant human Wnt4 protein. In another particular embodiment, the medium comprises recombinant human Wnt5a protein.

In another aspect the invention provides a cell culture comprising a human iPSC-derived beta-like cell and Wnt4 or Wnt5a protein. In an embodiment, the human iPSC-derived beta-like cell is in a three-dimensional matrix comprising gellan gum. In an embodiment, the Wnt4 or Wnt5a protein is a recombinant human Wnt4 or Wnt5a protein. In a particular embodiment, the medium comprises recombinant human Wnt4 protein. In another particular embodiment, the medium comprises recombinant human Wnt5a protein.

In another aspect, the invention provides a pancreatic islet organoid comprising an iPSC-derived beta-like cell cultured in medium comprising Wnt4 or Wnt5a protein, wherein the organoid is vascularized and exhibits glucose-stimulated insulin secretion (GSIS). In an embodiment, the organoid further exhibits KCl-stimulated insulin secretion or glucose stimulated insulin secretion. In an embodiment, the pancreatic islet organoid expresses Fltp and Esrrg genes. In an embodiment, the Wnt4 or Wnt5a protein is a recombinant human Wnt4 or Wnt5a protein. In a particular embodiment, the medium comprises recombinant human Wnt4 protein. In another particular embodiment, the medium comprises recombinant human Wnt5a protein.

In another aspect, the invention provides a non-human organism transplanted with the organoid defined in the above described aspects.

In another aspect, the invention provides a method of enhancing self organization of adipose-derived stem cells (ADSCs) for generating an induced pluripotent stem cell (iPSC)-derived organoid, the method comprising culturing the ADSCs in a 3-dimensional (3-D) culture matrix medium comprising a Wnt5a protein. In an embodiment of the method, the ADSCs are cultured in a 3-D culture matrix comprising gellan gum. In an embodiment, the ADSCs are cultured in the 3-D culture matrix medium comprising a Wnt5 protein and an iPSC-derived cell selected from an iPSC-derived beta-like cell, an iPSC-derived exocrine component cell, an iPSC-derived hepatocyte, an iPSC-derived cardiomyocyte, or an iPSC-derived intestinal cell. In an embodiment of the method, the iPSC-derived organoid is selected from a pancreatic islet organoid, pancreatic organoid, a liver organoid, a heart organoid, or an intestinal organoid. In an embodiment of the method, the induced pluripotent stem cell (iPSC)-derived organoid is a human induced pluripotent stem cell (hiPSC)-derived organoid. In an embodiment of the method, the Wnt5a protein is a recombinant human Wnt5a protein. In an embodiment of the method, the pancreatic islet organoid, pancreatic organoid, liver organoid, heart organoid, or intestinal organoid is derived from an iPSC-derived cell selected from an iPSC-derived beta-like cell, an iPSC-derived exocrine component cell, an iPSC-derived hepatocyte, an iPSC-derived cardiomyocyte, or an iPSC-derived intestinal cell, respectively. In an embodiment, of any of the above, the iPSC-derived cell is human. In another aspect, the invention provides a method of enhancing self organization of adipose-derived stem cells (ADSCs) for generating a pancreatic islet or pancreatic organoid, comprising culturing ADSCs in medium comprising Wnt5a protein. In an embodiment, the ADSCs are cultured in a 3-dimensional matrix comprising gellan gum. In another embodiment, the Wnt5a protein a recombinant human Wnt5a protein. In another aspect, the invention provides a pancreatic islet organoid, pancreatic organoid, a liver organoid, a heart organoid, or intestinal organoid produced by any of the above-delineated methods and embodiments thereof.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “AFP polypeptide” or “alpha-fetoprotein” is meant a protein or fragment thereof having at least 85% amino acid sequence identity to the sequence provided at NCBI Accession No. NP_001125.1 and having a biological activity of an AFP polypeptide. Exemplary biological activities of an AFP polypeptide include binding to copper, nickel, fatty acids, and bilirubin. The amino acid sequence provided at NCBI Accession No. NP_001125.1 is shown below:

| (SEQ ID NO: 1) | |
|---|---|
| 1 | mkwvesifli fllnftesrt lhrneygias ildsyqctae isladlatif faqfvqeaty |
| 61 | kevskmvkda ltaiekptgd eqssgclenq lpafleelch ekeilekygh sdccsqseeg |
| 121 | rhncflahkk ptpasiplfq vpepvtscea yeedretfmn kfiyeiarrh pflyaptill |
| 181 | waarydkiip scckaenave cfqtkaatvt kelresslln qhacavmknf gtrtfqaitv |
| 241 | tklsqkftkv nfteiqklvl dvahvhehcc rgdvldclqd gekimsyics qqdtlsnkit |
| 301 | eccklttler gqciihaend ekpeglspnl nrflgdrdfn qfssgeknif lasfvheysr |
| 361 | rhpqlaysvi lrvakgygel lekcfqtenp lecqdkgeee lqkyiqesqa lakrscglfq |
| 421 | klgeyylqna flvaytkkap qltsselmai trkmaataat ccqlsedkll acgegaadii |
| 481 | ighlcirhem tpvnpgvgqc ctssyanrrp cfsslvvdet yvppafsddk fifhkdlcqa |
| 541 | qgvalgtmkg eflinlvkqk pqiteeqlea viadfsglle kccqgqeqev cfaeegqkli |
| 601 | sktraalgv |

By “AFP polynucleotide” is meant a polynucleotide encoding a AFP polypeptide or fragment thereof. An exemplary AFP polynucleotide sequence is provided at NCBI Ref: NM_001134.2. The sequence provided at NCBI Ref: NM_001134.2 is reproduced below:

| (SEQ ID NO: 2) | |
|---|---|
| 1 | atattgtgct tccaccactg ccaataacaa aataactagc aaccatgaag tgggtggaat |
| 61 | caattttttt aattttccta ctaaatttta ctgaatccag aacactgcat agaaatgaat |
| 121 | atggaatagc ttccatattg gattcttacc aatgtactgc agagataagt ttagctgacc |
| 181 | tggctaccat attttttgcc cagtttgttc aagaagccac ttacaaggaa gtaagcaaaa |
| 241 | tggtgaaaga tgcattgact gcaattgaga aacccactgg agatgaacag tcttcagggt |
| 301 | gtttagaaaa ccagctacct gcctttctgg aagaactttg ccatgagaaa gaaattttgg |
| 361 | agaagtacgg acattcagac tgctgcagcc aaagtgaaga gggaagacat aactgttttc |
| 421 | ttgcacacaa aaagcccact ccagcatcga tcccactttt ccaagttcca gaacctgtca |
| 481 | caagctgtga agcatatgaa gaagacaggg agacattcat gaacaaattc atttatgaga |
| 541 | tagcaagaag gcatcccttc ctgtatgcac ctacaattct tctttgggct gctcgctatg |
| 601 | acaaaataat tccatcttgc tgcaaagctg aaaatgcagt tgaatgcttc caaacaaagg |
| 661 | cagcaacagt tacaaaagaa ttaagagaaa gcagcttgtt aaatcaacat gcatgtgcag |
| 721 | taatgaaaaa ttttgggacc cgaactttcc aagccataac tgttactaaa ctgagtcaga |
| 781 | agtttaccaa agttaatttt actgaaatcc agaaactagt cctggatgtg gcccatgtac |
| 841 | atgagcactg ttgcagagga gatgtgctgg attgtctgca ggatggggaa aaaatcatgt |
| 901 | cctacatatg ttctcaacaa gacactctgt caaacaaaat aacagaatgc tgcaaactga |
| 961 | ccacgctgga acgtggtcaa tgtataattc atgcagaaaa tgatgaaaaa cctgaaggtc |
| 1021 | tatctccaaa tctaaacagg tttttaggag atagagattt taaccaattt tcttcagggg |
| 1081 | aaaaaaatat cttcttggca agttttgttc atgaatattc aagaagacat cctcagcttg |
| 1141 | ctgtctcagt aattctaaga gttgctaaag gataccagga gttattggag aagtgtttcc |
| 1201 | agactgaaaa ccctcttgaa tgccaagata aaggagaaga agaattacag aaatacatcc |
| 1261 | aggagagcca agcattggca aagcgaagct gcggcctctt ccagaaacta ggagaatatt |
| 1321 | acttacaaaa tgcgtttctc gttgcttaca caaagaaagc cccccagctg acctcgtcgg |
| 1381 | agctgatggc catcaccaga aaaatggcag ccacagcagc cacttgttgc caactcagtg |
| 1441 | aggacaaact attggcctgt ggcgagggag cggctgacat tattatcgga cacttatgta |
| 1501 | tcagacatga aatgactcca gtaaaccctg gtgttggcca gtgctgcact tcttcatatg |
| 1561 | ccaacaggag gccatgcttc agcagcttgg tggtggatga aacatatgtc cctcctgcat |
| 1621 | tctctgatga caagttcatt ttccataagg atctgtgcca agctcagggt gtagcgctgc |
| 1681 | aaacgatgaa gcaagagttt ctcattaacc ttgtgaagca aaagccacaa ataacagagg |
| 1741 | aacaacttga ggctgtcatt gcagatttct caggcctgtt ggagaaatgc tgccaaggcc |
| 1801 | aggaacagga agtctgcttt gctgaagagg gacaaaaact gatttcaaaa actcgtgctg |
| 1861 | ctttgggagt ttaaattact tcaggggaag agaagacaaa acgagtcttt cattcggtgt |
| 1921 | gaacttttct ctttaatttt aactgattta acactttttg tgaattaatg aaatgataaa |
| 1981 | gacttttatg tgagatttcc ttatcacaga aataaaatat ctccaaatgt ttccttttca |
| 2041 | aaaaaaaaaa aaaaaaa |

By “ALB polypeptide” or “albumin” is meant a protein or fragment thereof having at least 85% amino acid sequence identity to the sequence provided at NCBI Accession No. NP_000468.1 and having a biological activity of ALB polypeptide. Exemplary biological activities of ALB polypeptide include binding to fatty acids, calcium ions, sodium ions, potassium ions, hormones, and bilirubin; stabilization of extracellular fluid volume; and, transport of plasma zinc. The amino acid sequence provided at NCBI Accession No. NP_000468.1 is shown below:

| (SEQ ID NO: 3) | |
|---|---|
| 1 | mkwvtfisll flfssaysrg vfrrdahkse vahrfkdlge enfkalvlia faqylqqcpf |
| 61 | edhvklvnev tefaktcvad esaencdksl htlfgdklct vatlretyge madccakqep |
| 121 | ernecflqhk ddnpnlprlv rpevdvmcta fhdneetflk kylyeiarrh pyfyapellf |
| 181 | fakrykaaft eccqaadkaa cllpkldelr degkassakq rlkcaslqkf gerafkawav |
| 241 | arlsqrfpka efaevsklvt dltkvhtecc hgdllecadd radlakyice nqdsissklk |
| 301 | eccekpllek shciaevend empadlpsla adfveskdvc knyaeakdvf lgmflyeyar |
| 361 | rhpdysvvll lrlaktyett lekccaaadp hecyakvfde fkplveepqn likqncelfe |
| 421 | qlgeykfqna llvrytkkvp qvstptlvev srnlgkvgsk cckhpeakrm pcaedylsvv |
| 481 | lnqlcvlhek tpvsdrvtkc cteslvnrrp cfsalevdet yvpkefnaet ftfhadictl |
| 541 | sekergikkg talvelvkhk pkatkeqlka vmddfaafve kcckaddket cfaeegkklv |
| 601 | aasqaalgl |

By “ALB polynucleotide” is meant a polynucleotide encoding a ALB polypeptide or fragment thereof. An exemplary AFP polynucleotide sequence is provided at NCBI Ref: NM_000477.5. The sequence provided at NCBI Ref: NM_000477.5 is reproduced below:

| (SEQ ID NO: 4) | |
|---|---|
| 1 | agtatattag tgctaatttc cctccgtttg tcctagcttt tctcttctgt caaccccaca |
| 61 | cgcctttggc acaatgaagt gggtaacctt tatttccctt ctttttctct ttagctcggc |
| 121 | ttattccagg ggtgtgtttc gtcgagatgc acacaagagt gaggttgctc atcggtttaa |
| 181 | agatttggga gaagaaaatt tcaaagcctt ggtgttgatt gcctttgctc agtatcttca |
| 241 | gcagtgtcca tttgaagatc atgtaaaatt agtgaatgaa gtaactgaat ttgcaaaaac |
| 301 | atgtgttgct gatgagtcag ctgaaaattg tgacaaatca cttcataccc tttttggaga |
| 361 | caaattatgc acagttgcaa ctcttcgtga aacctatggt gaaatggctg actgctgtgc |
| 421 | aaaacaagaa cctgagagaa atgaatgctt cttgcaacac aaagatgaca acccaaacct |
| 481 | cccccgattg gtgagaccag aggttgatgt gatgtgcact gcttttcatg acaatgaaga |
| 541 | gacatttttg aaaaaatact tatatgaaat tgccagaaga catccttact tttatgcccc |
| 601 | ggaactcctt ttctttgcta aaaggtataa agctgctttt acagaatgtt gccaagctgc |
| 661 | tgataaagct gcctgcctgt tgccaaagct cgatgaactt cgggatgaag ggaaggcttc |
| 721 | gtctgccaaa cagagactca agtgtgccag tctccaaaaa tttggagaaa gagctttcaa |
| 781 | agcatgggca gtagctcgcc tgagccagag atttcccaaa gctgagtttg cagaagtttc |
| 841 | caagttagtg acagatctta ccaaagtcca cacggaatgc tgccatggag atctgcttga |
| 901 | atgtgctgat gacagggcgg accttgccaa gtatatctgt gaaaatcaag attcgatctc |
| 961 | cagtaaactg aaggaatgct gtgaaaaacc tctgttggaa aaatcccact gcattgccga |
| 1021 | agtggaaaat gatgagatgc ctgctgactt gccttcatta gctgctgatt ttgttgaaag |
| 1081 | taaggatgtt tgcaaaaact atgctgaggc aaaggatgtc ttcctgggca tgtttttgta |
| 1141 | tgaatatgca agaaggcatc ctgattactc tgtcgtgctg ctgctgagac ttgccaagac |
| 1201 | atatgaaacc actctagaga agtgctgtgc cgctgcagat cctcatgaat gctatgccaa |
| 1261 | agtgttcgat gaatttaaac ctcttgtgga agagcctcag aatttaatca aacaaaattg |
| 1321 | tgagcttttt gagcagcttg gagagtacaa attccagaat gcgctattag ttcgttacac |
| 1381 | caagaaagta ccccaagtgt caactccaac tcttgtagag gtctcaagaa acctaggaaa |
| 1441 | agtgggcagc aaatgttgta aacatcctga agcaaaaaga atgccctgtg cagaagacta |
| 1501 | tctatccgtg gtcctgaacc agttatgtgt gttgcatgag aaaacgccag taagtgacag |
| 1561 | agtcaccaaa tgctgcacag aatccttggt gaacaggcga ccatgctttt cagctctgga |
| 1621 | agtcgatgaa acatacgttc ccaaagagtt taatgctgaa acattcacct tccatgcaga |
| 1681 | tatatgcaca ctttctgaga aggagagaca aatcaagaaa caaactgcac ttgttgagct |
| 1741 | cgtgaaacac aagcccaagg caacaaaaga gcaactgaaa gctgttatgg atgatttcgc |
| 1801 | agcttttgta gagaagtgct gcaaggctga cgataaggag acctgctttg ccgaggaggg |
| 1861 | taaaaaactt gttgctgcaa gtcaagctgc cttaggctta taacatcaca tttaaaagca |
| 1921 | tctcagccta ccatgagaat aagagaaaga aaatgaagat caaaagctta ttcatctgtt |
| 1981 | tttctttttc gttggtgtaa agccaacacc ctgtctaaaa aacataaatt tctttaatca |
| 2041 | ttttgcctct tttctctgtg cttcaattaa taaaaaatgg aaagaatcta atagagtggt |
| 2101 | acagcactgt tatttttcaa agatgtgttg ctatcctgaa aattctgtag gttctgtgga |
| 2161 | agttccagtg ttctctctta ttccacttcg gtagaggatt tctagtttct tgtgggctaa |
| 2221 | ttaaataaat cattaatact cttctaaaaa aaaaaaaaaa aaaa |

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “altered” is meant an increase or decrease. An increase is any positive change, e.g., by at least about 5%, 10%, or 20%; by at least about 25%, 50%, 75%, or even by 100%, 200%, 300% or more. A decrease is a negative change, e.g., a decrease by at least about 5%, 10%, or 20%; by at least about 25%, 50%, 75%; or even an increase by 100%, 200%, 300% or more.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “CDX2 polypeptide” is meant a protein or fragment thereof having at least 85% amino acid sequence identity to the sequence provided at NCBI Accession No. NP_001256.3and having transcription factor activity. The amino acid sequence provided at NCBI Accession No. NP_001256.3 is shown below:

| (SEQ ID NO: 5) | |
|---|---|
| 1 | myvsylldkd vsmypssvrh sgglnlapqn fvsppqypdy ggyhvaaaaa aaanldsaqs |
| 61 | pgpswpaayg aplredwngy apggaaaaan avahglnggs paaamgyssp adyhphhhph |
| 121 | hhphhpaaap scasgllgtl npgppgpaat aaaeqlspgg qrrnlcewmr kpaqqslgsq |
| 181 | vktrtkdkyr vvytdhqrle lekefhysry itirrkaela atlglserqv kiwfqnrrak |
| 241 | erkinkkklq qqqqqqppqp pppppqppqp qpgplrsvpe plspvsslqa sysgsvpgvl |
| 301 | gptggvinpt vtq |

By “CDX2 polynucleotide” is meant a polynucleotide encoding a CDX2 polypeptide or fragment thereof. An exemplary CDX2 polynucleotide sequence is provided at NCBI Ref: NM_001265.4. The sequence provided at NCBI Ref: NM_001265.4 is reproduced below:

| (SEQ ID NO: 6) | |
|---|---|
| 1 | ctccaaccat tggtgtctgt gtcattacta atagagtctt gtaaacactc gttaatcacg |
| 61 | gaaggccgcc ggcctggggc tccgcacgcc agcctgtggc gggtcttccc cgcctctgca |
| 121 | gcctagtggg aaggaggtgg gaggaaagaa ggaagaaagg gagggaggga ggaggcaggc |
| 181 | cagagggagg gaccgcctcg gaggcagaag agccgcgagg agccagcgga gcaccgcggg |
| 241 | ctggggcgca gccacccgcc gctcctcgag tcccctcgcc cctttccctt cgtgcccccc |
| 301 | ggcagcctcc agcgtcggtc cccaggcagc atggtgaggt ctgctcccgg accctcgcca |
| 361 | ccatgtacgt gagctacctc ctggacaagg acgtgagcat gtaccctagc tccgtgcgcc |
| 421 | actctggcgg cctcaacctg gcgccgcaga acttcgtcag ccccccgcag tacccggact |
| 481 | acggcggtta ccacgtggcg gccgcagctg cagcggcagc gaacttggac agcgcgcagt |
| 541 | ccccggggcc atcctggccg gcagcgtatg gcgccccact ccgggaggac tggaatggct |
| 601 | acgcgcccgg aggcgccgcg gccgccgcca acgccgtggc tcacggcctc aacggtggct |
| 661 | ccccggccgc agccatgggc tacagcagcc ccgcagacta ccatccgcac caccacccgc |
| 721 | atcaccaccc gcaccacccg gccgccgcgc cttcctgcgc ttctgggctg ctgcaaacgc |
| 781 | tcaaccccgg ccctcctggg cccgccgcca ccgctgccgc cgagcagctg tctcccggcg |
| 841 | gccagcggcg gaacctgtgc gagtggatgc ggaagccggc gcagcagtcc ctcggcagcc |
| 901 | aagtgaaaac caggacgaaa gacaaatatc gagtggtgta cacggaccac cagcggctgg |
| 961 | agctggagaa ggagtttcac tacagtcgct acatcaccat ccggaggaaa gccgagctag |
| 1021 | ccgccacgct ggggctctct gagaggcagg ttaaaatctg gtttcagaac cgcagagcaa |
| 1081 | aggagaggaa aatcaacaag aagaagttgc agcagcaaca gcagcagcag ccaccacagc |
| 1141 | cgcctccgcc gccaccacag cctccccagc ctcagccagg tcctctgaga agtgtcccag |
| 1201 | agcccttgag tccggtgtct tccctgcaag cctcagtgtc tggctctgtc cctggggttc |
| 1261 | tggggccaac tgggggggtg ctaaacccca ccgtcaccca gtgacccacc gggttctgca |
| 1321 | gcggcagagc aattccaggc tgagccatga ggagcgtgga ctctgctaga ctcctcagga |
| 1381 | gagacccctc ccctcccacc cacagccata gacctacaga cctggctctc agaggaaaaa |
| 1441 | tgggagccag gagtaagaca agtgggattt ggggcctcaa gaaatatact ctcccagatt |
| 1501 | tttacttttt cccatctggc tttttctgcc actgaggaga cagaaagcct ccgctgggct |
| 1561 | tcattccgga ctggcagaag cattgcctgg actgaccaca ccaaccaggc cttcatcctc |
| 1621 | ctccccagct cttctcttcc tagatctgca ggctgcacct ctggctagag ccgaggggag |
| 1681 | agagggactc aagggaaagg caagcttgag gccaagatgg ctgctgcctg ctcatggccc |
| 1741 | tcggaggtcc agctgggcct cctgcctccg ggcaggcaag gtttacactg cggaagccaa |
| 1801 | aggcagctaa gatagaaagc tggactgacc aaagactgca gaacccccag gtggcctgcg |
| 1861 | tcttttttct cttcccttcc cagaccagga aaggcttggc tggtgtatgc acagggtgtg |
| 1921 | gtatgagggg gtggttattg gactccaggc ctgaccaggg ggcccgaaca gggacttgtt |
| 1981 | tagagagcct gtcaccagag cttctctggg ctgaatgtat gtcagtgcta taaatgccag |
| 2041 | agccaacctg gacttcctgt cattttcaca atcttggggc tgatgaagaa gggggtgggg |
| 2101 | ggagtttgtg ttgttgttgc tgctgtttgg gttgttggtc tgtgtaacat ccaagccaga |
| 2161 | gtttttaaag ccttctggat ccatgggggg agaagtgata tggtgaaggg aagtggggag |
| 2221 | tatttgaaca cagttgaatt ttttctaaaa agaaaaagag ataaatgagc tttccagatt |
| 2281 | tcagattctg tatttatctt cagattttgt ctgcaactat tttttatttt ttaaagaaat |
| 2341 | gaaatatctt caaaaaaaaa aaaaaaaaaa |

By “CYP3A7 polypeptide” or “cytochrome P450” is meant a protein or fragment thereof having at least 85% amino acid sequence identity to the sequence provided at NCBI Accession No. NP_000756.3 and having monooxygenase activity. The amino acid sequence provided at NCBI Accession No. NP_000756.3 is shown below:

| (SEQ ID NO: 7) | |
|---|---|
| 1 | mdlipnlave twlllavsli llylygtrth glfkklgipg ptplpflgna lsfrkgywtf |
| 61 | dmecykkyrk vwgiydcqqp mlaitdpdmi ktvlvkecys vftnrrpfgp vgfmknaisi |
| 121 | aedeewkrir sllsptftsg klkemvpiia qygdvlvrnl rreaetgkpv tlkhvfgays |
| 181 | mdvitstsfg vsidslnnpq dpfventkkl lrfnpldpfv lsikvfpflt pilealnitv |
| 241 | fprkvisflt ksvkqikegr lketqkhrvd flqlmidsqn skdsethkal sdlelmaqsi |
| 301 | ififagyett ssvlsfiiye lathpdvqqk vqkeidtvlp nkapptydtv lqleyldmvv |
| 361 | netlrlfpva mrlervckkd veingmfipk gvvvmipsyv lhhdpkywte pekflperfs |
| 421 | kknkdnidpy iytpfgsgpr ncigmrfalv nmklalvrvl qnfsfkpcke tqiplklrfg |
| 481 | gllltekpiv lkaesrdetv sga |

By “CYP3A7 polynucleotide” is meant a polynucleotide encoding a CYP3A7 40 polypeptide or fragment thereof. An exemplary AFP polynucleotide sequence is provided at NCBI Ref: NM_000765.4. The sequence provided at NCBI Ref: NM_000765.4 is reproduced below:

| (SEQ ID NO: 8) | |
|---|---|
| 1 | aatcactgct gtgcagggca ggaaagctcc acacacacag cccagcaaac agcagcacgc |
| 61 | tgctgaaaaa aagactcaga ggagagagat aaggaaggaa agtagtgatg gatctcatcc |
| 121 | caaacttggc cgtggaaacc tggcttctcc tggctgtcag cctgatactc ctctatctat |
| 181 | atggaacccg tacacatgga ctttttaaga agcttggaat tccagggccc acacctctgc |
| 241 | cttttttggg aaatgctttg tccttccgta agggctattg gacgtttgac atggaatgtt |
| 301 | ataaaaagta tagaaaagtc tggggtattt atgactgtca acagcctatg ctggctatca |
| 361 | cagatcccga catgatcaaa acagtgctag tgaaagaatg ttattctgtc ttcacaaacc |
| 421 | ggaggccttt cgggccagtg ggatttatga aaaatgccat ctctatagct gaggatgaag |
| 481 | aatggaagag aatacgatca ttgctgtctc caacattcac cagcggaaaa ctcaaggaga |
| 541 | tggtccctat cattgcccag tatggagatg tgttggtgag aaatctgagg cgggaagcag |
| 601 | agacaggcaa gcctgtcacc ttgaaacacg tctttggggc ctacagcatg gatgtgatca |
| 661 | ctagcacatc atttggagtg agcatcgact ctctcaacaa tccacaagac ccctttgtgg |
| 721 | aaaacaccaa gaagctttta agatttaatc cattagatcc attcgttctc tcaataaaag |
| 781 | tctttccatt ccttacccca attcttgaag cattaaatat cactgtgttt ccaagaaaag |
| 841 | ttataagttt tctaacaaaa tctgtaaaac agataaaaga aggtcgcctc aaagagacac |
| 901 | aaaagcaccg agtggatttc cttcagctga tgattgactc tcagaattca aaagactctg |
| 961 | agacccacaa agctctgtct gatctggagc tcatggccca atcaattatc tttatttttg |
| 1021 | ctggctatga aaccacgagc agtgttctct ccttcattat atatgaactg gccactcacc |
| 1081 | ctgatgtcca gcagaaagtg cagaaggaaa ttgatacagt tttacccaat aaggcaccac |
| 1141 | ccacctatga tactgtgcta cagttggagt atcttgacat ggtggtgaat gaaacactca |
| 1201 | gattattccc agttgctatg agacttgaga gggtctgcaa aaaagatgtt gaaatcaatg |
| 1261 | ggatgtttat tcccaaaggg gtggtggtga tgattccaag ctatgttctt catcatgacc |
| 1321 | caaagtactg gacagagcct gagaagttcc tccctgaaag gttcagtaaa aagaacaagg |
| 1381 | acaacataga tccttacata tacacaccct ttggaagtgg acccagaaac tgcattggca |
| 1441 | tgaggtttgc tctcgtgaac atgaaacttg ctctagtcag agtccttcag aacttctcct |
| 1501 | tcaaaccttg taaagaaaca cagatccccc tgaaattacg ctttggagga cttcttctaa |
| 1561 | cagaaaaacc cattgttcta aaggctgagt caagggatga gaccgtaagt ggagcctgat |
| 1621 | ttccctaagg acttctggtt tgctctttaa gaaagctgtg ccccagaaca ccagagacct |
| 1681 | caaattactt tacaaataga accctgaaat gaagacgggc ttcatccaat gtgctgcata |
| 1741 | aataatcagg gattctgtac gtgcattgtg ctctctcatg gtctgtatag agtgttatac |
| 1801 | ttggtaatat agaggagatg accaaatcag tgctggggaa gtagatttgg cttctctgct |
| 1861 | tctcatagga ctatctccac cacccccagt tagcaccatt aactcctcct gagctctgat |
| 1921 | aacataatta acatttctca ataatttcaa ccacaatcat taataaaaat aggaattatt |
| 1981 | ttgatggctc taacagtgac atttatatca tgtgttatat ctgtagtatt ctatagtaag |
| 2041 | ctttatatta agcaaatcaa taaaaacctc tttacaaaag taaaaaaaaa aaaaaaaaa |

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

“Differentiation” refers to the developmental process of lineage commitment. Differentiation can be assayed by measuring an increase in one or more cell specific markers relative to their expression in a corresponding undifferentiated control cell. A “lineage” refers to a pathway of cellular development, in which precursor or “progenitor” cells undergo progressive physiological changes to become a specified cell type having a characteristic function. In some embodiments, the cell type is a beta cell. In some embodiments, the cell type is an alpha cell, delta cell, or duct cell. In some other embodiments, the cell type is a hepatocyte. In still other embodiments, the cell type is a cardiomyocyte. In some embodiments, the cell type is a intestinal cell. Differentiation occurs in stages, whereby cells gradually become more specified until they reach full maturity, which is also referred to as “terminal differentiation.” A “terminally differentiated cell” is a cell that has committed to a specific lineage, and has reached the end stage of differentiation (i.e., a cell that has fully matured). In some embodiments, an induced pluripotent stem cell (iPSC) is differentiated into a beta-like cell, an alpha-like cell, a delta-like cell, or a duct-like cell. In some other embodiments, an induced pluripotent stem cell (iPSC) is differentiated into a hepatocyte, cardiomyocyte, or intestinal cell.

A “de-differentiated cell” is a cell in which the process of differentiation has been, at least to some degree, reversed. De-differentiation can be assayed, for example, by identifying a reduction in the expression of one or more cell specific markers relative to their expression in a corresponding control cell. Alternatively, de-differentiation can be assayed by measuring an increase in one or more markers typically expressed in an embryonic stem cell, a pluripotent or multi-potent cell type, or expressed at an earlier stage of development. In some embodiments, the de-differentiated cell is an induced pluripotent stem cell (iPSC). In certain embodiments, the de-differentiated cell is a human induced pluripotent stem cell (iPSC).

By “disease” is meant any condition or disorder that damages or interferes with the normal function of a cell, tissue, or organ. Examples of diseases include type 1 diabetes, type 2 diabetes, and pancreatic cancer.

By “effective amount” is meant the amount of a therapeutic agent or organoid required to ameliorate the symptoms of a disease in a subject relative to an untreated subject. The effective amount of a therapeutic used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In some embodiments, the therapeutic organoid is a pancreatic islet organoid. In some other embodiments, an effective amount of a pancreatic islet organoid is administered to a subject having type 1 or type 2 diabetes.

By “ESRRG polypeptide” is meant a protein or fragment thereof having at least 85% amino acid sequence identity to the sequence provided at NCBI Accession No. NP_001230448.1 and having nuclear hormone receptor activity. The amino acid sequence provided at NCBI Accession No. NP_001230448.1 is shown below:

| (SEQ ID NO: 9) | |
|---|---|
| 1 | msnkdrhids scssfiktep sspasltdsv nhhspggssd asgsysstmn ghqngldspp |
| 61 | lypsapilgg sgpvrklydd csstivedpq tkceymlnsm pkrlclvcgd iasgyhygva |
| 121 | sceackaffk rtiqgnieys cpatneceit krrrkscqac rfmkclkvgm lkegvrldrv |
| 181 | rggrqkykrr idaenspyln pqlvqpakkp ynkivshllv aepekiyamp dptvpdsdik |
| 241 | altticdlad relvviigwa khipgfstls ladqmsllqs awmeililgv vyrslsfede |
| 301 | lvyaddyimd edqsklagll dlnnailqlv kkyksmklek eefvtlkaia lansdsmhie |
| 361 | dveavqklqd vlhealqdye agqhmedprr agkmlmtlpl lrgtstkavq hfyniklegk |
| 421 | vpmhklflem leakv |

By “ESRRG polynucleotide” is meant a polynucleotide encoding a ESRRG polypeptide or fragment thereof. An exemplary ESRRG polynucleotide sequence is provided at NCBI Ref: NM_001243519.1. The sequence provided at NCBI Ref: NM_001243519.1 is reproduced below:

| (SEQ ID NO: 10) | |
|---|---|
| 1 | aagctccaat cggggcttta agtccttgat taggagagtg tgagagcttt ggtcccaact |
| 61 | ggctgtgcct ataggcttgt cactaggaga acatttgtgt taattgcact gtgctctgtc |
| 121 | aaggaaactt tgatttatag ctggggtgca caaataatgg ttgccggtcg cacatggatt |
| 181 | cggtagaact ttgccttcct gaatcttttt ccctgcacta cgaggaagag tagacttgaa |
| 241 | tgagacctgc ctcatcagtc atgggatcat agtgtcacag atggaaaagc aactatcagc |
| 301 | tgaattgtac tgaactacac acttggctaa ttcatcttat tgctctacac atctaaagga |
| 361 | aggctcattc tgttcttgga gtctagacag catcaggagt tgggctcagt gaacaaaact |
| 421 | ttaatgtcta gagcatttat gagggtttta atgattggaa aatctatcct gagaatgtgg |
| 481 | tcaccatatg tgacagcctt gctttctatc ttgtcttcag tttctggggc ttctctgcag |
| 541 | aatgtcaaac aaagatcgac acattgattc cagctgttcg tccttcatca agacggaacc |
| 601 | ttccagccca gcctccctga cggacagcgt caaccaccac agccctggtg gctcttcaga |
| 661 | cgccagtggg agctacagtt caaccatgaa tggccatcag aacggacttg actcgccacc |
| 721 | tctctaccct tctgctccta tcctgggagg tagtgggcct gtcaggaaac tgtatgatga |
| 781 | ctgctccagc accattgttg aagatcccca gaccaagtgt gaatacatgc tcaactcgat |
| 841 | gcccaagaga ctgtgtttag tgtgtggtga catcgcttct gggtaccact atggggtagc |
| 901 | atcatgtgaa gcctgcaagg cattcttcaa gaggacaatt caaggcaata tagaatacag |
| 961 | ctgccctgcc acgaatgaat gtgaaatcac aaagcgcaga cgtaaatcct gccaggcttg |
| 1021 | ccgcttcatg aagtgtttaa aagtgggcat gctgaaagaa ggggtgcgtc ttgacagagt |
| 1081 | acgtggaggt cggcagaagt acaagcgcag gatagatgcg gagaacagcc catacctgaa |
| 1141 | ccctcagctg gttcagccag ccaaaaagcc atataacaag attgtctcac atttgttggt |
| 1201 | ggctgaaccg gagaagatct atgccatgcc tgaccctact gtccccgaca gtgacatcaa |
| 1261 | agccctcact acactgtgtg acttggccga ccgagagttg gtggttatca ttggatgggc |
| 1321 | gaagcatatt ccaggcttct ccacgctgtc cctggcggac cagatgagcc ttctgcagag |
| 1381 | tgcttggatg gaaattttga tccttggtgt cgtataccgg tctctttcgt ttgaggatga |
| 1441 | acttgtctat gcagacgatt atataatgga cgaagaccag tccaaattag caggccttct |
| 1501 | tgatctaaat aatgctatcc tgcagctggt aaagaaatac aagagcatga agctggaaaa |
| 1561 | agaagaattt gtcaccctca aagctatagc tcttgctaat tcagactcca tgcacataga |
| 1621 | agatgttgaa gccgttcaga agcttcagga tgtcttacat gaagcgctgc aggattatga |
| 1681 | agctggccag cacatggaag accctcgtcg agctggcaag atgctgatga cactgccact |
| 1741 | cctgaggcag acctctacca aggccgtgca gcatttctac aacatcaaac tagaaggcaa |
| 1801 | agtcccaatg cacaaacttt ttttggaaat gttggaggcc aaggtctgac taaaagctcc |
| 1861 | ctgggccttc ccatccttca tgttgaaaaa gggaaaataa acccaagagt gatgtcgaag |
| 1921 | aaacttagag tttagttaac aacatcaaaa atcaacagac tgcactgata atttagcagc |
| 1981 | aagactatga agcagctttc agattcctcc ataggttcct gatgagtttc tttctacttt |
| 2041 | ctccatcatc ttctttcctc tttcttccca catttctctt tctctttatt ttttctcctt |
| 2101 | ttcttctttc acctccctta tttctttgct tctttcattc ctagttccca ttctccttta |
| 2161 | ttttcttccc gtctgcctgc cttctttctt ttctttacct actctcattc ctctcttttc |
| 2221 | tcatccttcc ccttttttct aaatttgaaa tagctttagt ttaaaaaaaa atcctccctt |
| 2281 | ccccctttcc tttccctttc tttccttttt ccctttcctt ttccctttcc tttcctttcc |
| 2341 | tcttgacctt ctttccatct ttctttttct tccttctgct gctgaacttt taaaagaggt |
| 2401 | ctctaactga agagagatgg aagccagccc tgccaaagga tggagatcca taatatggat |
| 2461 | gccagtgaac ttattgtgaa ccatactgtc cccaatgact aaggaatcaa agagagagaa |
| 2521 | ccaacgttcc taaaagtaca gtgcaacata tacaaattga ctgagtgcag tattagattt |
| 2581 | catgggagca gcctctaatt agacaactta agcaacgttg catcggctgc ttcttatcat |
| 2641 | tgcttttcca tctagatcag ttacagccat ttgattcctt aattgttttt tcaagtcttc |
| 2701 | caggtatttg ttagtttagc tactatgtaa ctttttcagg gaatagttta agctttattc |
| 2761 | attcatgcaa tactaaagag aaataagaat actgcaattt tgtgctggct ttgaacaatt |
| 2821 | acgaacaata atgaaggaca aatgaatcct gaaggaagat ttttaaaaat gttttgtttc |
| 2881 | ttcttacaaa tggagatttt tttgtaccag ctttaccact tttcagccat ttattaatat |
| 2941 | gggaatttaa cttactcaag caatagttga agggaaggtg catattatca cggatgcaat |
| 3001 | ttatgttgtg tgccagtctg gtcccaaaca tcaatttctt aacatgagct ccagtttacc |
| 3061 | taaatgttca ctgacacaaa ggatgagatt acacctacag tgactctgag tagtcacata |
| 3121 | tataagcact gcacatgaga tatagatccg tagaattgtc aggagtgcac ctctctactt |
| 3181 | gggaggtaca attgccatat gatttctagc tgccatggtg gttaggaatg tgatactgcc |
| 3241 | tgtttgcaaa gtcacagacc ttgcctcaga aggagctgtg agccagtatt catttaagag |
| 3301 | gcaataaggc aaatgccaga attaaaaaaa aaaatcatca aagacagaaa atgcctgacc |
| 3361 | aaattctaaa acctaatcca tataagttta ttcatttagg aatgttcgtt taaattaatc |
| 3421 | tgcagttttt accaagagct aagccaatat atgtgctttt caaccagtat tgtcacagca |
| 3481 | tgaaagtcaa gtcaggttcc agactgttaa gaggtgtaat ctaatgaaga aatcaattag |
| 3541 | atgccccgaa atctacagtc gctgaataac caataaacag taacctccat caaatgctat |
| 3601 | accaatggac cagtgttagt agctgctccc tgtattatgt gaacagtctt attctatgta |
| 3661 | cacagatgta attaaaattg taatcctaac aaacaaaaga aatgtagttc agcttttcaa |
| 3721 | tgtttcatgt ttgctgtgct tttctgaatt ttatgttgca ttcaaagact gttgtcttgt |
| 3781 | tcttgtggtg tttggattct tgtggtgtgt gcttttagac acagggtaga attagagaca |
| 3841 | atattggatg tacaattcct caggagacta cagtagtata ttctattcct taccagtaat |
| 3901 | aaggttcttc ctaataataa ttaagagatt gaaactccaa acaagtattc attatgaaca |
| 3961 | gatacacatc aaaatcataa taatattttc aaaacaagga ataatttctc taatggttta |
| 4021 | ttatagaata ccaatgtata gcttagaaat aaaactttga atatttcaag aatatagata |
| 4081 | agtctaattt ttaaatgctg tatatatggc tttcactcaa tcatctctca gatgttgtta |
| 4141 | ttaactcgct ctgtgttgtt gcaaaacttt ttggtgcaga ttcgtttcca aaactattgc |
| 4201 | tactttgtgt gctttaaaca aaataccttg ggttgatgaa acatcaaccc agtgctagga |
| 4261 | atactgtgta tctatcatta gctatatggg actatattgt agattgtggt ttctcagtag |
| 4321 | agaagtgact gtagtgtgat tctagataaa tcatcattag caattcattc agatggtcaa |
| 4381 | taacttgaaa tttatagctg tgataggagt tcagaaattg gcacatccct ttaaaaataa |
| 4441 | caacagaaaa tacaactcct gggaaaaaag gtgctgattc tataagatta tttatatatg |
| 4501 | taagtgttta aaaagattat tttccagaaa gtttgtgcag ggtttaagtt gctactattc |
| 4561 | aactacacta tatataaata aaatatatac aatatataca ttgttttcac tgtatcacat |
| 4621 | taaagtactt gggcttcaga agtaagagcc aaccaactga aaacctgaga tggagatatg |
| 4681 | ttcaaagaat gagatacaat tttttagttt tcagtttaag taactctcag cattacaaaa |
| 4741 | gagtaagtat ctcacaaata ggaaataaaa ctaaaacgtg gatttaaaaa gaactgcacg |
| 4801 | ggctttaggg taaatgctca tcttaaacct cactagaggg aagtcttctc aagtttcaag |
| 4861 | caagaccatt tacttaatgt gaagttttgg aaagttataa aggtgtatgt tttagccata |
| 4921 | tgattttaat tttaattttg cttcttttag gttcgttctt atttaaagca atatgattgt |
| 4981 | gtgactcctt gtagttacac ttgtgtttca atcagatcag attgttgtat ttattccact |
| 5041 | attttgcatt taaatgataa cataaaagat ataaaaaatt taaaactgct atttttctta |
| 5101 | tagaagagaa aatgggtgtt ggtgattgta ttttaattat ttaagcgtct ctgtttacct |
| 5161 | gcctaggaaa acattttatg gcagtcttat gtgcaaagat cgtaaaagga caaaaaattt |
| 5221 | aaactgctta taataatcca ggagttgcat tatagccagt agtaaaaata ataataataa |
| 5281 | taataaaacc atgtctatag ctgtagatgg gcttcacatc tgtaaagcaa tcaattgtat |
| 5341 | atttttgtga tgtgtaccat actgtgtgct ccagcaaatg tccatttgtg taaatgtatt |
| 5401 | tattttatat tgtatatatt gttaaatgca aaaaggagat atgattctgt aactccaatc |
| 5461 | agttcagatg tgtaactcaa attattatgc ctttcaggat gatggtagag caatattaaa |
| 5521 | caagcttcca cttttgactg ctaaaaaaaa aaaaaaaaa |

As used herein, “endocrine” refers to secretion of an agent (e.g., a hormone) into a bloodstream. “Exocrine” refers to secretion of an agent into an epithelial surface by way of a duct.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “FOXA2 polypeptide” is meant a protein or fragment thereof having at least 85% amino acid sequence identity to the sequence provided at NCBI Accession No. NP_068556.2 and having transcription factor activity. The amino acid sequence provided at NCBI Accession No. NP_068556.2 is shown below:

| (SEQ ID NO: 11) | |
|---|---|
| 1 | mhsassmlga vkmeghepsd wssyyaepeg yssvsnmnag lgmngmntym smsaaamgsg |
| 61 | sgnmsagsmn mssyvgagms pslagmspga gamagmggsa gaagvagmgp hlspslsplg |
| 121 | gqaagamggl apyanmnsms pmygqaglsr ardpktyrrs ythakppysy islitmaigq |
| 181 | spnkmltlse iyqwimdlfp fyrqnqqrwq nsirhslsfn dcflkvprsp dkpgkgsfwt |
| 241 | lhpdsgnmfe ngcylrrqkr fkcekqlalk eaagaagsgk kaaagagasq aqlgeaagpa |
| 301 | setpagtesp hssaspcqeh krgglgelkg tpaaalsppe papspgqqqq aaahllgpph |
| 361 | hpglppeahl kpehhyafnh pfsinnlmss eqqhhhshhh hqphkmdlka yeqvmhypgy |
| 421 | gspmpgslam gpvtnktgld asplaadtsy yqgvysrpim nss |

By “FOXA2 polynucleotide” is meant a polynucleotide encoding a FOXA2 polypeptide or fragment thereof. An exemplary FOXA2 polynucleotide sequence is provided at NCBI Ref: NM_021784.4. The sequence provided at NCBI Ref: NM_021784.4 is reproduced below:

| (SEQ ID NO: 12) | |
|---|---|
| 1 | cccgcccact tccaactacc gcctccggcc tgcccaggga gagagaggga gtggagccca |
| 61 | gggagaggga gcgcgagaga gggagggagg aggggacggt gctttggctg actttttttt |
| 121 | aaaagagggt gggggtgggg ggtgattgct ggtcgtttgt tgtggctgtt aaattttaaa |
| 181 | ctgccatgca ctcggcttcc agtatgctgg gagcggtgaa gatggaaggg cacgagccgt |
| 241 | ccgactggag cagctactat gcagagcccg agggctactc ctccgtgagc aacatgaacg |
| 301 | ccggcctggg gatgaacggc atgaacacgt acatgagcat gtcggcggcc gccatgggca |
| 361 | gcggctcggg caacatgagc gcgggctcca tgaacatgtc gtcgtacgtg ggcgctggca |
| 421 | tgagcccgtc cctggcgggg atgtcccccg gcgcgggcgc catggcgggc atgggcggct |
| 481 | cggccggggc ggccggcgtg gcgggcatgg ggccgcactt gagtcccagc ctgagcccgc |
| 541 | tcggggggca ggcggccggg gccatgggcg gcctggcccc ctacgccaac atgaactcca |
| 601 | tgagccccat gtacgggcag gcgggcctga gccgcgcccg cgaccccaag acctacaggc |
| 661 | gcagctacac gcacgcaaag ccgccctact cgtacatctc gctcatcacc atggccatcc |
| 721 | agcagagccc caacaagatg ctgacgctga gcgagatcta ccagtggatc atggacctct |
| 781 | tccccttcta ccggcagaac cagcagcgct ggcagaactc catccgccac tcgctctcct |
| 841 | tcaacgactg tttcctgaag gtgccccgct cgcccgacaa gcccggcaag ggctccttct |
| 901 | ggaccctgca ccctgactcg ggcaacatgt tcgagaacgg ctgctacctg cgccgccaga |
| 961 | agcgcttcaa gtgcgagaag cagctggcgc tgaaggaggc cgcaggcgcc gccggcagcg |
| 1021 | gcaagaaggc ggccgccgga gcccaggcct cacaggctca actcggggag gccgccgggc |
| 1081 | cggcctccga gactccggcg ggcaccgagt cgcctcactc gagcgcctcc ccgtgccagg |
| 1141 | agcacaagcg agggggcctg ggagagctga aggggacgcc ggctgcggcg ctgagccccc |
| 1201 | cagagccggc gccctctccc gggcagcagc agcaggccgc ggcccacctg ctgggcccgc |
| 1261 | cccaccaccc gggcctgccg cctgaggccc acctgaagcc ggaacaccac tacgccttca |
| 1321 | accacccgtt ctccatcaac aacctcatgt cctcggagca gcagcaccac cacagccacc |
| 1381 | accaccacca accccacaaa atggacctca aggcctacga acaggtgatg cactaccccg |
| 1441 | gctacggttc ccccatgcct ggcagcttgg ccatgggccc ggtcacgaac aaaacgggcc |
| 1501 | tggacgcctc gcccctggcc gcagatacct cctactacca gggggtgtac tcccggccca |
| 1561 | ttatgaactc ctcttaagaa gacgacggct tcaggcccgg ctaactctgg caccccggat |
| 1621 | cgaggacaag tgagagagca agtgggggtc gagactttgg ggagacggtg ttgcagagac |
| 1681 | gcaagggaga agaaatccat aacaccccca ccccaacacc cccaagacag cagtcttctt |
| 1741 | cacccgctgc agccgttccg tcccaaacag agggccacac agatacccca cgttctatat |
| 1801 | aaggaggaaa acgggaaaga atataaagtt aaaaaaaagc ctccggtttc cactactgtg |
| 1861 | tagactcctg cttcttcaag cacctgcaga ttctgatttt tttgttgttg ttgttctcct |
| 1921 | ccattgctgt tgttgcaggg aagtcttact taaaaaaaaa aaaaaatttt gtgagtgact |
| 1981 | cggtgtaaaa ccatgtagtt ttaacagaac cagagggttg tactattgtt taaaaacagg |
| 2041 | aaaaaaaata atgtaagggt ctgttgtaaa tgaccaagaa aaagaaaaaa aaagcattcc |
| 2101 | caatcttgac acggtgaaat ccaggtctcg ggtccgatta atttatggtt tctgcgtgct |
| 2161 | ttatttatgg cttataaatg tgtattctgg ctgcaagggc cagagttcca caaatctata |
| 2221 | ttaaagtgtt atacccggtt ttatcccttg aatcttttct tccagatttt tcttttcttt |
| 2281 | acttggctta caaaatatac aggcttggaa attatttcaa gaaggaggga gggataccct |
| 2341 | gtctggttgc aggttgtatt ttattttggc ccagggagtg ttgctgtttt cccaacattt |
| 2401 | tattaataaa attttcagac ataaaaaa |

By “GATA6 polypeptide” is meant a protein or fragment thereof having at least 85% amino acid sequence identity to the sequence provided at NCBI Accession No. NP_005248.2 and having transcription factor activity. The amino acid sequence provided at NCBI Accession No. NP_005248.2 is shown below:

| (SEQ ID NO: 13) | |
|---|---|
| 1 | maltdggwcl pkrfgaagad asdsrafpar epstppspis ssssscsrgg ergpggasnc |
| 61 | gtpqldteaa agpparslll ssyashpfga phgpsapgva gpggnlsswe dlllftdldq |
| 121 | aataskllws srgaklspfa peqpeemyqt laalssqgpa aydgapggfv hsaaaaaaaa |
| 181 | aaasspvyvp ttrvgsmlpg lpyhlqgsgs gpanhaggag ahpgwpqasa dsppygsggg |
| 241 | aagggaagpg gagsaaahvs arfpyspspp mangaarepg gyaaagsgga ggvsgggssl |
| 301 | aamggrepqy sslsaarpln gtyhhhhhhh hhhpspyspy vgapltpawp agpfetpvlh |
| 361 | slqsragapl pvprgpsadl ledlsesrec vncgsiqtpl wrrdgtghyl cnacglyskm |
| 421 | nglsrplikp qkrvpssrrl glscanchtt tttlwrrnae gepvcnacgl ymklhgvprp |
| 481 | lamkkegiqt rkrkpknink sktcsgnsnn sipmtptsts snsddcsknt spttqptasg |
| 541 | agapvmtgag estnpensel kysgqdglyi gvslaspaev tssvrpdswc alala |

By “GATA6 polynucleotide” is meant a polynucleotide encoding a GATA6 polypeptide or fragment thereof. An exemplary KCNK3 polynucleotide sequence is provided at NCBI Ref: NM_005257.5. The sequence provided at NCBI Ref: NM_005257.5 is reproduced below:

| (SEQ ID NO: 14) | |
|---|---|
| 1 | agttccgacc cacagcctgg cacccttcgg cgagcgctgt ttgtttaggg ctcggtgagt |
| 61 | ccaatcagga gcccaggctg cagttttccg gcagagcagt aagaggcgcc tcctctctcc |
| 121 | tttttattca ccagcagcgc ggcgcagacc ccggactcgc gctcgcccgc tggcgccctc |
| 181 | ggcttctctc cgcgcctggg agcaccctcc gccgcggccg ttctccatgc gcagcgcccg |
| 241 | cccgaggagc tagacgtcag cttggagcgg cgccggaccg tggatggcct tgactgacgg |
| 301 | cggctggtgc ttgccgaagc gcttcggggc cgcgggtgcg gacgccagcg actccagagc |
| 361 | ctttccagcg cgggagccct ccacgccgcc ttcccccatc tcttcctcgt cctcctcctg |
| 421 | ctcccggggc ggagagcggg gccccggcgg cgccagcaac tgcgggacgc ctcagctcga |
| 481 | cacggaggcg gcggccggac ccccggcccg ctcgctgctg ctcagttcct acgcttcgca |
| 541 | tcccttcggg gctccccacg gaccttcggc gcctggggtc gcgggccccg ggggcaacct |
| 601 | gtcgagctgg gaggacttgc tgctgttcac tgacctcgac caagccgcga ccgccagcaa |
| 661 | gctgctgtgg tccagccgcg gcgccaagct gagccccttc gcacccgagc agccggagga |
| 721 | gatgtaccag accctcgccg ctctctccag ccagggtccg gccgcctacg acggcgcgcc |
| 781 | cggcggcttc gtgcactctg cggccgcggc ggcagcagcc gcggcggcgg ccagctcccc |
| 841 | ggtctacgtg cccaccaccc gcgtgggttc catgctgccc ggcctaccgt accacctgca |
| 901 | ggggtcgggc agtgggccag ccaaccacgc gggcggcgcg ggcgcgcacc ccggctggcc |
| 961 | tcaggcctcg gccgacagcc ctccatacgg cagcggaggc ggcgcggctg gcggcggggc |
| 1021 | cgcggggcct ggcggcgctg gctcagccgc ggcgcacgtc tcggcgcgct tcccctactc |
| 1081 | tcccagcccg cccatggcca acggcgccgc gcgggagccg ggaggctacg cggcggcggg |
| 1141 | cagtgggggc gcgggaggcg tgagcggcgg cggcagtagc ctggcggcca tgggcggccg |
| 1201 | cgagccccag tacagctcgc tgtcggccgc gcggccgctg aacgggacgt accaccacca |
| 1261 | ccaccaccac caccaccacc atccgagccc ctactcgccc tacgtggggg cgccactgac |
| 1321 | gcctgcctgg cccgccggac ccttcgagac cccggtgctg cacagcctgc agagccgcgc |
| 1381 | cggagccccg ctcccggtgc cccggggtcc cagtgcagac ctgctggagg acctgtccga |
| 1441 | gagccgcgag tgcgtgaact gcggctccat ccagacgccg ctgtggcggc gggacggcac |
| 1501 | cggccactac ctgtgcaacg cctgcgggct ctacagcaag atgaacggcc tcagccggcc |
| 1561 | cctcatcaag ccgcagaagc gcgtgccttc atcacggcgg cttggattgt cctgtgccaa |
| 1621 | ctgtcacacc acaactacca ccttatggcg cagaaacgcc gagggtgaac ccgtgtgcaa |
| 1681 | tgcttgtgga ctctacatga aactccatgg ggtgcccaga ccacttgcta tgaaaaaaga |
| 1741 | gggaattcaa accaggaaac gaaaacctaa gaacataaat aaatcaaaga cttgctctgg |
| 1801 | taatagcaat aattccattc ccatgactcc aacttccacc tcttctaact cagatgattg |
| 1861 | cagcaaaaat acttccccca caacacaacc tacagcctca ggggcgggtg ccccggtgat |
| 1921 | gactggtgcg ggagagagca ccaatcccga gaacagcgag ctcaagtatt cgggtcaaga |
| 1981 | tgggctctac ataggcgtca gtctcgcctc gccggccgaa gtcacgtcct ccgtgcgacc |
| 2041 | ggattcctgg tgcgccctgg ccctggcctg agcccacgcc gccaggaggc agggagggct |
| 2101 | ccgccgcggg cctcactcca ctcgtgtctg cttttgtgca gcggtccaga cagtggcgac |
| 2161 | tgcgctgaca gaacgtgatt ctcgtgcctt tattttgaaa gagatgtttt tcccaagagg |
| 2221 | cttgctgaaa gagtgagaga agatggaagg gaagggccag tgcaactggg cgcttgggcc |
| 2281 | actccagcca gcccgcctcc ggggcggacc ctgctccact tccagaagcc aggactagga |
| 2341 | cctgggcctt gcctgctatg gaatattgag agagattttt taaaaaagat tttgcatttt |
| 2401 | gtccaaaatc atgtgcttct tctgatcaat tttggttgtt ccagaatttc ttcatacctt |
| 2461 | ttccacatcc agatttcatg tgcgttcatg gagaagatca cttgaggcca tttggtacac |
| 2521 | atctctggag gctgagtcgg ttcatgaggt ctcttatcaa aaatattact cagtttgcaa |
| 2581 | gactgcattg taactttaac atacactgtg actgacgttt ctcaaagttc atattgtgtg |
| 2641 | gctgatctga agtcagtcgg aatttgtaaa cagggtagca aacaagatat ttttcttcca |
| 2701 | tgtatacaat aattttttta aaaagtgcaa tttgcgttgc agcaatcagt gttaaatcat |
| 2761 | ttgcataaga tttaacagca ttttttataa tgaatgtaaa cattttaact taatggtact |
| 2821 | taaaataatt taaaagaaaa atgttaactt agacattctt atgcttcttt tacaactaca |
| 2881 | tcccatttta tatttccaat tgttaaagaa aaatatttca agaacaaatc ttctctcagg |
| 2941 | aaaattgcct ttctctattt gttaagaatt tttatacaag aacaccaata tacccccttt |
| 3001 | attttactgt ggaatatgtg ctggaaaaat tgcaacaaca ctttactacc taacggatag |
| 3061 | catttgtaaa tactctaggt atctgtaaac actctgatga agtctgtata gtgtgactaa |
| 3121 | cccacaggca ggttggttta cattaatttt tttttttgaa tgggatgtcc tatggaaacc |
| 3181 | tatttcacca gagttttaaa aataaaaagg gtattgtttt gtcttctgta cagtgagttc |
| 3241 | cttccctttt caaagctttc tttttatgct gtatgtgact atagatattc atataaaaca |
| 3301 | agtgcacgtg aagtttgcaa aatgctttaa ggccttcctt tcaaagcata gtccttttgg |
| 3361 | agccgttttg taccttttat accttggctt atttgaagtt gacacatggg gttagttact |
| 3421 | actctccatg tgcattgggg acagttttta taagtgggaa ggactcagta ttattatatt |
| 3481 | tgagatgata agcattttgt ttgggaacaa tgcttaaaaa tattccagaa agttcagatt |
| 3541 | ttttttcttt gtgaatgaaa tatattctgg cccacgaaca gggcgatttc ctttcagttt |
| 3601 | tttccttttg caacgtgcct tgaagtctca aagctcacct gaggttgcag acgttacccc |
| 3661 | caacagaaga taggtagaaa tgattccagt ggcctctttg tattttcttc attgttgagt |
| 3721 | agatttcagg aaatcaggag gtgtttcaca atacagaatg atggccttta actgtgaaaa |
| 3781 | aaaaa |

By “gellan gum” is meant a polysaccharide having a straight chain with a repeating unit that has any one of the following molecular structures:

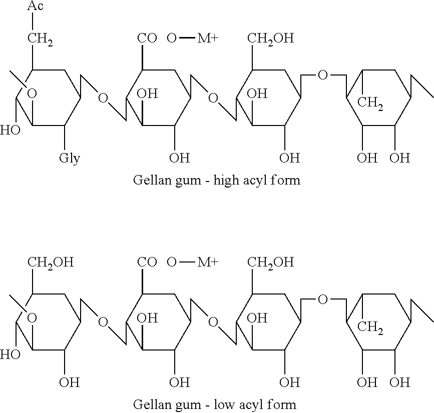

In the foregoing structures, “Ac” refers to an acetate group and “Gly” refers to a glycerate group and “M+” is a monovalent cation. In some embodiments, the gellan gum is KELCOGEL® gellan gum.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immunosuppressive agent” or “immunosuppressant” is meant an agent that inhibits or prevents an immune reaction, such as rejection, of a transplanted organ or organoid in a subject. Examples of immunosuppressants include, but are not limited to, basilizimab, antithymocyte globulin, alemtuzumab, prednisone, azathioprine, mycophenolate, cyclosporine, sirolimus, methotrexate, interferon, and tacrolimus.

By “induced pluripotent stem cell” or “iPSC” is meant a differentiated somatic cell that acquires pluripotency by the exogenous expression of one or more transcription factors in the cell. An “iPSC-derived cell” is a cell derived from an induced pluripotent stem cell. An “iPSC-derived beta-like cell,” “iPSC-derived alpha-like cell,” “iPSC-derived delta-like cell,” or “iPSC-derived duct-like cell” is a cell derived from an induced pluripotent stem cell and has characteristics of a beta cell, alpha cell, delta cell, or duct cell, respectively.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. The preparation can be at least 75%, at least 90%, and at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “KCNK3 polypeptide” is meant a protein or fragment thereof having at least 85% amino acid sequence identity to the sequence provided at NCBI Accession No. NP_002237.1 and having potassium channel activity. The amino acid sequence provided at NCBI Accession No. NP_002237.1 is shown below:

| (SEQ ID NO: 15) | |
|---|---|
| 1 | mkrqnvrtla livctftyll vgaavfdale sepelierqr lelrqqelra rynlsqggye |
| 61 | elervvlrlk phkagvqwrf agsfyfaitv ittigyghaa pstdggkvfc mfyallgipl |
| 121 | tlvmfqslge rintlvryll hrakkglgmr radvsmanmv ligffscist lcigaaafsh |
| 181 | yehwtffqay yycfitltti gfgdyvalqk dgalgtqpqy vafsfvyilt gltvigafln |
| 241 | lvvlrfmtmn aedekrdaeh ralltrngqa gggggggsah ttdtasstaa aggggfrnvy |
| 301 | aevlhfqsmc sclwyksrek lqysipmiip rdlstsdtcv eqshsspggg grysdtpsrr |
| 361 | clcsgaprsa issystglhs lstfrglmkr rssv |

By “KCNK3 polynucleotide” is meant a polynucleotide encoding a KCNK3 polypeptide or fragment thereof. An exemplary KCNK3 polynucleotide sequence is provided at NCBI Ref: NM_002246.2. The sequence provided at NCBI Ref: NM_002246.2 is reproduced below:

| (SEQ ID NO: 16) | |
|---|---|
| 1 | ggcggcggcg gcggcggcgg ccccgggcgc tgagcgggtg cccggcgcgg agagcggcga |
| 61 | gcgcagccat gccccaggcc gcctccgggg cagcagcagc ggcggccggg gccgaggcgc |
| 121 | gggccggggg cgccgggggg ccggcggcgg cccgggcggg acgatgaagc ggcagaacgt |
| 181 | gcgcacgctg gcgctcatcg tgtgcacctt cacctacctg ctggtgggcg ccgcggtctt |
| 241 | cgacgcgctg gagtcggagc ccgagctgat cgagcggcag cggctggagc tgcggcagca |
| 301 | ggagctgcgg gcgcgctaca acctcagcca gggcggctac gaggagctgg agcgcgtcgt |
| 361 | gctgcgcctc aagccgcaca aggccggcgt gcagtggcgc ttcgccggct ccttctactt |
| 421 | cgccatcacc gtcatcacca ccatcggcta cgggcacgcg gcacccagca cggatggcgg |
| 481 | caaggtgttc tgcatgttct acgcgctgct gggcatcccg ctcacgctcg tcatgttcca |
| 541 | gagcctgggc gagcgcatca acaccttggt gaggtacctg ctgcaccgcg ccaagaaggg |
| 601 | gctgggcatg cggcgcgccg acgtgtccat ggccaacatg gtgctcatcg gcttcttctc |
| 661 | gtgcatcagc acgctgtgca tcggcgccgc cgccttctcc cactacgagc actggacctt |
| 721 | cttccaggcc tactactact gcttcatcac cctcaccacc atcggcttcg gcgactacgt |
| 781 | ggcgctgcag aaggaccagg ccctgcagac gcagccgcag tacgtggcct tcagcttcgt |
| 841 | ctacatcctt acgggcctca cggtcatcgg cgccttcctc aacctcgtgg tgctgcgctt |
| 901 | catgaccatg aacgccgagg acgagaagcg cgacgccgag caccgcgcgc tgctcacgcg |
| 961 | caacgggcag gcgggcggcg gcggaggggg tggcagcgcg cacactacgg acaccgcctc |
| 1021 | atccacggcg gcagcgggcg gcggcggctt ccgcaacgtc tacgcggagg tgctgcactt |
| 1081 | ccagtccatg tgctcgtgcc tgtggtacaa gagccgcgag aagctgcagt actccatccc |
| 1141 | catgatcatc ccgcgggacc tctccacgtc cgacacgtgc gtggagcaga gccactcgtc |
| 1201 | gccgggaggg ggcggccgct acagcgacac gccctcgcga cgctgcctgt gcagcggggc |
| 1261 | gccacgctcc gccatcagct cggtgtccac gggtctgcac agcctgtcca ccttccgcgg |
| 1321 | cctcatgaag cgcaggagct ccgtgtgact gccccgaggg gcctggagca cctgggggcg |
| 1381 | cgggcggggg acccctgctg ggaggccagg agactgcccc tgctgccttc tgcccagtgg |
| 1441 | gaccccgcac aacatccctc accactctcc cccagcaccc ccatctccga ctgtgcctgc |
| 1501 | ttgcaccagc cggcaggagg ccgggctctg aggacccctg gggcccccat cggagccctg |
| 1561 | caaattccga gaaatgtgaa acttggtggg gtcagggagg aaaggcagaa gctgggagcc |
| 1621 | tcccttccct ttgaaaatct aagaagctcc cagtcctcag agaccctgct ggtacccaga |
| 1681 | cccccacctt cggaggggac ttcatgttcc gtgtacgttt gcatctctat ttatacctct |
| 1741 | gtcctgctag gtctcccacc ttcccttggt tccaaaagcc agggtgtcta tgtccaagtc |
| 1801 | acccctactc agccccactc cccttcctca tccccagctg tgtctcccaa cctcccttcg |
| 1861 | tgttgttttg catggctttg cagttatgga gaaagtggaa acccagcagt ccctaaagct |
| 1921 | ggtccccaga aagcaggaca gaaagaagga gggacaggca ggcagcagga ggggcgagct |
| 1981 | gggaggcagg aggcagcggc ctgtcagtct gcagaatggt cgcactggag gttcaagcta |
| 2041 | actggcctcc agccacattc tcatagcagg taggacttca gccttccaga cactgccctt |
| 2101 | agaatctgga acagaagact tcagactcac cataattgct gataattacc cactcttaaa |
| 2161 | tttgtcgagt gatttttagc ctctgaaaac tctatgctgg ccactgattc ctttgagtct |
| 2221 | cacaaaaccc tacttaggtc atcagggcag gagttctcac tcccatttta cagatgagaa |
| 2281 | tactgaggcc tggacaggtg aagtgaccag agagcaaaag gcaaaggggt gggggctggg |
| 2341 | tgcagtggct cacacctgta ttcccaacac ttttggaggc tgaggttgga ggattgcttg |
| 2401 | agcccaggaa tttgagacca gcctaggtga catagtgaga ccccatctct acaaaaaata |
| 2461 | aaaaattaac caggtgtggt ggcacgtgcc tgggagtccc agcgacttgg gaggctgagg |
| 2521 | tgggaggatt gtttgagcct gggaggtcga ggctgtagtg agccctgatt gcaccactgt |
| 2581 | actccagcct gggtgacagg gcaagaccct gtctcaaaaa aaaaaaaaaa aatggcaaag |
| 2641 | ggagacaaga gcccagcctg cttgttgcta gccaaagtgt tctttccttc cagcttggcc |
| 2701 | tgctcttaaa agcaaagctc ctgcagtgta catcctggca ttgtgtggct acctgggttt |
| 2761 | taaaccagaa tcagaagtcc cggatcagag ggcactgctg aggttcagcc tcttctcttc |
| 2821 | ttggccagga ggcagcagct ctgaatgggc ccctgaggct gcacaggggc ctttgtcact |
| 2881 | ggggcgcatg cttacaaaca gtgcagttct tgggaccgag gtaagcaggg ctgggtctca |
| 2941 | tggcagaaag gccaggatct ggggctctag gaatttggga attgggcaga gtggccaaga |
| 3001 | aagctggcag gcatatccta tgggacatca cacctggcac cattgtcatt gttggtgcct |
| 3061 | gtgtcccaag tagctagtga taagctgagg ctgcagcaag aaacaccctt cccaggtggg |
| 3121 | ggagtttgga ccagaggtgc cctctgccca ccacacctgc aacccagaag cccagatgga |
| 3181 | acgcagctga cgaaggtgat gcttgaggct cacttttggg gccccacagc tggagccggt |
| 3241 | ataatgactg ggacaacatc aaggggtgga tgaggggcct ctcctcccgc aacactgcct |
| 3301 | tcccatgctg ttcccctgcc agctccttaa cactgccgac caaggccagc cctggcattc |
| 3361 | agggaaattg gagggcagca cccgtagggt ggccagcctc aggccccacc ccagctgtgt |
| 3421 | cctctagtct ctggggaccc ctggggggaa gaagtctacc ctgcttgtga gtcccgtctc |
| 3481 | agtgtggagg aactggctgc acgtgggacc tgaaggtgcc ctctgtgttt atgttggggg |
| 3541 | tgggggggca gtgctggctg cctctgtcct gtgtgtgacc ctgccctcga agggtcctgt |
| 3601 | cctgtcagtc ccgagggagc cacaaccaaa gctgcggaga gaaggtgggg aagggtgcag |
| 3661 | aatggccgtg gggcacagcg tggcagactg ttcagtctct gctgggtctt tcctagggac |
| 3721 | ctggaaggcc agtgttgctt ccccctcact ccctttcact gcaggcagcc tctctgcttc |
| 3781 | cccaatgcct tatgcctggg cacactgcca cagaatatgc aatatgtgtg ggtgaccatg |
| 3841 | ccctcacgac cacaccccca ccccgggcag cccccggact ccaaaggtcg tggctgccac |
| 3901 | agcctccctc agctcttcct gcctatctgt cttcacactg agaatggcgc ccaataaatg |
| 3961 | ctatccacgg agaccagg |

By “KCNQ1 polypeptide” is meant a protein or fragment thereof having at least 85% amino acid sequence identity to the sequence provided at NCBI Accession No. NP_000209.2 (isoform 1) or NP_861463.1 (isoform 2) and having potassium channel activity. The amino acid sequence provided at NCBI Accession No. NP_000209.2 is shown below:

| (SEQ ID NO: 17) | |
|---|---|
| 1 | maaassppra erkrwgwgrl pgarrgsagl akkcpfslel aeggpaggal yapiapgapg |
| 61 | pappaspaap aappvasdlg prppvsldpr vsiystrrpv larthvqgry ynflerptgw |
| 121 | kcfvyhfavf livlvclifs vlstieqyaa latgtlfwme ivlvvffgte yvvrlwsagc |
| 181 | rskyvglwgr lrfarkpisi idlivvvasm vvlcvgskgq vfatsairgi rflqilrmlh |
| 241 | vdrqggtwrl lgsvvfihrq elittlyigf lglifssyfv ylaekdavne sgrvefgsya |
| 301 | dalwwgvvtv ttigygdkvp qtwvgktias cfsvfaisff alpagilgsg falkvqqkqr |
| 361 | qkhfnrqipa aasliqtawr cyaaenpdss twkiyirkap rshtllspsp kpkksvvvkk |
| 421 | kkfkldkdng vtpgekmltv phitcdppee rrldhfsvdg ydssvrkspt llevsmphfm |
| 481 | rtnsfaedld legetlltpi thisqlrehh ratikvirrm qyfvakkkfq qarkpydvrd |
| 541 | vieqysqghl nlmvrikelq rrldqsigkp slfisvseks kdrgsntiga rlnrvedkvt |
| 601 | qldqrlalit dmlhqllslh ggstpgsggp preggahitq pcgsggsvdp elflpsntlp |
| 661 | tyeqltvprr gpdegs |

By “KCNQ1 polynucleotide” is meant a polynucleotide encoding a KCNQ1 polypeptide or fragment thereof. An exemplary KCNQ1 polynucleotide sequence is provided at NCBI Ref: NM_000218.2. The sequence provided at NCBI Ref: NM_000218.2 is reproduced below:

| (SEQ ID NO: 18) | |
|---|---|
| 1 | gcggcggggc tggcagcagt ggctgcccgc actgcgcccg ggcgctcgcc ttcgctgcag |
| 61 | ctcccggtgc cgccgctcgg gccggccccc cggcaggccc tcctcgttat ggccgcggcc |
| 121 | tcctccccgc ccagggccga gaggaagcgc tggggttggg gccgcctgcc aggcgcccgg |
| 181 | cggggcagcg cgggcctggc caagaagtgc cccttctcgc tggagctggc ggagggcggc |
| 241 | ccggcgggcg gcgcgctcta cgcgcccatc gcgcccggcg ccccaggtcc cgcgccccct |
| 301 | gcgtccccgg ccgcgcccgc cgcgccccca gttgcctccg accttggccc gcggccgccg |
| 361 | gtgagcctag acccgcgcgt ctccatctac agcacgcgcc gcccggtgtt ggcgcgcacc |
| 421 | cacgtccagg gccgcgtcta caacttcctc gagcgtccca ccggctggaa atgcttcgtt |
| 481 | taccacttcg ccgtcttcct catcgtcctg gtctgcctca tcttcagcgt gctgtccacc |
| 541 | atcgagcagt atgccgccct ggccacgggg actctcttct ggatggagat cgtgctggtg |
| 601 | gtgttcttcg ggacggagta cgtggtccgc ctctggtccg ccggctgccg cagcaagtac |
| 661 | gtgggcctct gggggcggct gcgctttgcc cggaagccca tttccatcat cgacctcatc |
| 721 | gtggtcgtgg cctccatggt ggtcctctgc gtgggctcca aggggcaggt gtttgccacg |
| 781 | tcggccatca ggggcatccg cttcctgcag atcctgagga tgctacacgt cgaccgccag |
| 841 | ggaggcacct ggaggctcct gggctccgtg gtcttcatcc accgccagga gctgataacc |
| 901 | accctgtaca tcggcttcct gggcctcatc ttctcctcgt actttgtgta cctggctgag |
| 961 | aaggacgcgg tgaacgagtc aggccgcgtg gagttcggca gctacgcaga tgcgctgtgg |
| 1021 | tggggggtgg tcacagtcac caccatcggc tatggggaca aggtgcccca gacgtgggtc |
| 1081 | gggaagacca tcgcctcctg cttctctgtc tttgccatct ccttctttgc gctcccagcg |
| 1141 | gggattcttg gctcggggtt tgccctgaag gtgcagcaga agcagaggca gaagcacttc |
| 1201 | aaccggcaga tcccggcggc agcctcactc attcagaccg catggaggtg ctatgctgcc |
| 1261 | gagaaccccg actcctccac ctggaagatc tacatccgga aggccccccg gagccacact |
| 1321 | ctgctgtcac ccagccccaa acccaagaag tctgtggtgg taaagaaaaa aaagttcaag |
| 1381 | ctggacaaag acaatggggt gactcctgga gagaagatgc tcacagtccc ccatatcacg |
| 1441 | tgcgaccccc cagaagagcg gcggctggac cacttctctg tcgacggcta tgacagttct |
| 1501 | gtaaggaaga gcccaacact gctggaagtg agcatgcccc atttcatgag aaccaacagc |
| 1561 | ttcgccgagg acctggacct ggaaggggag actctgctga cacccatcac ccacatctca |
| 1621 | cagctgcggg aacaccatcg ggccaccatt aaggtcattc gacgcatgca gtactttgtg |
| 1681 | gccaagaaga aattccagca agcgcggaag ccttacgatg tgcgggacgt cattgagcag |
| 1741 | tactcgcagg gccacctcaa cctcatggtg cgcatcaagg agctgcagag gaggctggac |
| 1801 | cagtccattg ggaagccctc actgttcatc tccgtctcag aaaagagcaa ggatcgcggc |
| 1861 | agcaacacga tcggcgcccg cctgaaccga gtagaagaca aggtgacgca gctggaccag |
| 1921 | aggctggcac tcatcaccga catgcttcac cagctgctct ccttgcacgg tggcagcacc |
| 1981 | cccggcagcg gcggcccccc cagagagggc ggggcccaca tcacccagcc ctgcggcagt |
| 2041 | ggcggctccg tcgaccctga gctcttcctg cccagcaaca ccctgcccac ctacgagcag |
| 2101 | ctgaccgtgc ccaggagggg ccccgatgag gggtcctgag gaggggatgg ggctggggga |
| 2161 | tgggcctgag tgagagggga ggccaagagt ggccccacct ggccctctct gaaggaggcc |
| 2221 | acctcctaaa aggcccagag agaagagccc cactctcaga ggccccaata ccccatggac |
| 2281 | catgctgtct ggcacagcct gcacttgggg gctcagcaag gccacctctt cctggccggt |
| 2341 | gtgggggccc cgtctcaggt ctgagttgtt accccaagcg ccctggcccc cacatggtga |
| 2401 | tgttgacatc actggcatgg tggttgggac ccagtggcag ggcacagggc ctggcccatg |
| 2461 | tatggccagg aagtagcaca ggctgagtgc aggcccaccc tgcttggccc agggggcttc |
| 2521 | ctgaggggag acagagcaac ccctggaccc cagcctcaaa tccaggaccc tgccaggcac |
| 2581 | aggcagggca ggaccagccc acgctgacta cagggccgcc ggcaataaaa gcccaggagc |
| 2641 | ccatttggag ggcctgggcc tggctccctc actctcagga aatgctgacc catgggcagg |
| 2701 | agactgtgga gactgctcct gagcccccag cttccagcag gagggacagt ctcaccattt |
| 2761 | ccccagggca cgtggttgag tggggggaac gcccacttcc ctgggttaga ctgccagctc |
| 2821 | ttcctagctg gagaggagcc ctgcctctcc gcccctgagc ccactgtgcg tggggctccc |
| 2881 | gcctccaacc cctcgcccag tcccagcagc cagccaaaca cacagaaggg gactgccacc |
| 2941 | tccccttgcc agctgctgag ccgcagagaa gtgacggttc ctacacagga caggggttcc |
| 3001 | ttctgggcat tacatcgcat agaaatcaat aatttgtggt gatttggatc tgtgttttaa |
| 3061 | tgagtttcac agtgtgattt tgattattaa ttgtgcaagc ttttcctaat aaacgtggag |
| 3121 | aatcacaggc tgggctgggc actgctctca ccttggttcc tggggcatcc atggggtctc |
| 3181 | tcacagacag gacccctgca gttcccctgg aagcagtgcc caggtggctg tggaatagga |
| 3241 | acgctaaaaa aaaaaaaaaa aa |

By “LGR5 polypeptide” is meant a protein or fragment thereof having at least 85% amino acid sequence identity to the sequence provided at NCBI Accession No. NP_003658.1 (isoform 1), NP_001264155.1 (isoform 2), or NP_001264156.1 (isoform 3) and having transmembrane signaling receptor activity or G-protein coupled receptor activity. The amino acid sequence provided at NCBI Accession No. NP_003658.1 is shown below:

| (SEQ ID NO: 19) | |
|---|---|
| 1 | mdtsrlgvll slpvllqlat ggssprsgvl lrgcpthchc epdgrmllrv dcsdlglsel |
| 61 | psnlsvftsy ldlsmnnisq llpnplpslr fleelrlagn altyipkgaf tglyslkvlm |
| 121 | lqnnqlrhvp tealqnlrsl qslrldanhi syvppscfsg lhslrhlwld dnalteipvq |
| 181 | afrslsalqa mtlalnkihh ipdyafgnls slvvlhlhnn rihslgkkcf dglhsletld |
| 241 | lnynnldefp tairtlsnlk elgfhsnnir sipekafvgn pslitihfyd npiqfvgrsa |
| 301 | fghlpelrtl tlngasqite fpdltgtanl esltltgaqi sslpqtvcnq lpnlqvldls |
| 361 | ynlledlpsf svcqklqkid lrhneiyeik vdtfqqllsl rslnlawnki aiihpnafst |
| 421 | lpslikldls snllssfpit glhglthlkl tgnhalqsli ssenfpelkv iempyayqcc |
| 481 | afgvcenayk isnqwnkgdn ssmddlhkkd agmfqaqder dledflldfe edlkalhsvq |
| 541 | cspspgpfkp cehlldgwli rigvwtiavl altcnalvts tvfrsplyis piklligvia |
| 601 | avnmltgvss avlagvdaft fgsfarhgaw wengvgchvi gflsifases svflltlaal |
| 661 | ergfsvkysa kfetkapfss lkviillcal laltmaavpl lggskygasp lclplpfgep |
| 721 | stmgymvali llnslcflmm tiaytklycn ldkgdleniw dcsmvkhial llftncilnc |
| 781 | pvaflsfssl inltfispev ikfillvvvp lpaclnplly ilfnphfked lvslrkqtyv |
| 841 | wtrskhpslm sinsddvekq scdstqalvt ftsssitydl ppssvpspay pvteschlss |
| 901 | vafvpcl |

By “LGR5 polynucleotide” is meant a polynucleotide encoding a LGR5 polypeptide or fragment thereof. An exemplary LGRS polynucleotide sequence is provided at NCBI Ref: NM_003667.3. The sequence provided at NCBI Ref: NM_003667.3 is reproduced below:

| (SEQ ID NO: 20) | |
|---|---|
| 1 | aaaaaacgag cgtgcaagca gagatgctgc tccacaccgc tcaggccgcg agcagcagca |
| 61 | aggcgcaccg ccactgtcgc cgctgcagcc agggctgctc cgaaggccgg cgtggcggca |
| 121 | accggcacct ctgtccccgc cgcgcttctc ctcgccgccc acgccgtggg gtcaggaacg |
| 181 | cggcgtctgg cgctgcagac gcccgctgag ttgcagaagc ccacggagcg gcgcccggcg |
| 241 | cgccacggcc cgtagcagtc cggtgctgct ctccgcccgc gtccggctcg tggcccccta |
| 301 | cttcgggcac catggacacc tcccggctcg gtgtgctcct gtccttgcct gtgctgctgc |
| 361 | agctggcgac cgggggcagc tctcccaggt ctggtgtgtt gctgaggggc tgccccacac |
| 421 | actgtcattg cgagcccgac ggcaggatgt tgctcagggt ggactgctcc gacctggggc |
| 481 | tctcggagct gccttccaac ctcagcgtct tcacctccta cctagacctc agtatgaaca |
| 541 | acatcagtca gctgctcccg aatcccctgc ccagtctccg cttcctggag gagttacgtc |
| 601 | ttgcgggaaa cgctctgaca tacattccca agggagcatt cactggcctt tacagtctta |
| 661 | aagttcttat gctgcagaat aatcagctaa gacacgtacc cacagaagct ctgcagaatt |
| 721 | tgcgaagcct tcaatccctg cgtctggatg ctaaccacat cagctatgtg cccccaagct |
| 781 | gtttcagtgg cctgcattcc ctgaggcacc tgtggctgga tgacaatgcg ttaacagaaa |
| 841 | tccccgtcca ggcttttaga agtttatcgg cattgcaagc catgaccttg gccctgaaca |
| 901 | aaatacacca cataccagac tatgcctttg gaaacctctc cagcttggta gttctacatc |
| 961 | tccataacaa tagaatccac tccctgggaa agaaatgctt tgatgggctc cacagcctag |
| 1021 | agactttaga tttaaattac aataaccttg atgaattccc cactgcaatt aggacactct |
| 1081 | ccaaccttaa agaactagga tttcatagca acaatatcag gtcgatacct gagaaagcat |
| 1141 | ttgtaggcaa cccttctctt attacaatac atttctatga caatcccatc cagtttgttg |
| 1201 | ggagatctgc ttttcaacat ttacctgaac taagaacact gactctgaat ggtgcctcac |
| 1261 | aaataactga atttcctgat ttaactggaa ctgcaaacct ggagagtctg actttaactg |
| 1321 | gagcacagat ctcatctctt cctcaaaccg tctgcaatca gttacctaat ctccaagtgc |
| 1381 | tagatctgtc ttacaaccta ttagaagatt tacccagttt ttcagtctgc caaaagcttc |
| 1441 | agaaaattga cctaagacat aatgaaatct acgaaattaa agttgacact ttccagcagt |
| 1501 | tgcttagcct ccgatcgctg aatttggctt ggaacaaaat tgctattatt caccccaatg |
| 1561 | cattttccac tttgccatcc ctaataaagc tggacctatc gtccaacctc ctgtcgtctt |
| 1621 | ttcctataac tgggttacat ggtttaactc acttaaaatt aacaggaaat catgccttac |
| 1681 | agagcttgat atcatctgaa aactttccag aactcaaggt tatagaaatg ccttatgctt |
| 1741 | accagtgctg tgcatttgga gtgtgtgaga atgcctataa gatttctaat caatggaata |
| 1801 | aaggtgacaa cagcagtatg gacgaccttc ataagaaaga tgctggaatg tttcaggctc |
| 1861 | aagatgaacg tgaccttgaa gatttcctgc ttgactttga ggaagacctg aaagcccttc |
| 1921 | attcagtgca gtgttcacct tccccaggcc ccttcaaacc ctgtgaacac ctgcttgatg |
| 1981 | gctggctgat cagaattgga gtgtggacca tagcagttct ggcacttact tgtaatgctt |
| 2041 | tggtgacttc aacagttttc agatcccctc tgtacatttc ccccattaaa ctgttaattg |
| 2101 | gggtcatcgc agcagtgaac atgctcacgg gagtctccag tgccgtgctg gctggtgtgg |
| 2161 | atgcgttcac ttttggcagc tttgcacgac atggtgcctg gtgggagaat ggggttggtt |
| 2221 | gccatgtcat tggttttttg tccatttttg cttcagaatc atctgttttc ctgcttactc |
| 2281 | tggcagccct ggagcgtggg ttctctgtga aatattctgc aaaatttgaa acgaaagctc |
| 2341 | cattttctag cctgaaagta atcattttgc tctgtgccct gctggccttg accatggccg |
| 2401 | cagttcccct gctgggtggc agcaagtatg gcgcctcccc tctctgcctg cctttgcctt |
| 2461 | ttggggagcc cagcaccatg ggctacatgg tcgctctcat cttgctcaat tccctttgct |
| 2521 | tcctcatgat gaccattgcc tacaccaagc tctactgcaa tttggacaag ggagacctgg |
| 2581 | agaatatttg ggactgctct atggtaaaac acattgccct gttgctcttc accaactgca |
| 2641 | tcctaaactg ccctgtggct ttcttgtcct tctcctcttt aataaacctt acatttatca |
| 2701 | gtcctgaagt aattaagttt atccttctgg tggtagtccc acttcctgca tgtctcaatc |
| 2761 | cccttctcta catcttgttc aatcctcact ttaaggagga tctggtgagc ctgagaaagc |
| 2821 | aaacctacgt ctggacaaga tcaaaacacc caagcttgat gtcaattaac tctgatgatg |
| 2881 | tcgaaaaaca gtcctgtgac tcaactcaag ccttggtaac ctttaccagc tccagcatca |
| 2941 | cttatgacct gcctcccagt tccgtgccat caccagctta tccagtgact gagagctgcc |
| 3001 | atctttcctc tgtggcattt gtcccatgtc tctaattaat atgtgaagga aaatgttttc |
| 3061 | aaaggttgag aacctgaaaa tgtgagattg agtatatcag agcagtaatt aataagaaga |
| 3121 | gctgaggtga aactcggttt aaaaaccaaa aaagaatctc tcagttagta agaaaaggct |
| 3181 | gaaaacctct tgatacttga gagtgaatat aagtctaaat gctgctttgt ataatttgtt |
| 3241 | cagctaaggg atagatcgat cacactattt aagtgagccc agatcaaaaa agcagattga |
| 3301 | aattttcttt agaaaagatt ctccatgatt tgaattgcat tctctttaaa ctcaccaatg |
| 3361 | taatcatttt gggaggaggg agaacccact tgctttccaa atgggtttat ttaaacccac |
| 3421 | aaactcaaga ggttgttggg ggaattagga aaataagggt tttcaatgac ctacattgct |
| 3481 | aggtagaggc tgtgatccat gggatttcat tctaatgacc atgtgaagat gtttgagtcc |
| 3541 | tcctttgcct ttcctcagaa agaatccttc taaggcacaa atcccttaga tggataatgt |
| 3601 | aaggtattgt taactcactc atattgagat catttttaga gataccaggt tttatgtatc |
| 3661 | agcactagat ggttccaccc tcatgggata aaactgctta caagtatttt gaaagaaaaa |
| 3721 | ctgaccaaaa ttcttaaatt gttactaagg caatcatgca caggtgacgt atgtcttatc |
| 3781 | tgatttgttt ttaactcctt ggtgcccaaa gctcagaagg gaattccact gccagcaatg |
| 3841 | aacatacctg gaaaagaaag taagcaatct gggatttttt ttctgggtta gtaaagaatt |
| 3901 | tttgcaataa gttttatcag ttgattcaaa ctgatgtgca tcttaatgat caaatgtgca |
| 3961 | cattacataa attaagtcca ctgatacaac ttcttacaca tgtatctcta gtagctctgg |
| 4021 | caaacccaat atctgacacc actttggact caagagactc agtaacgtat tatcctgttt |
| 4081 | atttagcttg gttttagctg tgttctctct ggataaccca cttgatgtta ggaacattac |
| 4141 | ttctctgctt attccatatt aatactgtgt taggtatttt aagaagcaag ttattaaata |
| 4201 | agaaaagtca aagtattaat tcttaccttc tattatccta tattagcttc aatacatcca |
| 4261 | aaccaaatgg ctgttaggta gatttatttt tatataagca tgtttatttt gatcagatgt |
| 4321 | tttaacttgg atttgaaaaa atacatttat gagatgtttt ataagatgtg taaatataga |
| 4381 | actgtattta ttactatagt aaaggttcag taacattaag gaccatgata atgataataa |
| 4441 | accttgtaca gtggcatatt ctttgattta tattgtgttt ctctgcccat tttctttaaa |
| 4501 | ttcattaact gtatatatgt aaatatatag tacttgtaaa tagattccaa atttgctttt |
| 4561 | ctattgggta aaaaataaat ttgtaataaa atgtgtgact atgaaacaaa aaaaaaaaaa |
| 4621 | aaaaa |

By “LDHA polypeptide” or “lactate dehydrogenase A polypeptide” is meant a protein or fragment thereof having at least 85% amino acid sequence identity to the sequence provided at NCBI Accession No. NP_005557.1 (isoform 1), NP_001128711.1 (isoform 2), NP_001158886.1 (isoform 3), NP_001158887.1 (isoform 4), or NP_001158888.1 (isoform 5) and having dehydrogenase activity. The amino acid sequence provided at NCBI Accession No. NP_005557.1 is shown below:

| (SEQ ID NO: 21) | |
|---|---|
| 1 | matlkdqliy nllkeeqtpq nkitvvgvga vgmacaisil mkdladelal vdviedklkg |
| 61 | emmdlqhgsl flrtpkivsg kdynvtansk lviitagarq qegesrlnlv qrnvnifkfi |
| 121 | ipnvvkyspn ckllivsnpv diltyvawki sgfpknrvig sgcnldsarf rylmgerlgv |
| 181 | hplschgwvl gehgdssvpv wsgmnvagvs lktlhpdlgt dkdkeqwkev hkqvvesaye |
| 241 | viklkgytsw aiglsvadla esimknlrrv hpvstmikgl ygikddvfls vpcilgqngi |
| 301 | sdlvkvtlts eeearlkksa dtlwgiqkel qf |

By “LDHA polynucleotide” or “lactate dehydrogenase A polynucleotide” is meant a polynucleotide encoding a LDHA polypeptide or fragment thereof. An exemplary LDHA polynucleotide sequence is provided at NCBI Ref: NM_005566.3. The sequence provided at NCBI Ref: NM_005566.3 is reproduced below:

| (SEQ ID NO: 22) | |
|---|---|
| 1 | gtctgccggt cggttgtctg gctgcgcgcg ccacccgggc ctctccagtg ccccgcctgg |
| 61 | ctcggcatcc acccccagcc cgactcacac gtgggttccc gcacgtccgc cggccccccc |
| 121 | cgctgacgtc agcatagctg ttccacttaa ggcccctccc gcgcccagct cagagtgctg |
| 181 | cagccgctgc cgccgattcc ggatctcatt gccacgcgcc cccgacgacc gcccgacgtg |
| 241 | cattcccgat tccttttggt tccaagtcca atatggcaac tctaaaggat cagctgattt |
| 301 | ataatcttct aaaggaagaa cagacccccc agaataagat tacagttgtt ggggttggtg |
| 361 | ctgttggcat ggcctgtgcc atcagtatct taatgaagga cttggcagat gaacttgctc |
| 421 | ttgttgatgt catcgaagac aaattgaagg gagagatgat ggatctccaa catggcagcc |
| 481 | ttttccttag aacaccaaag attgtctctg gcaaagacta taatgtaact gcaaactcca |
| 541 | agctggtcat tatcacggct ggggcacgtc agcaagaggg agaaagccgt cttaatttgg |
| 601 | tccagcgtaa cgtgaacatc tttaaattca tcattcctaa tgttgtaaaa tacagcccga |
| 661 | actgcaagtt gcttattgtt tcaaatccag tggatatctt gacctacgtg gcttggaaga |
| 721 | taagtggttt tcccaaaaac cgtgttattg gaagcggttg caatctggat tcagcccgat |
| 781 | tccgttacct aatgggggaa aggctgggag ttcacccatt aagctgtcat gggtgggtcc |
| 841 | ttggggaaca tggagattcc agtgtgcctg tatggagtgg aatgaatgtt gctggtgtct |
| 901 | ctctgaagac tctgcaccca gatttaggga ctgataaaga taaggaacag tggaaagagg |
| 961 | ttcacaagca ggtggttgag agtgcttatg aggtgatcaa actcaaaggc tacacatcct |
| 1021 | gggctattgg actctctgta gcagatttgg cagagagtat aatgaagaat cttaggcggg |
| 1081 | tgcacccagt ttccaccatg attaagggtc tttacggaat aaaggatgat gtcttcctta |
| 1141 | gtgttccttg cattttggga cagaatggaa tctcagacct tgtgaaggtg actctgactt |
| 1201 | ctgaggaaga ggcccgtttg aagaagagtg cagatacact ttgggggatc caaaaggagc |
| 1261 | tgcaatttta aagtcttctg atgtcatatc atttcactgt ctaggctaca acaggattct |
| 1321 | aggtggaggt tgtgcatgtt gtccttttta tctgatctgt gattaaagca gtaatatttt |
| 1381 | aagatggact gggaaaaaca tcaactcctg aagttagaaa taagaatggt ttgtaaaatc |
| 1441 | cacagctata tcctgatgct ggatggtatt aatcttgtgt agtcttcaac tggttagtgt |
| 1501 | gaaatagttc tgccacctct gacgcaccac tgccaatgct gtacgtactg catttgcccc |
| 1561 | ttgagccagg tggatgttta ccgtgtgtta tataacttcc tggctccttc actgaacatg |
| 1621 | cctagtccaa cattttttcc cagtgagtca catcctggga tccagtgtat aaatccaata |
| 1681 | tcatgtcttg tgcataattc ttccaaagga tcttattttg tgaactatat cagtagtgta |
| 1741 | cattaccata taatgtaaaa agatctacat acaaacaatg caaccaacta tccaagtgtt |
| 1801 | ataccaacta aaacccccaa taaaccttga acagtgacta ctttggttaa ttcattatat |
| 1861 | taagatataa agtcataaag ctgctagtta ttatattaat ttggaaatat taggctattc |
| 1921 | ttgggcaacc ctgcaacgat tttttctaac agggatatta ttgactaata gcagaggatg |
| 1981 | taatagtcaa ctgagttgta ttggtaccac ttccattgta agtcccaaag tattatatat |
| 2041 | ttgataataa tgctaatcat aattggaaag taacattcta tatgtaaatg taaaatttat |
| 2101 | ttgccaactg aatataggca atgatagtgt gtcactatag ggaacacaga tttttgagat |
| 2161 | cttgtcctct ggaagctggt aacaattaaa aacaatctta aggcagggaa aaaaaaaaaa |
| 2221 | aaaaaa |

By “MAFA polypeptide” is meant a protein or fragment thereof having at least 85% amino acid sequence identity to the sequence provided at NCBI Accession No. NP_963883.2 and having transcription factor activity. The amino acid sequence provided at NCBI Accession No. NP_963883.2 is shown below:

| (SEQ ID NO: 23) | |
|---|---|
| 1 | maaelamgae lpssplaiey vndfdlmkfe vkkeppeaer fchrlppgsl sstplstpcs |
| 61 | svpsspsfca pspgtggggg agggggssqa ggapgppsgg pgavggtsgk paledlywms |
| 121 | gyqhhlnpea lnltpedave aligsghhga hhgahhpaaa aayeafrgpg faggggaddm |
| 181 | gaghhhgahh aahhhhaahh hhhhhhhhgg aghgggaghh vrleerfsdd qlvsmsvrel |
| 241 | nrqlrgfske evirlkqkrr tlknrgyaqs crfkrvqqrh ilesekcqlq sqveqlklev |
| 301 | grlakerdly kekyeklagr ggpgsaggag fprepsppqa gpggakgtad ffl |

By “MAFA polynucleotide” is meant a polynucleotide encoding a MAFA polypeptide or fragment thereof. An exemplary MAFA polynucleotide sequence is provided at NCBI Ref: NM_201589.3. The sequence provided at NCBI Ref: NM_201589.3 is reproduced below:

| (SEQ ID NO: 24) | |
|---|---|
| 1 | gcgcggccgg gcgcgggccc cgggcgatgg ccgcggagct ggcgatgggc gccgagctgc |
| 61 | ccagcagccc gctggccatc gagtacgtca acgacttcga cctgatgaag ttcgaggtga |
| 121 | agaaggagcc tcccgaggcc gagcgcttct gccaccgcct gccgccaggc tcgctgtcct |
| 181 | cgacgccgct cagcacgccc tgctcctccg tgccctcctc gcccagcttc tgcgcgccca |
| 241 | gcccgggcac cggcggcggc ggcggcgcgg ggggcggcgg cggctcgtct caggccgggg |
| 301 | gcgcccccgg gccgccgagc gggggccccg gcgccgtcgg gggcacctcg gggaagccgg |
| 361 | cgctggagga tctgtactgg atgagcggct accagcatca cctcaacccc gaggcgctca |
| 421 | acctgacgcc cgaggacgcg gtggaggcgc tcatcggcag cggccaccac ggcgcgcacc |
| 481 | acggcgcgca ccacccggcg gccgccgcag cctacgaggc tttccgcggc ccgggcttcg |
| 541 | cgggcggcgg cggagcggac gacatgggcg ccggccacca ccacggcgcg caccacgccg |
| 601 | cccaccatca ccacgccgcc caccaccacc accaccacca ccaccaccat ggcggcgcgg |
| 661 | gacacggcgg tggcgcgggc caccacgtgc gcctggagga gcgcttctcc gacgaccagc |
| 721 | tggtgtccat gtcggtgcgc gagctgaacc ggcagctccg cggcttcagc aaggaggagg |
| 781 | tcatccggct caagcagaag cggcgcacgc tcaagaaccg cggctacgcg cagtcctgcc |
| 841 | gcttcaagcg ggtgcagcag cggcacattc tggagagcga gaagtgccaa ctccagagcc |
| 901 | aggtggagca gctgaagctg gaggtggggc gcctggccaa agagcgggac ctgtacaagg |
| 961 | agaaatacga gaagctggcg ggccggggcg gccccgggag cgcgggcggg gccggtttcc |
| 1021 | cgcgggagcc ttcgccgccg caggccggtc ccggcggggc caagggcacg gccgacttct |
| 1081 | tcctgtaggc gccggacccc gagcccgcgc cgccgtcgcc ggggacaagt tcgcgcaggc |
| 1141 | ctctcggggc ctcggctcgg actccgcggt acaggacgtg gacaccaggc ccggcccggc |
| 1201 | cgtgctggcc ccggtgccaa gtctgcgggc gcggggctgg aggccccttc gctcccggtc |
| 1261 | cccgttcgcg cgcgtcggcc cgggtcgccg tcctgaggtt gagcggagaa cggtgatttc |
| 1321 | taaggaaact tgagccaggt ctaacttctt tccaagcgtc cgcttgtaca tacgttgaac |
| 1381 | gtggttctcc gttcccacct tcgccctgcc agcctagagg gaccgcgctg ccgtcccttc |
| 1441 | ccgggtggcc cctgcctgcc cccgccctcc ttcgttctct tctcagcctc cctttccttg |
| 1501 | ccttttttaa cttcccctcc ccgttttaaa atcggtctta ttttcgaagt atttataatt |
| 1561 | attatgcttg gtgattagaa aagaaaacct tggaggaagc cccttctttc cccagccggg |
| 1621 | gtccgccctc agtcgcgagt cacagcatga gtcgctcgcc aggaggggcc cggcccctgc |
| 1681 | ctgccccctc cccgcttgcc cccgaccctg ctaccggcgt tccttggagg tcgaagccag |
| 1741 | ggacgtcacc cgtgctgtgt ccaggcctgc tgtcctacta tgctcaaccg ggggtggggg |
| 1801 | gaggggggtg agtcctgtgc tcagtcgggt gggggctggc ccggatcccg agctgctgtc |
| 1861 | tctctatgca ccagaacata tctgtaactc ctggggaaat acatcttgtt ttaaccttca |
| 1921 | agagaagtga aagaaaaaag taatgcacag tatttctagc agaaaatttt tttttttaag |
| 1981 | aggaggcttg ggccagagcc ttctggcatg gggcgggtgg agaaagtgtt tttattttaa |
| 2041 | tttaaattgt gtttcgtttt gtttgtggaa tctttcttta atgcttcgtc gctctttgga |
| 2101 | ctagccggga gagagggcga ggaggcgggt gctccaggcc ctgtaggctg ggccaggcgc |
| 2161 | ctgggggatc tgcccgtttt cggaggccct caggggccat cagtgggatt ccagccgctc |
| 2221 | cacacccctc ccctgagcac tcggagtgga aggcgcgccg actcgttgaa agttttgttg |
| 2281 | tgtagttggt tttcgttgag ttcttttttc atttgctacg aaactgagaa aaagaaaaaa |
| 2341 | atacacaaaa taaatctgtt cagatccaag tca |

As used herein, a “marker” is meant any protein or polynucleotide having an alteration in expression level or activity that is associated with a disease or disorder or that is associated with a particular cell type. In some embodiments, a marker for a beta cell is Pdx1, MafA, Pax4, Pax6, NeuroD1, Nkx6-1,Gata6, or Foxa2. In some embodiments, a marker for a hepatocyte is AFP, ALB, or Cyp3a7. In some other embodiments, a marker for a cardiomyocyte is hMlc2a, hNkx2-5, alphaMHC or KCNQ1. In still other embodiments, a marker for a small intestine cell is CDX2, Muc2, or Lgr5.

By “alphaMHC polypeptide” or “myosin heavy chain (MHC) alpha polypeptide” is meant a protein or fragment thereof having at least 85% amino acid sequence identity to the sequence provided at NCBI Accession No. NP_002462.2 and having actin binding activity. The amino acid sequence provided at NCBI Accession No. NP_002462.2 is shown below:

| (SEQ ID NO: 25) | |
|---|---|
| 1 | mtdaqmadfg aaaqylrkse kerleaqtrp fdirtecfvp ddkeefvkak ilsreggkvi |
| 61 | aetengktvt vkedqvlqqn ppkfdkiedm amltflhepa vlfnlkerya awmiytysgl |
| 121 | fcvtvnpykw lpvynaevva ayrgkkrsea pphifsisdn ayqymltdre nqsilitges |
| 181 | gagktvntkr viqyfasiaa igdrgkkdna nankgtledq iiqanpalea fgnaktvrnd |
| 241 | nssrfgkfir ihfgatgkla sadietylle ksrvifqlka ernyhifyqi lsnkkpelld |
| 301 | mllvtnnpyd yafvsqgevs vasiddseel matdsafdvl gftseekagv ykltgaimhy |
| 361 | gnmkfkqkqr eeqaepdgte dadksaylmg lnsadllkgl chprvkvgne yvtkggsvgq |
| 421 | vyysigalak avyekmfnwm vtrinatlet kqprgyfigv ldiagfeifd fnsfeqlcin |
| 481 | ftneklqqff nhhmfvleqe eykkegiewt fidfgmdlqa cidliekpmg imsileeecm |
| 541 | fpkatdmtfk aklydnhlgk snnfqkprni kgkqeahfsl ihyagtvdyn ilgwleknkd |
| 601 | pinetvvaly qksslklmat lfssyatadt gdsgkskggk kkgssfqtvs alhrenlnkl |
| 661 | mtnlrtthph fvrciipner kapgvmdnpl vmhqlrongv legiricrkg fpnrilygdf |
| 721 | rqryrilnpv aipegqfids rkgtekllss ldidhnqykf ghtkvffkag llglleemrd |
| 781 | erlsriitrm gagargqlmr iefkkiverr dallviqwni rafmgvknwp wmklyfkikp |
| 841 | llksaeteke matmkeefgr iketleksea rrkeleekmv sllgekndlq lqvgaegdnl |
| 901 | ndaeercdql iknkiqleak vkemnerled eeemnaelta kkrkledecs elkkdiddle |
| 961 | ltlakvekek hatenkvknl teemagldei iakltkekka lqeahqqald dlqveedkvn |
| 1021 | slskskvkle qqvddlegsl egekkvrmdl erakrklegd lkltqesimd lendklqlee |
| 1081 | klkkkefdin qqnskiedeq vlalqlqkkl kenqarieel eeeleaerta rakveklrsd |
| 1141 | lsreleeise rleeaggats vqiemnkkre aefqkmrrdl eeatlqheat aaalrkkhad |
| 1201 | svaelgeqid nlqrvkqkle keksefklel ddvtsnmeqi ikakanlekv srtledqane |
| 1261 | yrvkleeaqr slndfttqra klqtengela rqleekeali sqltrgklsy tqqmedlkrq |
| 1321 | leeegkakna lahalqsarh dcdllregye eeteakaelq rvlskansev aqwrtkyetd |
| 1381 | aiqrteelee akkklaqrlq daeeaveavn akcsslektk hrlgneiedl mvdversnaa |
| 1441 | aaaldkkqrn fdkilaewkq kyeesqsele ssqkearsls telfklknay eeslehletf |
| 1501 | krenknlqee isdlteqlge ggknvhelek vrkqlevekl elqsaleeae asleheegki |
| 1561 | lraglefnqi kaeierklae kdeemeqakr nhqrvvdslq tsldaetrsr nevlrvkkkm |
| 1621 | egdlnemeiq lshanrmaae aqkqvkslqs llkdtgiqld davranddlk eniaiverrn |
| 1681 | nllqaeleel ravveqters rklaeqelie tservqllhs qntslinqkk kmesdltqlq |
| 1741 | seveeavqec rnaeekakka itdaammaee lkkeqdtsah lermkknmeq tikdlqhrld |
| 1801 | eaeqialkgg kkqlqklear vrelegelea eqkrnaesvk gmrkserrik eltyqteedk |
| 1861 | knllrlqdlv dklqlkvkay krqaeeaeeq antnlskfrk vqheldeaee radiaesqvn |
| 1921 | klraksrdig akqkmhdee |

By “alphaMHC polynucleotide” is meant a polynucleotide encoding a alphaMHC polypeptide or fragment thereof. An exemplary alphaMHC polynucleotide sequence is provided at NCBI Ref: NM_002471.3. The sequence provided at NCBI Ref: NM_002471.3 is reproduced below:

| (SEQ ID NO: 26) | |
|---|---|
| 1 | agatagagag actcctgcgg cccagattct tcaggattct ccgtgaaggg ataaccaggg |
| 61 | gaagcaccaa gatgaccgat gcccagatgg ctgactttgg ggcagcggcc cagtacctcc |
| 121 | gcaagtcaga gaaggagcgt ctagaggccc agacccggcc ctttgacatt cgcactgagt |
| 181 | gcttcgtgcc cgatgacaag gaagagtttg tcaaagccaa gattttgtcc cgggagggag |
| 241 | gcaaggtcat tgctgaaacc gagaatggga agacggtgac tgtgaaggag gaccaggtgt |
| 301 | tgcagcagaa cccacccaag ttcgacaaga ttgaggacat ggccatgctg accttcctgc |
| 361 | acgagcccgc ggtgcttttc aacctcaagg agcgctacgc ggcctggatg atatatacct |
| 421 | actcgggcct cttctgtgtc actgtcaacc cctacaagtg gctgccggtg tacaatgccg |
| 481 | aggtggtggc cgcctaccgg ggcaagaaga ggagtgaggc cccgccccac atcttctcca |
| 541 | tctccgacaa cgcctatcag tacatgctga cagatcggga gaaccagtcc atcctcatca |
| 601 | cgggagaatc cggggcgggg aagactgtga acaccaagcg tgtcatccag tactttgcca |
| 661 | gcattgcagc cataggtgac cgtggcaaga aggacaatgc caatgcgaac aagggcaccc |
| 721 | tggaggacca gatcatccag gccaaccccg ctctggaggc cttcggcaat gccaagactg |
| 781 | tccggaacga caactcctcc cgctttggga aattcattag gatccacttt ggggccactg |
| 841 | gaaagctggc ttctgcagac atagagacct acctgctgga gaagtcccgg gtgatcttcc |
| 901 | agctgaaagc tgagagaaac taccacatct tctaccagat tctgtccaac aagaagccgg |
| 961 | agttgctgga catgctgctg gtcaccaaca atccctacga ctacgccttc gtgtctcagg |
| 1021 | gagaggtgtc cgtggcctcc attgatgact ccgaggagct catggccacc gatagtgcct |
| 1081 | ttgacgtgct gggcttcact tcagaggaga aagctggcgt ctacaagctg acgggagcca |
| 1141 | tcatgcacta cgggaacatg aagttcaagc agaagcagcg ggaggagcag gcggagccag |
| 1201 | acggcaccga agatgctgac aagtcggcct acctcatggg gctgaactca gctgacctgc |
| 1261 | tcaaggggct gtgccaccct cgggtgaaag tgggcaacga gtatgtcacc aaggggcaga |
| 1321 | gcgtgcagca ggtgtactac tccatcgggg ctctggccaa ggcagtgtat gagaagatgt |
| 1381 | tcaactggat ggtgacgcgc atcaacgcca ccctggagac caagcagcca cgccagtact |
| 1441 | tcataggagt cctggacatc gctggcttcg agatcttcga cttcaacagc tttgagcagc |
| 1501 | tctgcatcaa cttcaccaac gagaagctgc agcagttctt caaccaccac atgttcgtgc |
| 1561 | tggagcagga ggagtacaag aaggagggca ttgagtggac attcattgac tttggcatgg |
| 1621 | acctgcaggc ctgcattgac ctcatcgaga agcccatggg catcatgtcc atcctggagg |
| 1681 | aggagtgcat gttccccaag gccactgaca tgaccttcaa ggccaagctg tacgacaacc |
| 1741 | acctgggcaa gtccaacaat ttccagaagc cacgcaacat caaggggaag caggaagccc |
| 1801 | acttctccct gatccactac gccggcactg tggactacaa catcctgggc tggctggaaa |
| 1861 | aaaacaagga tcctctcaac gagactgttg tggccctgta ccagaagtcc tccctcaagc |
| 1921 | tcatggccac tctcttctcc tcctacgcaa ctgccgatac tggggacagt ggtaaaagca |
| 1981 | aaggaggcaa gaaaaagggc tcatccttcc agacggtgtc ggctctccac cgggaaaatc |
| 2041 | tcaacaagct aatgaccaac ctgaggacca cccatcctca ctttgtgcgt tgcatcatcc |
| 2101 | ccaatgagcg gaaggctcca ggggtgatgg acaaccccct ggtcatgcac cagctgcgct |
| 2161 | gcaatggcgt gctggagggc atccgcatct gcaggaaggg cttccccaac cgcatcctct |
| 2221 | acggggactt ccggcagagg tatcgcatcc tgaacccagt ggccatccct gagggacagt |
| 2281 | tcattgatag caggaagggg acagagaagc tgctcagctc tctggacatt gatcacaacc |
| 2341 | agtacaagtt tggccacacc aaggtgttct tcaaggcagg gctgcttggg ctgctggagg |
| 2401 | agatgcggga tgagaggctg agccgcatca tcacgcgcat gcaggcccaa gcccggggcc |
| 2461 | agctcatgcg cattgagttc aagaagatag tggaacgcag ggatgccctg ctggtaatcc |
| 2521 | agtggaacat tcgggccttc atgggggtca agaattggcc ctggatgaag ctctacttca |
| 2581 | agatcaagcc gctgctgaag agcgcagaga cggagaagga gatggccacc atgaaggaag |
| 2641 | agttcgggcg catcaaagag acgctggaga agtccgaggc tcgccgcaag gagctggagg |
| 2701 | agaagatggt gtccctgctg caggagaaga atgacctgca gctccaagtg caggcggaac |
| 2761 | aagacaacct caatgatgct gaggagcgct gcgaccagct gatcaaaaac aagattcagc |
| 2821 | tggaggccaa agtaaaggag atgaatgaga ggctggagga tgaggaggag atgaacgcgg |
| 2881 | agctcactgc caagaagcgc aagctggaag acgagtgctc agagctcaag aaggacattg |
| 2941 | atgacctgga gctgacactg gccaaggtgg agaaggagaa gcatgcaaca gagaacaagg |
| 3001 | tgaagaacct aacagaggag atggctgggc tggatgaaat catcgctaag ctgaccaagg |
| 3061 | agaagaaagc tctacaagag gcccatcagc aggccctgga tgaccttcag gttgaggaag |
| 3121 | acaaggtcaa cagcctgtcc aagtctaagg tcaagctgga gcagcaggtg gatgatctgg |
| 3181 | agggatccct agagcaagag aagaaggtgc gcatggacct ggagcgagca aagcggaaac |
| 3241 | tggagggcga cctgaagctg acccaggaga gcatcatgga cctggaaaat gataaactgc |
| 3301 | agctggaaga aaagcttaag aagaaggagt ttgacattaa tcagcagaac agtaagattg |
| 3361 | aggatgagca ggtgctggcc cttcaactac agaagaaact gaaggaaaac caggcacgca |
| 3421 | tcgaggagct ggaggaggag ctggaggccg agcgcaccgc cagggctaag gtggagaagc |
| 3481 | tgcgctcaga cctgtctcgg gagctggagg agatcagcga gcggctggaa gaggccggcg |
| 3541 | gggccacgtc cgtgcagatc gagatgaaca agaagcgcga ggccgagttc cagaagatgc |
| 3601 | ggcgggacct ggaggaggcc acgctgcagc acgaggccac tgccgcggcc ctgcgcaaga |
| 3661 | agcacgccga cagcgtggcc gagctgggcg agcagatcga caacctgcag cgggtgaagc |
| 3721 | agaagctgga gaaggagaag agcgagttca agctggagct ggatgacgtc acctccaaca |
| 3781 | tggagcagat catcaaggcc aaggcaaacc tggagaaagt gtctcggacg ctggaggacc |
| 3841 | aggccaatga gtaccgcgtg aagctagaag aggcccaacg ctccctcaat gatttcacca |
| 3901 | cccagcgagc caagctgcag accgagaatg gagagttggc ccggcagcta gaggaaaagg |
| 3961 | aggcgctaat ctcgcagctg acccggggga agctctctta tacccagcaa atggaggacc |
| 4021 | tcaaaaggca gctggaggag gagggcaagg cgaagaacgc cctggcccat gcactgcagt |
| 4081 | cggcccggca tgactgcgac ctgctgcggg agcagtacga ggaggagaca gaggccaagg |
| 4141 | ccgagctgca gcgcgtcctg tccaaggcca actcggaggt ggcccagtgg aggaccaagt |
| 4201 | atgagacgga cgccattcag cggactgagg agctcgaaga ggccaaaaag aagctggccc |
| 4261 | agcggctgca ggatgccgag gaggccgtgg aggctgttaa tgccaagtgc tcctcactgg |
| 4321 | agaagaccaa gcaccggcta cagaatgaga tagaggactt gatggtggac gtagagcgct |
| 4381 | ccaatgctgc tgctgcagcc ctggacaaga agcagagaaa ctttgacaag atcctggccg |
| 4441 | agtggaagca gaagtatgag gagtcgcagt ctgagctgga gtcctcacag aaggaggctc |
| 4501 | gctccctcag cacagagctc ttcaagctca agaacgccta cgaggagtcc ctggagcacc |
| 4561 | tagagacctt caagcgggag aacaagaacc ttcaggagga aatctcggac cttactgagc |
| 4621 | agctaggaga aggaggaaag aatgtgcatg agctggagaa ggtccgcaaa cagctggagg |
| 4681 | tggagaagct ggagctgcag tcagccctgg aggaggcaga ggcctccctg gagcacgagg |
| 4741 | agggcaagat cctccgggcc cagctagagt tcaaccagat caaggcagag atcgagcgga |
| 4801 | agctggcaga gaaggacgag gagatggaac aggccaagcg caaccaccag cgggtggtgg |
| 4861 | actcgctgca gacctccctg gatgcagaga cacgcagccg caacgaggtc ctgagggtga |
| 4921 | agaagaagat ggaaggagac ctcaatgaga tggagatcca gctcagccac gccaaccgca |
| 4981 | tggctgccga ggcccagaag caagtcaaga gcctccagag cttgctgaag gacacccaga |
| 5041 | tccagctgga cgatgcggtc cgtgccaacg acgacctgaa ggagaacatc gccatcgtgg |
| 5101 | agcggcgcaa caacctgctg caggctgagc tggaggagct gcgtgccgtg gtggagcaga |
| 5161 | cagagcggtc ccggaagctg gcggagcagg agctgattga gaccagcgag cgggtgcagc |
| 5221 | tgctgcattc ccagaacacc agcctcatca accagaagaa gaagatggag tcggatctga |
| 5281 | cccagctcca gtcggaagtg gaggaggcag tgcaggagtg cagaaacgcc gaggagaagg |
| 5341 | ccaagaaggc catcacggat gccgccatga tggcagagga gctgaagaag gagcaggaca |
| 5401 | ccagcgccca cctggagcgc atgaagaaga acatggagca gaccattaag gacctgcagc |
| 5461 | accggctgga cgaggccgag cagatcgccc tcaagggagg caagaagcag ctgcagaagc |
| 5521 | tggaagcgcg ggtgcgggag ctggagggtg agctggaggc cgagcagaag cgcaacgcag |
| 5581 | agtcggtgaa gggcatgagg aagagcgagc ggcgcatcaa ggagctcacc taccagacag |
| 5641 | aggaagacaa aaagaacctg ctgcggctac aggacctggt ggacaagctg caactgaagg |
| 5701 | tcaaggccta caagcgccag gccgaggagg cggaggagca agccaacacc aacctgtcca |
| 5761 | agttccgcaa ggtgcagcat gagctggatg aggcagagga gcgggcggac atcgctgagt |
| 5821 | cccaggtcaa caagcttcga gccaagagcc gtgacattgg tgccaagcaa aaaatgcacg |
| 5881 | atgaggagtg acactgcctc gggaacctca ctcttgccaa cctgtaataa atatgagtgc |
| 5941 | c |

By “MLC2A polypeptide” or “human MLSC2A (hMLC2A) polypeptide” is meant a protein or fragment thereof having at least 85% amino acid sequence identity to the sequence provided at NCBI Accession No. NP_067046. land having calcium binding activity. The amino acid sequence provided at NCBI Accession No. NP_067046.1 is shown below:

| (SEQ ID NO: 27) | |
|---|---|
| 1 | masrkagtrg kvaatkqaqr gssnvfsmfe qaqiqefkea fscidqnrdg iickadlret |
| 61 | ysqlgkvsvp eeeldamlqe gkgpinftvf ltlfgeklng tdpeeailsa frmfdpsgkg |
| 121 | vvnkdefkql lltqadkfsp aeveqmfalt pmdlagnidy kslcyiithg dekee |

By “MLC2A polynucleotide” is meant a polynucleotide encoding a MLC2A polypeptide or fragment thereof. An exemplary MLC2A polynucleotide sequence is provided at NCBI Ref: NM_021223.2. The sequence provided at NCBI Ref: NM_021223.2 is reproduced below:

| (SEQ ID NO: 28) | |
|---|---|
| 1 | tctgcagaga gaatggccag caggaaggcg gggacccggg gcaaggtggc agccaccaag |
| 61 | caggcccaac gtggttcttc caacgtcttt tccatgtttg aacaagccca gatacaggag |
| 121 | ttcaaagaag ccttcagctg tatcgaccag aatcgtgatg gcatcatctg caaggcagac |
| 181 | ctgagggaga cctactccca gctggggaag gtgagtgtcc cagaggagga gctggacgcc |
| 241 | atgctgcaag agggcaaggg ccccatcaac ttcaccgtct tcctcacgct ctttggggag |
| 301 | aagctcaatg ggacagaccc cgaggaagcc atcctgagtg ccttccgcat gtttgacccc |
| 361 | agcggcaaag gggtggtgaa caaggatgag ttcaagcagc ttctcctgac ccaggcagac |
| 421 | aagttctctc cagctgaggt ggagcagatg ttcgccctga cacccatgga cctggcgggg |
| 481 | aacatcgact acaagtcact gtgctacatc atcacccatg gagacgagaa agaggaatga |
| 541 | ggggcagggc caggcccacg ggggggcacc tcaataaact ctgttgcaaa attggaaaaa |
| 601 | aaaaaaaaaa aaaaaaaaa |

By “MUC2 polypeptide” is meant a protein or fragment thereof having at least 85% amino acid sequence identity to the sequence provided at NCBI Accession No. NP_002448.3 and having and having a biological activity of a MUC2 polypeptide. Exemplary biological activities of a MUC2 polypeptide include polymerization into a gel and coating of epithelia of the intestines and other mucus membrane-containing organs. The amino acid sequence provided at NCBI Accession No. NP_002448.3 is shown below:

| (SEQ ID NO: 29) | |
|---|---|
| 1 | mglplarlaa vclalslagg selqtegrtr nhghnvcstw gnfhyktfdg dvfrfpgpcd |
| 61 | ynfasdcrgs ykefavhlkr gpgqaeapag vesilltikd dtiyltrhla vlngavvstp |
| 121 | hyspglliek sdaytkvysr agltlmwnre dalmleldtk frnhtcglcg dynglqsyse |
| 181 | flsdgvlfsp lefgnmqkin qpdvvcedpe eevapascse hraecerllt aeafadcgdl |
| 241 | vplepylrac qqdrcrcpgg dtcvcstvae fsrqcshagg rpgnwrtatl cpktcpgnlv |
| 301 | ylesgspcmd tcshlevssl ceehrmdgcf cpegtvyddi gdsgcvpvsq chcrlhghly |
| 361 | tpgqeitndc eqcvcnagrw vckdlpcpgt caleggshit tfdgktytfh gdcyyvlakg |
| 421 | dhndsyallg elapcgstdk qtclktvvll adkkknvvvf ksdgsvllne lqvnlphvta |
| 481 | sfsvfrpssy himvsmaigv rlqvglapvm qlfvtldgas qgqvgglogn fnglegddfk |
| 541 | tasglveatg agfantwkaq stchdkldwl ddpcslnies anyaehwcsl lkktetpfgr |
| 601 | chsavdpaey ykrckydtcn cqnnedclca alssyaract akgvmlwgwr ehvcnkdvgs |
| 661 | cpnsqvflyn lttcqqtcrs lseadshcle gfapvdgcgc pdhtfldekg rcvplakcsc |
| 721 | yhrglyleag dvvvrqeerc vcrdgrlhcr qirligqsct apkihmdcsn ltalatskpr |
| 781 | alscqtlaag yyhtecvsgc vcpdglmddg rggcvvekec pcvhnndlys sgakikvdcn |
| 841 | tctckrgrwv ctqavchgtc siygsghyit fdgkyydfdg hcsyvavgdy cgqnsslgsf |
| 901 | siitenvpcg ttgvtcskai kifmgrtelk ledkhrvviq rdeghhvayt trevgqylvv |
| 961 | esstgiiviw dkrttvfikl apsykgtvcg lcgnfdhrsn ndfttrdhmv vsseldfgns |
| 1021 | wkeaptcpdv stnpepcsln phrrswaekq csilkssvfs ichskvdpkp fyeacvhdsc |
| 1081 | scdtggdcec fcsavasyaq ectkegacvf wrtpdlcpif cdyynpphec ewhyepcgnr |
| 1141 | sfetcrting ihsnisysyl egcyprcpkd rpiyeedlkk cvtadkcgcy vedthyppga |
| 1201 | svpteetcks cvctnssqvv crpeegkiln qtqdgafcyw eicgpngtve khfnicsitt |
| 1261 | rpstlttftt itlpttpttf ttttttttpt sstvlsttpk lcclwsdwin edhpssgsdd |
| 1321 | gdretfdgvc gapediecrs vkdphlsleq lgqkvqcdvs vgficknedq fgngpfglcy |
| 1381 | dykirvnccw pmdkcittps pptttpsppp tstttlpptt tpsppttttt tppptttpsp |
| 1441 | pitttttppp tttpsppist tttppptttp spptttpspp tttpsppttt tttppptttp |
| 1501 | spptttpitp pastttlppt ttpspptttt ttppptttps pptttpitpp tstttlpptt |
| 1561 | tpsppptttt tppptttpsp pttttpsppt ittttppptt tpsppttttt tppptttpsp |
| 1621 | ptttpitppt stttlppttt pspppttttt ppptttpspp ttttpsppit ttttpppttt |
| 1681 | psspitttps pptttmttps ptttpsspit ttttpssttt pspppttmtt psptttpspp |
| 1741 | tttmttlppt ttsspltttp lppsitpptf spfstttptt pcvplcnwtg wldsgkpnfh |
| 1801 | kpggdtelig dvcgpgwaan iscratmypd vpigglgqtv vcdvsvglic knedqkpggv |
| 1861 | ipmafclnye invqccecvt qpttmttttt enptpptttp itttttvtpt ptptgtqtpt |
| 1921 | ttpitttttv tptptptgtq tptttpittt ttvtptptpt gtqtptttpi tttttvtptp |
| 1981 | tptgtqtptt tpitttttvt ptptptgtqt ptttpitttt tvtptptptg tqtptttpit |
| 2041 | ttttvtptpt ptgtqtpttt pitttttvtp tptptgtqtp tttpittttt vtptptptgt |
| 2101 | qtptttpitt tttvtptptp tgtqtptttp itttttvtpt ptptgtqtpt ttpitttttv |
| 2161 | tptptptgtq tptttpittt ttvtptptpt gtqtptttpi tttttvtptp tptgtqtptt |
| 2221 | tpitttttvt ptptptgtqt ptttpitttt tvtptptptg tqtptttpit ttttvtptpt |
| 2281 | ptgtqtpttt pitttttvtp tptptgtqtp tttpittttt vtptptptgt qtptttpitt |
| 2341 | tttvtptptp tgtqtptttp itttttvtpt ptptgtqtpt ttpitttttv tptptptgtq |
| 2401 | tptttpittt ttvtptptpt gtqtptttpi tttttvtptp tptgtqtptt tpitttttvt |
| 2461 | ptptptgtqt ptttpitttt tvtptptptg tqtptttpit ttttvtptpt ptgtqtpttt |
| 2521 | pitttttvtp tptptgtqtp tttpittttt vtptptptgt qtptttpitt tttvtptptp |
| 2581 | tgtqtptttp itttttvtpt ptptgtqtpt ttpitttttv tptptptgtq tptttpittt |
| 2641 | ttvtptptpt gtqtptttpi tttttvtptp tptgtqtptt tpitttttvt ptptptgtqt |
| 2701 | ptttpitttt tvtptptptg tqtptttpit ttttvtptpt ptgtqtpttt pitttttvtp |
| 2761 | tptptgtqtp tttpittttt vtptptptgt qtptttpitt tttvtptptp tgtqtptttp |
| 2821 | itttttvtpt ptptgtqtpt ttpitttttv tptptptgtq tptttpittt ttvtptptpt |
| 2881 | gtqtptttpi tttttvtptp tptgtqtptt tpitttttvt ptptptgtqt ptttpitttt |
| 2941 | tvtptptptg tqtptttpit ttttvtptpt ptgtqtpttt pitttttvtp tptptgtqtp |
| 3001 | tttpittttt vtptptptgt qtptttpitt tttvtptptp tgtqtptttp itttttvtpt |
| 3061 | ptptgtqtpt ttpitttttv tptptptgtq tptttpittt ttvtptptpt gtqtptttpi |
| 3121 | tttttvtptp tptgtqtptt tpitttttvt ptptptgtqt ptttpitttt tvtptptptg |
| 3181 | tqtptttpit ttttvtptpt ptgtqtpttt pitttttvtp tptptgtqtp tttpittttt |
| 3241 | vtptptptgt qtptttpitt tttvtptptp tgtqtptttp itttttvtpt ptptgtqtpt |
| 3301 | ttpitttttv tptptptgtq tptttpittt ttvtptptpt gtqtptttpi tttttvtptp |
| 3361 | tptgtqtptt tpitttttvt ptptptgtqt ptttpitttt tvtptptptg tqtptttpit |
| 3421 | ttttvtptpt ptgtqtpttt pitttttvtp tptptgtqtp tttpittttt vtptptptgt |
| 3481 | qtptttpitt tttvtptptp tgtqtptttp itttttvtpt ptptgtqtpt ttpitttttv |
| 3541 | tptptptgtq tptttpittt ttvtptptpt gtqtptttpi tttttvtptp tptgtqtptt |
| 3601 | tpitttttvt ptptptgtqt ptttpitttt tvtptptptg tqtptttpit ttttvtptpt |
| 3661 | ptgtqtpttt pitttttvtp tptptgtqtp tttpittttt vtptptptgt qtptttpitt |
| 3721 | tttvtptptp tgtqtptttp itttttvtpt ptptgtqtpt ttpitttttv tptptptgtq |
| 3781 | tptttpittt ttvtptptpt gtqtptttpi tttttvtptp tptgtqtptt tpitttttvt |
| 3841 | ptptptgtqt ptttpitttt tvtptptptg tqtptttpit ttttvtptpt ptgtqtpttt |
| 3901 | pitttttvtp tptptgtqtp tttpittttt vtptptptgt qtptttpitt tttvtptptp |
| 3961 | tgtqtptttp itttttvtpt ptptgtqtpt ttpitttttv tptptptgtq tptttpittt |
| 4021 | ttvtptptpt gtqtptttpi tttttvtptp tptgtqtptt tpitttttvt ptptptgtqt |
| 4081 | ptttpitttt tvtptptptg tqtptttpit ttttvtptpt ptgtqtpttt pitttttvtp |
| 4141 | tptptgtqtp tttpittttt vtptptptgt qtptttpitt tttvtptptp tgtqtgppth |
| 4201 | tstapiaelt tsnpppesst pqtsrstssp ltesttllst lppaiemtst appstptapt |
| 4261 | ttsgghtlsp ppstttsppg tptrgtttgs ssaptpstvq ttttsawtpt ptplstpsii |
| 4321 | rttglrpyps svliccvind tyyapgeevy ngtygdtcyf vncslsctle fynwscpstp |
| 4381 | sptptpskst ptpskpsstp skptpgtkpp ecpdfdpprq enetwwlcdc fmatckynnt |
| 4441 | veivkvecep ppmptcsngl qpvrvedpdg ccwhwecdcy ctgwgdphyv tfdglyysyq |
| 4501 | gnctyvlvee ispsvdnfgv yidnyhcdpn dkvscprtli vrhetqevli ktvhmmpmqv |
| 4561 | qvgvnrgava lpykkyglev yqsginyvvd ipelgvlvsy nglsfsvrlp yhrfgnntkg |
| 4621 | qcgtctntts ddcilpsgei vsnceaaadq wlvndpskph cphsssttkr pavtvpgggk |
| 4681 | ttphkdctps ploglikdsl faqchalvpp qhyydacvfd scfmpgssle caslqayaal |
| 4741 | caqqnicldw rnhthgaclv ecpshreyqa cgpaeeptck ssssqqnntv lvegcfcpeg |
| 4801 | tmnyapgfdv cvktcgcvgp dnvprefgeh fefdckncvc leggsgiicq pkrcsqkpvt |
| 4861 | hcvedgtyla tevnpadtcc nitvckcnts lckekpsvcp lgfevkskmv pgrccpfywc |
| 4921 | eskgvcvhgn aeyqpgspvy sskcqdcvct dkvdnntlln viacthvpcn tscspgfelm |
| 4981 | eapgecckkc eqthciikrp dnqhvilkpg dfksdpknnc tffscvkihn qlissysnit |
| 5041 | cpnfdasici pgsitfmpng ccktctprne trvpcstvpv ttevsyagct ktvlmnhcsg |
| 5101 | scgtfvmysa kaqaldhscs cckeektsqr evvlscpngg slthtythie scqcqdtvcg |
| 5161 | lptgtsrrar rsprhlgsg |

By “MUC2 polynucleotide” is meant a polynucleotide encoding a MUC2 polypeptide or fragment thereof. An exemplary MUC2 polynucleotide sequence is provided at NCBI Ref: NM_002457.3. The sequence provided at NCBI Ref: NM_002457.3 is reproduced below:

| (SEQ ID NO: 30) | |
|---|---|
| 1 | caacccacac cgcccctgcc agccaccatg gggctgccac tagcccgcct ggcggctgtg |
| 61 | tgcctggccc tgtctttggc agggggctcg gagctccaga cagagggcag aacccgaaac |
| 121 | cacggccaca acgtctgcag cacctggggc aacttccact acaagacctt cgacggggac |
| 181 | gtcttccgct tccccggccc ctgcgactac aacttcgcct ccgactgccg aggctcctac |
| 241 | aaggaatttg ctgtgcacct gaagcggggt ccgggccagg ctgaggcccc cgccggggtg |
| 301 | gagtccatcc tgctgaccat caaggatgac accatctacc tcacccgcca cctggctgtg |
| 361 | cttaacgggg ccgtggtcag caccccgcac tacagccccg ggctgctcat tgagaagagc |
| 421 | gatgcctaca ccaaagtcta ctcccgcgcc ggcctcaccc tcatgtggaa ccgggaggat |
| 481 | gcactcatgc tggagctgga cactaagttc cggaaccaca cctgtggcct ctgcggggac |
| 541 | tacaacggcc tgcagagcta ttcagaattc ctctctgacg gcgtgctctt cagtcccctg |
| 601 | gagtttggga acatgcagaa gatcaaccag cccgatgtgg tgtgtgagga tcccgaggag |
| 661 | gaggtggccc ccgcatcctg ctccgagcac cgcgccgagt gtgagaggct gctgaccgcc |
| 721 | gaggccttcg cggactgtca ggacctggtg ccgctggagc cgtatctgcg cgcctgccag |
| 781 | caggaccgct gccggtgccc gggcggtgac acctgcgtct gcagcaccgt ggccgagttc |
| 841 | tcccgccagt gctcccacgc cggcggccgg cccgggaact ggaggaccgc cacgctctgc |
| 901 | cccaagacct gccccgggaa cctggtgtac ctggagagcg gctcgccctg catggacacc |
| 961 | tgctcacacc tggaggtgag cagcctgtgc gaggagcacc gcatggacgg ctgtttctgc |
| 1021 | ccagaaggca ccgtatatga cgacatcggg gacagtggct gcgttcctgt gagccagtgc |
| 1081 | cactgcaggc tgcacggaca cctgtacaca ccgggccagg agatcaccaa tgactgcgag |
| 1141 | cagtgtgtct gtaacgctgg ccgctgggtg tgcaaagacc tgccctgccc cggcacctgt |
| 1201 | gccctggaag gcggctccca catcaccacc ttcgatggga agacgtacac cttccacggg |
| 1261 | gactgctact atgtcctggc caagggtgac cacaacgatt cctacgctct cctgggcgag |
| 1321 | ctggccccct gtggctccac agacaagcag acctgcctga agacggtggt gctgctggct |
| 1381 | gacaagaaga agaatgtggt ggtcttcaag tccgatggca gtgtactgct caacgagctg |
| 1441 | caggtgaacc tgccccacgt gaccgcgagc ttctctgtct tccgcccgtc ttcctaccac |
| 1501 | atcatggtga gcatggccat tggcgtccgg ctgcaggtgc agctggcccc agtcatgcaa |
| 1561 | ctctttgtga cactggacca ggcctcccag gggcaggtgc agggcctctg cgggaacttc |
| 1621 | aacggcctgg aaggtgacga cttcaagacg gccagcgggc tggtggaggc cacgggggcc |
| 1681 | ggctttgcca acacctggaa ggcacagtca acctgccatg acaagctgga ctggttggac |
| 1741 | gatccctgct ccctgaacat cgagagcgcc aactacgccg agcactggtg ctccctcctg |
| 1801 | aagaagacag agaccccctt tggcaggtgc cactcggctg tggaccctgc tgagtattac |
| 1861 | aagaggtgca aatatgacac gtgtaactgt cagaacaatg aggactgcct gtgcgccgcc |
| 1921 | ctgtcctcct acgcgcgcgc ctgcaccgcc aagggcgtca tgctgtgggg ctggcgggag |
| 1981 | catgtctgca acaaggatgt gggctcctgc cccaactcgc aggtcttcct gtacaacctg |
| 2041 | accacctgcc agcagacctg ccgctccctc tccgaggccg acagccactg tctcgagggc |
| 2101 | tttgcgcctg tggacggctg cggctgccct gaccacacct tcctggacga gaagggccgc |
| 2161 | tgcgtacccc tggccaagtg ctcctgttac caccgcggtc tctacctgga ggcgggggac |
| 2221 | gtggtcgtca ggcaggaaga acgatgtgtg tgccgggatg ggcggctgca ctgtaggcag |
| 2281 | atccggctga tcggccagag ctgcacggcc ccaaagatcc acatggactg cagcaacctg |
| 2341 | actgcactgg ccacctcgaa gccccgagcc ctcagctgcc agacgctggc cgccggctat |
| 2401 | taccacacag agtgtgtcag tggctgtgtg tgccccgacg ggctgatgga tgacggccgg |
| 2461 | ggtggctgcg tggtggagaa ggaatgccct tgcgtccata acaacgacct gtattcttcc |
| 2521 | ggcgccaaga tcaaggtgga ctgcaatacc tgcacctgca agagaggacg ctgggtgtgc |
| 2581 | acccaggctg tgtgccatgg cacctgctcc atttacggga gtggccacta catcaccttt |
| 2641 | gacgggaagt actacgactt tgacggacac tgctcctacg tggctgttca ggactactgc |
| 2701 | ggccagaact cctcactggg ctcattcagc atcatcaccg agaacgtccc ctgtggcact |
| 2761 | acgggcgtca cctgctccaa ggccatcaag atcttcatgg ggaggacgga gctgaagttg |
| 2821 | gaagacaagc accgtgtggt gatccagcgt gatgagggtc accacgtggc ctacaccacg |
| 2881 | cgggaggtgg gccagtacct ggtggtggag tccagcacgg gcatcatcgt catctgggac |
| 2941 | aagaggacca ccgtgttcat caagctggct ccctcctaca agggcaccgt gtgtggcctg |
| 3001 | tgtgggaact ttgaccaccg ctccaacaac gacttcacca cgcgggacca catggtggtg |
| 3061 | agcagcgagc tggacttcgg gaacagctgg aaggaggccc ccacctgccc agatgtgagc |
| 3121 | accaaccccg agccctgcag cctgaacccg caccgccgct cctgggccga gaagcagtgc |
| 3181 | agcatcctca aaagcagcgt gttcagcatc tgccacagca aggtggaccc caagcccttc |
| 3241 | tacgaggcct gtgtgcacga ctcgtgctcc tgtgacacgg gtggggactg tgagtgcttc |
| 3301 | tgctctgccg tggcctccta cgcccaggag tgtaccaaag agggggcctg cgtgttctgg |
| 3361 | aggacgccgg acctgtgccc catattctgc gactactaca accctccgca tgagtgtgag |
| 3421 | tggcactatg agccatgtgg gaaccggagc ttcgagacct gcaggaccat caatggcatc |
| 3481 | cactccaaca tctccgtgtc ctacctggag ggctgctacc cccggtgccc caaggacagg |
| 3541 | cccatctatg aggaggatct gaagaagtgt gtcactgcag acaagtgtgg ctgctatgtc |
| 3601 | gaggacaccc actacccacc tggagcatcg gttcccaccg aggagacctg caagtcctgc |
| 3661 | gtgtgtacca actcctccca agtcgtctgc aggccggagg aaggaaagat tcttaaccag |
| 3721 | acccaggatg gcgccttctg ctactgggag atctgtggcc ccaacgggac ggtggagaag |
| 3781 | cacttcaaca tctgttccat tacgacacgc ccgtccaccc tgaccacctt caccaccatc |
| 3841 | accctcccca ccacccccac caccttcacc actaccacca ccaccaccac cccgacctcc |
| 3901 | agcacagttt tatcaacaac tccgaagctg tgctgcctct ggtctgactg gatcaatgag |
| 3961 | gaccacccca gcagtggcag cgacgacggt gaccgagaaa catttgatgg ggtctgcggg |
| 4021 | gcccctgagg acatcgagtg caggtcggtc aaggatcccc acctcagctt ggagcagcta |
| 4081 | ggccagaagg tgcagtgtga tgtctctgtt gggttcattt gcaagaatga agaccagttt |
| 4141 | ggaaatggac catttggact gtgttacgac tacaagatac gtgtcaattg ttgctggccc |
| 4201 | atggataagt gtatcaccac tcccagccct ccaactacca ctcccagccc tccaccaacc |
| 4261 | agcacgacca cccttccacc aaccaccacc cccagccctc caaccaccac cacaaccacc |
| 4321 | cctccaccaa ccaccacccc cagccctcca ataaccacca cgaccacccc tccaccaacc |
| 4381 | accactccca gccctccaat aagcaccaca accacccctc caccaaccac cactcccagc |
| 4441 | cctccaacca ccactcccag ccctccaacc accactccca gccctccaac aaccaccaca |
| 4501 | accacccctc caccaaccac cactcccagc cctccaacga ctacgcccat cactccacca |
| 4561 | gccagcacta ccacccttcc accaaccacc actcccagcc ctccaacaac caccacaacc |
| 4621 | acccctccac caaccaccac tcccagtcct ccaacgacta cgcccatcac tccaccaacc |
| 4681 | agcactacta cccttccacc aaccaccact cccagccctc caccaaccac cacaaccacc |
| 4741 | cctccaccaa ccaccactcc cagccctcca acaaccacca ctcccagtcc tccaacaatc |
| 4801 | accacaacca cccctccacc aaccaccact cccagccctc caacaacgac cacaaccacc |
| 4861 | cctccaccaa ccaccactcc cagccctcca acgactacac ccatcactcc accaaccagc |
| 4921 | actaccaccc ttccaccaac caccactccc agccctccac caaccaccac aaccacccct |
| 4981 | ccaccaacca ccactcccag ccctccaaca accaccactc ccagccctcc aataaccacc |
| 5041 | acaaccaccc ctccaccaac caccactccc agctctccaa taaccaccac tcccagccct |
| 5101 | ccaacaacca ccatgaccac cccttcacca accaccaccc ccagctctcc aataaccacc |
| 5161 | acaaccaccc cttcctcaac taccactccc agccctccac caaccaccat gaccacccct |
| 5221 | tcaccaacca ccactcccag ccctccaaca accaccatga ccacccttcc accaaccacc |
| 5281 | acttccagcc ctctaacaac tactcctcta cctccatcaa taactcctcc tacattttca |
| 5341 | ccattctcaa cgacaacccc tactacccca tgcgtgcctc tctgcaattg gactggctgg |
| 5401 | ctggattctg gaaaacccaa ctttcacaaa ccaggtggag acacagaatt gattggagac |
| 5461 | gtctgtggac caggctgggc agctaacatc tcttgcagag ccaccatgta tcctgatgtt |
| 5521 | cccattggac agcttggaca aacagtggtg tgtgatgtct ctgtggggct gatatgcaaa |
| 5581 | aatgaagacc aaaagccagg tggggtcatc cctatggcct tctgcctcaa ctacgagatc |
| 5641 | aacgttcagt gctgtgagtg tgtcacccaa cccaccacca tgacaaccac caccacagag |
| 5701 | aacccaactc cgccaaccac gacacccatc accaccacca ctacggtgac cccaacccca |
| 5761 | acacccaccg gcacacagac cccaaccacg acacccatca ccaccaccac tacggtgacc |
| 5821 | ccaaccccaa cacccaccgg cacacagacc ccaaccacga cacccatcac caccaccact |
| 5881 | acggtgaccc caaccccaac acccaccggc acacagaccc caaccacgac acccatcacc |
| 5941 | accaccacta cggtgacccc aaccccaaca cccaccggca cacagacccc aaccacgaca |
| 6001 | cccatcacca ccaccactac ggtgacccca accccaacac ccaccggcac acagacccca |
| 6061 | accacgacac ccatcaccac caccactacg gtgaccccaa ccccaacacc caccggcaca |
| 6121 | cagaccccaa ccacgacacc catcaccacc accactacgg tgaccccaac cccaacaccc |
| 6181 | accggcacac agaccccaac cacgacaccc atcaccacca ccactacggt gaccccaacc |
| 6241 | ccaacaccca ccggcacaca gaccccaacc acgacaccca tcaccaccac cactacggtg |
| 6301 | accccaaccc caacacccac cggcacacag accccaacca cgacacccat caccaccacc |
| 6361 | actacggtga ccccaacccc aacacccacc ggcacacaga ccccaaccac gacacccatc |
| 6421 | accaccacca ctacggtgac cccaacccca acacccaccg gcacacagac cccaaccacg |
| 6481 | acacccatca ccaccaccac tacggtgacc ccaaccccaa cacccaccgg cacacagacc |
| 6541 | ccaaccacga cacccatcac caccaccact acggtgaccc caaccccaac acccaccggc |
| 6601 | acacagaccc caaccacgac acccatcacc accaccacta cggtgacccc aaccccaaca |
| 6661 | cccaccggca cacagacccc aaccacgaca cccatcacca ccaccactac ggtgacccca |
| 6721 | accccaacac ccaccggcac acagacccca accacgacac ccatcaccac caccactacg |
| 6781 | gtgaccccaa ccccaacacc caccggcaca cagaccccaa ccacgacacc catcaccacc |
| 6841 | accactacgg tgaccccaac cccaacaccc accggcacac agaccccaac cacgacaccc |
| 6901 | atcaccacca ccactacggt gaccccaacc ccaacaccca ccggcacaca gaccccaacc |
| 6961 | acgacaccca tcaccaccac cactacggtg accccaaccc caacacccac cggcacacag |
| 7021 | accccaacca cgacacccat caccaccacc actacggtga ccccaacccc aacacccacc |
| 7081 | ggcacacaga ccccaaccac gacacccatc accaccacca ctacggtgac cccaacccca |
| 7141 | acacccaccg gcacacagac cccaaccacg acacccatca ccaccaccac tacggtgacc |
| 7201 | ccaaccccaa cacccaccgg cacacagacc ccaaccacga cacccatcac caccaccact |
| 7261 | acggtgaccc caaccccaac acccaccggc acacagaccc caaccacgac acccatcacc |
| 7321 | accaccacta cggtgacccc aaccccaaca cccaccggca cacagacccc aaccacgaca |
| 7381 | cccatcacca ccaccactac ggtgacccca accccaacac ccaccggcac acagacccca |
| 7441 | accacgacac ccatcaccac caccactacg gtgaccccaa ccccaacacc caccggcaca |
| 7501 | cagaccccaa ccacgacacc catcaccacc accactacgg tgaccccaac cccaacaccc |
| 7561 | accggcacac agaccccaac cacgacaccc atcaccacca ccactacggt gaccccaacc |
| 7621 | ccaacaccca ccggcacaca gaccccaacc acgacaccca tcaccaccac cactacggtg |
| 7681 | accccaaccc caacacccac cggcacacag accccaacca cgacacccat caccaccacc |
| 7741 | actacggtga ccccaacccc aacacccacc ggcacacaga ccccaaccac gacacccatc |
| 7801 | accaccacca ctacggtgac cccaacccca acacccaccg gcacacagac cccaaccacg |
| 7861 | acacccatca ccaccaccac tacggtgacc ccaaccccaa cacccaccgg cacacagacc |
| 7921 | ccaaccacga cacccatcac caccaccact acggtgaccc caaccccaac acccaccggc |
| 7981 | acacagaccc caaccacgac acccatcacc accaccacta cggtgacccc aaccccaaca |
| 8041 | cccaccggca cacagacccc aaccacgaca cccatcacca ccaccactac ggtgacccca |
| 8101 | accccaacac ccaccggcac acagacccca accacgacac ccatcaccac caccactacg |
| 8161 | gtgaccccaa ccccaacacc caccggcaca cagaccccaa ccacgacacc catcaccacc |
| 8221 | accactacgg tgaccccaac cccaacaccc accggcacac agaccccaac cacgacaccc |
| 8281 | atcaccacca ccactacggt gaccccaacc ccaacaccca ccggcacaca gaccccaacc |
| 8341 | acgacaccca tcaccaccac cactacggtg accccaaccc caacacccac cggcacacag |
| 8401 | accccaacca cgacacccat caccaccacc actacggtga ccccaacccc aacacccacc |
| 8461 | ggcacacaga ccccaaccac gacacccatc accaccacca ctacggtgac cccaacccca |
| 8521 | acacccaccg gcacacagac cccaaccacg acacccatca ccaccaccac tacggtgacc |
| 8581 | ccaaccccaa cacccaccgg cacacagacc ccaaccacga cacccatcac caccaccact |
| 8641 | acggtgaccc caaccccaac acccaccggc acacagaccc caaccacgac acccatcacc |
| 8701 | accaccacta cggtgacccc aaccccaaca cccaccggca cacagacccc aaccacgaca |
| 8761 | cccatcacca ccaccactac ggtgacccca accccaacac ccaccggcac acagacccca |
| 8821 | accacgacac ccatcaccac caccactacg gtgaccccaa ccccaacacc caccggcaca |
| 8881 | cagaccccaa ccacgacacc catcaccacc accactacgg tgaccccaac cccaacaccc |
| 8941 | accggcacac agaccccaac cacgacaccc atcaccacca ccactacggt gaccccaacc |
| 9001 | ccaacaccca ccggcacaca gaccccaacc acgacaccca tcaccaccac cactacggtg |
| 9061 | accccaaccc caacacccac cggcacacag accccaacca cgacacccat caccaccacc |
| 9121 | actacggtga ccccaacccc aacacccacc ggcacacaga ccccaaccac gacacccatc |
| 9181 | accaccacca ctacggtgac cccaacccca acacccaccg gcacacagac cccaaccacg |
| 9241 | acacccatca ccaccaccac tacggtgacc ccaaccccaa cacccaccgg cacacagacc |
| 9301 | ccaaccacga cacccatcac caccaccact acggtgaccc caaccccaac acccaccggc |
| 9361 | acacagaccc caaccacgac acccatcacc accaccacta cggtgacccc aaccccaaca |
| 9421 | cccaccggca cacagacccc aaccacgaca cccatcacca ccaccactac ggtgacccca |
| 9481 | accccaacac ccaccggcac acagacccca accacgacac ccatcaccac caccactacg |
| 9541 | gtgaccccaa ccccaacacc caccggcaca cagaccccaa ccacgacacc catcaccacc |
| 9601 | accactacgg tgaccccaac cccaacaccc accggcacac agaccccaac cacgacaccc |
| 9661 | atcaccacca ccactacggt gaccccaacc ccaacaccca ccggcacaca gaccccaacc |
| 9721 | acgacaccca tcaccaccac cactacggtg accccaaccc caacacccac cggcacacag |
| 9781 | accccaacca cgacacccat caccaccacc actacggtga ccccaacccc aacacccacc |
| 9841 | ggcacacaga ccccaaccac gacacccatc accaccacca ctacggtgac cccaacccca |
| 9901 | acacccaccg gcacacagac cccaaccacg acacccatca ccaccaccac tacggtgacc |
| 9961 | ccaaccccaa cacccaccgg cacacagacc ccaaccacga cacccatcac caccaccact |
| 10021 | acggtgaccc caaccccaac acccaccggc acacagaccc caaccacgac acccatcacc |
| 10081 | accaccacta cggtgacccc aaccccaaca cccaccggca cacagacccc aaccacgaca |
| 10141 | cccatcacca ccaccactac ggtgacccca accccaacac ccaccggcac acagacccca |
| 10201 | accacgacac ccatcaccac caccactacg gtgaccccaa ccccaacacc caccggcaca |
| 10261 | cagaccccaa ccacgacacc catcaccacc accactacgg tgaccccaac cccaacaccc |
| 10321 | accggcacac agaccccaac cacgacaccc atcaccacca ccactacggt gaccccaacc |
| 10381 | ccaacaccca ccggcacaca gaccccaacc acgacaccca tcaccaccac cactacggtg |
| 10441 | accccaaccc caacacccac cggcacacag accccaacca cgacacccat caccaccacc |
| 10501 | actacggtga ccccaacccc aacacccacc ggcacacaga ccccaaccac gacacccatc |
| 10561 | accaccacca ctacggtgac cccaacccca acacccaccg gcacacagac cccaaccacg |
| 10621 | acacccatca ccaccaccac tacggtgacc ccaaccccaa cacccaccgg cacacagacc |
| 10681 | ccaaccacga cacccatcac caccaccact acggtgaccc caaccccaac acccaccggc |
| 10741 | acacagaccc caaccacgac acccatcacc accaccacta cggtgacccc aaccccaaca |
| 10801 | cccaccggca cacagacccc aaccacgaca cccatcacca ccaccactac ggtgacccca |
| 10861 | accccaacac ccaccggcac acagacccca accacgacac ccatcaccac caccactacg |
| 10921 | gtgaccccaa ccccaacacc caccggcaca cagaccccaa ccacgacacc catcaccacc |
| 10981 | accactacgg tgaccccaac cccaacaccc accggcacac agaccccaac cacgacaccc |
| 11041 | atcaccacca ccactacggt gaccccaacc ccaacaccca ccggcacaca gaccccaacc |
| 11101 | acgacaccca tcaccaccac cactacggtg accccaaccc caacacccac cggcacacag |
| 11161 | accccaacca cgacacccat caccaccacc actacggtga ccccaacccc aacacccacc |
| 11221 | ggcacacaga ccccaaccac gacacccatc accaccacca ctacggtgac cccaacccca |
| 11281 | acacccaccg gcacacagac cccaaccacg acacccatca ccaccaccac tacggtgacc |
| 11341 | ccaaccccaa cacccaccgg cacacagacc ccaaccacga cacccatcac caccaccact |
| 11401 | acggtgaccc caaccccaac acccaccggc acacagaccc caaccacgac acccatcacc |
| 11461 | accaccacta cggtgacccc aaccccaaca cccaccggca cacagacccc aaccacgaca |
| 11521 | cccatcacca ccaccactac ggtgacccca accccaacac ccaccggcac acagacccca |
| 11581 | accacgacac ccatcaccac caccactacg gtgaccccaa ccccaacacc caccggcaca |
| 11641 | cagaccccaa ccacgacacc catcaccacc accactacgg tgaccccaac cccaacaccc |
| 11701 | accggcacac agaccccaac cacgacaccc atcaccacca ccactacggt gaccccaacc |
| 11761 | ccaacaccca ccggcacaca gaccccaacc acgacaccca tcaccaccac cactacggtg |
| 11821 | accccaaccc caacacccac cggcacacag accccaacca cgacacccat caccaccacc |
| 11881 | actacggtga ccccaacccc aacacccacc ggcacacaga ccccaaccac gacacccatc |
| 11941 | accaccacca ctacggtgac cccaacccca acacccaccg gcacacagac cccaaccacg |
| 12001 | acacccatca ccaccaccac tacggtgacc ccaaccccaa cacccaccgg cacacagacc |
| 12061 | ccaaccacga cacccatcac caccaccact acggtgaccc caaccccaac acccaccggc |
| 12121 | acacagaccc caaccacgac acccatcacc accaccacta cggtgacccc aaccccaaca |
| 12181 | cccaccggca cacagacccc aaccacgaca cccatcacca ccaccactac ggtgacccca |
| 12241 | accccaacac ccaccggcac acagacccca accacgacac ccatcaccac caccactacg |
| 12301 | gtgaccccaa ccccaacacc caccggcaca cagaccccaa ccacgacacc catcaccacc |
| 12361 | accactacgg tgaccccaac cccaacaccc accggcacac agaccccaac cacgacaccc |
| 12421 | atcaccacca ccactacggt gaccccaacc ccaacaccca ccggcacaca gaccccaacc |
| 12481 | acgacaccca tcaccaccac cactacggtg accccaaccc caacacccac cggcacacag |
| 12541 | accccaacca cgacacccat caccaccacc actacggtga ccccaacccc aacacccacc |
| 12601 | ggcacacaga ccgggccccc cacccacaca agcacagcac cgattgctga gttgaccaca |
| 12661 | tccaatcctc cgcctgagtc ctcaacccct cagacctctc ggtccacctc ttcccctctc |
| 12721 | acggagtcaa ccacccttct gagtacccta ccacctgcca ttgagatgac cagcacggcc |
| 12781 | ccaccctcca cacccacggc acccacgacc acgagcggag gccacacact gtctccaccg |
| 12841 | cccagcacca ccacgtcccc tccaggcacc cccactcgcg gtaccacgac tgggtcatct |
| 12901 | tcagccccca cccccagcac tgtgcagacg accaccacca gtgcctggac ccccacgccg |
| 12961 | accccactct ccacacccag catcatcagg accacaggcc tgaggcccta cccttcctct |
| 13021 | gtgcttatct gctgtgtcct gaacgacacc tactacgcac caggtgagga ggtgtacaac |
| 13081 | ggcacatacg gagacacctg ttatttcgtc aactgctcac tgagctgtac gttggagttc |
| 13141 | tataactggt cctgcccatc cacgccctcc ccaacaccca cgccctccaa gtcgacgccc |
| 13201 | acgccttcca agccatcgtc cacgccctcc aagccgacgc ccggcaccaa gccccccgag |
| 13261 | tgcccagact ttgatcctcc cagacaggag aacgagactt ggtggctgtg cgactgcttc |
| 13321 | atggccacgt gcaagtacaa caacacggtg gagatcgtga aggtggagtg tgagccgccg |
| 13381 | cccatgccca cctgctccaa cggcctccaa cccgtgcgcg tcgaggaccc cgacggctgc |
| 13441 | tgctggcact gggagtgcga ctgctactgc acgggctggg gcgacccgca ctatgtcacc |
| 13501 | ttcgacggac tctactacag ctaccagggc aactgcacct acgtgctggt ggaggagatc |
| 13561 | agcccctccg tggacaactt cggagtttac atcgacaact accactgcga tcccaacgac |
| 13621 | aaggtgtcct gcccccgcac cctcatcgtg cgccacgaga cccaggaggt gctgatcaag |
| 13681 | accgtgcata tgatgcccat gcaggtgcag gtgcaggtga acaggcaggc ggtggcactg |
| 13741 | ccctacaaga agtacgggct ggaggtgtac cagtctggca tcaactacgt ggtggacatc |
| 13801 | cccgagctgg gtgtcctcgt ctcctacaat ggcctgtcct tctccgtcag gctgccctac |
| 13861 | caccggtttg gcaacaacac caagggccag tgtggcacct gcaccaacac cacctccgac |
| 13921 | gactgcattc tgcccagcgg ggagatcgtc tccaactgtg aggctgcggc tgaccagtgg |
| 13981 | ctggtgaacg acccctccaa gccacactgc ccccacagca gctccacgac caagcgcccg |
| 14041 | gccgtcactg tgcccggggg cggtaaaacg accccacaca aggactgcac cccatctccc |
| 14101 | ctctgccagc tcatcaagga cagcctgttt gcccagtgcc acgcactggt gcccccgcag |
| 14161 | cactactacg atgcctgcgt gttcgacagc tgcttcatgc cgggctcgag cctggagtgc |
| 14221 | gccagtctgc aggcctacgc agccctctgt gcccagcaga acatctgcct cgactggcgg |
| 14281 | aaccacacgc atggggcctg cttggtggag tgcccatctc acagggagta ccaggcctgt |
| 14341 | ggccctgcag aagagcccac gtgcaaatcc agctcctccc agcagaacaa cacagtcctg |
| 14401 | gtggaaggct gcttctgtcc tgagggcacc atgaactacg ctcctggctt tgatgtctgc |
| 14461 | gtgaagacct gcggctgtgt gggacctgac aatgtgccca gagagtttgg ggagcacttc |
| 14521 | gagttcgact gcaagaactg tgtctgcctg gagggtggaa gtggcatcat ctgccaaccc |
| 14581 | aagaggtgca gccagaagcc cgttacccac tgcgtggaag acggcaccta cctcgccacg |
| 14641 | gaggtcaacc ctgccgacac ctgctgcaac attaccgtct gcaagtgcaa caccagcctg |
| 14701 | tgcaaagaga agccctccgt gtgcccgctg ggattcgaag tgaagagcaa gatggtgcct |
| 14761 | ggaaggtgct gtcctttcta ctggtgtgag tccaaggggg tgtgtgttca cgggaatgct |
| 14821 | gagtaccagc ccggttctcc agtttattcc tccaagtgcc aggactgcgt gtgcacggac |
| 14881 | aaggtggaca acaacaccct gctcaacgtc atcgcctgca cccacgtgcc ctgcaacacc |
| 14941 | tcctgcagcc ctggcttcga actcatggag gcccccgggg agtgctgtaa gaagtgtgaa |
| 15001 | cagacgcact gtatcatcaa acggcccgac aaccagcacg tcatcctgaa gcccggggac |
| 15061 | ttcaagagcg acccgaagaa caactgcaca ttcttcagct gcgtgaagat ccacaaccag |
| 15121 | ctcatctcgt ccgtctccaa catcacctgc cccaactttg atgccagcat ttgcatcccg |
| 15181 | ggctccatca cattcatgcc caatggatgc tgcaagacct gcacccctcg caatgagacc |
| 15241 | agggtgccct gctccaccgt ccccgtcacc acggaggttt cgtacgccgg ctgcaccaag |
| 15301 | accgtcctca tgaatcattg ctccgggtcc tgcgggacat ttgtcatgta ctcggccaag |
| 15361 | gcccaggccc tggaccacag ctgctcctgc tgcaaagagg agaaaaccag ccagcgtgag |
| 15421 | gtggtcctga gctgccccaa tggcggctcg ctgacacaca cctacaccca catcgagagc |
| 15481 | tgccagtgcc aggacaccgt ctgcgggctc cccaccggca cctcccgccg ggcccggcgc |
| 15541 | tcccctaggc atctggggag cgggtgagcg gggtgggcac agcccccttc actgccctcg |
| 15601 | acagctttac ctcccccgga ccctctgagc ctcctaagct cggcttcctc tcttcagata |
| 15661 | tttattgtct gagtctttgt tcagtccttg ctttccaata ataaactcag ggggacatgc |

By “NKX2-5 polypeptide” or “human NKX2-5 (hNKX2-5) polypeptide” is meant a protein or fragment thereof having at least 85% amino acid sequence identity to the sequence provided at NCBI Accession No. NP_004378.1 (isoform 1), NP_001159647.1 (isoform 2), or NP_001159648.1 (isoform 3) and having transcription factor activity. The amino acid sequence provided at NCBI Accession No. NP_004378.1 is shown below:

| (SEQ ID NO: 31) | |
|---|---|
| 1 | mfpspaltpt pfsvkdilnl eqqqrslaaa gelsarleat lapsscmlaa fkpeayagpe |
| 61 | aaapglpelr aelgrapspa kcasafpaap afypraysdp dpakdpraek kelcalqkav |
| 121 | elekteadna erprarrrrk prvlfsqaqv yelerrfkgq rylsaperdq lasvlkltst |
| 181 | qvkiwfqnrr ykckrqrqdq tlelvglppp pppparriav pvlvrdgkpc lgdsapyapa |
| 241 | ygvglnpygy naypaypgyg gaacspgysc taaypagpsp agpataaann nfvnfgvgdl |
| 301 | navqspgipq snsgvstlhg iraw |

By “NKX2-5 polynucleotide” is meant a polynucleotide encoding a NKX2-5 polypeptide or fragment thereof. An exemplary NKX2-5 polynucleotide sequence is provided at NCBI Ref: NM_004387.3. The sequence provided at NCBI Ref: NM_004387.3 is reproduced below:

| (SEQ ID NO: 32) | |
|---|---|
| 1 | gctcctgtca tcgaggcccc tggcccaatg gcaggctgag tccccctcct ctggcctggt |
| 61 | cccgcctctc ctgccccttg tgctcagcgc tacctgctgc ccggacacat ccagagctgg |
| 121 | ccgacgggtg cgcgggcggg cggcggcacc atgcagggaa gctgccaggg gccgtgggca |
| 181 | gcgccgcttt ctgccgccca cctggcgctg tgagactggc gctgccacca tgttccccag |
| 241 | ccctgctctc acgcccacgc ccttctcagt caaagacatc ctaaacctgg aacagcagca |
| 301 | gcgcagcctg gctgccgccg gagagctctc tgcccgcctg gaggcgaccc tggcgccctc |
| 361 | ctcctgcatg ctggccgcct tcaagccaga ggcctacgct gggcccgagg cggctgcgcc |
| 421 | gggcctccca gagctgcgcg cagagctggg ccgcgcgcct tcaccggcca agtgtgcgtc |
| 481 | tgcctttccc gccgcccccg ccttctatcc acgtgcctac agcgaccccg acccagccaa |
| 541 | ggaccctaga gccgaaaaga aagagctgtg cgcgctgcag aaggcggtgg agctggagaa |
| 601 | gacagaggcg gacaacgcgg agcggccccg ggcgcgacgg cggaggaagc cgcgcgtgct |
| 661 | cttctcgcag gcgcaggtct atgagctgga gcggcgcttc aagcagcagc ggtacctgtc |
| 721 | ggcccccgaa cgcgaccagc tggccagcgt gctgaaactc acgtccacgc aggtcaagat |
| 781 | ctggttccag aaccggcgct acaagtgcaa gcggcagcgg caggaccaga ctctggagct |
| 841 | ggtggggctg cccccgccgc cgccgccgcc tgcccgcagg atcgcggtgc cagtgctggt |
| 901 | gcgcgatggc aagccatgcc taggggactc ggcgccctac gcgcctgcct acggcgtggg |
| 961 | cctcaatccc tacggttata acgcctaccc cgcctatccg ggttacggcg gcgcggcctg |
| 1021 | cagccctggc tacagctgca ctgccgctta ccccgccggg ccttccccag cgcagccggc |
| 1081 | cactgccgcc gccaacaaca acttcgtgaa cttcggcgtc ggggacttga atgcggttca |
| 1141 | gagccccggg attccgcaga gcaactcggg agtgtccacg ctgcatggta tccgagcctg |
| 1201 | gtagggaagg gacccgcgtg gcgcgaccct gaccgatccc acctcaacag ctccctgact |
| 1261 | ctcgggggga gaaggggctc ccaacatgac cctgagtccc ctggattttg cattcactcc |
| 1321 | tgcggagacc taggaacttt ttctgtccca cgcgcgtttg ttcttgcgca cgggagagtt |
| 1381 | tgtggcggcg attatgcagc gtgcaatgag tgatcctgca gcctggtgtc ttagctgtcc |
| 1441 | ccccaggagt gccctccgag agtccatggg cacccccggt tggaactggg actgagctcg |
| 1501 | ggcacgcagg gcctgagatc tggccgccca ttccgcgagc cagggccggg cgcccgggcc |
| 1561 | tttgctatct cgccgtcgcc cgcccacgca cccacccgta tttatgtttt tacctattgc |
| 1621 | tgtaagaaat gacgatcccc ttcccattaa agagagtgcg ttgaccccg |

By “NEUROD1 polypeptide” is meant a protein or fragment thereof having at least 85% amino acid sequence identity to the sequence provided at NCBI Accession No. NP_002491.2 and having transcription factor activity. The amino acid sequence provided at NCBI Accession No. NP_002491.2 is shown below:

| (SEQ ID NO: 33) | |
|---|---|
| 1 | mtksysesgl mgepqpqgpp swtdeclssq deeheadkke ddleamnaee dslrnggeee |
| 61 | dededleeee eeeeedddqk pkrrgpkkkk mtkarlerfk lrrmkanare rnrmhglnaa |
| 121 | ldnlrkvvpc ysktqklski etlrlaknyi walseilrsg kspdlvsfvq tlckglsgpt |
| 181 | tnlvagclql nprtflpeqn qdmpphlpta sasfpvhpys yqspglpspp ygtmdsshvf |
| 241 | hvkppphays aalepffesp ltdctspsfd gplspplsin gnfsfkheps aefeknyaft |
| 301 | mhypaatlag aqshgsifsg taaprceipi dnimsfdshs hhervmsaql naifhd |

By “NEUROD1 polynucleotide” is meant a polynucleotide encoding a NEUROD1 polypeptide or fragment thereof. An exemplary NEUROD1 polynucleotide sequence is provided at NCBI Ref: NM_002500.4. The sequence provided at NCBI Ref: NM_002500.4 is reproduced below:

| (SEQ ID NO: 34) | |
|---|---|
| 1 | ggggaggagg ggagaacggg gagcgcacag cctggacgcg tgcgcaggcg tcaggcgcat |
| 61 | agacctgcta gcccctcagc tagcggcccc gcccgcgctt agcatcacta actgggctat |
| 121 | ataacctgag cgcccgcgcg gccacgacac gaggaattcg cccacgcagg aggcgcggcg |
| 181 | tccggaggcc ccagggttat gagactatca ctgctcagga cctactaaca acaaaggaaa |
| 241 | tcgaaacatg accaaatcgt acagcgagag tgggctgatg ggcgagcctc agccccaagg |
| 301 | tcctccaagc tggacagacg agtgtctcag ttctcaggac gaggagcacg aggcagacaa |
| 361 | gaaggaggac gacctcgaag ccatgaacgc agaggaggac tcactgagga acgggggaga |
| 421 | ggaggaggac gaagatgagg acctggaaga ggaggaagaa gaggaagagg aggatgacga |
| 481 | tcaaaagccc aagagacgcg gccccaaaaa gaagaagatg actaaggctc gcctggagcg |
| 541 | ttttaaattg agacgcatga aggctaacgc ccgggagcgg aaccgcatgc acggactgaa |
| 601 | cgcggcgcta gacaacctgc gcaaggtggt gccttgctat tctaagacgc agaagctgtc |
| 661 | caaaatcgag actctgcgct tggccaagaa ctacatctgg gctctgtcgg agatcctgcg |
| 721 | ctcaggcaaa agcccagacc tggtctcctt cgttcagacg ctttgcaagg gcttatccca |
| 781 | acccaccacc aacctggttg cgggctgcct gcaactcaat cctcggactt ttctgcctga |
| 841 | gcagaaccag gacatgcccc cccacctgcc gacggccagc gcttccttcc ctgtacaccc |
| 901 | ctactcctac cagtcgcctg ggctgcccag tccgccttac ggtaccatgg acagctccca |
| 961 | tgtcttccac gttaagcctc cgccgcacgc ctacagcgca gcgctggagc ccttctttga |
| 1021 | aagccctctg actgattgca ccagcccttc ctttgatgga cccctcagcc cgccgctcag |
| 1081 | catcaatggc aacttctctt tcaaacacga accgtccgcc gagtttgaga aaaattatgc |
| 1141 | ctttaccatg cactatcctg cagcgacact ggcaggggcc caaagccacg gatcaatctt |
| 1201 | ctcaggcacc gctgcccctc gctgcgagat ccccatagac aatattatgt ccttcgatag |
| 1261 | ccattcacat catgagcgag tcatgagtgc ccagctcaat gccatatttc atgattagag |
| 1321 | gcacgccagt ttcaccattt ccgggaaacg aacccactgt gcttacagtg actgtcgtgt |
| 1381 | ttacaaaagg cagccctttg ggtactactg ctgcaaagtg caaatactcc aagcttcaag |
| 1441 | tgatatatgt atttattgtc attactgcct ttggaagaaa caggggatca aagttcctgt |
| 1501 | tcaccttatg tattattttc tatagctctt ctatttaaaa aataaaaaaa tacagtaaag |
| 1561 | tttaaaaaat acaccacgaa tttggtgtgg ctgtattcag atcgtattaa ttatctgatc |
| 1621 | gggataacaa aatcacaagc aataattagg atctatgcaa tttttaaact agtaatgggc |
| 1681 | caattaaaat atatataaat atatattttt caaccagcat tttactactt gttacctttc |
| 1741 | ccatgctgaa ttattttgtt gtgattttgt acagaatttt taatgacttt ttataatgtg |
| 1801 | gatttcctat tttaaaacca tgcagcttca tcaattttta tacatatcag aaaagtagaa |
| 1861 | ttatatctaa tttatacaaa ataatttaac taatttaaac cagcagaaaa gtgcttagaa |
| 1921 | agttattgtg ttgccttagc acttctttcc tctccaattg taaaaaaaaa aaaaaaaaaa |
| 1981 | aaaaaaaaaa aaaaattgca caatttgagc aattcatttc actttaaagt ctttccgtct |
| 2041 | ccctaaaata aaaaccagaa tcataatttt caagagaaga aaaaattaag agatacattc |
| 2101 | cctatcaaaa catatcaatt caacacatta cttgcacaag cttgtatata catattataa |
| 2161 | ataaatgcca acataccctt ctttaaatca aaagctgctt gactatcaca tacaatttgc |
| 2221 | actgttactt tttagtcttt tactcctttg cattccatga ttttacagag aatctgaagc |
| 2281 | tattgatgtt tccagaaaat ataaatgcat gattttatac atagtcacaa aaatggtggt |
| 2341 | ttgtcatata ttcatgtaat aaatctgagc ctaaatctaa tcaggttgtt aatgttggga |
| 2401 | tttatatcta tagtagtcaa ttagtacagt agcttaaata aattcaaacc atttaattca |
| 2461 | taattagaac aatagctatt gcatgtaaaa tgcagtccag aataagtgct gtttgagatg |
| 2521 | tgatgctggt accactggaa tcgatctgta ctgtaatttt gtttgtaatc ctgtatatta |
| 2581 | tggtgtaatg cacaatttag aaaacattca tccagttgca ataaaatagt attgaaagtg |
| 2641 | agagcaattg ttgcatttct tcttaaaggg attctgtttt tatttttggg gaaagtagtt |
| 2701 | gcttttttgc tgagttaaaa aatactaaac actatatgta gaataaaaga aaagaaaaaa |
| 2761 | gtttaccttg gcatatgctc ttgtctgttt atcttgcaca gggagtcacc agttctatgt |
| 2821 | agataatgaa aagacctaac tgatatttca ttatttggaa tatgggactg gacggcagta |
| 2881 | caaacagtgt gtttttttct ttgttttaag tggcttagcc tttaggtttt ttatttccat |
| 2941 | ttttaaaaat gattgttaca tgttttcttc tatttctttt tttaaaaggt ggattttaat |
| 3001 | aa |

By “NKX6-1 polypeptide” is meant a protein or fragment thereof having at least 85% amino acid sequence identity to the sequence provided at NCBI Accession No. NP_006159.2 and having transcription factor activity. The amino acid sequence provided at NCBI Accession No. NP_006159.2 is shown below:

| (SEQ ID NO: 35) | |
|---|---|
| 1 | mlavgamegt rqsafllssp plaalhsmae mktplypaay pplpagppss sssssssssp |
| 61 | spplgthnpg glkppatggl sslgsppqql saatphgind ilsrpsmpva sgaalpsasp |
| 121 | sgssssssss asassasaaa aaaaaaaaaa sspagllagl prfsslsppp pppglyfsps |
| 181 | aaavaavgry pkplaelpgr tpifwpgvmq sppwrdarla ctphqgsill dkdgkrkhtr |
| 241 | ptfsgqqifa lektfeqtky lagperarla yslgmtesqv kvwfqnrrtk wrkkhaaema |
| 301 | takkkqdset erlkgasene eedddynkpl dpnsddekit qllkkhksss ggggglllha |
| 361 | sepesss |

By “NKX6-1 polynucleotide” is meant a polynucleotide encoding a NKX6-1 polypeptide or fragment thereof. An exemplary NKX6-1 polynucleotide sequence is provided at NCBI Ref: NM_006168.2. The sequence provided at NCBI Ref: NM_006168.2 is reproduced below:

| (SEQ ID NO: 36) | |
|---|---|
| 1 | cgtgggatgt tagcggtggg ggcaatggag ggcacccggc agagcgcatt cctgctcagc |
| 61 | agccctcccc tggccgccct gcacagcatg gccgagatga agaccccgct gtaccctgcc |
| 121 | gcgtatcccc cgctgcctgc cggccccccc tcctcctcgt cctcgtcgtc gtcctcctcg |
| 181 | tcgccctccc cgcctctggg cacccacaac ccaggcggcc tgaagccccc ggccacgggg |
| 241 | gggctctcat ccctcggcag ccccccgcag cagctctcgg ccgccacccc acacggcatc |
| 301 | aacgatatcc tgagccggcc ctccatgccc gtggcctcgg gggccgccct gccctccgcc |
| 361 | tcgccctccg gttcctcctc ctcctcttcc tcgtccgcct ctgcctcctc cgcctctgcc |
| 421 | gccgccgcgg ctgctgccgc ggccgcagcc gccgcctcat ccccggcggg gctgctggcc |
| 481 | ggactgccac gctttagcag cctgagcccg ccgccgccgc cgcccgggct ctacttcagc |
| 541 | cccagcgccg cggccgtggc cgccgtgggc cggtacccca agccgctggc tgagctgcct |
| 601 | ggccggacgc ccatcttctg gcccggagtg atgcagagcc cgccctggag ggacgcacgc |
| 661 | ctggcctgta cccctcatca aggatccatt ttgttggaca aagacgggaa gagaaaacac |
| 721 | acgagaccca ctttttccgg acagcagatc ttcgccctgg agaagacttt cgaacaaaca |
| 781 | aaatacttgg cggggcccga gagggctcgt ttggcctatt cgttggggat gacagagagt |
| 841 | caggtcaagg tctggttcca gaaccgccgg accaagtgga ggaagaagca cgctgccgag |
| 901 | atggccacgg ccaagaagaa gcaggactcg gagacagagc gcctcaaggg ggcctcggag |
| 961 | aacgaggaag aggacgacga ctacaataag cctctggatc ccaactcgga cgacgagaaa |
| 1021 | atcacgcagc tgttgaagaa gcacaagtcc agcagcggcg gcggcggcgg cctcctactg |
| 1081 | cacgcgtccg agccggagag ctcatcctga acgccg |

By “NDUFA4 polypeptide” is meant a protein or fragment thereof having at least 85% amino acid sequence identity to the sequence provided at NCBI Accession No. NP_002480.1 and having NADH dehydrogenase activity and oxidoreductase activity. The amino acid sequence provided at NCBI Accession No. NP_002480.1 is shown below:

| (SEQ ID NO: 37) | |
|---|---|
| 1 | maaelamgae lpssplaiey vndfdlmkfe vkkeppeaer fchrlppgsl sstplstpcs |
| 61 | svpsspsfca pspgtggggg agggggssqa ggapgppsgg pgavggtsgk paledlywms |
| 121 | gyqhhlnpea lnltpedave aligsghhga hhgahhpaaa aayeafrgpg faggggaddm |
| 181 | gaghhhgahh aahhhhaahh hhhhhhhhgg aghgggaghh vrleerfsdd qlvsmsvrel |
| 241 | nrqlrgfske evirlkqkrr tlknrgyaqs crfkrvqqrh ilesekcqlq sqveqlklev |
| 301 | grlakerdly kekyeklagr ggpgsaggag fprepsppqa gpggakgtad ffl |

By “NDUFA4 polynucleotide” is meant a polynucleotide encoding a NDUFA4 polypeptide or fragment thereof. An exemplary NDUFA4 polynucleotide sequence is provided at NCBI Ref: NM_002489.3. The sequence provided at NCBI Ref: NM_002489.3 is reproduced below:

| (SEQ ID NO: 38) | |
|---|---|
| 1 | gggtccttca ggtaggaggt cctgggtgac tttggaagtc cgtagtgtct cattgcagat |
| 61 | aatttttagc ttagggcctg gtggctaggt cggttctctc ctttccagtc ggagacctct |
| 121 | gccgcaaaca tgctccgcca gatcatcggt caggccaaga agcatccgag cttgatcccc |
| 181 | ctctttgtat ttattggaac tggagctact ggagcaacac tgtatctctt gcgtctggca |
| 241 | ttgttcaatc cagatgtttg ttgggacaga aataacccag agccctggaa caaactgggt |
| 301 | cccaatgatc aatacaagtt ctactcagtg aatgtggatt acagcaagct gaagaaggaa |
| 361 | cgtccagatt tctaaatgaa atgtttcact ataacgctgc tttagaatga aggtcttcca |
| 421 | gaagccacat ccgcacaatt ttccacttaa ccaggaaata tttctcctct aaatgcatga |
| 481 | aatcatgttg gagatctcta ttgtaatctc tattggagat tacaatgatt aaatcaataa |
| 541 | ataactgaaa cttgatatgt gtcacttttt tatgctgaaa gtatgctctg aactttagag |
| 601 | tataggaaat taactattag aatttaaaga atttcttgaa tttctgtagt ttgaaaatac |
| 661 | gactttaagc tgctttagta aaacacttcc attttgtgta tagactgttg gtaacttcac |
| 721 | tagagcatac ataacaactg gaactggaaa ttatacaaaa gtaaattggg aaggatactc |
| 781 | cagcatctga cactggcaaa atggaaacct ttgagtttct cttactggct gttgaagtgt |
| 841 | gtgcagtttt taacaatggt ttttacttgg catctctttg ttgtgatttt caaggttata |
| 901 | agttgctttg gtcctaggat tgaagttgaa atctgagttt atcagtgcta accatggtgc |
| 961 | tagtagtcaa gagatcttga gaattttggc tgctgagtct tggtgcaggg tgcaggtttt |
| 1021 | cttttctttt ttcttttttt tttttttgag atagtctctg tcacccaggc tggagtgcag |
| 1081 | tggtacaaac atggatcact gcagcctcta cctcccgggc ttaagtgatc ctcctgcctc |
| 1141 | agcccctaag tagccgggac tacaggtatg tgccaccatg cccagttaat ttttgtaatt |
| 1201 | ttttttagag acagggtttt gccatgttgc ccaggctggt ctcaaactct tgagctcaag |
| 1261 | cgatccattc tcctcagcct cccagggtgc tgggattaca ggcgtgagcc attgcgctta |
| 1321 | gccatggtgc aggttttcaa aggccaggaa gtatattcat aattttaaga tggggaatat |
| 1381 | agcaagtttt cacataggtg tgtgtaagtc atcacatcat agaaacttga ggaattcagt |
| 1441 | gacattaatt ttggattttc atacgtaagt atacaattaa atgtttacag ggtagtagaa |
| 1501 | gcacatttta aatgtcagga actgaactaa gtatttgaat tacgtggatt atctcaaaaa |
| 1561 | ttttgaaatt gttaaacgag ttgaattact tgaattcatt ctgttagtca aatggtggat |
| 1621 | atttacaccc atgtagtttt gaatttagag tgtgtagagt gttttcagtt accagactcc |
| 1681 | atgcttttac ctcctatgtg tcaggtataa tttgaacctc taagaacagg gtttctcaac |
| 1741 | cttgccactg ttgactattt ctgaaagaca gtttggttta gcagaccatc ccatgcgctt |
| 1801 | tagcttgttt agtagctaac ttgggctctg ccactacaga caaaaagcac tctttccctc |
| 1861 | caattcccac aggctatgag aagaatggag acattaccaa atgtccattg gtgggcaaaa |
| 1921 | ttgcttcatt cctacctctg ttgagaatta ctctagatcc tttggcacaa attacctcaa |
| 1981 | agtttaaaat tgtgtaaaca aacagtgtgt catgtaattg aaaaacatta agcaactcca |
| 2041 | aataaatgct acattaag |

As used herein, “obtaining” as in “obtaining an agent” includes synthesizing, purchasing, or otherwise acquiring the agent.

By “organ” is meant a collection of cells that perform a biological function. In one embodiment, an organ includes, but is not limited to, bladder, brain, nervous tissue, glial tissue, esophagus, fallopian tube, heart, pancreas, intestines, gallbladder, kidney, liver, lung, ovaries, prostate, spinal cord, spleen, stomach, testes, thymus, thyroid, trachea, urogenital tract, ureter, urethra, uterus, breast, skeletal muscle, skin, bone, and cartilage. The biological function of an organ can be assayed using standard methods known to the skilled artisan.

By “organoid” is meant an in vitro generated body that mimics organ structure and function. “Organoid” and “mini organ” are used interchangeably herein. A “pancreatic islet organoid” is an in vitro generated cell cluster that mimics structure and function of a pancreatic islet. Exemplary functions of a pancreatic islet include, without limitation, glucose-stimulated insulin secretion (GSIS), potassium chloride (KCl)-stimulated insulin secretion, GLP-1 stimulated insulin secretion, somatostatin secretion, or glucagon secretion. “Pancreatic islet organoid” and “mini pancreatic islet” are used interchangeably herein. A “pancreatic organoid” is an in vitro generated body that mimics structure and function of a pancreas. Exemplary functions of a pancreas include, without limitation, endocrine secretion of hormones, such as glucose and glucagon, that regulate glucose metabolism and blood glucose concentration, and exocrine secretion of digestive enzymes that help break down carbohydrates, proteins, and lipids. “Pancreatic organoid” and “mini pancreas” are used interchangeably herein.

By “PAX4 polypeptide” is meant a protein or fragment thereof having at least 85% amino acid sequence identity to the sequence provided at NCBI Accession No. NP_006184.2 and having transcription factor activity. The amino acid sequence provided at NCBI Accession No. NP_006184.2 is shown below:

| (SEQ ID NO: 39) | |
|---|---|
| 1 | mnqlgglfvn grplpldtrq qivrlavsgm rpcdisrilk vsngcvskil gryyrtgvle |
| 61 | pkgiggskpr latppvvari aqlkgecpal faweiqrqlc aeglctqdkt psvssinrvl |
| 121 | ralqedqglp ctrlrspavl apavltphsg setprgthpg tghrnrtifs psqaealeke |
| 181 | fqrgqypdsv argklatats lpedtvrvwf snrrakwrrq eklkwemqlp gasqgltvpr |
| 241 | vapgiisaqq spgsvptaal paleplgpsc yqlcwatape rclsdtppka clkpcwghlp |
| 301 | pqpnsldsgl lclpcpsshc hlaslsgsqa llwpgcplly gle |

By “PAX4 polynucleotide” is meant a polynucleotide encoding a PAX4 polypeptide or fragment thereof. An exemplary PAX4 polynucleotide sequence is provided at NCBI Ref: NM_006193.2. The sequence provided at NCBI Ref: NM_006193.2 is reproduced below:

| (SEQ ID NO: 40) | |
|---|---|
| 1 | caaagactca cccgtgagcc agctctcaaa gaaagcagct tgcgttgaca gcctgggggc |
| 61 | agcaaggatg cagtctccca ggagaggatg cactcggtgg tgggaagcca ggctggaggg |
| 121 | gcctgagtga ccctctccac aggcgggcag ggcagtggga gaggtggtgt gtggatacct |
| 181 | ctgtctcacg cccagggatc agcagcatga accagcttgg ggggctcttt gtgaatggcc |
| 241 | ggcccctgcc tctggatacc cggcagcaga ttgtgcggct agcagtcagt ggaatgcggc |
| 301 | cctgtgacat ctcacggatc cttaaggtat ctaatggctg tgtgagcaag atcctagggc |
| 361 | gttactaccg cacaggtgtc ttggagccaa agggcattgg gggaagcaag ccacggctgg |
| 421 | ctacaccccc tgtggtggct cgaattgccc agctgaaggg tgagtgtcca gccctctttg |
| 481 | cctgggaaat ccaacgccag ctttgtgctg aagggctttg cacccaggac aagactccca |
| 541 | gtgtctcctc catcaaccga gtcctgcggg cattacagga ggaccaggga ctaccgtgca |
| 601 | cacggctcag gtcaccagct gttttggctc cagctgtcct cactccccat agtggctctg |
| 661 | agactccccg gggtacccac ccagggaccg gccaccggaa tcggactatc ttctccccaa |
| 721 | gccaagcaga ggcactggag aaagagttcc agcgtgggca gtatcctgat tcagtggccc |
| 781 | gtggaaagct ggctactgcc acctctctgc ctgaggacac ggtgagggtc tggttttcca |
| 841 | acagaagagc caaatggcgt cggcaagaga agctcaagtg ggaaatgcag ctgccaggtg |
| 901 | cttcccaggg gctgactgta ccaagggttg ccccaggaat catctctgca cagcagtccc |
| 961 | ctggcagtgt gcccacagca gccctgcctg ccctggaacc actgggtccc tcctgctatc |
| 1021 | agctgtgctg ggcaacagca ccagaaaggt gtctgagtga caccccacct aaagcctgtc |
| 1081 | tcaagccctg ctggggccac ttgcccccac agccgaattc cctggactca ggactgcttt |
| 1141 | gccttccttg cccttcctcc cactgtcacc tggccagtct tagtggctct caggccctgc |
| 1201 | tctggcctgg ctgcccacta ctgtatggct tggaatgagg caggagtggg aaggagatgg |
| 1261 | catagagaag atctaatacc atcctgccca ttgtccttac cgtcctgccc atacagactg |
| 1321 | tggctccttc ctccttcctg tgattgctcc ctcctgtgtg gacgttgcct ggccctgcct |
| 1381 | cgatgcctct ctggcgcatc acctgattgg aggggctggt aaagcaacac ccacccactt |
| 1441 | ctcacactag ccttaagagg cctccactca gcagtaataa aagctgtttt tattagcagt |
| 1501 | agttctgttg tccatcatgt tttccctatg agcaccccta tgcccactct aatattcaac |
| 1561 | aattatagac aatttgccct atcatttatt tacatctatg tatctaccat ctaatctatg |
| 1621 | catgtatgta ggcaatacat gtatctaaac aatgtatttg tcaatgcatc aatttaccta |
| 1681 | ctctatgtat gcatctatat gtgtattatg tatgcgtgca tgcgtgcgcg cacacacaca |
| 1741 | cacacacaca cacactgaca ttatatcatg gcattttatt cctaaatctt ccagcatgca |
| 1801 | tccccaaaaa acaagaaact tgtcttacat aatcacaata atatatccac atctaagaaa |
| 1861 | atttactgta acttcttaat ctaagaaaat tatgtatttt tgtcatatgt attttgtcat |
| 1921 | atgtattttg tatttgcata tgtattttgt atttgcatat gtatttttgt catagcagca |
| 1981 | aacagagtga aatgccattt ttcatattct |

By “PAX6 polypeptide” is meant a protein or fragment thereof having at least 85% amino acid sequence identity to the sequence provided at NCBI Accession No. NP_001297090.1 and having transcription factor activity. The amino acid sequence provided at NCBI Accession No. NP_001297090.1 is shown below:

| (SEQ ID NO: 41) | |
|---|---|
| 1 | mgadgmydkl rmlngqtgsw gtrpgwypgt svpgqptqdg cqqqegggen tnsissnged |
| 61 | sdeaqmrlql krklqrnrts ftqeqieale keferthypd vfarerlaak idlpeariqv |
| 121 | wfsnrrakwr reeklrnqrr qasntpship isssfstsvy qpipqpttpv ssftsgsmlg |
| 181 | rtdtaltnty salppmpsft mannlpmqpp vpsqtssysc mlptspsvng rsydtytpph |
| 241 | mqthmnsqpm gtsgttstgl ispgvsvpvq vpgsepdmsq ywprlq |

By “PAX6 polynucleotide” is meant a polynucleotide encoding a PAX6 polypeptide or fragment thereof. An exemplary PAX6 polynucleotide sequence is provided at NCBI Ref: NM_001310161.1. The sequence provided at NCBI Ref: NM_001310161.1 is reproduced below:

| (SEQ ID NO: 42) | |
|---|---|
| 1 | cttttcaatt agccttccat gcatgatccg gagcgacttc cgcctatttc cagaaattaa |
| 61 | gctcaaactt gacgtgcagc tagttttatt ttaaagacaa atgtcagaga ggctcatcat |
| 121 | attttccccc ctcttctata tttggagctt atttattgct aagaagctca ggctcctggc |
| 181 | gtcaatttat cagtaggctc caaggagaag agaggagagg agaggagagc tgaacaggga |
| 241 | gccacgtctt ttcctgggag ggctgctatc taagtcgggg ctgcaggtca cagcggagtg |
| 301 | aatcagctcg gtggtgtctt tgtcaacggg cggccactgc cggactccac ccggcagaag |
| 361 | attgtagagc tagctcacag cggggcccgg ccgtgcgaca tttcccgaat tctgcagacc |
| 421 | catgcagatg caaaagtcca agtgctggac aatcaaaacg tgtccaacgg atgtgtgagt |
| 481 | aaaattctgg gcaggtatta cgagactggc tccatcagac ccagggcaat cggtggtagt |
| 541 | aaaccgagag tagcgactcc agaagttgta agcaaaatag cccagtataa gcgggagtgc |
| 601 | ccgtccatct ttgcttggga aatccgagac agattactgt ccgagggggt ctgtaccaac |
| 661 | gataacatac caagcgtgtc atcaataaac agagttcttc gcaacctggc tagcgaaaag |
| 721 | caacagatgg gcgcagacgg catgtatgat aaactaagga tgttgaacgg gcagaccgga |
| 781 | agctggggca cccgccctgg ttggtatccg gggacttcgg tgccagggca acctacgcaa |
| 841 | gatggctgcc agcaacagga aggaggggga gagaatacca actccatcag ttccaacgga |
| 901 | gaagattcag atgaggctca aatgcgactt cagctgaagc ggaagctgca aagaaataga |
| 961 | acatccttta cccaagagca aattgaggcc ctggagaaag agtttgagag aacccattat |
| 1021 | ccagatgtgt ttgcccgaga aagactagca gccaaaatag atctacctga agcaagaata |
| 1081 | caggtatggt tttctaatcg aagggccaaa tggagaagag aagaaaaact gaggaatcag |
| 1141 | agaagacagg ccagcaacac acctagtcat attcctatca gcagtagttt cagcaccagt |
| 1201 | gtctaccaac caattccaca acccaccaca ccggtttcct ccttcacatc tggctccatg |
| 1261 | ttgggccgaa cagacacagc cctcacaaac acctacagcg ctctgccgcc tatgcccagc |
| 1321 | ttcaccatgg caaataacct gcctatgcaa cccccagtcc ccagccagac ctcctcatac |
| 1381 | tcctgcatgc tgcccaccag cccttcggtg aatgggcgga gttatgatac ctacaccccc |
| 1441 | ccacatatgc agacacacat gaacagtcag ccaatgggca cctcgggcac cacttcaaca |
| 1501 | ggactcattt cccctggtgt gtcagttcca gttcaagttc ccggaagtga acctgatatg |
| 1561 | tctcaatact ggccaagatt acagtaaaaa aaaaaaaaaa aaaaaaaagg aaaggaaata |
| 1621 | ttgtgttaat tcagtcagtg actatgggga cacaacagtt gagctttcag gaaagaaaga |
| 1681 | aaaatggctg ttagagccgc ttcagttcta caattgtgtc ctgtattgta ccactgggga |
| 1741 | aggaatggac ttgaaacaag gacctttgta tacagaaggc acgatatcag ttggaacaaa |
| 1801 | tcttcatttt ggtatccaaa cttttattca ttttggtgta ttatttgtaa atgggcattt |
| 1861 | gtatgttata atgaaaaaaa gaacaatgta gactggatgg atgtttgatc tgtgttggtc |
| 1921 | atgaagttgt tttttttttt tttaaaaaga aaaccatgat caacaagctt tgccacgaat |
| 1981 | ttaagagttt tatcaagata tatcgaatac ttctacccat ctgttcatag tttatggact |
| 2041 | gatgttccaa gtttgtatca ttcctttgca tataattaaa cctggaacaa catgcactag |
| 2101 | atttatgtca gaaatatctg ttggttttcc aaaggttgtt aacagatgaa gtttatgtgc |
| 2161 | aaaaaagggt aagatataaa ttcaaggaag aaaaaaagtt gatagctaaa aggtagagtg |
| 2221 | tgtcttcgat ataatccaat ttgttttatg tcaaaatgta agtatttgtc ttccctagaa |
| 2281 | atcctcagaa tgatttctat aataaagtta atttcattta tatttgacaa gaatatagat |
| 2341 | gttttataca cattttcatg caatcatacg tttctttttt ggccagcaaa agttaattgt |
| 2401 | tcttagatat agttgtatta ctgttcacgg tccaatcatt ttgtgcatct agagttcatt |
| 2461 | cctaatcaat taaaagtgct tgcaagagtt ttaaacttaa gtgttttgaa gttgttcaca |
| 2521 | actacatatc aaaattaacc attgttgatt gtaaaaaacc atgccaaagc ctttgtattt |
| 2581 | cctttattat acagttttct ttttaacctt atagtgtggt gttacaaatt ttatttccat |
| 2641 | gttagatcaa cattctaaac caatggttac tttcacacac actctgtttt acatcctgat |
| 2701 | gatccttaaa aaataatcct tatagatacc ataaatcaaa aacgtgttag aaaaaaattc |
| 2761 | cacttacagc agggtgtaga tctgtgccca tttataccca caacatatat acaaaatggt |
| 2821 | aacatttccc agttagccat ttaattctaa agctcaaagt ctagaaataa tttaaaaatg |
| 2881 | caacaagcga ttagctagga attgtttttt gaattaggac tggcattttc aatctgggca |
| 2941 | gatttccatt gtcagcctat ttcaacaatg atttcactga agtatattca aaagtagatt |
| 3001 | tcttaaagga gactttctga aagctgttgc ctttttcaaa taggccctct cccttttctg |
| 3061 | tctccctccc ctttgcacaa gaggcatcat ttcccattga accactacag ctgttcccat |
| 3121 | ttgaatcttg ctttctgtgc ggttgtggat ggttggaggg tggagggggg atgttgcatg |
| 3181 | tcaaggaata atgagcacag acacatcaac agacaacaac aaagcagact gtgactggcc |
| 3241 | ggtgggaatt aaaggccttc agtcattggc agcttaagcc aaacattccc aaatctatga |
| 3301 | agcagggccc attgttggtc agttgttatt tgcaatgaag cacagttctg atcatgttta |
| 3361 | aagtggaggc acgcagggca ggagtgcttg agcccaagca aaggatggaa aaaaataagc |
| 3421 | ctttgttggg taaaaaagga ctgtctgaga ctttcatttg ttctgtgcaa catataagtc |
| 3481 | aatacagata agtcttcctc tgcaaacttc actaaaaagc ctgggggttc tggcagtcta |
| 3541 | gattaaaatg cttgcacatg cagaaacctc tggggacaaa gacacacttc cactgaatta |
| 3601 | tactctgctt taaaaaaatc cccaaaagca aatgatcaga aatgtagaaa ttaatggaag |
| 3661 | gatttaaaca tgaccttctc gttcaatatc tactgttttt tagttaagga attacttgtg |
| 3721 | aacagataat tgagattcat tgctccggca tgaaatatac taataatttt attccaccag |
| 3781 | agttgctgca catttggaga caccttccta agttgcagtt tttgtatgtg tgcatgtagt |
| 3841 | tttgttcagt gtcagcctgc actgcacagc agcacatttc tgcaggggag tgagcacaca |
| 3901 | tacgcactgt tggtacaatt gccggtgcag acatttctac ctcctgacat tttgcagcct |
| 3961 | acattccctg agggctgtgt gctgagggaa ctgtcagaga agggctatgt gggagtgcat |
| 4021 | gccacagctg ctggctggct tacttcttcc ttctcgctgg ctgtaatttc caccacggtc |
| 4081 | aggcagccag ttccggccca cggttctgtt gtgtagacag cagagacttt ggagacccgg |
| 4141 | atgtcgcacg ccaggtgcaa gaggtgggaa tgggagaaaa ggagtgacgt gggagcggag |
| 4201 | ggtctgtatg tgtgcacttg ggcacgtata tgtgtgctct gaaggtcagg attgccaggg |
| 4261 | caaagtagca cagtctggta tagtctgaag aagcggctgc tcagctgcag aagccctctg |
| 4321 | gtccggcagg atgggaacgg ctgccttgcc ttctgcccac accctaggga catgagctgt |
| 4381 | ccttccaaac agagctccag gcactctctt ggggacagca tggcaggctc tgtgtggtag |
| 4441 | cagtgcctgg gagttggcct tttactcatt gttgaaataa tttttgttta ttatttattt |
| 4501 | aacgatacat atatttatat atttatcaat ggggtatctg cagggatgtt ttgacaccat |
| 4561 | cttccaggat ggagattatt tgtgaagact tcagtagaat cccaggacta aacgtctaaa |
| 4621 | ttttttctcc aaacttgact gacttgggaa aaccaggtga atagaataag agctgaatgt |
| 4681 | tttaagtaat aaacgttcaa actgctctaa gtaaaaaaat gcattttact gcaatgaatt |
| 4741 | tctagaatat ttttccccca aagctatgcc tcctaaccct taaatggtga acaactggtt |
| 4801 | tcttgctaca gctcactgcc atttcttctt actatcatca ctaggtttcc taagattcac |
| 4861 | tcatacagta ttatttgaag attcagcttt gttctgtgaa tgtcatctta ggattgtgtc |
| 4921 | tatattcttt tgcttatttc tttttactct gggcctctca tactagtaag attttaaaaa |
| 4981 | gccttttctt ctctgtatgt ttggctcacc aaggcgaaat atatattctt ctctttttca |
| 5041 | tttctcaaga ataaacctca tctgcttttt tgtttttctg tgttttggct tggtactgaa |
| 5101 | tgactcaact gctcggtttt aaagttcaaa gtgtaagtac ttagggttag tactgcttat |
| 5161 | ttcaataatg ttgacggtga ctatctttgg aaagcagtaa catgctgtct tagaaatgac |
| 5221 | attaataatg ggcttaaaca aatgaatagg ggggtccccc cactctcctt ttgtatgcct |
| 5281 | atgtgtgtct gatttgttaa aagatggaca gggaattgat tgcagagtgt cgcttccttc |
| 5341 | taaagtagtt ttattttgtc tactgttagt atttaaagat cctggaggtg gacataagga |
| 5401 | ataaatggaa gagaaaagta gatattgtat ggtggctact aaaaggaaat tcaaaaagtc |
| 5461 | ttagaacccg agcacctgag caaactgcag tagtcaaaat atttatctca tgttaaagaa |
| 5521 | aggcaaatct agtgtaagaa atgagtacca tatagggttt tgaagttcat atactagaaa |
| 5581 | cacttaaaag atatcatttc agatattacg tttggcattg ttcttaagta tttatatctt |
| 5641 | tgagtcaagc tgataattaa aaaaaatctg ttaatggagt gtatatttca taatgtatca |
| 5701 | aaatggtgtc tatacctaag gtagcattat tgaagagaga tatgtttatg tagtaagtta |
| 5761 | ttaacataat gagtaacaaa taatgtttcc agaagaaagg aaaacacatt ttcagagtgc |
| 5821 | gtttttatca gaggaagaca aaaatacaca cccctctcca gtagcttatt tttacaaagc |
| 5881 | cggcccagtg aattagaaaa acaaagcact tggatatgat ttttggaaag cccaggtaca |
| 5941 | cttattattc aaaatgcact tttactgagt ttgaaaagtt tcttttatat ttaaaataag |
| 6001 | ggttcaaata tgcatattca atttttatag tagttatcta tttgcaaagc atatattaac |
| 6061 | tagtaattgg ctgttaattt tatagacatg gtagccaggg aagtatatca atgacctatt |
| 6121 | aagtattttg acaagcaatt tacatatctg atgacctcgt atctcttttt cagcaagtca |
| 6181 | aatgctatgt aattgttcca ttgtgtgttg tataaaatga atcaacacgg taagaaaaag |
| 6241 | gttagagtta ttaaaataat aaactgacta aaatactcat ttgaatttat tcagaatgtt |
| 6301 | cataatgctt tcaaaggaca tagcagagct tttgtggagt atccgcacaa cattatttat |
| 6361 | tatctatgga ctaaatcaat tttttgaagt tgctttaaaa tttaaaagca cctttgctta |
| 6421 | atataaagcc ctttaatttt aactgacaga tcaattctga aactttattt tgaaaagaaa |
| 6481 | atggggaaga atctgtgtct ttagaattaa aagaaatgaa aaaaataaac ccgacattct |
| 6541 | aaaaaaatag aataagaaac ctgattttta gtactaatga aatagcgggt gacaaaatag |
| 6601 | ttgtcttttt gattttgatc acaaaaaata aactggtagt gacaggatat gatggagaga |
| 6661 | tttgacatcc tggcaaatca ctgtcattga ttcaattatt ctaattctga ataaaagctg |
| 6721 | tatacagtaa aa |

By “PDX1 polypeptide” is meant a protein or fragment thereof having at least 85% amino acid sequence identity to the sequence provided at NCBI Accession No. NP_000200.1 and having transcription factor activity. The amino acid sequence provided at NCBI Accession No. NP_000200.1 is shown below:

| (SEQ ID NO: 43) | |
|---|---|
| 1 | mngeeqyyaa tqlykdpcaf qrgpapefsa sppaclymgr qpppppphpf pgalgaleqg |
| 61 | sppdispyev ppladdpava hlhhhlpaql alphppagpf pegaepgvle epnrvqlpfp |
| 121 | wmkstkahaw kgqwaggaya aepeenkrtr taytraqlle lekeflfnky isrprrvela |
| 181 | vmlnlterhi kiwfqnrrmk wkkeedkkrg ggtavggggv aepeqdcavt sgeellalpp |
| 241 | ppppggavpp aapvaaregr lppglsaspq pssvaprrpq epr |

By “PDX1 polynucleotide” is meant a polynucleotide encoding a PDX1 polypeptide or fragment thereof. An exemplary PDX1 polynucleotide sequence is provided at NCBI Ref: NM_000209.3. The sequence provided at NCBI Ref: NM_000209.3 is reproduced below:

| (SEQ ID NO: 44) | |
|---|---|
| 1 | gggtggcgcc gggagtggga acgccacaca gtgccaaatc cccggctcca gctcccgact |
| 61 | cccggctccc ggctcccggc tcccggtgcc caatcccggg ccgcagccat gaacggcgag |
| 121 | gagcagtact acgcggccac gcagctttac aaggacccat gcgcgttcca gcgaggcccg |
| 181 | gcgccggagt tcagcgccag cccccctgcg tgcctgtaca tgggccgcca gcccccgccg |
| 241 | ccgccgccgc acccgttccc tggcgccctg ggcgcgctgg agcagggcag ccccccggac |
| 301 | atctccccgt acgaggtgcc ccccctcgcc gacgaccccg cggtggcgca ccttcaccac |
| 361 | cacctcccgg ctcagctcgc gctcccccac ccgcccgccg ggcccttccc ggagggagcc |
| 421 | gagccgggcg tcctggagga gcccaaccgc gtccagctgc ctttcccatg gatgaagtct |
| 481 | accaaagctc acgcgtggaa aggccagtgg gcaggcggcg cctacgctgc ggagccggag |
| 541 | gagaacaagc ggacgcgcac ggcctacacg cgcgcacagc tgctagagct ggagaaggag |
| 601 | ttcctattca acaagtacat ctcacggccg cgccgggtgg agctggctgt catgttgaac |
| 661 | ttgaccgaga gacacatcaa gatctggttc caaaaccgcc gcatgaagtg gaaaaaggag |
| 721 | gaggacaaga agcgcggcgg cgggacagct gtcgggggtg gcggggtcgc ggagcctgag |
| 781 | caggactgcg ccgtgacctc cggcgaggag cttctggcgc tgccgccgcc gccgcccccc |
| 841 | ggaggtgctg tgccgcccgc tgcccccgtt gccgcccgag agggccgcct gccgcctggc |
| 901 | cttagcgcgt cgccacagcc ctccagcgtc gcgcctcggc ggccgcagga accacgatga |
| 961 | gaggcaggag ctgctcctgg ctgaggggct tcaaccactc gccgaggagg agcagagggc |
| 1021 | ctaggaggac cccgggcgtg gaccacccgc cctggcagtt gaatggggcg gcaattgcgg |
| 1081 | ggcccacctt agaccgaagg ggaaaacccg ctctctcagg cgcatgtgcc agttggggcc |
| 1141 | ccgcgggtag atgccggcag gccttccgga agaaaaagag ccattggttt ttgtagtatt |
| 1201 | ggggccctct tttagtgata ctggattggc gttgtttgtg gctgttgcgc acatccctgc |
| 1261 | cctcctacag cactccacct tgggacctgt ttagagaagc cggctcttca aagacaatgg |
| 1321 | aaactgtacc atacacattg gaaggctccc taacacacac agcggggaag ctgggccgag |
| 1381 | taccttaatc tgccataaag ccattcttac tcgggcgacc cctttaagtt tagaaataat |
| 1441 | tgaaaggaaa tgtttgagtt ttcaaagatc ccgtgaaatt gatgccagtg gaatacagtg |
| 1501 | agtcctcctc ttcctcctcc tcctcttccc cctccccttc ctcctcctcc tcttcttttc |
| 1561 | cctcctcttc ctcttcctcc tgctctcctt tcctccccct cctcttttcc ctcctcttcc |
| 1621 | tcttcctcct gctctccttt cctccccctc ctctttctcc tcctcctcct cttcttcccc |
| 1681 | ctcctctccc tcctcctctt cttccccctc ctctccctcc tcctcttctt ctccctcctc |
| 1741 | ttcctcttcc tcctcttcca cgtgctctcc tttcctcccc ctcctcttgc tccccttctt |
| 1801 | ccccgtcctc ttcctcctcc tcctcttctt ctccctcctc ttcctcctcc tctttcttcc |
| 1861 | tgacctcttt ctttctcctc ctcctccttc tacctcccct tctcatccct cctcttcctc |
| 1921 | ttctctagct gcacacttca ctactgcaca tcttataact tgcacccctt tcttctgagg |
| 1981 | aagagaacat cttgcaaggc agggcgagca gcggcagggc tggcttagga gcagtgcaag |
| 2041 | agtccctgtg ctccagttcc acactgctgg cagggaaggc aaggggggac gggcctggat |
| 2101 | ctgggggtga gggagaaaga tggacccctg ggtgaccact aaaccaaaga tattcggaac |
| 2161 | tttctattta ggatgtggac gtaattcctg ttccgaggta gaggctgtgc tgaagacaag |
| 2221 | cacagtggcc tggtgcgcct tggaaaccaa caactattca cgagccagta tgaccttcac |
| 2281 | atctttagaa attatgaaaa cgtatgtgat tggagggttt ggaaaaccag ttatcttatt |
| 2341 | taacatttta aaaattacct aacagttatt tacaaacagg tctgtgcatc ccaggtctgt |
| 2401 | cttcttttca aggtctgggc cttgtgctcg ggttatgttt gtgggaaatg cttaataaat |
| 2461 | actgataata tgggaagaga tgaaaactga ttctcctcac tttgtttcaa acctttctgg |
| 2521 | cagtgggatg attcgaattc acttttaaaa ttaaattagc gtgttttgtt ttg |

By “PTF1 polypeptide” is meant a protein or fragment thereof having at least 85% amino acid sequence identity to the sequence provided at NCBI Accession No. NP_835455.1 and having transcription factor activity. The amino acid sequence provided at NCBI Accession No. NP_835455.1 is shown below:

| (SEQ ID NO: 45) | |
|---|---|
| 1 | mdavllehfp ggldafpssy fdeddfftdq ssrdpledgd elladeqaev eflshqlhey |
| 61 | cyrdgaclll qpappaapla lappssgglg epddgggggy ccetgappgg fpyspgspps |
| 121 | claypcagaa vlspgarlrg lsgaaaaaar rrrrvrseae lqqlrqaanv rerrrmqsin |
| 181 | dafeglrshi ptlpyekrls kvdtlrlaig yinflselvq adlplrggga ggcggpgggg |
| 241 | rlggdspgsq aqkviichrg trspspsdpd yglpplaghs lswtdekqlk eqniirtakv |
| 301 | wtpedprkln skssfnnien eppfefvs |

By “PTF1 polynucleotide” is meant a polynucleotide encoding a PTF1 polypeptide or fragment thereof. An exemplary PTF1 polynucleotide sequence is provided at NCBI Ref: NM_178161.2. The sequence provided at NCBI Ref: NM_178161.2 is reproduced below:

| (SEQ ID NO: 46) | |
|---|---|
| 1 | atggacgcgg tgttgctgga gcacttcccc gggggcctag acgcctttcc ttcttcgtac |
| 61 | ttcgacgagg acgacttctt caccgaccag tcttcacggg accccctgga ggacggcgat |
| 121 | gagctgctgg cggacgagca ggccgaggtg gagttcctta gccaccagct ccacgagtac |
| 181 | tgctaccgcg acggggcgtg cctgctgctg cagcccgcgc ccccggccgc cccgctagcg |
| 241 | ctcgccccgc cgtcctcggg gggcctcggt gagccagacg acggcggcgg cggcggctac |
| 301 | tgctgcgaga cgggggcgcc cccaggcggc ttcccctact cgcccggctc gccgccctcg |
| 361 | tgcctggcct acccgtgcgc cggggcggca gtactgtctc ccggggcgcg gctgcgcggc |
| 421 | ctgagcggag cggcggctgc ggcggcgcgg cgccggcggc gggtgcgctc cgaggcggag |
| 481 | ctgcagcagc tgcggcaggc ggccaacgtg cgcgagcggc ggcgcatgca gtccatcaac |
| 541 | gacgccttcg aggggctgcg ctcgcacatc cccacgctgc cctacgagaa gcgcctctcc |
| 601 | aaggtggaca cgctgcgcct ggccatcggc tacatcaact tcctcagcga gctcgtgcag |
| 661 | gccgacctgc ccttgcgcgg cggtggcgcg ggcggctgcg gggggccggg cggcggcggg |
| 721 | cgcctgggcg gggacagccc gggcagccag gcccagaagg tcatcatctg ccatcggggc |
| 781 | acccggtccc cctcccccag cgaccctgat tatggcctcc ctcccctagc aggacactct |
| 841 | ctctcatgga ctgatgaaaa acaactcaag gaacaaaata ttatccgaac agccaaagtc |
| 901 | tggaccccag aggaccccag aaaactcaac agcaaatctt ccttcaacaa catagaaaac |
| 961 | gaaccaccat ttgagtttgt gtcctgagaa gtcccagact cggctgaaga tctgattatg |
| 1021 | tctctgtgca tattgtacat gtaaatatct ataatgtaaa tgtaatttaa gaatcaaatt |
| 1081 | tttcgaatgg caatcaactg tttattattt atctatttat tatcctgttg agttgatgaa |
| 1141 | atagatgatt tctttttaaa tatataattt atataactta tcctgatttt ctgaaaatat |
| 1201 | gcaatagcct atgattttcc tgaactctgt gttgttggga gaactctggc cagaaaacgt |
| 1261 | cctgcttatt tattgccaga tatggtttat ttctaagcgt tgtcaataaa tgctatttac |
| 1321 | accttttcct gaaaaaaaa |

By “Wnt3a polynucleotide” is meant a polynucleotide encoding a Wnt3a polypeptide or a fragment thereof, or a polynucleotide having at least 85% sequence identity to the human Wnt3a polynucleotide sequence. An exemplary human Wnt3a polynucleotide sequence is provided at NCBI GenBank Accession No. AB060284.1. The polynucleotide sequence provided at NCBI GenBank Accession No. AB060284.1 is reproduced below:

| (SEQ ID NO: 47) | |
|---|---|
| 1 | cggcgatggc cccactcgga tacttcttac tcctctgcag cctgaagcag gctctgggca |
| 61 | gctacccgat ctggtggtcg ctggctgttg ggccacagta ttcctccctg ggctcgcagc |
| 121 | ccatcctgtg tgccagcatc ccgggcctgg tccccaagca gctccgcttc tgcaggaact |
| 181 | acgtggagat catgcccagc gtggccgagg gcatcaagat tggcatccag gagtgccagc |
| 241 | accagttccg cggccgccgg tggaactgca ccaccgtcca cgacagcctg gccatcttcg |
| 301 | ggcccgtgct ggacaaagct accagggagt cggcctttgt ccacgccatt gcctcagccg |
| 361 | gtgtggcctt tgcagtgaca cgctcatgtg cagaaggcac ggccgccatc tgtggctgca |
| 421 | gcagccgcca ccagggctca ccaggcaagg gctggaagtg gggtggctgt agcgaggaca |
| 481 | tcgagtttgg tgggatggtg tctcgggagt tcgccgacgc ccgggagaac cggccagatg |
| 541 | cccgctcagc catgaaccgc cacaacaacg aggctgggcg ccaggccatc gccagccaca |
| 601 | tgcacctcaa gtgcaagtgc cacgggctgt cgggcagctg cgaggtgaag acatgctggt |
| 661 | ggtcgcaacc cgacttccgc gccatcggtg acttcctcaa ggacaagtac gacagcgcct |
| 721 | cggagatggt ggtggagaag caccgggagt cccgcggctg ggtggagacc ctgcggccgc |
| 781 | gctacaccta cttcaaggtg cccacggagc gcgacctggt ctactacgag gcctcgccca |
| 841 | acttctgcga gcccaaccct gagacgggct ccttcggcac gcgcgaccgc acctgcaacg |
| 901 | tcagctcgca cggcatcgac ggctgcgacc tgctgtgctg cggccgcggc cacaacgcgc |
| 961 | gagcggagcg gcgccgggag aagtgccgct gcgtgttcca ctggtgctgc tacgtcagct |
| 1021 | gccaggagtg cacgcgcgtc tacgacgtgc acacctgcaa gtaggcaccg gccgcggctc |
| 1081 | cccctggacg gggcgggccc tgcctgaggg tgggcttttc cctgggtgga gcaggactcc |
| 1141 | cacctaaacg gggcagtact cctccctggg ggcgggactc ctccctgggg gtggggctcc |
| 1201 | tacctggggg cagaactcct acctgaaggc agggctcctc cctggagcta gtgtctcctc |
| 1261 | tctggtggct gggctgctcc tgaatgaggc ggagctccag gatggggagg ggctctgcgt |
| 1321 | tggcttctcc ctggggacgg ggctcccctg gacagaggcg gggctacaga ttgggcgggg |
| 1381 | cttctcttgg gtgggacagg gcttctcctg cgggggcgag gcccctccca gtaagggcgt |
| 1441 | ggctctgggt gggcggggca ctaggtaggc ttctacctgc aggcggggct cctcctgaag |
| 1501 | gaggcggggc tctaggatgg ggcacggctc tggggtaggc tgctccctga gggcg |

By “Wnt3a polypeptide” is meant a Wnt3a polypeptide or a fragment thereof, or a polypeptide having at least 85% sequence identity to the human Wnt3a polypeptide sequence. An exemplary human Wnt3a polypeptide sequence is provided at NCBI GenBank: AAI03924.1. The sequence provided at GenBank: AAI03924.1 is reproduced below:

| (SEQ ID NO: 48) | |
|---|---|
| 1 | maplgyflll cslkqalgsy piwwslavgp qysslgsqpi lcasipglvp kqlrfcrnyv |
| 61 | eimpsvaegi kigiqecqhq frgrrwnctt vhdslaifgp vldkatresa fvhaiasagv |
| 121 | afavtrscae gtaaicgcss rhqgspgkgw kwggcsedie fggmvsrefa darenrpdar |
| 181 | samnrhnnea grqaiashmh lkckchglsg scevktcwws qpdfraigdf lkdkydsase |
| 241 | mvvekhresr gwvetlrpry tyfkvpterd lvyyeaspnf cepnpetgsf gtrdrtcnvs |
| 301 | shgidgcdll ccgrghnara errrekcrcv fhwccyvscq ectrvydvht cknpgsragn |
| 361 | sahqpphpqp pvrfhpplrr agkvp |

By “Wnt4 polynucleotide” is meant a polynucleotide encoding Wnt4 polypeptide or a fragment thereof, or a polynucleotide having at least 85% sequence identity to the human Wnt4 polynucleotide sequence. An exemplary human Wnt4 polynucleotide sequence is provided at NCBI GenBank Accession No. AY009398.1. Accession number NCBI Ref NG_008974.1 is a reference standard Wnt4a polynucleotide sequence. The polynucleotide sequence provided at NCBI GenBank Accession No. AY009398.1 is reproduced below:

| (SEQ ID NO: 49) | |
|---|---|
| 1 | atgagtcccc gctcgtgcct gcgttcgctg cgcctcctcg tcttcgccgt cttctcagcc |
| 61 | gccgcgagca actggctgta cctggccaag ctgtcgtcgg tggggagcat ctcagaggag |
| 121 | gagacgtgcg agaaactcaa gggcctgatc cagaggcagg tgcagatgtg caagcggaac |
| 181 | ctggaagtca tggactcggt gcgccgcggt gcccagctgg ccattgagga gtgccagtac |
| 241 | cagttccgga accggcgctg gaactgctcc acactcgact ccttgcccgt cttcggcaag |
| 301 | gtggtgacgc aagggattcg ggaggcggcc ttggtgtacg ccatctcttc ggcaggtgtg |
| 361 | gcctttgcag tgacgcgggc gtgcagcagt ggggagctgg agaagtgcgg ctgtgacagg |
| 421 | acagtgcatg gggtcagccc acagggcttc cagtggtcag gatgctctga caacatcgcc |
| 481 | tacggtgtgg ccttctcaca gtcgtttgtg gatgtgcggg agagaagcaa gggggcctcg |
| 541 | tccagcagag ccctcatgaa cctccacaac aatgaggccg gcaggaaggc catcctgaca |
| 601 | cacatgcggg tggaatgcaa gtgccacggg gtgtcaggct cctgtgaggt aaagacgtgc |
| 661 | tggcgagccg tgccgccctt ccgccaggtg ggtcacgcac tgaaggagaa gtttgatggt |
| 721 | gccactgagg tggagccacg ccgcgtgggc tcctccaggg cactggtgcc acgcaacgca |
| 781 | cagttcaagc cgcacacaga tgaggacttg gtgtacttgg agcctagccc cgacttctgt |
| 841 | gagcaggaca tgcgcagcgg cgtgctgggc acgaggggcc gcacatgcaa caagacgtcc |
| 901 | aaggccatcg acggctgtga gctgctgtgc tgtggccgcg gcttccacac ggcgcaggtg |
| 961 | gagctggctg aacgctgcag ctgcaaattc cactggtgct gcttcgtcaa gtgccggcag |
| 1021 | tgccagcggc tcgtggagtt gcacacgtgc cgatga |

By “Wnt4 polypeptide” is meant a Wnt4 polypeptide or a fragment thereof, or a polypeptide having at least 85% sequence identity to the human Wnt4 polypeptide sequence. An exemplary human Wnt4 polypeptide sequence is provided at NCBI GenBank Accession No.: AAG38658.1. The sequence provided at GenBank Accession No.: AAG38658.1 is reproduced below:

| (SEQ ID NO: 50) | |
|---|---|
| 1 | msprsclrsl rllvfavfsa aasnwlylak lssvgsisee etceklkgli qrqvqmckrn |
| 61 | levmdsvrrg aqlaieecqy qfrnrrwncs tldslpvfgk vvtqgireaa lvyaissagv |
| 121 | afavtracss gelekcgcdr tvhgvspqgf qwsgcsdnia ygvafsqsfv dvrerskgas |
| 181 | ssralmnlhn neagrkailt hmrveckchg vsgscevktc wravppfrqv ghalkekfdg |
| 241 | ateveprrvg ssralvprna qfkphtdedl vylepspdfc eqdmrsgvlg trgrtcnkts |
| 301 | kaidgcellc cgrgfhtaqv elaercsckf hwccfvkcrq cqrlvelhtc r |

By “Wnt5a polynucleotide” is meant a polynucleotide encoding Wnt5a polypeptide or a fragment thereof, or a polynucleotide having at least 85% sequence identity to the human Wnt5a polynucleotide sequence. An exemplary polynucleotide sequence coding for human Wnt5a is provided at NCBI Ref: GenBank NM_003392, a reference standard sequence. Nucleotides 658-1800 of the Wnt5a genomic sequence having 6194 nucleotides codes for a human Wnt5a polypeptide. The polynucleotide sequence of the human Wnt5a coding sequence provided at bases 658-1800 of NCBI Ref: GenBank NM_003392 is reproduced below:

| (SEQ ID NO: 51) | |
|---|---|
| 658 | atg |
| 661 | aagaagtcca ttggaatatt aagcccagga gttgctttgg ggatggctgg aagtgcaatg |
| 721 | tcttccaagt tcttcctagt ggctttggcc atatttttct ccttcgccca ggttgtaatt |
| 781 | gaagccaatt cttggtggtc gctaggtatg aataaccctg ttcagatgtc agaagtatat |
| 841 | attataggag cacagcctct ctgcagccaa ctggcaggac tttctcaagg acagaagaaa |
| 901 | ctgtgccact tgtatcagga ccacatgcag tacatcggag aaggcgcgaa gacaggcatc |
| 961 | aaagaatgcc agtatcaatt ccgacatcga aggtggaact gcagcactgt ggataacacc |
| 1021 | tctgtttttg gcagggtgat gcagataggc agccgcgaga cggccttcac atacgcggtg |
| 1081 | agcgcagcag gggtggtgaa cgccatgagc cgggcgtgcc gcgagggcga gctgtccacc |
| 1141 | tgcggctgca gccgcgccgc gcgccccaag gacctgccgc gggactggct ctggggcggc |
| 1201 | tgcggcgaca acatcgacta tggctaccgc tttgccaagg agttcgtgga cgcccgcgag |
| 1261 | cgggagcgca tccacgccaa gggctcctac gagagtgctc gcatcctcat gaacctgcac |
| 1321 | aacaacgagg ccggccgcag gacggtgtac aacctggctg atgtggcctg caagtgccat |
| 1381 | ggggtgtccg gctcatgtag cctgaagaca tgctggctgc agctggcaga cttccgcaag |
| 1441 | gtgggtgatg ccctgaagga gaagtacgac agcgcggcgg ccatgcggct caacagccgg |
| 1501 | ggcaagttgg tacaggtcaa cagccgcttc aactcgccca ccacacaaga cctggtctac |
| 1561 | atcgacccca gccctgacta ctgcgtgcgc aatgagagca ccggctcgct gggcacgcag |
| 1621 | ggccgcctgt gcaacaagac gtcggagggc atggatggct gcgagctcat gtgctgcggc |
| 1681 | cgtggctacg accagttcaa gaccgtgcag acggagcgct gccactgcaa gttccactgg |
| 1741 | tgctgctacg tcaagtgcaa gaagtgcacg gagatcgtgg accagtttgt gtgcaagtag |

By “Wnt5a polypeptide” is meant a Wnt5a polypeptide or a fragment thereof, or a polypeptide having at least 85% sequence identity to the human Wnt5a polypeptide sequence. An exemplary human Wnt5a (isoform 1) polypeptide sequence is provided at UniProtKB Identifier: P41221-1. The sequence provided at UniProtKB Identifier: P41221-1 is reproduced below:

| (SEQ ID NO: 52) | |
|---|---|
| 1 | mkksigilsp gvalgmagsa msskfflval aiffsfaqvv ieanswwslg |
| 51 | mnnpvqmsev yiigaqplcs qlaglsqgqk klchlyqdhm qyigegaktg |
| 101 | ikecqyqfrh rrwncstvdn tsvfgrvmqi gsretaftya vsaagvvnam |
| 151 | sracregels tcgcsraarp kdlprdwlwg gcgdnidygy rfakefvdar |
| 201 | ererihakgs yesarilmnl hnneagrrtv ynladvackc hgvsgscslk |
| 251 | tcwlqladfr kvgdalkeky dsaaamrlns rgklvqvnsr fnspttqdlv |
| 301 | yidpspdycv rnestgslgt qgrlcnktse gmdgcelmcc grgydqfktv |
| 351 | qterchckfh wccyvkckkc teivdqfvck |

By “progenitor cell” is meant a cell that a multipotent stem cell that is capable of generating (e.g., by differentiation or division) an endothelial cell. A progenitor cell that is capable of generating an endothelial cell may express this capability when grown under appropriate in vitro or in vivo conditions, such as those described herein.

By “progeny” is meant a cell derived from a multipotent stem cell of the invention. Progeny include without limitation progenitor cells, differentiated cells, and terminally differentiated cells.

By “derived from” is meant the process of obtaining a progeny cell.

By “reduces” is meant a negative alteration of at least 10%, 25%, 50%, 75%, or 100%.

By “reference” or “control” is meant a standard condition. For example, an untreated cell, tissue, or organ that is used as a reference.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, or at least about 25 amino acids. The length of the reference polypeptide sequence can be about 35 amino acids, about 50 amino acids, or about 100 amino acids. For nucleic acids, the length of the reference nucleic acid sequence will generally be at least about 50 nucleotides, at least about 60 nucleotides, or at least about 75 nucleotides. The length of the reference nucleic acid sequence can be about 100 nucleotides, about 300 nucleotides or any integer thereabout or therebetween.

A “somatic” cell refers to a cell that is obtained from a tissue of a subject. Such subjects are at a post-natal stage of development (e.g., adult, infant, child). In contrast, an “embryonic cell” or “embryonic stem cell” is derived from an embryo at a pre-natal stage of development.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., at least about 37° C., and at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In yet another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., at least about 42° C., and at least about 68° C. In one embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In yet another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196: 180, 1977); Grunstein and Hogness (Proc. Natl. Acad. Sci., USA 72: 3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence is at least 60%, at least 80%, at least 85%, at least 90%, at least 95% or even at least 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “self renewal” as used herein refers to the process by which a stem cell divides to generate one (asymmetric division) or two (symmetric division) daughter cells with development potentials that are indistinguishable from those of the mother cell. Self renewal involves both proliferation and the maintenance of an undifferentiated state.

The term “stem cell” is meant a pluripotent cell or multipotent stem cell having the capacity to self-renew and to differentiate into multiple cell lineages.

By “subject” is meant a mammal, including, but not limited to, a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “tissue” is meant a collection of cells having a similar morphology and function.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “vascularized” is meant having a blood vessel. In some embodiments, the pancreatic islet organoid or pancreatic organoid is vascularized.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a schematic showing the various symptoms of human disease that can be modeled in a dish. Generation of functional human organs provides new therapeutic strategies in drug-screening and disease modeling. Described herein is a novel technique to generate 3D human mini-organs in a dish. Using this technique, human type 2 diabetes can be modeled in a dish to find effective drugs in genetic, patients or environmental specific diseases such as human type 2 diabetes.

FIG. 2A is a set of micrographs showing the utility of human adipose-derived stem cells (hADSC) in organogenesis. In studies described herein, human adipose-derived stem cells were found to be a novel resource for generation of self-organized organoids (organ bud). FIG. 2 compares the appearance of human iPSC-derived pancreatic progenitors (PPs. Day 15-day 19), human umbilical vein endothelial cells (HUVEC), and hADSCs cultured on plates (2D) with those cultured in the 3 dimensional matrigel (Matrigel® system for 24 hours in PP cell differentiation media, endothelial cell growth media, and hADSC growth media, respectively. FIG. 2B is a set of micrographs demonstrating the ability of ADSCs to progressively self-organize when cultured in Matrigel. FIG. 2C is a set of images of hADSCs, seeded at the indicated density, that demonstrate the minimum number of cells required for sphere formation when grown in Matrigel. FIGS. 2D and 2D-1 to 2D-4 show transcriptional changes occurring in hADSCs during 48 hours of culture in Matrigel, depicted as a heatmap. Statistically different changes in gene expression were determined by RNA-Seq analyses of cells after the indicated time in Matrigel. Biological pathways altered during culture in Matrigel were identified using DAVID software. FIG. 2E is a schematic describing the generation of human islet-like organoids by culturing in Matrigel. FIG. 2F is an image of human islet-like organoids in a single well of a 24 well plate. FIG. 2G is a set of images of a human islet-like organoid generated by co-culturing hiPSC-derived pancreatic progenitors, HUVECs, and hADSCs for 1-5 days in Matrigel. GFP is used to indicate human insulin expression (green, 1^{st} panel), mCherry to label HUVEC cells (red, 2^{nd} panel), brightfield image (3^{rd} panel), and an overlay of GFP and mCherry images (4^{th} panel).

FIGS. 3A-3F are plots, images, and a schematic showing characterization of insulin secretion of INS-1 cells, mouse islets, and human islets using a proinsulin luciferase reporter system as a quantitative insulin secretion assay. FIG. 3A is a schematic showing the generation of a stably-expressed insulin reporter in the rat beta cell line INS-1 cells, where luciferase expression is under the control of the proinsulin promoter. FIG. 3B is a plot showing luciferase activity induced in the INS-1 reporter cells in response to treatment with 3 mM glucose (G3), 20 mM glucose (G20), 20 mM glucose (G20) plus 100 nM Exendin-4 (Ex-4), or 20 mM potassium chloride (KCl). FIG. 3C is an image showing a single mouse islet in one well of a 96-well plate. The mouse islet cells were infected with the proinsulin-luciferase lentivirus reporter construct 2 days before assaying. FIG. 3D is a set of plots showing the luciferase activity from individual mouse islets infected with the proinsulin luciferase reporter in response to 3 mM glucose (G3 mM) and 20 mM glucose (G20 mM) (left), and the average of the individual assays (right). FIG. 3E is an image showing a single human islet in one well of a 96-well plate. The human islet cells were infected with the proinsulin-luciferase lentivirus reporter construct 2 days before assaying. FIG. 3F is a set of plots showing the luciferase activity from individual human islets infected with the proinsulin luciferase reporter in response to 3 mM glucose (G3 mM) and 20 mM glucose (G20 mM) (left), and the average of the individual assays (right). FIGS. 3D and 3F show that glucose-stimulated insulin secretion (GSIS) can be measured from single mouse and human islets, respectively, after the infection with the lentiviral luciferase reporter.

FIGS. 4A-4I are plots, images, graphs and a schematic showing generation of functional, vascularized human pancreatic islets in a dish. FIG. 4A is a schematic showing a scheme for the generation of functional, vascularized human pancreatic islets in Gellan gum. FIG. 4B shows human islet-like mini organs. Top panels show insulin positive cells (green fluorescent protein expression driven by the insulin promoter (left)) and phase contrast images (left) of islet-like organoids grown in 3D Gellan gum suspensions (bottom panel). Electron microscopy images reveal insulin granules in the (3-like cells and lipid droplets in the hADSCs. FIG. 4C shows that human islet-like-mini organs generated by methods described herein are morphologically identical to human islets. FIG. 4D and FIG. 4E show relative expression of genes associated with β cell determination (FIG. 4D), and mitochondrial function (FIG. 4E), as measured by qPCR. Islet-like cell clusters (derived from pancreatic progenitors cultured in gellan gum, day 35) and islet-like organoids (derived from pancreatic progenitors co-cultured with HUVECs and hADSCs in gellan gum) were FACS stored into insulin expressing (GFP positive) and non-expressing (GFP negative) cells prior to analysis, and compared with gene expression in human islets. FIG. 4F shows glucose-stimulated insulin secretion, measured by the fold change in c-peptide secretion 30 minutes after exposure to 20 mM glucose, in selected islet-like organoid preparations prepared in 3D gellan gum cultures and human islets. FIG. 4G shows that human islet-like mini organs generated by the methods described herein can develop functional vascularization. Human islet-like organoids were transferred to matrigel and grown in the presence of endothelial growth media. Cells expressing insulin are visualized as green fluorescence. Top panels are fluorescent images of cells showing HUVEC cell outgrowth 24 and 48 hours after stimulation by endothelial cell growth medium (ECM). The bottom panel is a schematic summarizing the experiment and the finding. FIG. 4H shows a graph illustrating that human islet-like mini organs generated by the methods described herein can regulate blood glucose in a known mouse model of type 1 diabetes, NODSCID. In this mouse model, the SCID (Severe Combined Immune Deficiency) mutation has been transferred onto a diabetes-susceptible Non-Obese Diabetic (NOD) background. The multiple immunological defects in this mouse model provide a system for reconstituting the animal with human hematopoietic cells. The graph in FIG. 4H shows the blood glucose levels in NODSCID mice treated with streptozotocin (STZ) (180 mg/kg) to induce type 1 diabetes after transplantation into the kidney capsule of hiPSC-derived human islet-like organoids (n=1000), (dotted line with squares); human islets (n=1000), (dashed line); or mock treatment (solid line with white circles). FIG. 4I shows a bar graph illustrating that human islet-like mini organs (“human islets organoids”) generated by the methods described herein are able to secrete insulin postprandially. The graph of FIG. 4I shows the serum levels of human c-peptide (pmol/L) in NODSCID mice 4 weeks after the transplantations described in FIG. 4H under random fed (left bar), 8 hour fasted (middle bar), and refed (right bar) conditions. Human c-peptide levels provide a measure of insulin secretion from the transplants that is distinct from endogenous murine insulin.

FIGS. 5A-5E are diagrams and plots showing the generation of functional islet-like organoids in 3D gellan gum cultures. FIG. 5A is a schematic describing the generation of hiPSCs stably incorporating dual reporters for insulin expression (GFP) and insulin secretion (luciferase). FIG. 5B is a bar graph showing the increased expression of human insulin during the differentiation of hiPSCs incorporating the dual reporters. FIG. 5C is a bar graph comparing the glucose stimulated insulin secretion (GSIS) from human islet-like organoids generated using the methods described herein with human islets. GSIS, as measured using secreted luciferase, in single islet-like organoids or human islets in response to 3 mM and 20 mM glucose. Arrows indicate functional organoids capable of increasing insulin secretion in response to a glucose challenge. FIG. 5D is a bar graph comparing the insulin secretion of independent batches of islet-like organoids, prepared as described herein, to mouse islets as a negative control. Insulin secretion was measured in response to 3 mM glucose (G3 mM), 20 mM glucose (G20 mM), 20 mM glucose (G20 mM) and Exendin-4 (Ex4), or 20 mM potassium chloride (KCl20 mM) after 133 days in culture. Response was measured as secreted luciferase activity from pooled organoids (100 organoids/sample). FIG. 5E shows intracellular luciferase activity as a measure of intracellular insulin (100 organoids/sample).

FIGS. 6A-6H are schematics and images showing generation of functional human mini-organs in a dish. FIG. 6A is a schematic showing generation of functional human mini organs including human islets, pancreas, liver, heart, and intestine. FIGS. 6B-6C show the generation of a human heart organoid. FIG. 6B (top) provides a schematic illustrating the protocol for differentiation of a human pluripotent stem cell (hPSC) into a cardiomyocyte (when cultured in 2D) or into a mini heart (when co-cultured with hADSCs and HUVECs in 3D). FIG. 6B (bottom left) provides a plot showing relative expression of cardiomyocyte-specific genes human MLC2a (hMLC2a), human Nkx2-5 (hNkx2-5), alpha myosin heavy chain (alphaMHC), and KCNQ1 before (day 0) and at day 18 of the differentiation protocol, with and without the PPAR6 agonist GW501516. At the bottom right of FIG. 6B is a videomicrograph showing beating of the hiPSC-derived cardiomyocytes. FIG. 6C shows an image of a human mini heart-like organoid generated by culturing the hiPSC-derived cardiomyocytes with hADSC and HUVEC, as described in the schematic above. FIGS. 6D-6F show generation of a human liver organoid. FIG. 6D (top) provides a schematic illustrating the protocol for differentiation of a human pluripotent stem cell (hPSC) into hepatocytes. FIG. 6D (bottom left) provides a plot showing relative expression of hepatocyte-specific genes AFP, ALB, and Cyp3a7, during the differentiation of 6 independent preparations. At the bottom right of FIG. 6D is a micrograph showing hiPSC-derived hepatocytes. FIG. 6E is a set of microcrographs showing hiPSC-derived hepatocytes. Top panels show expression of Cyp7a1 (Cyp7a1-GFP reporter, left) and SREBP1c (SREBP1c-GFP reporter, right) in hiPSC-derived hepatocytes indicating functional maturation. The bottom panels show hiPSC-derived hepatocytes cultured with (right) or without (left) phosphatidic acid (PA) overnight. Higher magnification images shown in the bottom left reveal the accumulation of lipid droplets in the hepatocytes treated with phosphatidic acid. FIG. 6F shows a human mini liver-like organoid generated by culturing the hiPSC-derived hepatocytes with hADSCs and HUVECs in the gellan gum 3D culture system. FIG. 6G (top) provides a schematic illustrating the protocol for differentiation of human pluripotent stem cells (hPSCs) into intestinal organoids when co-cultured with hADSCs and HUVECs in the gellan gum 3D culture system. The lower images shows budding of the human intestinal organoid cultures, consistent with crypt-like structures and indicating functional organoids. FIG. 6H (top) provides a schematic illustrating the protocol for differentiation of human pluripotent stem cells (hPSCs) into a mini pancreas when co-cultured with hADSCs and HUVECs in the gellan gum 3D culture system. The middle panels show images of insulin positive β cells, marked by the expression of green fluorescent protein driven by the insulin promoter, and the equivalent light microscopy image of pancreatic organoids. Exocrine cells are the remaining unlabeled cells. The bottom panels show an image of a single pancreatic organoid in a 96 well plate.

FIG. 7 is a schematic showing modeling of human Type 2 diabetes in a dish. Generation of functional human organs such as a liver, pancreas, and islets provides new therapeutic strategies in drug-screening and modeling of human type 2 diabetes. For example, hepatic organoids with accumulated lipids can be used to study early insulin resistance phenotypes, while human islet-like organoids can be used to study β cell death seen in late stage type 2 diabetes. Inserted graph shows the response of islet-like organoids to increasing concentrations of human amyloid polypeptide (hIAPP). The increase in propidium iodide (PI) staining in G0/G1 stage cells indicates that hIAPP induces apoptosis in islet-like organoids.

FIGS. 8A and 8B are schematics showing platforms for drug screening and the subsequent evaluation of potential candidates for human type 2 diabetes and pancreatic cancer. FIG. 8A shows a scheme for screening potential drugs for use in human type 2 diabetes or human pancreatic cancer tumorigenesis in a dish. Organoid cultures are exposed to appropriate stress (e.g. high levels of free fatty acids (FFAs), high glucose levels, or relevant cytokines) to induce disease-like phenotypes prior to screening drug libraries for compounds that reverse or diminish disease indications. FIG. 8B shows approaches to evaluate potential drug candidates for type 2 diabetes and human pancreatic cancer tumorigenesis/metastasis in mice. Mice transplanted with individual (e.g. pancreas) or combinations of human organoids (e.g., pancreas and liver) are exposed to appropriate disease-inducing stressors (e.g. high fat/high cholesterol (HF/HC) diet) prior to treatment with potential disease altering drug candidates.

FIG. 9 is a set of schematics and images showing a structure of a pancreas and pancreatic tissue and images of a pancreatic islet-like organoid and pancreatic organoid generated herein. The schematics on the left of FIG. 9 depict the anatomy and structure of a pancreas (top) and pancreatic islets (bottom). The image on the top-right corner of FIG. 9 shows an iPSC-derived pancreatic organoid with pancreatic islets (as marked by green fluorescent protein expression driven by the insulin promoter) and an exocrine/duct component as indicated. The image in the middle-right of FIG. 9 shows the corresponding light microscopy image of the fluorescent image above. The image on the bottom-right corner of FIG. 9 shows a single pancreatic islet organoid.

FIGS. 10A-10D is a set of schematics, images, heatmaps, and bar graphs summarizing the generation of islet-like organoids from PSCs. FIG. 10A is a schematic describing the protocol to generate islet-like organoids by culturing in 3D in gellan gum. FIG. 10B is a series of images recording the growth and differentiation of hPSCs into pancreatic lineages in 3D Gellan gum cultures, as described herein. Insulin expression is indicated by the green fluorescence seen at day 21. FIG. 10C is a heatmap representation of changes in gene expression during the differentiation of hiPSCs into islet-like organoids. FIG. 10D is a set of bar graphs reporting the changes in relative expression of the pluripotency marker Nanog, the endocrine hormones insulin, somatostatin, and glucagon, and the β cell lineage marker Nkx6-1 in hiPSCs and two stem cell lines (HuES8 and H1ES) during differentiation as described in the methods herein. Gene expression was measured by qPCR.

FIGS. 11A and 11B show a heatmap and graph illustrating the role of WNT proteins in the hADSCs in 3D culture. FIG. 11A is a heatmap depiction of gene expression changes in hADSCs during the spontaneous self-organization that occurs in 3D culture. Genes that are induced in the WNT5a pathway are listed. FIG. 11B is a graph showing the relative levels of several individual WNT proteins in hADSC 3D culture over time, identifying the WNT5a protein as the predominant protein expressed.

FIGS. 12A-12H show a set of bar graphs and images illustrating the role of WNT proteins in the metabolic maturation of iPSC-derived islet organoids. FIG. 12A shows bar graphs comparing the expression of Fltp and Esrrg genes in iPSC-derived islet organoids (day 21, generated without co-culture with hADSCs or HUVECs) after treatment with PBS, WNT3a (500 ng/ml), WNT4 (100 ng/ml), or WNT5a (400 ng/ml) for 5 days. FIG. 12B is a bar graph showing the induction of Esrrg gene expression in hiPSC-derived islet organoids, generated in the absence of supporting hADSC or HUVECs, in response to increasing doses of WNT4 (0, 10, 25, 50, 100, 200 ng/ml) and WNT5a (0, 25, 50, 100, 200, 400 ng/ml). FIG. 12C is a bar graph showing the induction of mitochondrial genes involved in oxidative phosphorylation (Cox7a2, Ndufa1, Ndufa7), lactate dehydrogenase (Ldha) and Fltp (a Wnt/planar cell polarity (PCP) effector and reporter gene) in hiPSC-derived islet organoids, generated in the absence of supporting hADSC or HUVECs, in response to increasing doses of WNT4 (0, 10, 25, 50, 100, 200 ng/ml) and WNT5a (0, 25, 50, 100, 200, 400 ng/ml). FIG. 12D shows fluorescent images showing mitochondrial (Mitotracker; Mito-Red) and insulin (Insulin-GFP) levels in hiPSC-derived islet organoids (day 27) after 8 days treatment with PBS or WNT4 (100 ng/ml). FIG. 12E shows fluorescent images of FACS analysis of hiPSC-derived islet organoids (day 27) after 8 days treatment with PBS or WNT4 (100 ng/ml). FIGS. 12F, 12G, and 12H show a set of bar graphs illustrating the results of FACS analyses of hiPSC-derived islet organoids (day 27) after 8 days treatment with PBS, WNT4 (100 ng/ml), WNT5a (400 ng/ml), control fibroblast conditioned media (50%), or WNT5a secreting fibroblast conditioned media (50%). The WNT proteins used were recombinant human (rh) proteins.

FIG. 13 shows a bar graph demonstrating a role for WNT4 in the functional maturation of hiPSC-derived islet organoids. Human iPSC (hiPSC)-derived islet organoids (day 22) were treated with PBS (Vehicle, “Veh”) or WNT4 (100 ng/ml) for 12 days, and the secretion of human c-peptide was measured in response to low glucose (3 mM, “G3 mM”), high glucose (20 mM, “G20 mM”), or high KC1 levels (20 mM, “KCL20 mM”).

### DETAILED DESCRIPTION OF THE INVENTION

The invention features compositions and methods that are useful for generating scalable, functional, vascularized organoids in vitro, particularly human pancreatic or pancreatic islet organoids. The invention is based, at least in part, on the discovery that culturing iPSC-derived beta-like cells with human adipose-derived stem cells (hADSC) and human umbilical vein endothelial cells (Huvec) in a three-dimensional matrix containing gellan gum generated functional pancreatic and pancreatic islet organoids.

The organoids generated were vascularized and exhibited functional properties, such as glucose-stimulated insulin secretion (GSIS). Islet transplantation is known as the best therapy for curing insulin deficient diabetes such as type 1 and late stage of type 2 diabetes. Recent studies have shown the possibility of generating glucose responsive insulin producing beta-like cells from human Pluripotent Stem Cells (PSCs), however the generation of functional, vascularized pancreatic islets from PSCs capable of secreting insulin, glucagon and somatostatin in response to nutrients has not been previously achieved.

Studies described herein demonstrate that using the self-organizing function of human adipose-derived stem cells (hADSC), HUVEC, and human iPSC-derived beta-like cells allows for the in vitro generation of glucose-responsive insulin secreting islet-like organoids with the ability to form functional vasculature. Studies herein further demonstrate the successful scaling of islet-like organoids production through the use of Gellan gum based 3D culture systems. Using a Gaussia luciferase reporter to measure insulin secretion, the functional heterogeneity in hiPSC-derived islet-like organoids was characterized. Without intending to be bound by theory, results herein suggest that the novel human islet-like organoids may offer a therapeutic treatment for diabetes, as well as offer a platform for drug screening, genome editing, and the modeling of organogenesis and pathogenesis of diabetes.

### Pancreas

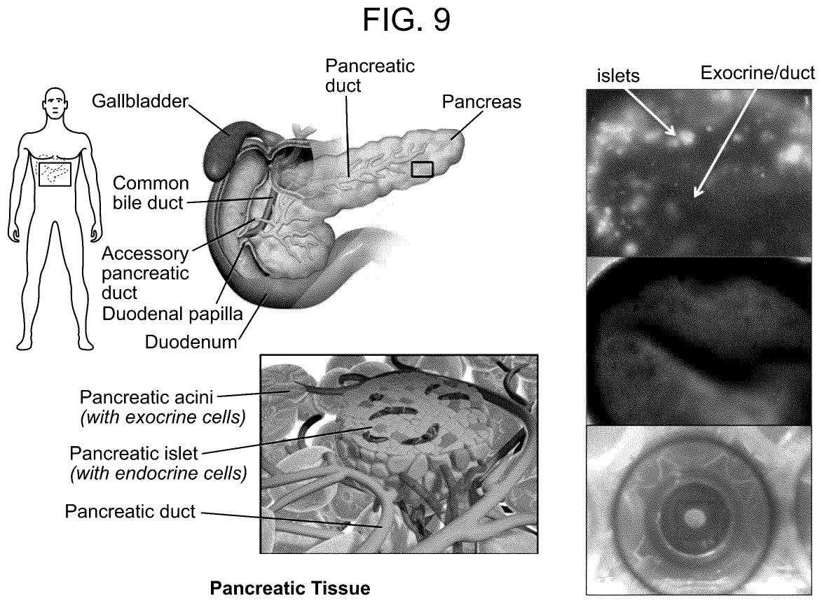

In some aspects, the invention provides a pancreatic organoid or a pancreatic islet organoid. The pancreas is an organ that lies in the abdomen and has endocrine and exocrine functions. FIG. 9 provides schematics showing the structure of the pancreas. The portion of the pancreas having an endocrine role are cell clusters called “pancreatic islets” (also known as islets of Langerhans). Pancreatic endocrine secretions include hormones that regulate glucose metabolism and blood glucose concentration. Four main cell types are present in the islets: alpha cells which secrete glucagon (a hormone that increases blood glucose concentration); beta cells which secrete insulin (a hormone that decreases blood glucose concentration); delta cells, which secrete somatostatin (a hormone that regulates alpha and beta cells), and gamma cells which secrete pancreatic polypeptide.

The portion of the pancreas that has an exocrine role is referred to as the exocrine component. The exocrine pancreatic secretions contain digestive enzymes that pass into the small intestine and help break down carbohydrates, proteins, and lipids. The exocrine component has ducts arranged in clusters called pancreatic acini. Pancreatic exocrine secretions are secreted into the lumen of the acinus, which accumulate and drain into the pancreatic duct and duodenum.

Pancreatic islet organoids and pancreatic organoids of the invention mimic the structure of a pancreatic islet and a pancreas, respectively. In some embodiments, the pancreatic islet organoid or pancreatic organoid of the invention contains any one or more of the following cells: an iPSC-derived beta-like cell, an iPSC-derived alpha-like cell, an iPSC derived delta-like cell, and an iPSC-derived duct-like cell. In some embodiments, the pancreatic organoid of the invention contains an iPSC-derived exocrine component. In some embodiments, the iPSC is a human iPSC (hiPSC). Human embryonic stem cells and human induced pluripotent stem cells are commercially available (e.g., from WiCell, which provides iPS(IMR-90)-1, iPS(IMR-90)-4 and iPS(Foreskin)-1). Human induced pluripotent stem cells can also be generated using methods known in the art from a variety of somatic cell types (Yu, J., K. Hu, et al. (2009). “Human induced pluripotent stem cells free of vector and transgene sequences.” Science 324(5928): 797-801).

Pancreatic islet organoids and pancreatic organoids of the invention also exhibit function(s) of a pancreatic islet and a pancreas. In certain embodiments, the pancreatic islet organoid or pancreatic organoid exhibits any one or more of the following functions: glucose-stimulated insulin secretion (GSIS), KCl-stimulated insulin secretion, GLP-1 stimulated insulin secretion, somatostatin secretion, and glucagon secretion. In some embodiments, the pancreatic islet or pancreatic organoid expresses any one or more of the transcription factors Pdx1, MafA, Pax4, Pax6, NeuroD1, Nkx6-1, Gata6, and Foxa2.

### Generation of Pancreatic and Pancreatic Islet Organoids

In some other aspects, the invention features methods of generating a pancreatic or pancreatic islet organoid. Recent studies have shown that while it was possible to generate glucose responsive insulin producing beta-like cells, efforts to generate pancreatic islets which are capable of secreting insulin, glucagon and somatostatin in response to nutrients, as well as efforts to obtain vascularization from stem cells, have not succeeded. Described herein are results demonstrating that using the self-organizing function of human Adipose-derived stem cells (hADSC), human umbilical vein endothelial cells (HUVEC), and human iPSC-derived beta-like cells, glucose responsive insulin secreting islet-like organoids capable of functional vascularization are successfully generated in vitro. Further, islet-like organoid generation methods were successfully scaled up using gellan gum based 3D culture systems. The functional heterogeneity in hiPSC-derived human islet-like organoids was also investigated using a Gaussia luciferase reporter to measure insulin secretion.

Generation of functional human organs provides new therapeutic strategies in drug-screening, disease modeling and inhibiting or preventing end point organ failure. Efficient stepwise differentiation methods from human embryonic stem cells (hESC) and human induced pluripotent stem cells (hiPSC) to insulin producing (3-like cells were demonstrated previously. D'Amour et al and Kroon E et al reported the efficient differentiation of hESCs into insulin producing cells which, after 4 to 5 months in vivo maturation, are able to secrete insulin in response to glucose (D'Amour et al., 2006, Nature biotechnology 24, 1392-1401; Kroon et al., 2008, Nature biotechnology 26, 443-452). Recently, Rezania et al. and Pagliuca et al. reported differentiation methods that induced formation of mature human beta-like cells in vitro in that they expressed terminal β-cells marker, MAFA and Nkx6-1 and exhibited partial functionality (e.g., insulin secretion) (Rezania et al., 2014, Nature Biotechnology Nov; 32(11):1121-33; Pagliuca et al., 2014, Cell 159, 428-439). However, in contrast to cadaveric human islets, those beta-like cells required in vivo functional maturation for a few months, and lacked the functionality provided by the other pancreatic islet cell types, such as glycemic control by α-cells (glucagon secrete) and δ-cells (somatostatin secretion). Further, the beta-like cells lacked both a mesenchyme and vascularized endothelial cells, which human islets naturally have. These crucial differences between hPSCs derived beta-like cells and human islets may compromise the ability of hPSCs based therapies to treat insulin dependent diabetes (such as type 1 or late stage type 2 diabetes).

Previously, it was identified that a metabolic transition occurs during the neonatal to adult maturation of β-cells in which the orphan nuclear receptor Estrogen-related receptor γ (ERRγ) regulates an increase in oxidative metabolism required for fully functional β cells. Consistent with this result, human iPSC-derived β like cells expressing insulin, MAFA, and Nkx6-1 can be metabolically matured through the overexpression of ERRγ to increase their oxidative metabolism and thereby enhance their glucose stimulated insulin secretion (GSIS) functionality. These results indicated that in addition to the expression of lineage determination factors such as PDX1, MAFA, Nkx6-1, and insulin, further cellular signaling which mature the β-cells' metabolism is required to generate fully functional β-cells.

During early pancreas organogenesis, newly specified pancreatic cells originate from the foregut endodermal sheet and form a pancreatic bud, a condensed tissue mass that is soon vascularized. A similar progression has been observed in liver organogenesis as well. Such large-scale morphogenetic changes depend on the exquisite orchestration of signals between endodermal epithelial, mesenchymal, and endothelial progenitors before blood perfusion. Takebe et al, successfully generated hepatic organ buds by culturing hepatic endoderm cells with endothelial and mesenchymal linages which rapidly vascularized and functional matured in vivo (Takebe et al., 2013, Nature 499, 481-484).

Previous work did not reveal the possibility of generating in vitro other organoid tissue types, such as pancreas organoids, which were mature, functional, and vascularized. Further, previous work showed a lack of scalability because the organoids were generated using MATRIGEL® matrix, which is not efficient to use for scaled-up production.

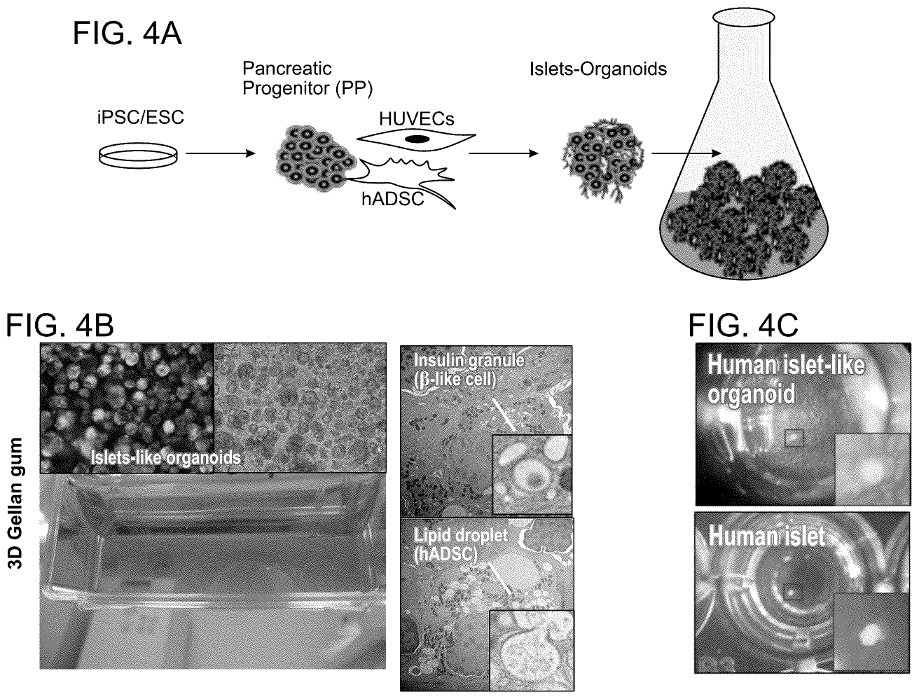

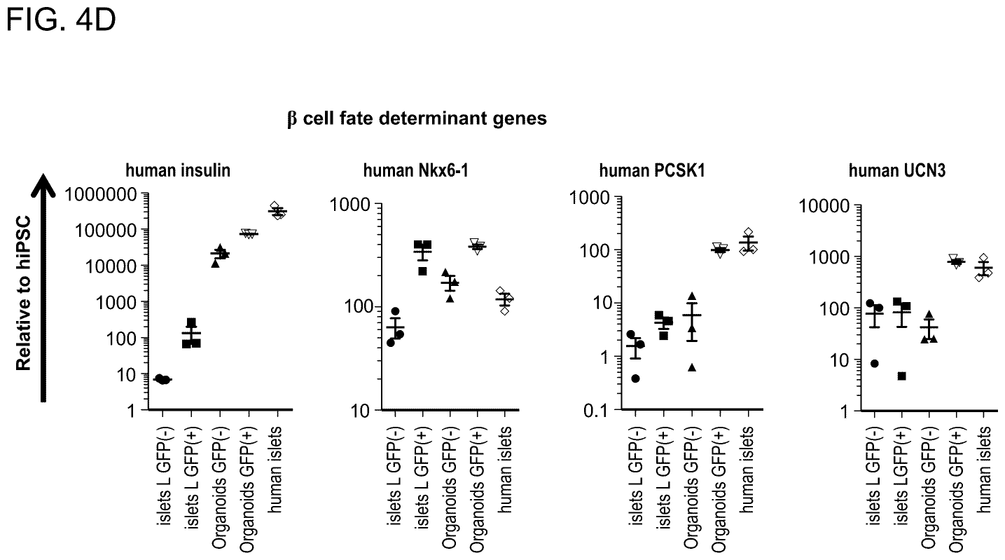

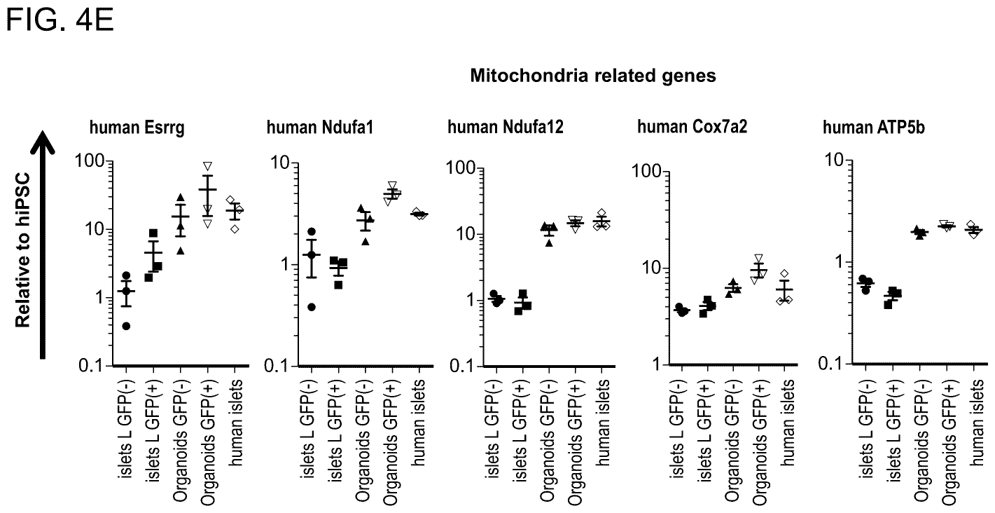

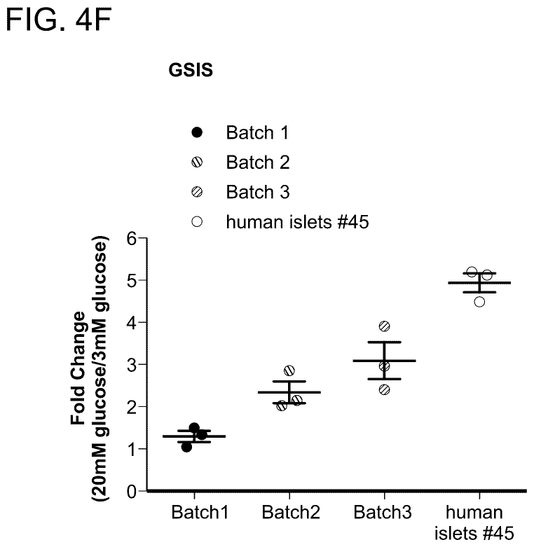

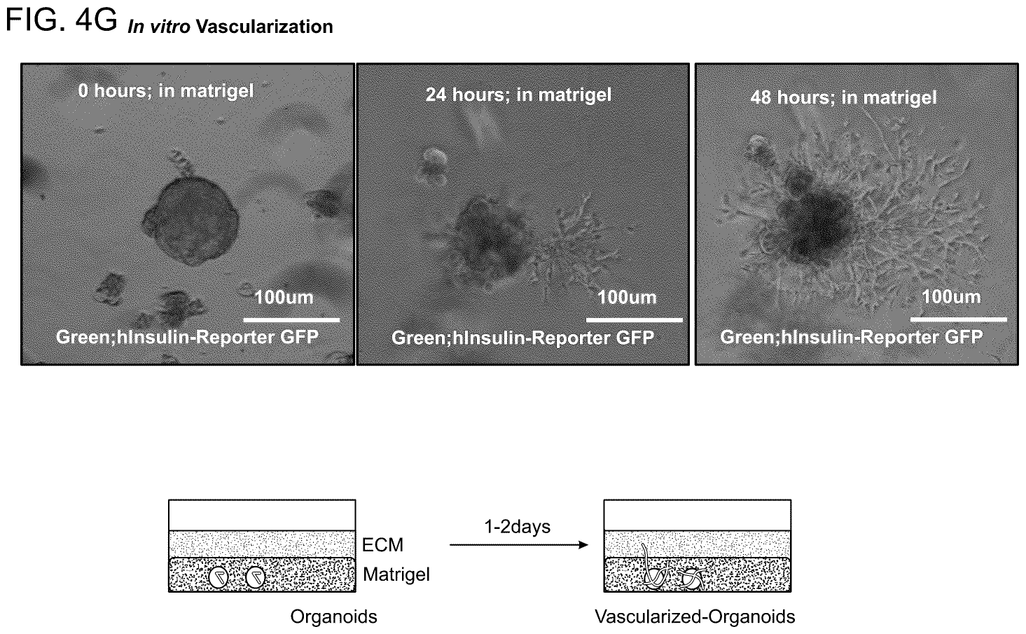

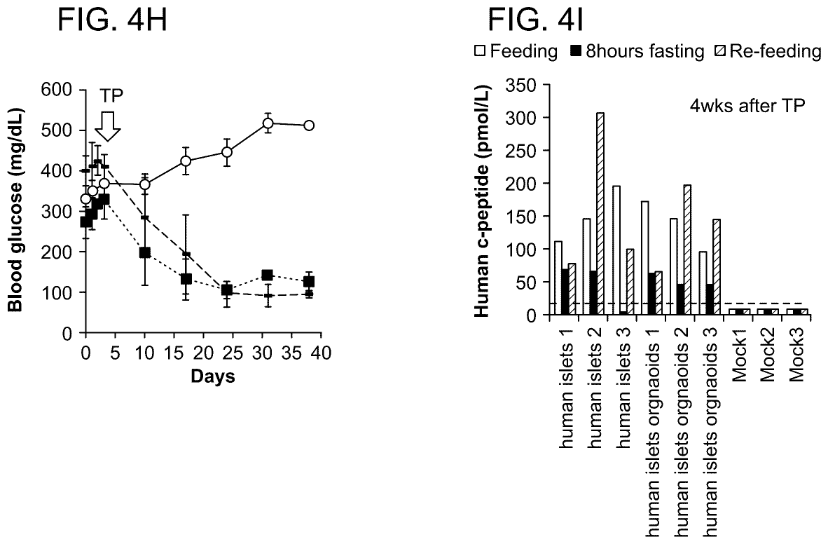

Described herein are studies demonstrating successful large-scale generation of human islet-like organoids which are capable of secreting insulin and which are vascularized, as seen in human islets. It is demonstrated herein that (1) human adipose derived stem cells (hADSCs) have a self-organizing capacity; (2) late stage pancreatic progenitors are capable of forming an islet-like cluster when co-cultured with HUVECs and hADSCs with comparable efficiency to beta-like cells; (3) human islet-like organoids had improved expression of lineage determination factors as well as metabolic regulatory genes including ERRγ; (4) islet insulin secretion assays, measured using a Gaussia Luciferase proinsulin system, revealed that human islet-like organoids contain functional cells capable of secreting insulin in response to glucose; (5) human islet-like organoids exhibited vascularization; (6) human islet-like organoids derived from hiPSC by the method described herein recaptured human islet organogenesis and pathogenesis of type 1 and type 2 diabetes in a dish; (7) human islet-like organoids derived from hiPSC by the method described herein offered a new replaceable resource for human islet transplantation to treat type 1 and type 2 diabetes; and (8) human islet-like organoids transplanted into an STZ-induced NODSCID mouse model of type 1 diabetes ameliorated type 1 diabetes in the recipient animals. (FIGS. 4H and 4I).

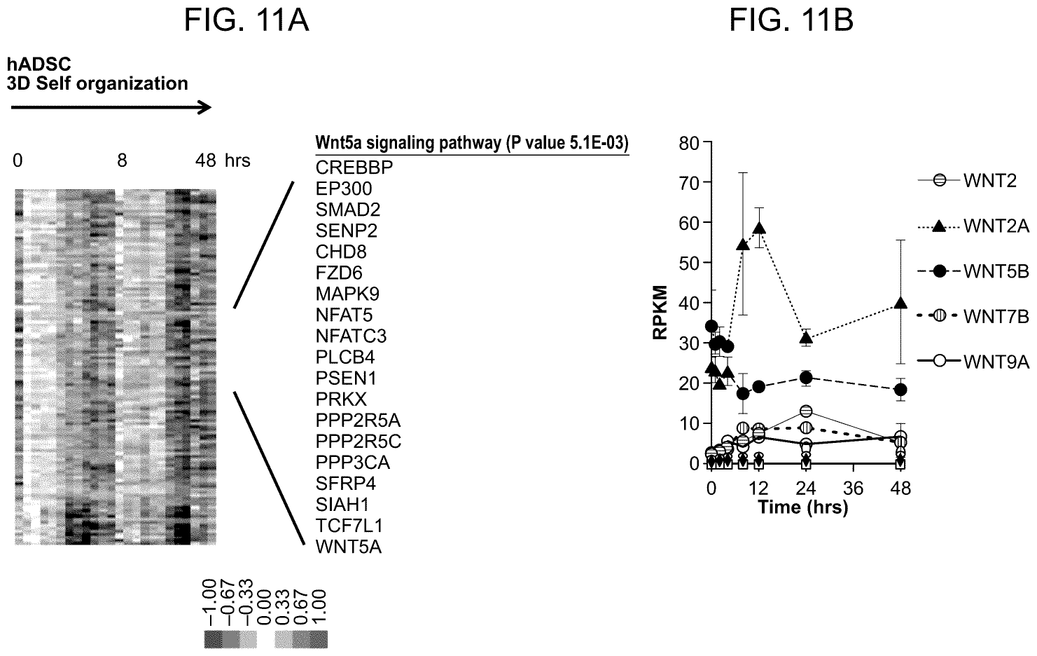

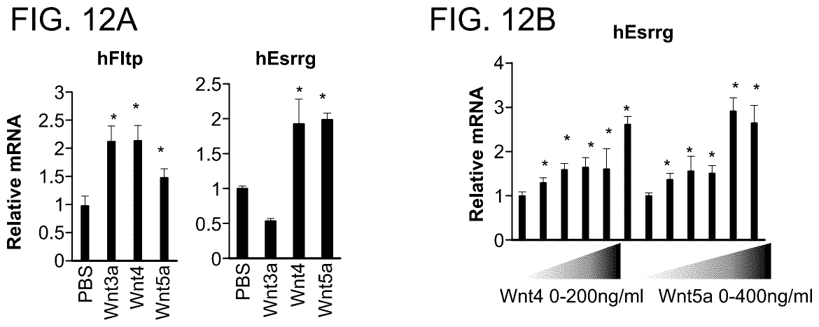

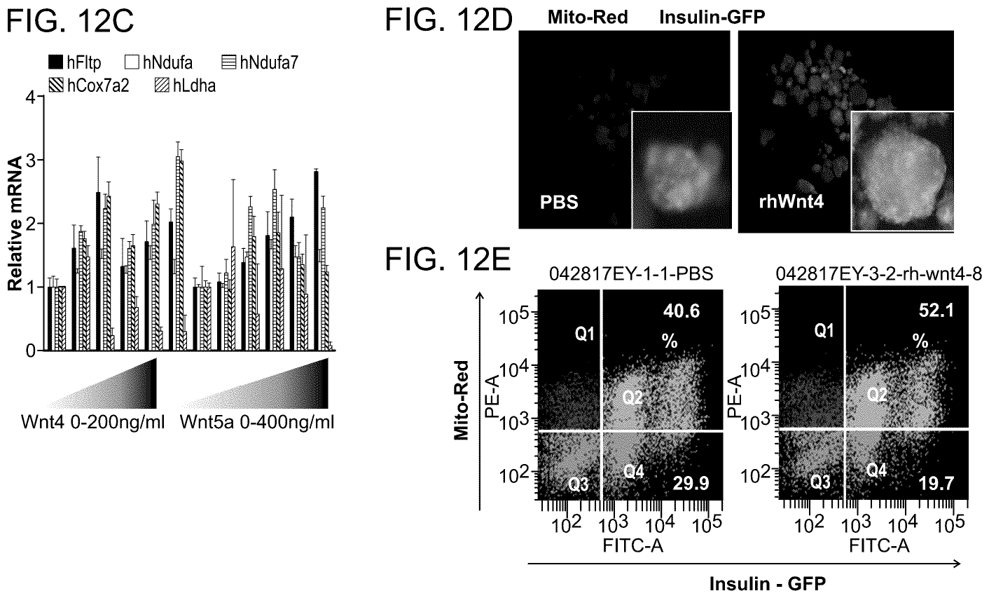

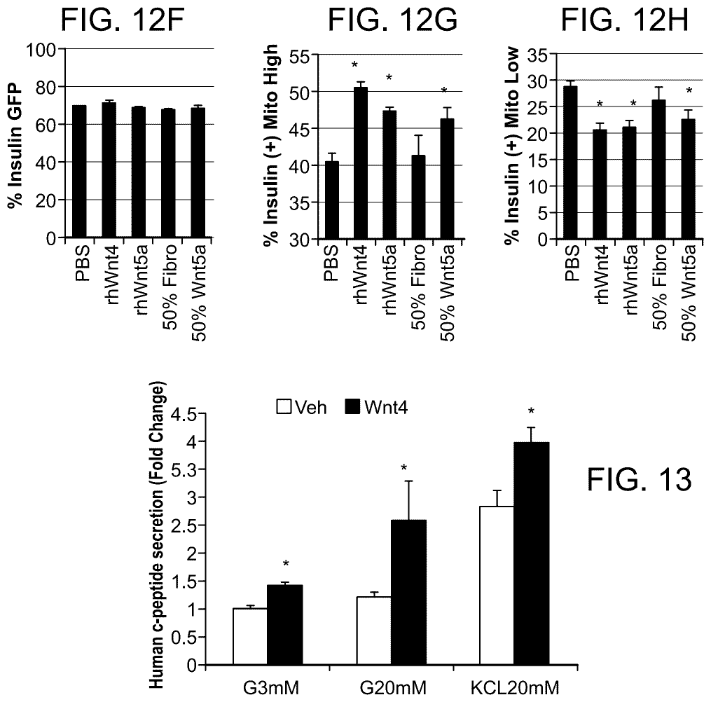

Also described herein are studies in which the role of certain Wnt (also “WNT” herein) proteins was assessed in developing human islet-like organoids which are capable of secreting insulin and which are vascularized, as seen in human islets. The WNT gene family consists of structurally related genes that encode secreted signaling proteins, which have been implicated in oncogenesis and in several developmental processes, including regulation of cell fate and patterning during embryogenesis. Wnt proteins comprise a major family of signaling molecules that orchestrate and influence a variety of cell biological and developmental processes. Wnt proteins undergo a complex set of posttranslational modifications involving several highly specialized processing enzymes. Upon release from the cell, the Wnt proteins interact with a number of molecules in the extracellular environment, such as glycans, protein-binding partners (e.g., WIF, Sfrp) and cell surface receptors. (Willert, K. et al., 2012, Cold Spring Harbor, Perspectives in Biology, 2012). It is demonstrated herein that (1) Wnt5a is the predominant Wnt protein that induces the self-organization of hADSCs (FIGS. 11A and 11B); (2) Wnt5a, as well as Wnt4, activate the ERRγ-mitochondrial metabolic pathway (FIGS. 12A-12H); (3) Wnt4 is sufficient to induce in vitro functional maturation of hiPSC-derived islet-like organoids in the absence of additional cell types such as hADSC and HUVECs (FIG. 13).

### Methods of Treatment

Islet transplantation is a therapy for treating insulin deficient diabetes such as type 1 and late stage type 2 diabetes. Thus, in another aspect, the present invention provides methods of treating a pancreatic disease such as type 1 or type 2 diabetes comprising administering a pancreatic or pancreatic islet organoid of the invention to a subject (e.g., a mammal such as a human) by transplantation. One embodiment is a method of treating a subject suffering from or susceptible to a pancreatic disease (e.g., type 1 diabetes) or disorder or symptom thereof. The method includes the step of transplanting a pancreatic or pancreatic islet organoid of the invention to the mammal sufficient to treat the disease or disorder or symptom thereof, under conditions such that the disease or disorder is treated.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration (in particular, transplantation) of an effective amount of a pancreatic or pancreatic islet organoid to a subject (e.g., animal, human) in need thereof, including a mammal, particularly a human. The administration of the pancreatic or pancreatic islet organoid may be by any suitable means that results in an amount of the organoid that, combined with other components, is effective in ameliorating, reducing, or stabilizing a pancreatic disease such as type 1 or type 2 diabetes.

In some aspects, the subject is further administered an immunosuppressant. The immunosuppressant can be administered to the subject before, during, or after the subject is administered (e.g., transplanted) with the organoid. The immunosuppressive agent can be an agent that inhibits or prevents rejection (e.g., acute rejection) of the transplanted organoid upon transplantation, or an agent that maintains immunosuppression after the transplantation. Immunosuppressants include, but are not limited to, basilizimab, antithymocyte globulin, alemtuzumab, prednisone, azathioprine, mycophenolate, cyclosporine, sirolimus, and tacrolimus.

In some embodiments, at least about 100,000, at least about 200,000, at least about 300,000, at least about 400,000, at least about 500,000, at least about 600,000, at least about 700,000, at least about 800,000, at least about 900,000 or at least about 1 million pancreatic islet organoids are transplanted into the subject. In some embodiments, islets of the subject are removed prior to transplanting the organoids of the invention. In some other embodiments, pancreatic islet organoids are transplanted into a subject by injection into the upper abdomen of the subjects. In some embodiments, the pancreatic islet organoids are injected into the liver. The pancreatic islet organoids can be injected into the subject using a catheter. In some other embodiments, the pancreatic organoid or pancreatic islet organoid is administered to the subject by surgery. In another embodiment, pancreatic islet organoids are transplanted onto the omentum. For omentum transplantation, a layering technique can be used in which the islet organoid (or cells thereof) are combined with autologous plasma and are laparoscopically layered onto the omentum. A solution (20 ml) containing recombinant thrombin (1000 U/ml) is next layered over the islet organoid, followed by another layer of autologous plasma to produce a biodegradable biologic scaffold that can survive and function in the patient for at least a year (See, e.g., Baidal, D. et al., 2017, N Engl. J. Med., 376:19). In another embodiment, hydrogel biomaterials that mitigate an immune response by the recipient can be used for islet organoid transplantation. (See, e.g., Vegas, A. et al., 2016, Nature Biotechnology, 34:345-352).

To further reduce an immune reaction to the transplanted organoid in the subject, the organoid can be encapsulated in a hydrogel and then transplanted in the subject. Such methods of transplantation are further described in Vegas et al., Nature Medicine 2016, doi:10.1038/nm.4030; Vegas et al., Nature Biotechnology 2016, doi:10.1038/nbt.3462. In some embodiments, the hydrogel contains an alginate or alginate derivative (e.g., triazole-thiomorpholine dioxide). Various modifications of alginate hydrogels that substantially reduce inflammatory or fibrotic effects of alginate hydrogels have also been identified (Vegas et al., Nature Biotechnology 2016, doi:10.1038/nbt.3462). Thus, in some other embodiments, the hydrogel contains a chemical modification that reduces an inflammatory effect of the transplanted organoid in the subject.

### Screening Assays

Pancreatic islet organoids and pancreatic organoids of the invention can be useful for modeling diseases of the pancreas in vitro or in vivo. Such pancreas disease models can be used to identify drugs that are useful for treatment of a pancreatic disease. Thus, in some aspects, the invention provides methods for identifying modulators, i.e., candidate or test compounds or agents (e.g., proteins, peptides, peptidomimetics, peptoids, polynucleotides, small molecules or other drugs) that are useful for the treatment of a pancreatic disease, particularly type 2 diabetes and/or pancreatic cancer. In one embodiment, the agent modulates an activity of an organoid of the invention.

The test agents of the present invention can be obtained singly or using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. (1994) et al., J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al (1994) Angew. Chem. Int. Ed. Engl. 33:2061; and Gallop et al. (1994) J. Med. Chem. 37:1233.

Libraries of compounds may be presented in solution (e.g., Houghten (1992), Biotechniques 13:412-421), or on beads (Lam (1991), Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

Chemical compounds to be used as test agents (i.e., potential inhibitor, antagonist, agonist) can be obtained from commercial sources or can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock (1989) Comprehensive Organic Transformations, VCH Publishers; T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Combinations of substituents and variables in compounds envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., transport, storage, assaying, therapeutic administration to a subject).

The compounds described herein can contain one or more asymmetric centers and thus occur as racemates and racemic mixtures, single enantiomers, individual diastereomers and diastereomeric mixtures. All such isomeric forms of these compounds are expressly included in the present invention. The compounds described herein can also be represented in multiple tautomeric forms, all of which are included herein. The compounds can also occur in cis-or trans-or E-or Z-double bond isomeric forms. All such isomeric forms of such compounds are expressly included in the present invention.

Test agents of the invention can also be peptides (e.g., growth factors, cytokines, receptor ligands) or polynucleotides encoding such peptides.

Screening methods of the invention identify agents that increase or decrease a biological activity of pancreatic islet organoids and pancreatic organoids of the invention. In some embodiments, a pancreatic disease, such as type 2 diabetes or pancreatic cancer, is induced or mimicked in the pancreatic islet organoid or pancreatic organoid. Type 2 diabetes in the pancreatic islet or pancreatic organoid can be induced, for example, by contacting the organoid with free fatty acids (FFAs), glucose, and cytokines (in particular, high levels of glucose and/or high levels of FFAs). In one embodiment, a pancreatic organoid is co-cultured with pancreatic cancer cells, stellate cells and immune cells to create a human pancreatic cancer microenvironment in vitro.

In some embodiments, the organoid is contacted with a candidate agent, and an effect of the candidate agent on a biological activity, function, or event is assayed. In some embodiments, the candidate agent is a drug approved by the Food and Drug Administration (FDA). For example, biological activities of a pancreatic islet organoid or pancreatic organoid assayed in the screening methods of the invention include insulin secretion (e.g., glucose-stimulated insulin secretion (GSIS)), beta cell apoptosis, LDHA activity, K(ATP) channel activity, mitochondrial function, level or activity of NDUFA4, ESRRG, KCNK3, or MAFA polypeptide or polynucleotide, cell death, cell growth, and metastasis. In some embodiments, the agent increases GSIS.

In some other embodiments, an organoid of the invention (e.g., pancreatic islet organoid or pancreatic organoid) is transplanted into a host to model pancreatic disease, such as type 2 diabetes or pancreatic cancer, in vivo. Methods of transplanting an organ or organoid are known in the art. The host can be any non-human mammal, such as a rat or mouse.

To reduce an immune reaction to the transplanted organoid in the host after, the organoid can be encapsulated in a hydrogel and then transplanted in the host. Such methods of transplantation are further described in Vegas et al., Nature Medicine 2016, doi:10.1038/nm.4030; Vegas et al., Nature Biotechnology 2016, doi:10.1038/nbt.3462. In some embodiments, the hydrogel contains an alginate or alginate derivative (e.g., triazole-thiomorpholine dioxide). Various modifications of alginate hydrogels that substantially reduce inflammatory or fibrotic effects of alginate hydrogels have also been identified (Vegas et al., Nature Biotechnology 2016, doi:10.1038/nbt.3462). Thus, in some other embodiments, the hydrogel contains a chemical modification that reduces an inflammatory effect of the transplanted organoid in the host.

In some embodiments, a pancreatic organoid and liver organoid are co-transplanted in the host. The liver is a major target organ for metastasis of pancreatic cancer. In mice in vivo endothelial cells in the mini pancreas and in the mini liver are connected to each other and create a pancreas-liver vasculature network for pancreatic cancer metastasis. Therefore, a host co-transplanted with a pancreatic organoid and liver organoid can be useful for studies of human pancreatic cancer metastasis into human liver.

In some embodiments, the host transplanted with an organoid of the invention is administered an environmental stress (e.g., administered a high fat/high glucose diet or administered pancreatic cancer cells) to induce or mimic a pancreatic disease in the host. In some other embodiments, the host is transplanted with a pancreatic islet or pancreatic organoid and/or a liver organoid where a disease (e.g., type 2 diabetes or pancreatic cancer) has been induced.

In some embodiments, the host is administered with a candidate agent. In certain embodiments, the candidate agent is a drug approved by the Food and Drug Administration (FDA). In some embodiments, an effect of the candidate agent on a host phenotype (such as biological activity or function associated with the pancreas, or activities associated with a disease) is assayed. Exemplary biological activities include insulin secretion (e.g., glucose-stimulated insulin secretion (GSIS)), beta cell apoptosis, LDHA activity, K(ATP) channel activity, mitochondrial function, level or activity of NDUFA4, ESRRG, or MAFA polypeptide or polynucleotide, cell death, cell growth, and metastasis. In some embodiments, the agent increases GSIS.

In any one of the embodiments herein, the effect of the candidate agent (i.e., ability to modulate a pancreatic activity or function) is measured relative to a reference. The reference can be, for example, an untreated pancreatic islet organoid or pancreatic organoid. In some embodiments, the reference is a host transplanted with an organoid of the invention, where the host is not administered with a candidate agent.

Agents useful in the methods of the invention can also be detected by identifying an increase in expression of a desirable marker (e.g., MAFA as a beta cell fate marker). The level of expression can be measured in a number of ways, including, but not limited to: measuring the mRNA encoded by the genetic markers; measuring the amount of protein encoded by the genetic markers; or measuring the activity of the protein encoded by the genetic markers.

The level of mRNA corresponding to a marker can be determined both by in situ and by in vitro formats. The isolated mRNA can be used in hybridization or amplification assays that include, but are not limited to, Southern or Northern analyses, polymerase chain reaction analyses and probe arrays. In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the markers described herein.

The level of mRNA in a sample can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Generation and Characterization of Pancreatic and Pancreatic Islet Organoids

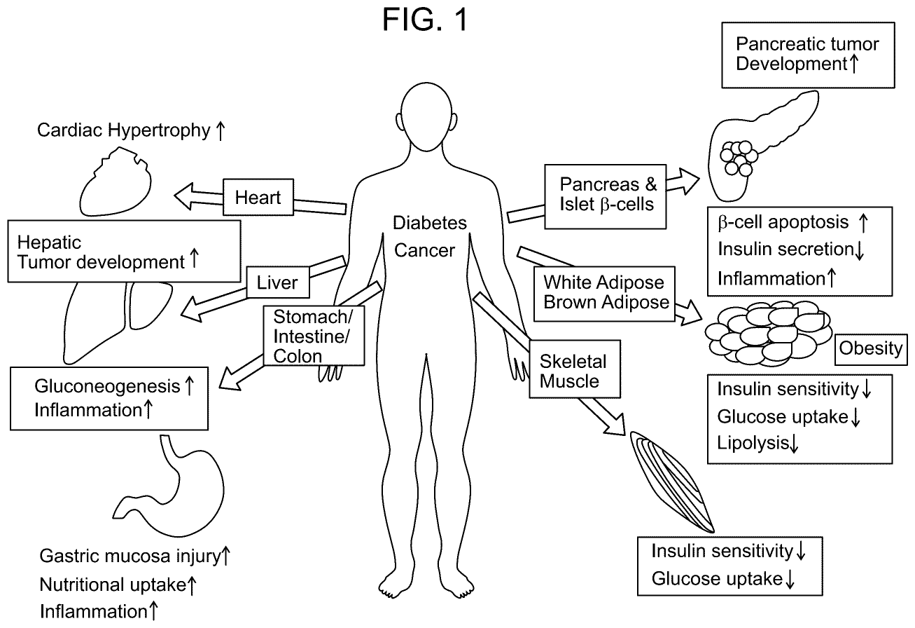

Although an animal disease model can yield insight into the pathogenesis of diseases, drugs identified from screens using animal models often fail to be adopted in human patients. Generation of functional human organoids provides a new therapeutic strategy in drug-screening and disease modeling (FIG. 1). Described herein is a novel technique to generate 3D human “mini-organs” or organoids in a dish. Using this technique, diseases such as human type 2 diabetes can be modeled in a dish to find effective drugs in genetic, patient or environmental specific diseases such as human type 2 diabetes.

### Developing Gellan Gum Based 3D Culture System for β-Like Cells Differentiation

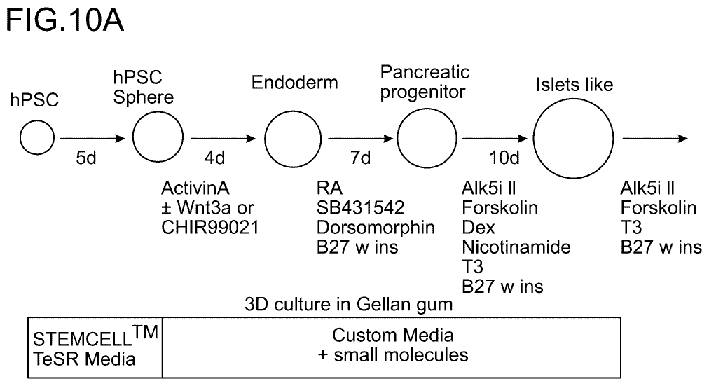

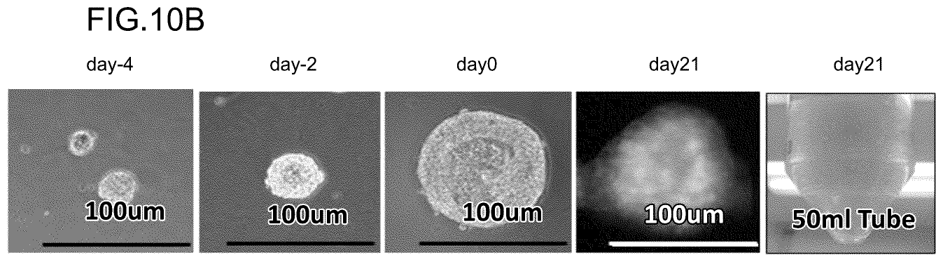

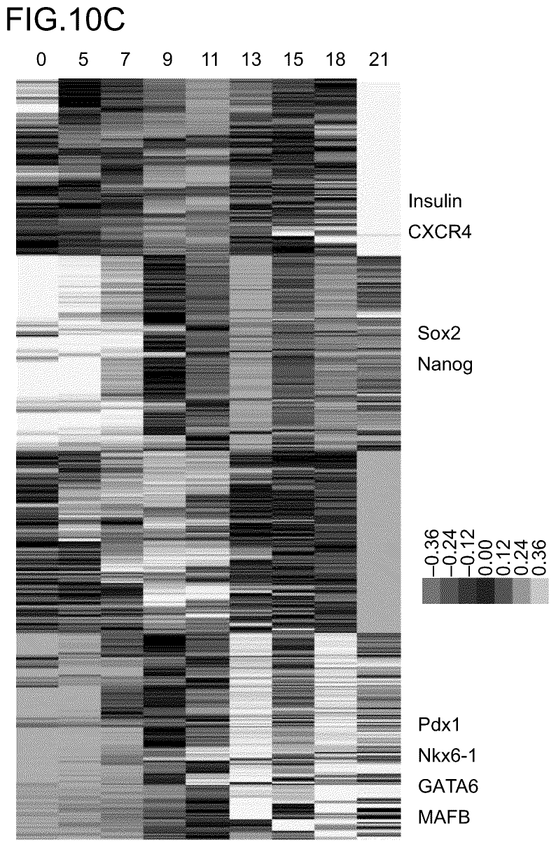

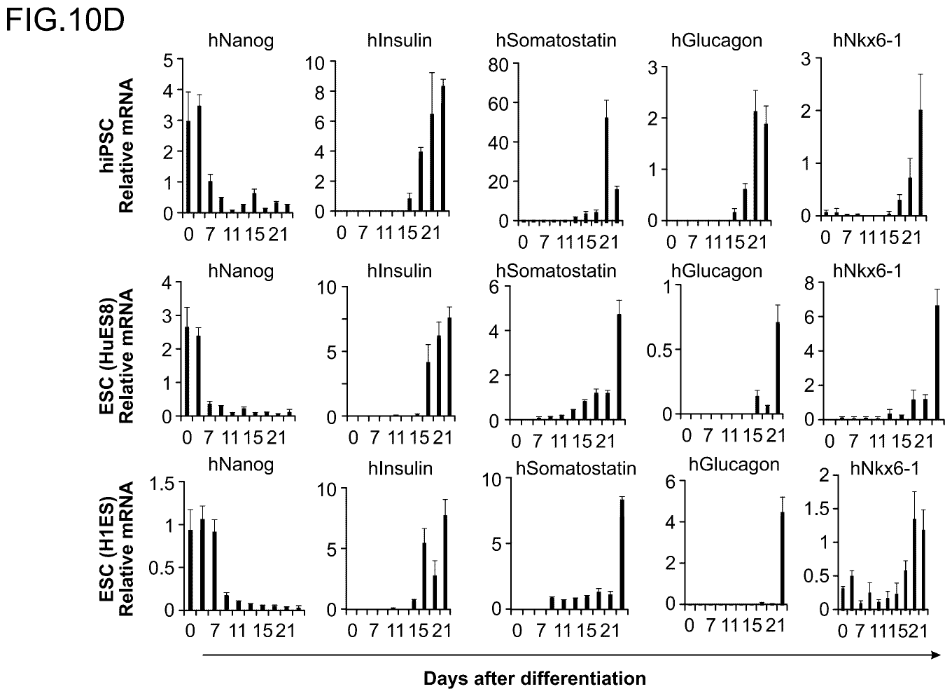

It is known that 3 dimensional (3D) culture systems contribute to facilitating self-organization and integration of cells. Therefore, MATRIGEL® matrix containing extracellular matrix components such as collagen and fibronectin is often used as the basement of a 3D culture system. However, MATRIGEL® matrix-based 3D culture systems are not ideal for large-scale human organoid generation because of their cost and difficulties in scale up. Described herein are Gellan-gum based 3D culture systems and methods for β-like cell differentiation, which are cost effective and easily scalable. Using a fully chemically-defined stepwise differentiation protocol (FIG. 10A) human pluripotent cells (hPSCs) are differentiated into insulin producing islet-like spherical cell clusters with high efficiency and reproducibility in Gellan-gum based 3D culture systems (FIG. 10B). Single dissociated pluripotent stem cells (PSCs) successfully formed into spheres within 5 days in Gellan gum containing STEMCELL™ TeSR™ media. Fifteen (15) to 21 days after differentiation in Gellan gum-containing Custom TeSR™ with defined small molecule stimulation, insulin positive GFP clusters were observed (FIG. 10B). Global transcriptome analysis by RNA-seq revealed the stepwise differentiation of hiPSCs into insulin positive cells expressing β cell lineage specific marker genes including Pdx1, Nkx6-1, GATA6 and MAFB (FIG. 10C). The differentiation of hiPSCs, as well as the human ESC lines HuES8 and H1ES, into islet-like cell clusters was further confirmed by the progressive loss of the pluripotent marker Nanog, the induction of the β cell specific marker Nkx6-1, and the progressive induction of the endocrine hormones insulin, somatostatin and glucagon, as determined by qPCR (FIG. 10D). These results demonstrate that the Gellan-gum based 3D culture systems is suitable for the generation of large-scale islet-like organoids from hPSCs.

### Generation of Scalable, Human Islet-Like Organoids In Vitro

β-like cells derived from human embryonic stem cells (hESC) or human induced pluripotent stem cells (hiPSC) have limited functionality and lack the morphological and functional feature of human islets. Previous studies revealed that co-culturing hiPSC derived hepatocyte with human umbilical vein endothelial cells (HUVECs) and human bone marrow-derived mesenchymal stem cells (hMSC) generates self-organized 3D liver-bud spheres in matrigel (Takebe et al., 2013, Nature 499, 481-484). This study found that the liver “organoids” had superior expression of lineage determinant factors compared to the differentiation of isolated hepatocytes and that these organoids rapidly vascularized and functionally matured in vivo.

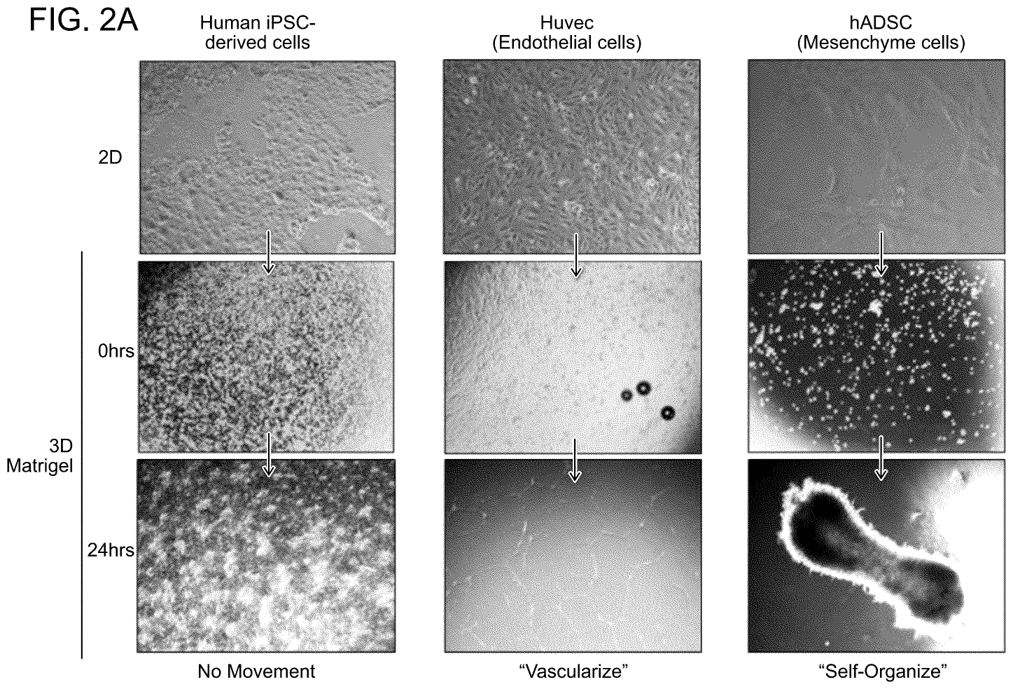

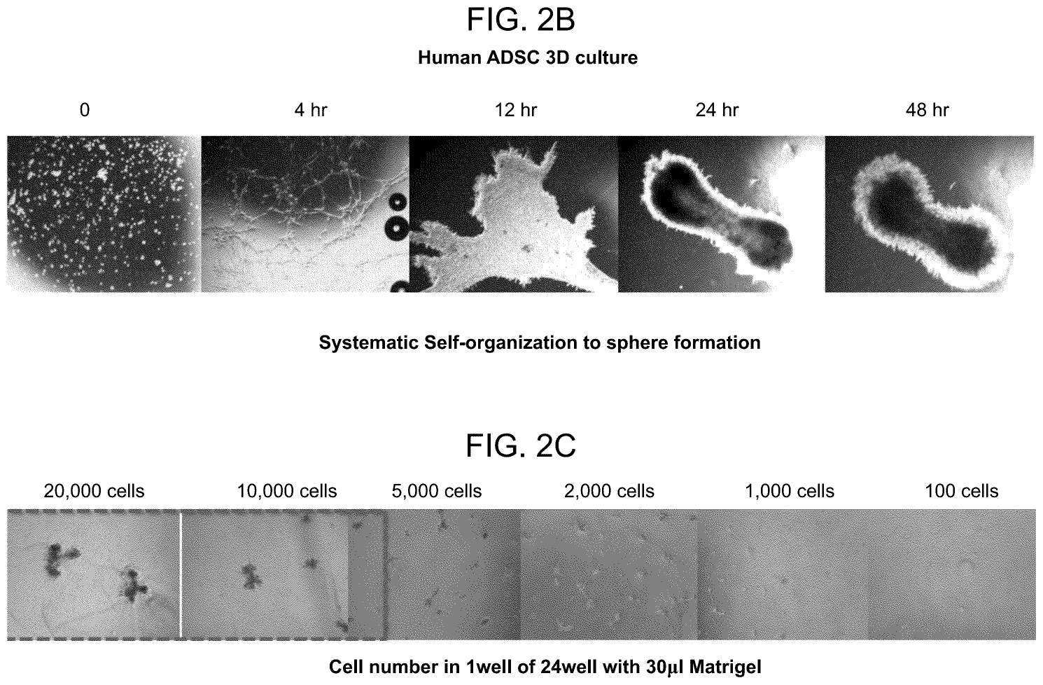

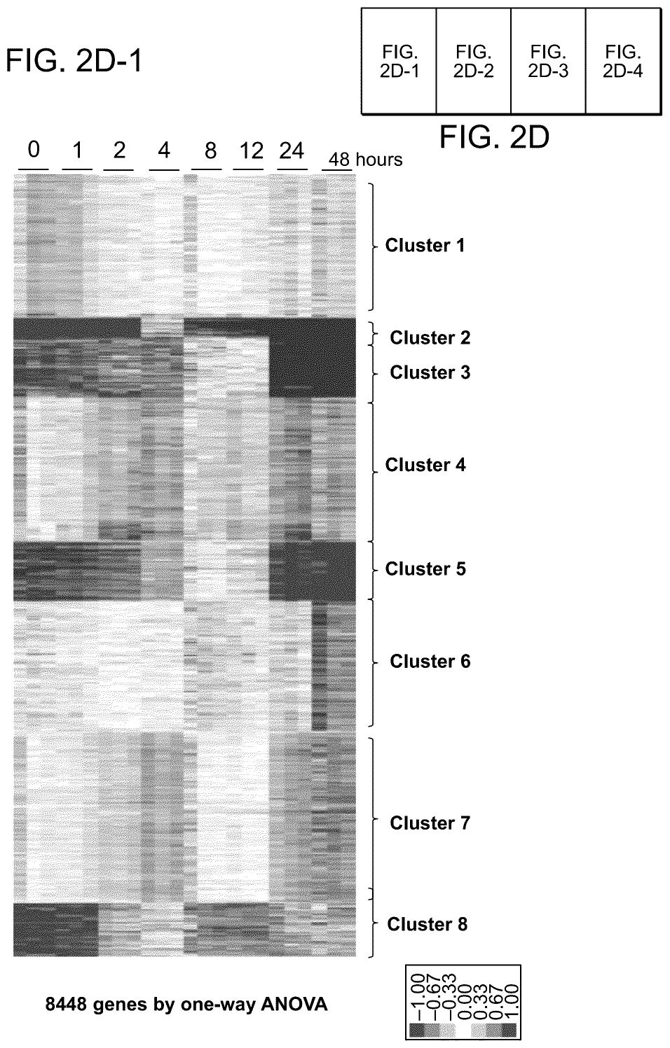

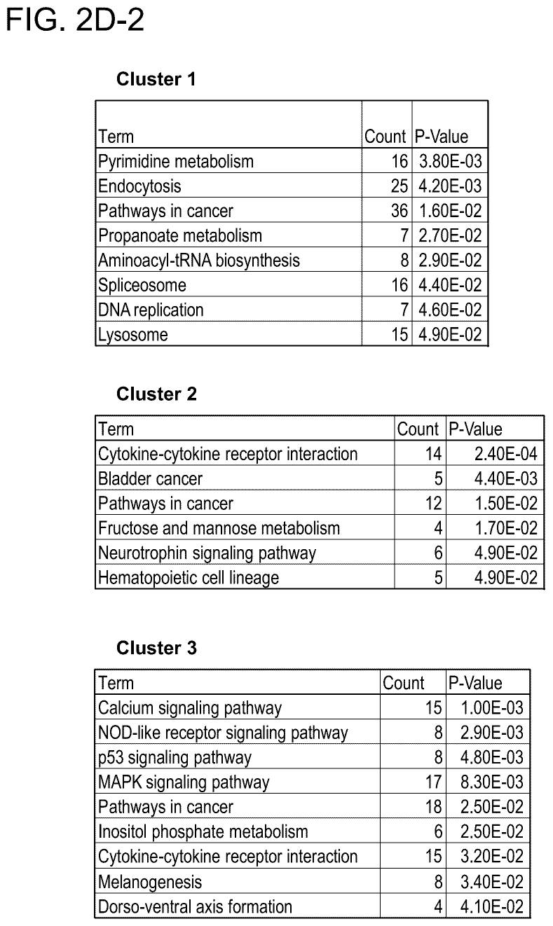

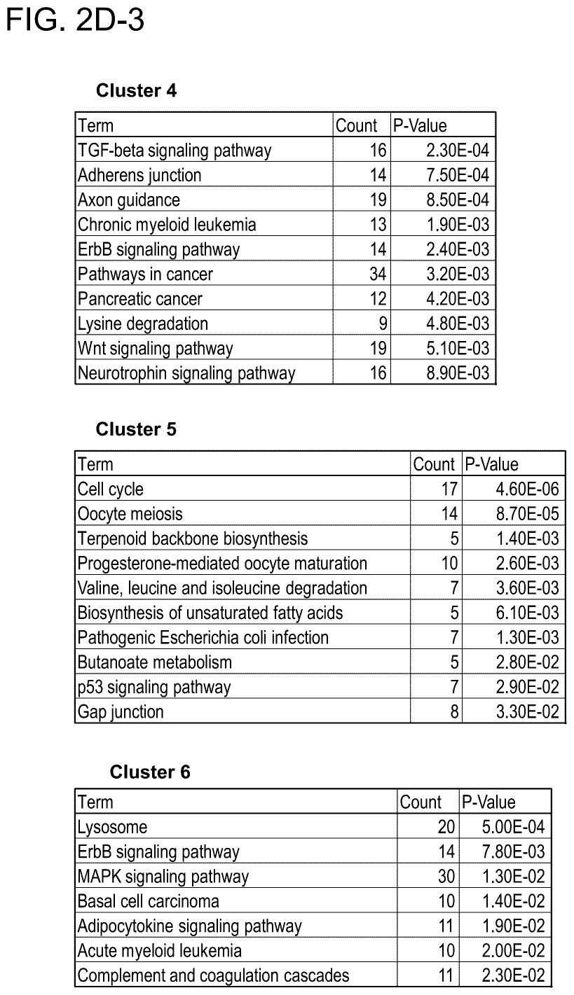

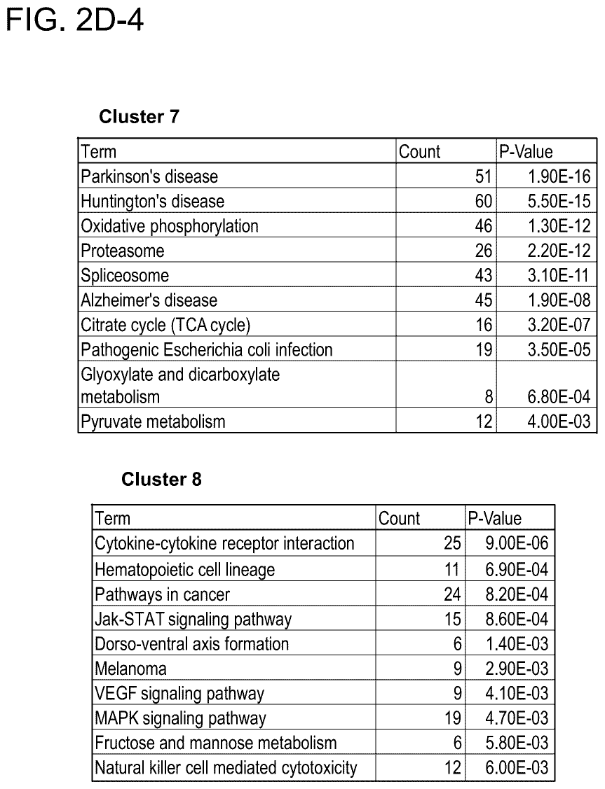

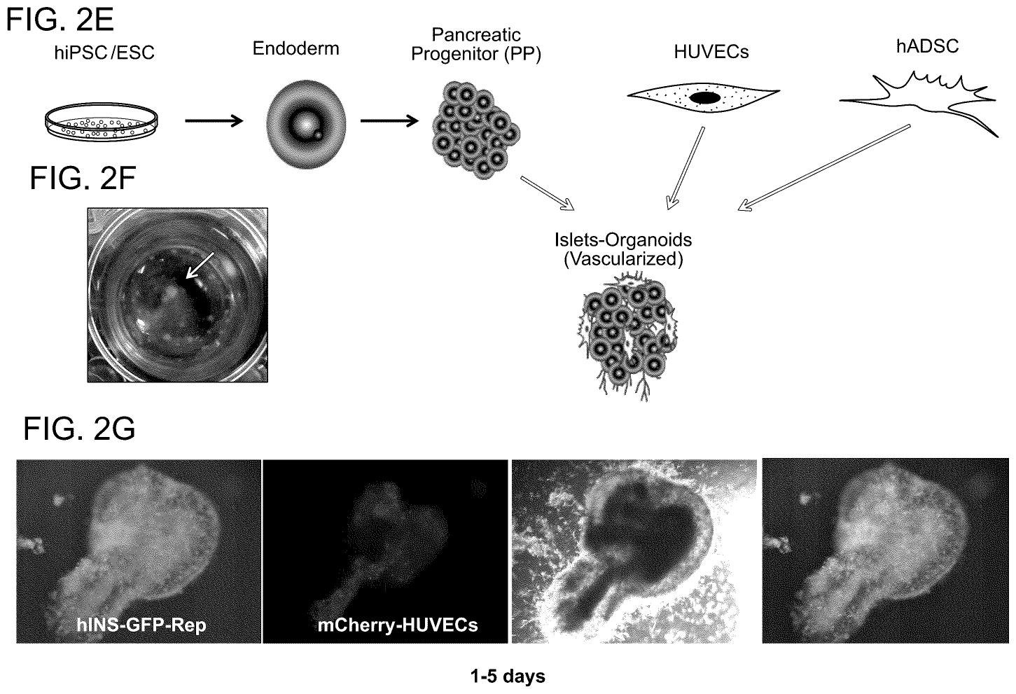

Studies herein found that hiPSC-derived pancreatic progenitor cells (hiPSC-PP) generated using a 2D differentiation protocol (Yoshihara et al, Cell Metab. 23, 622-634) did not self-organize in 3D MATRIGEL® matrix (FIG. 2A). In contrast, HUVEC cells rapidly formed a vasculature-like structure while human adipocyte-derived stem cells (hADSCs) self-organized in 3D MATRIGEL® matrix (FIG. 2A). In MATRIGEL® matrix, dispersed hADSC cells projected processes within 4 hours, formed a cloth-like wrapper within 12 hours, and adopted a sphere-like formation within 24 to 48 hours (FIG. 2B). Furthermore, a minimum cell density for self-organization was identified (i.e., ˜10,000-20,000 cells in 300 μl of MATRIGEL® matrix in ˜2 cm² well (FIG. 2C). RNA-seq analysis identified dynamic transcriptional changes during hADSC 3D self-organization, suggesting that the ability to self-organize under 3D culture conditions is an inherent feature of naïve hADSCs (FIG. 2D). These results identify the mesenchymal hADSC as a resource for generating self-organizing organoids.

To explore pancreatic organogenesis, hiPSC-PP (1×10⁶ cells) cells were co-cultured with HUVECs (7×10⁵ cells) and hADSCs (1-2×10⁵ cells) (FIG. 2E) in Matrigel matrix. This co-culture yielded macroscopically visible 3D cell clusters 48 hours after seeding (FIG. 2F). Furthermore, insulin expression, based on the expression of a GFP reporter, was detected 5 days after seeding and increased with time in culture in the human islet-like organoids. In addition, HUVECs-based endothelial cells are integrated inside the organoids as shown by fluorescence-labeled (mCherry) HUVECs (FIG. 2G).

The limitations of MATRIGEL® matrix for organoid production include high cost, difficult organoid recovery, scaling restrictions, and batch to batch variabilities.

Described herein are methods to generate morphologically identical human islet-like organoids using gellan gum based 3D cultures (FIGS. 4A-4G). FIG. 4A shows a scheme for generation of functional, vascularized human pancreatic islets in as dish. Human induced pluripotent stem cells derived—pancreatic progenitors (hiPSC-PPs) (1×10⁸ cells) were cultivated with a stromal cell population such as human umbilical vein endothelial cells (HUVECs) (2-7×10⁶ cells) and human adipose-derived stem cells (hADSCs) (2-7×10⁶) in 50 ml of gellan gum based 3D culture media (FIG. 4B). FIG. 4B shows that hiPSC-PP rapidly formed isle-like sphere formation with HUVECs and hADSCs within 5 days after seeding into the gellan gum based 3D culture media. Human islets like mini-organs expressed human insulin GFP reporter in 5 days after seeding with gradually enhancing GFP intensity. Co-culturing hiPSC-PP, hADSCs, and HUVECs according to this method, generated human islet-like organoids with high reproducibility that were morphologically similar to human islets (FIG. 4C). In addition, the generated human islet-like organoids contained insulin granules in β-like cells (FIG. 4B). Genes expression analyses revealed increased expression of β cell fate determinant genes (Insulin, Nkx6-1, PCSK1 and UCN3) and mitochondrial related metabolic genes (Esrrg, Ndufal, Ndufa 12, Cox7a2. Atp5b) in the insulin expressing cell population (GFP enriched (GFP+)) in islet-like organoids compared to those prepared without hADSC and HUVEC co-culture (FIGS. 4D & 4E). Glucose-stimulated human c-peptide secretion assay revealed that islet-like organoids generated by this method are able to secrete human c-peptide in response to high (20 mM) glucose (FIG. 4F).

An in vitro functional vascularization test was then performed. FIG. 4G shows in vitro functional vascularization tests performed. Islet-like mini organs generated in gellan gum were transferred to MATRIGEL® matrix and cultured in endothelial growth media (EGM). Green fluorescence indicates expression of insulin genes. Within 24 hours to 48 hours after stimulation by EGM, the outgrowth of HUVEC cells was observed, indicating that human islet-like organoids generated by the method described herein possessed the ability to form vascular structures.

### Establishment of Single Islet Insulin Secretion Assay Using Proinsulin-NanoLuc Gaussia Luciferase Assay System

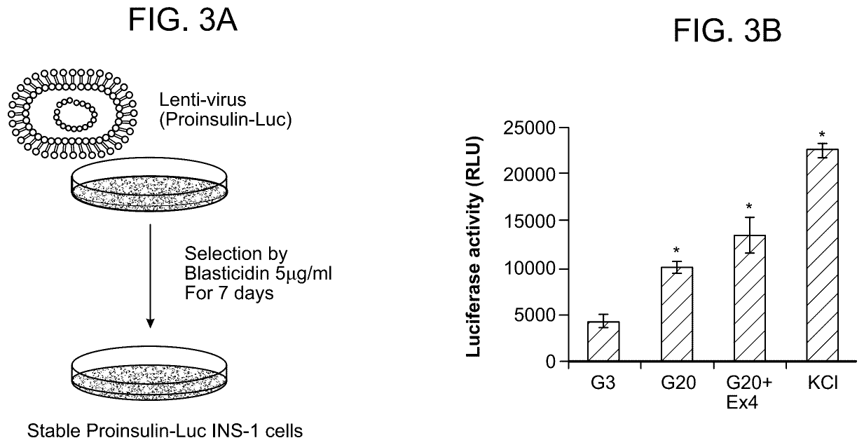

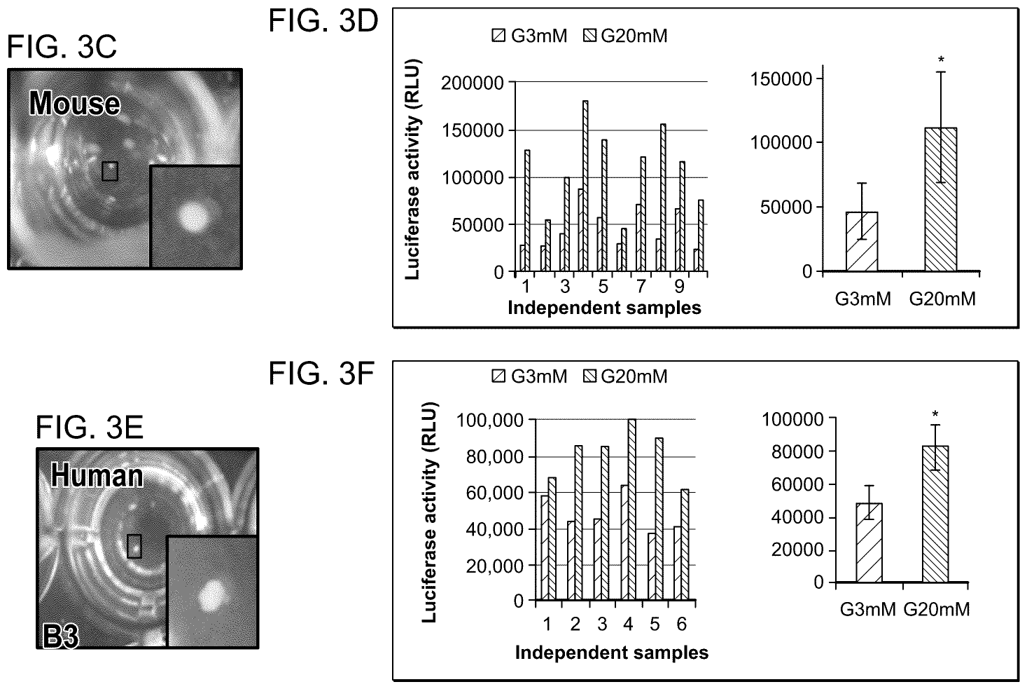

It was previously published that a reporter construct, in which the Gaussia luciferase is placed within the c-peptide portion of proinsulin accurately measures insulin secretion without affecting β-cell function (Burns et al., 2015, Cell metabolism 21, 126-137). Using a lentiviral system, INS-1 cells stably expressing this Gaussia luciferase were generated (FIGS. 3A-3F). Luciferase secretion from INS-1 cells stably expressing Proinsulin-NanoLuc increased with high-glucose (20 mM), high glucose with Exendin-4 (G20 mM+Ex4), and the depolarizing agent, potassium chloride (FIGS. 3A-3B), confirming the utility of this reporter system. Next, the usefulness of this reporter to measure insulin secretion in mouse or human islets transiently infected with the Proinsulin-NanoLuc reporter was evaluated. Luciferase secretion in response to 20 mM high glucose was detected in both transiently infected mouse and human islets were detected (FIGS. 3C-3F). Importantly, the assay sensitivity was sufficient that insulin secretion could be qualified at the level of single islets. These results indicate that the Proinsulin-NanoLuc luciferase reporter based insulin secretion assay is applicable to not only the rat beta cell line INS-1 cells, but also to primary mouse and human primary β cells.

### Establishment of hiPSC and hESC Cells Incorporating Dual Lineage and Functional Reporters

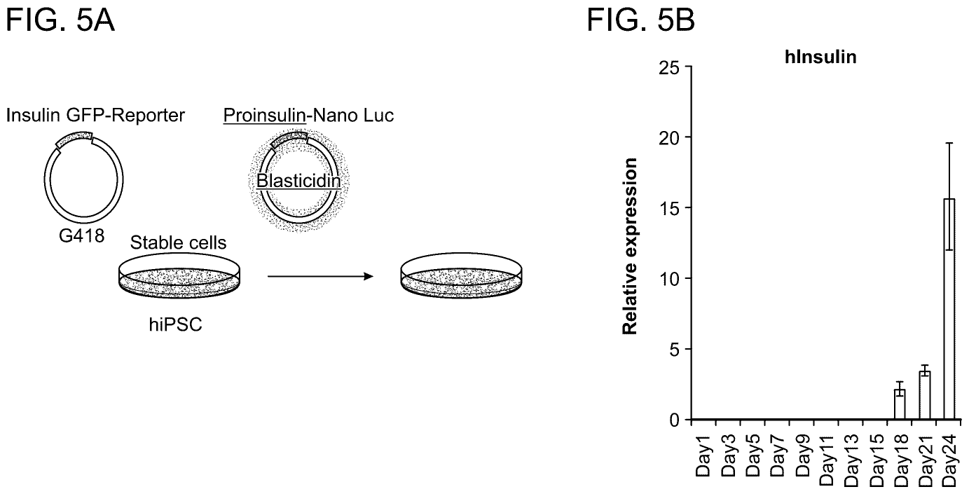

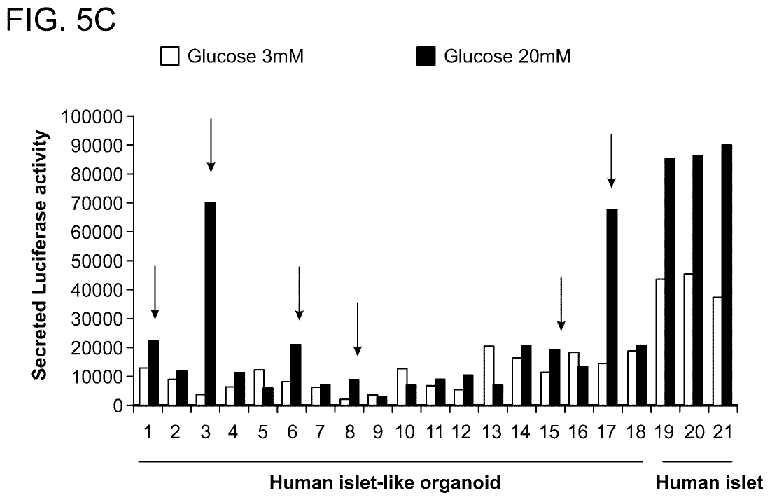

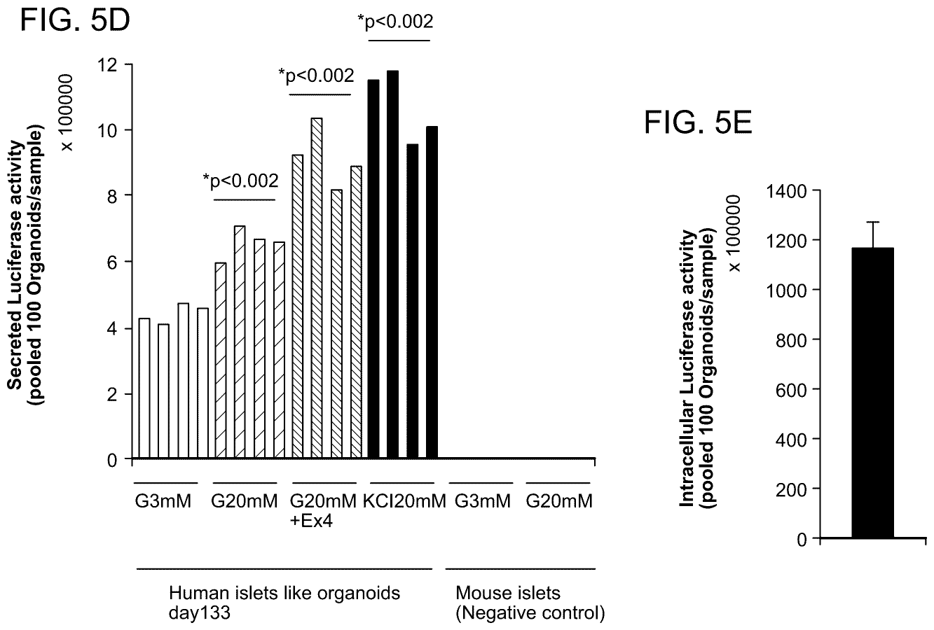

Human iPSCs and hESCs stably expressing reporters for β cell lineage (human insulin reporter) and β cell function (proinsulin-NanoLuc reporter) were generated, hiPSC^{hINS-GFP/Sec-Luc} and hESC^{hINS-GFP/Sec-Luc}, respectively (FIG. 5A). First, a neomycin resistant construct of human insulin GFP reporter was generated by inserting human insulin promoter sequence of pGreenZeo lenti-reporter (SR10028PA-1, System Bioscience) into pGreenFire Lenti-Reporter plasmid (TR019PA-1, System Bioscience) (named as hINS-GFP-EF1a-Neo). hINS-GFP-EF1a-Neo lenti virus was infected into hiPSC and hESC by spin fection (800 g, 1 hour, 37 degree) followed by a media changed to fresh STEMCELL™ TeSR™ media. Three (3) days after the first infection, the cells were treated with 100 μg/ml G418 in STEMCELL™ TeSR™ media for 7days. Selected hiPSC and hESC cells stably expressing hINS-GFP-EF1a-Neo were subsequently infected with the Proinsulin-NanoLuc (Addgene, Plasmid #62057) lenti-virus by spin fection (800 g, 1 hour, 37 degree) followed by a media change to fresh STEMCELL™ TeSR™ media. Three (3) days after the second infection, the cells were treated with 5 μg/ml blasticysin and 100 μg/ml G418 in STEMCELL™ TeSR™ media for 7 days. Subsequently, cells were maintained in STEMCELL™ TeSR™ media (FIG. 5A). The generated stable cell lines incorporating the dual reporters maintained self-renewal and pluripotency capabilities, as well as the capacity to differentiate into insulin producing β like cells (FIG. 5B).

### Pooled Human Islet-Like Organoid Cultures Display Consistent Insulin Secretion Despite Variable Functionality Seen in Individual Organoids

Recent studies have reported the generation of insulin producing β-like cells from hESC and hiPSC capable of secreting insulin in response to glucose (Pagliuca et al. 2014, Cell 159, 428-439; Rezania et al., 2014, Nature Biotechnology November; 32(11):1121-33; Russ et al., 2015, EMBO Journal 34, 1759-1772). However, fully functional human islet-like clusters able to appropriately secrete insulin in response to nutritional signals including glucose, amino acids, fatty acids and incretins such as GLP-1 have yet to be demonstrated. To date efforts have focused on the independent generation of insulin producing β-like cells, glucagon producing α-like cells, and somatostatin producing δ-like cells from hPSC. However, these approaches lack the supporting cells important for regulation, such as mesenchymal cells, adipose cells, and vasculature cells. Since the 3D structure of islets naturally enhances their function, these missing cellular components may compromise the functionality of islet-like cells clusters. In addition, organogenesis of pancreatic islets involves clonal expansion of β-cells, suggesting that these cells may have multiple functions in islet-like organoids. To test this idea, single organoid proinsulin secretion assays were performed. Human islet-like organoids generated by methods described herein are morphologically identical with human islet (FIG. 4C). However, significant variability was seen in the glucose-stimulated insulin secretion (GSIS) capabilities of individual human islet-like organoids compared to human islets, as measured by proinsulin luciferase secretion assay (FIG. 5C) Consistent GSIS functionality was demonstrated in pooled organoids (10 to 100 organoids for assay) (FIG. 5D). Furthermore, pooled human islet like organoids demonstrate enhanced GSIS when co-stimulation with GLP-1, as well as robust KCl-stimulated insulin secretion (FIG. 5D).

In vitro cultured iPSC-derived human pancreatic islet-like organoids generated herein retained their ability to respond to glucose, GLP1 and KCl after extended time (133 days) in culture (FIG. 5D).

### Example 2: Generation and Characterization of Human Organoids

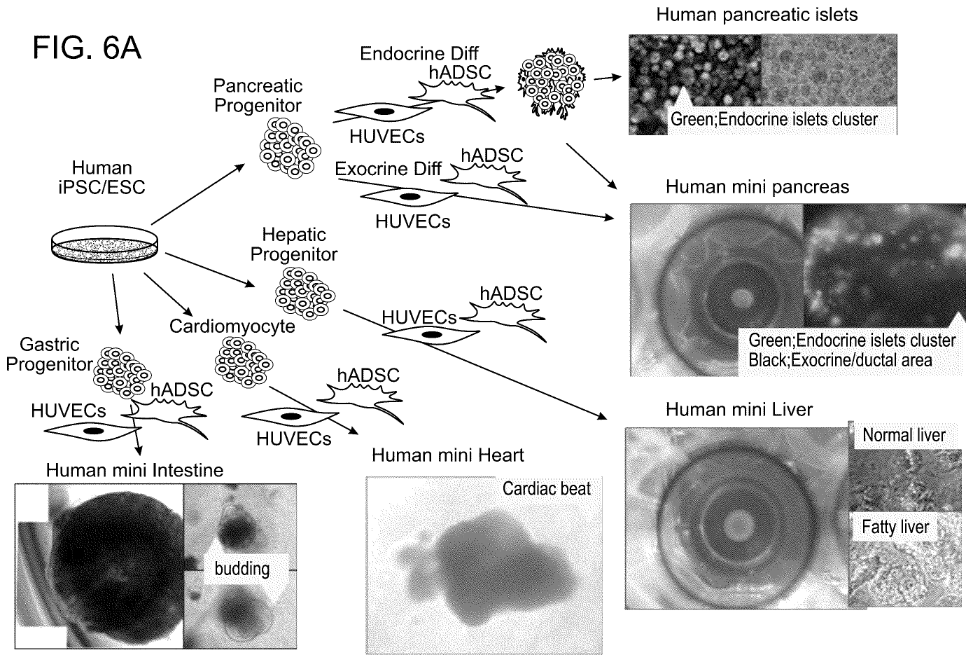

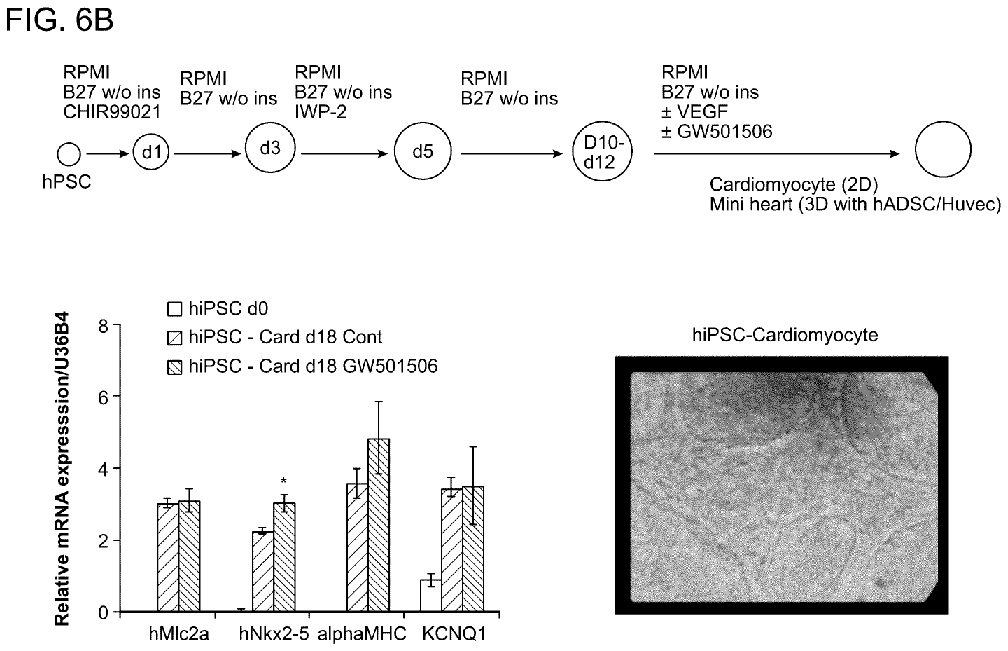

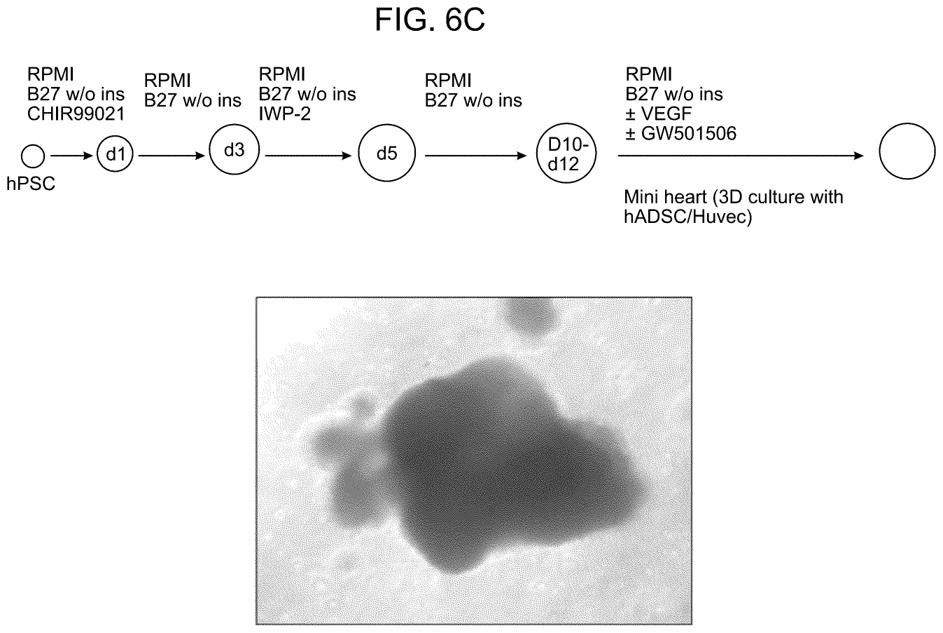

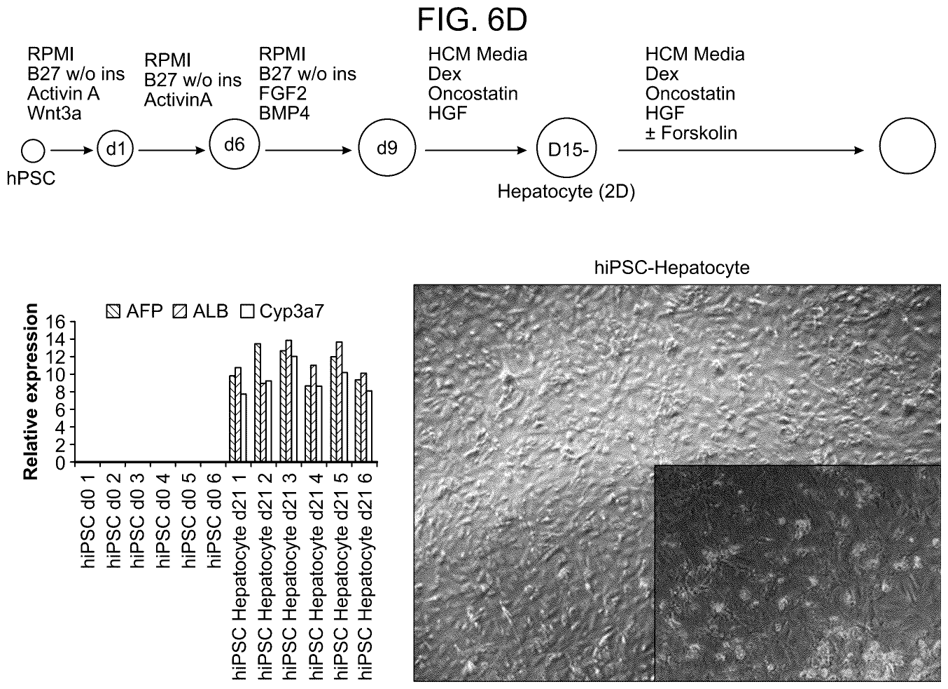

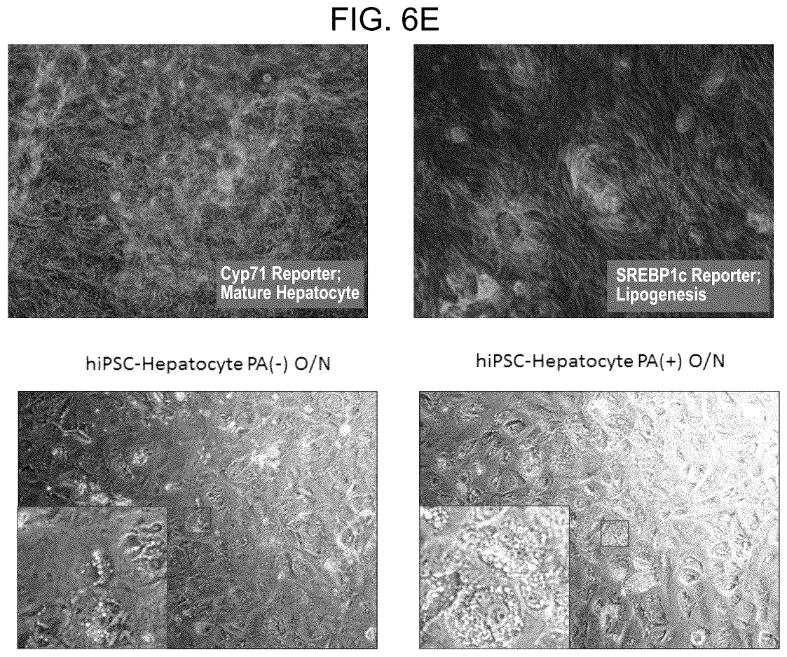

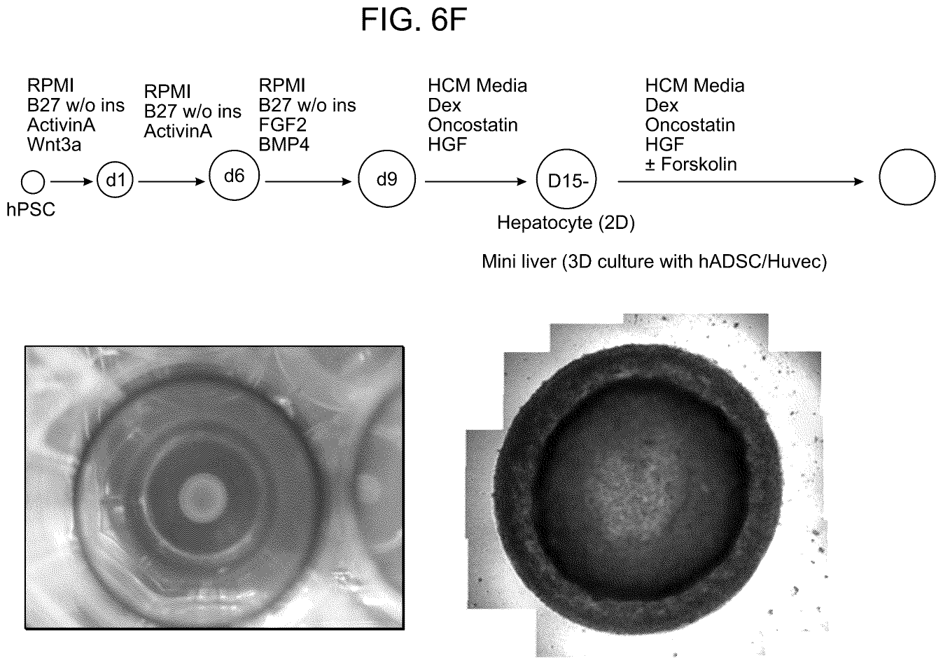

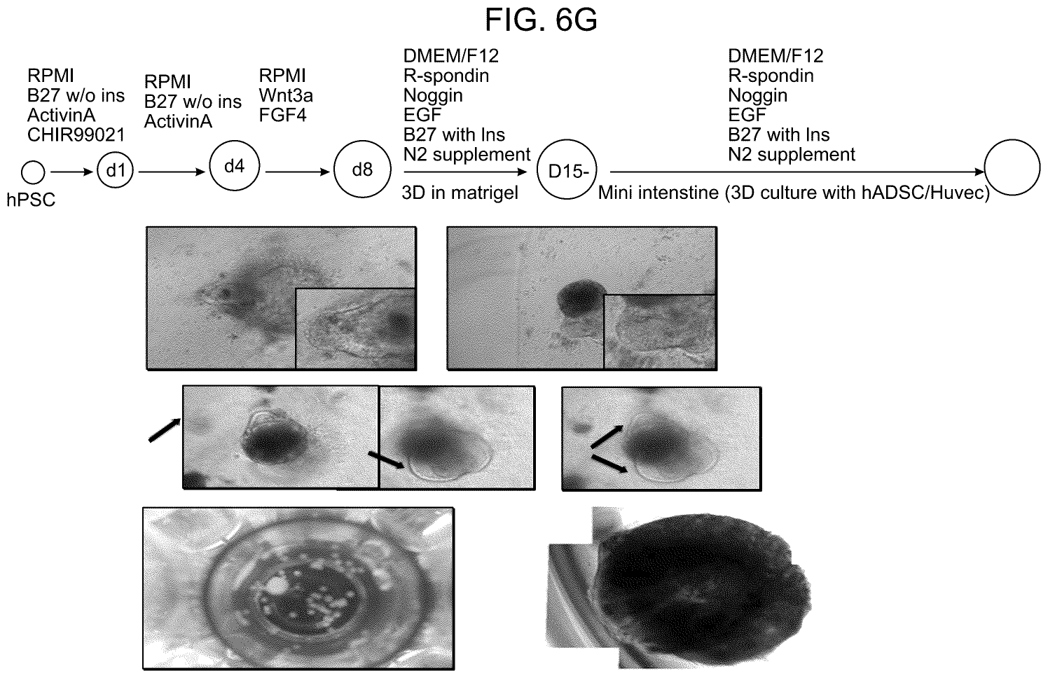

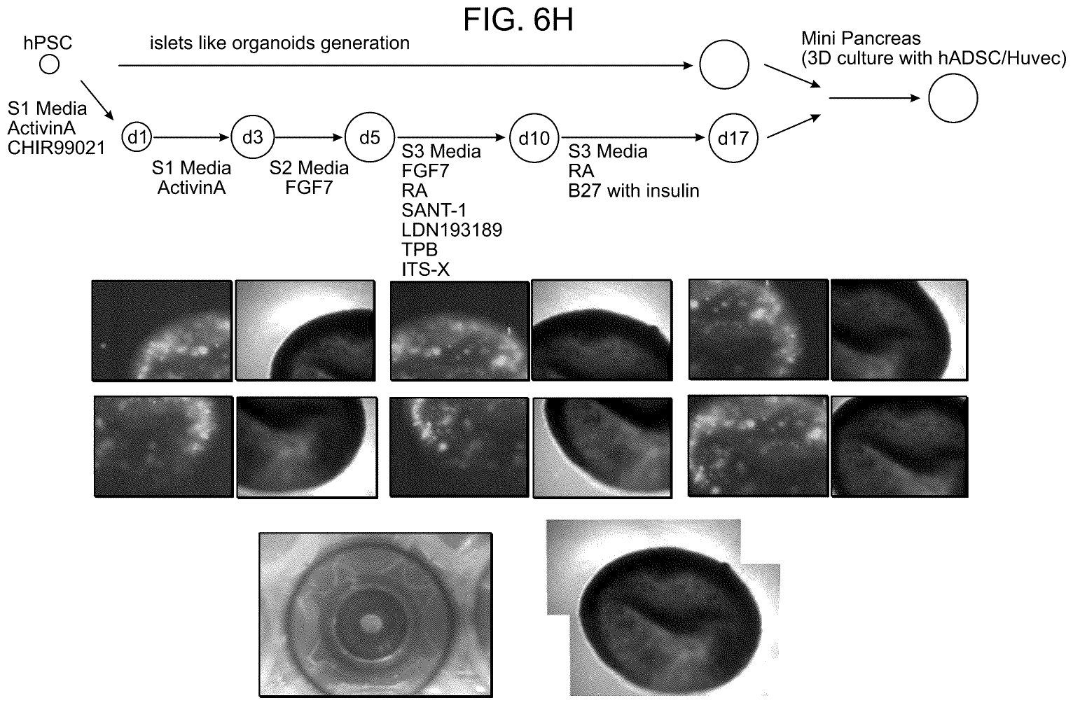

Functional human mini organs, including human islets, pancreas, liver, heart, and intestine, can be generated using the methods described herein (FIG. 6A). FIG. 9 shows a structure of a human pancreas and human pancreatic islets within the pancreas. Using the methods herein, human pancreatic islets mini organs or organoids were generated in about 30 days. The pancreatic islets generated contained human induced pluripotent stem cell (hiPSC)-derived beta cells, alpha cells, delta cells, duct cells, as well as endothelial cells and hADSCs. The pancreatic islet organoids generated express key beta cells transcription factors such as Insulin, Nkx6-1, PCSK1, and UCN3, as well as key mitochondrial metabolic genes including Esrrg, Ndufa 1, Ndufa 12, Cox7a2 and AtpSb (FIGS. 4D-4E). The pancreatic islet organoids exhibited at least partial GSIS, KCl-stimulated insulin secretion, GLP-1 stimulated insulin secretion, vascularization, somatostatin secretion, and glucagon secretion (FIGS. 4G, 5D).

A human mini pancreas or human pancreatic organoid was generated in about 30 days (FIG. 6H). The human pancreatic organoid contained hiPSC-derived islets clustered within the interior of the organoid and a hiPSC-derived exocrine component surrounding the islets (FIG. 6H). The pancreatic organoid also contained endothelial cells and hADSC. Function of the human pancreatic organoid was demonstrated using an amylase secretion test, among other tests.

A human mini liver was generated in about 15 days (FIGS. 6D-6F). The human mini liver contained hiPSC derived hepatocytes, endothelial cells, and hADSCs. Analysis of functional characteristics of the human mini liver revealed that the mini liver expressed AFP, ALB, and Cyp3a7 (FIG. 6D), as well as the mature hepatocyte marker Cyp7a1 and lipogenesis marker SREBP1c (FIG. 6E). The human mini liver also exhibited insulin signaling, insulin resistance by palmitic acids, and lipid accumulation. The human mini liver is further tested for gluconeogenesis and metabolic function for drug metabolism.

A human mini heart was generated in about 15 days (FIGS. 6B-6C). The human mini heart contained hiPSC derived cardiomyocytes, endothelial cells, and hADSCs. The human mini heart expressed key cardiomyocyte genes such as hMlc2a, hNkx2-5, alpha MHC and KCNQ1 (FIG. 6B), and was seen to beat in cultures.

A human mini intestine was generated in about 30 days (FIG. 6G). The human mini intestine contained hiPSC derived intestinal cells, endothelial cells, and hADSCs. The human mini intestine expressed small intestine markers CDX2, Muc2, and Lgr5, and exhibited budding of intestinal organoids in response to R-Spondin.

### Example 3: Transplantation of Functional Pancreatic Islet Organoids Rescued Type 1 Diabetic Mice

Expression of specific functional islets marker such as MAFA, UCN3 and mitochondrial oxidative genes such as ERRy (Esrrg), Ndufa 1, Ndufa 12, Cox7a2 and Atp5b in hiPSC-derived human islet-like organoids was observed. Notably, these islet-like organoids recapture both human islets development as well as the pathogenesis of diabetes in a dish. Transplantation of these functional islet-like organoids rescue type 1 diabetic mice with long survival, rapid vascularization, and reduced immune rejection.

### Example 4: Drug Screening and Disease Modeling in Human Islet-Like Organoids

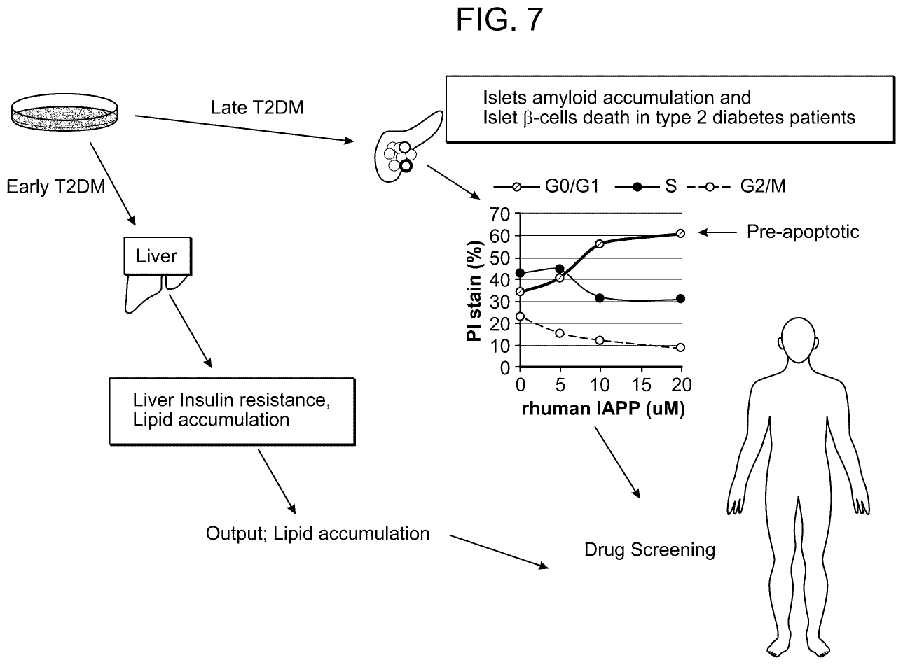

Generation of functional human organs according to methods described herein provides new strategies for drug-screening and disease modeling. Specifically, functional organoids can be used as models of type 2 diabetes for drug screening (FIG. 7). Human islet-like organoids responded to amyloid polypeptide (hIAPP) toxicity, an inducer of β cell loss in type 2 diabetic patients and islet dysfunction after transplantation in hyperglycemic patients, hIAPP dose-dependently induced G0/G1 arrest in 24 hours in human islet-like organoids (FIG. 7).

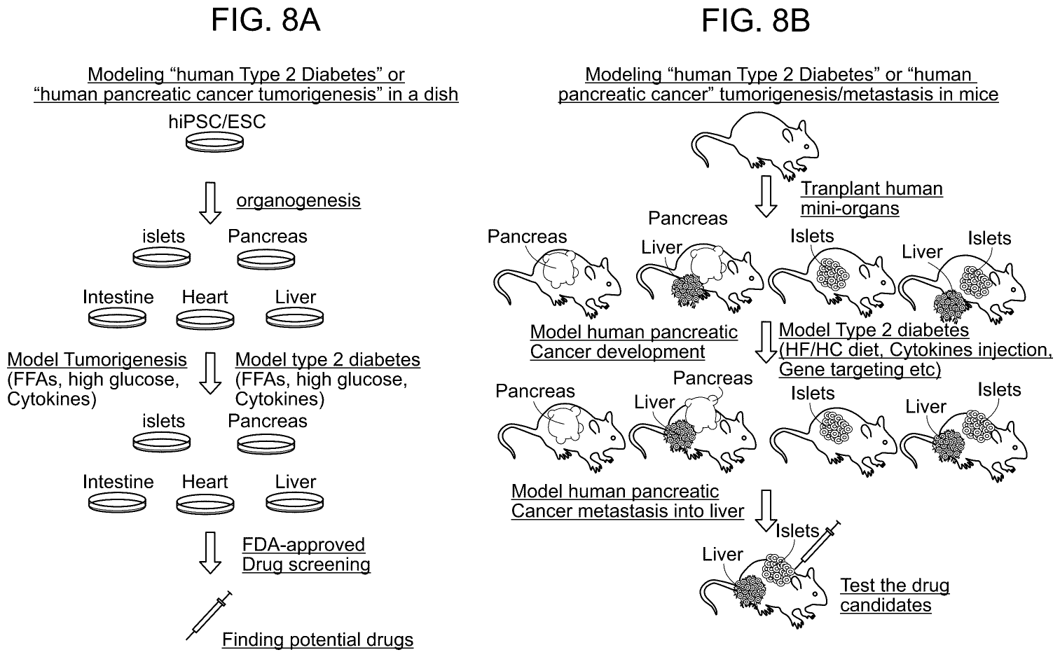

FIG. 8A provides a schematic showing experimental approaches to model type 2 diabetes and potentially screen for drugs using pancreatic islet and/or pancreatic organoids generated by the methods herein. In an exemplary assay, 3D mini organs are exposed to stressors that induce type 2 diabetes, such as high levels of free fatty acids (FFAs) and/or, glucose and selected cytokines. The stressed 3D mini organs are then treated with various drugs. In some embodiments, the drug is approved by the Food and Drug Administration (FDA).

As output, the following are assayed in human pancreatic islet organoids: insulin secretion, beta cell apoptosis (PI stain), lactate dehydrogenase A (LDHA) expression via a luciferase reporter, and changes in expression of marker genes including NDUFA4 (Mitochondrial oxidative phosphorylation), ESRRG (Mitochondrial function), KCNK3 (Katp channel activity) and MAFA (beta cell fate marker). For the human pancreas organoid, amylase secretion and apoptosis of exocrine cells (PI stain) are assayed. For the liver organoid, lipid accumulation is assayed using oil red O or histology. In the case of heart organoid, heart beat and heart size (hypertrophy) are measured. The intestine organoid is analyzed by measuring lipid accumulation using oil red O or histology.

FIG. 8A also shows modeling of human pancreatic cancer tumorigenesis and metastasis in a dish and the potential to screen for drugs that target those diseases. In an exemplary assay, a 3D mini human pancreas is co-cultured with pancreatic cancer cells, stellate cells, and immune cells to create human pancreatic cancer microenvironment in a dish. Various drugs (e.g., FDA-approved drugs) are then screened to find compounds which effectively suppress pancreatic cancer growth or metastasis in a mini human pancreas microenvironment. As output, the following are measured for the pancreas organoid: apoptosis of exocrine cells (PI stain), collagen synthesis (Trichrome stain) and stellate cells activation (GFAP-reporter). Potential candidate drugs identified in these assays are tested in pancreatic cancer tumorigenesis and metastasis mouse models. Genes expression and morphology as well as the degree of cell death, cell growth, and metastasis are investigated. FIG. 8B provides a schematic showing modeling of human Type 2 diabetes in mice.

In an exemplary assay, human islet organoids and/or human liver organoids are transplanted into mice. The mice are then administered various stressors that induce type 2 diabetes, such as a high fat diet (HFD) or cytokines injection. The potential candidate drugs identified in this assay are further tested in human type 2 diabetic mouse model. Genes expression and morphology as well as the degree of diabetes are investigated.

FIG. 8B also shows modeling of human pancreatic cancer tumorigenesis and metastasis in mice. In an exemplary assay, human pancreas organoids and/or human liver organoids are transplanted into mice. Mice transplanted with a mini pancreas are used to study human pancreatic cancer growth in human pancreas microenvironment. In another exemplary assay, a mini pancreas and mini liver are co-transplanted in mice. The liver is a major site for metastasis of pancreatic cancer. In vivo, endothelial cells in the mini pancreas and in the mini liver create a pancreas-liver vasculature network for pancreatic cancer metastasis. Thus, mice co-transplanted with a mini pancreas and mini liver are used to study the metastasis of human pancreatic cancer into the human liver.

An ultimate goal of the generation of functional organ-like clusters from pluripotent stem cells (PSC) is to gain insight into the mechanisms underlying human diseases. Although great advances have been made in terms of developing disease models in animals, many of these models fail to faithfully recapture the human condition. In the case of pancreatic islets, their development, cytoarchitecture, and physiology in rodents and human are notably different.

Results herein were obtained using the following materials and methods.

### 3D Kelcogel® (3DKG) Culture Media

Kelcogel® F low acyl gellan gum (GG-LA) obtained from Modernist Pantry is suspended in pure water 0.3% (w/v) and dissolved by stirring at 90° C. or by microwave. The aqueous solution is sterilized at 121° C. for 20 minutes in an autoclave. The solution is added to TeSR™ (Ludwid et al., Nature methods 3, 637-646) or custom TeSR™ media (800 ml DMEM/F12, 13.28 g BSA. 10 ml Glutamax, 560 mg NaHCO₃, 330 mg thiamine, 100 mg reduced glutathione, 3300 mg Vitamin C, 14 μg Selenium, 10 ml NEAA, 2 ml Trace element B, 1 ml Trace Element C, 7 μl β-ME, 2 ml DLC, 2 ml GABA, 2 ml LiCl, 129.7 μg pipecolic acid, Insulin 2 mg up to 1000 ml) at final concentration of 0.015%. Methylcellulose (MC) stock solution is added to a final concentration of 0.3% (R&D systems) (e.g., 0.3% Kelcogel® stock: Kelcogel® F low acyl GG-LA 300 mg+MilliQ water 100 ml; 3DKG Stem TeSR™ Base Media: STEMCELL™ TeSR™ 9 5ml+0.3% Kelcogel® stock 5 ml+MC stock solution 300 ul; 3DKG Custom TeSR™ Base Media: custom TeSR™ media 95 ml+0.3% Kelcogel® stock 5 ml+MC stock solution 300 ul; 1% final concentration of Penicillin/streptozocin is added for 3DKG media).

### Preparation of Human Pancreatic Endocrine Progenitors and β-Like Cells In Vitro

Pancreatic endocrine cells (hiPSC-PEs) were prepared from human iPSC using differentiation methods as previously described. Briefly, human induced pluripotent stem cells (hiPSC) derived from HUVECs were obtained from the Stem Cell Core (Salk Institute). Cells were maintained on MATRIGEL® (BD)-coated dishes in complete STEMCELL™ TeSR™ media at 37 degree in a humidified 5% CO₂ incubator. For pancreatic differentiation, hiPSC were infected with a human insulin reporter lentivirus (pGreenZero lenti reporter human insulin, System Biosciences) by Spinfection (800 g, 1 hour). Methods 1: Media was changed to 100 ng/ml human Activin (R&D Systems), 25 ng/ml recombinant human Wnt3a (R&D Systems) in custom TeSR™ media (800 ml DMEM/F12, 13.28 g BSA, 10 ml Glutamax, 560 mg NaHCO₃, 330 mg thiamine, 100 mg reduced glutathione, 3300 mg Vitamin C, 14 μg Selenium, 10 ml NEAA, 2 ml Trace Element B, lml Trace Element C, 7 μl β-ME, 2 ml DLC, 2 ml GABA, 2 ml LiCl, 129.7 μg PA, Insulin 2 mg up to 1000 ml) for 2 days and then 100 ng/ml human Activin in differentiation media for another 2 days (Stage 1, Pancreatic Endoderm). Subsequently, media was replaced with custom TeSR™ media with 1 uM dorsomorphin (Calbiochem), 2 μM Retinoic Acid (Sigma), 10 μM SB431542 and 1% of B27 supplement for 7 days (Stage 2). Media was then replaced with custom TeSR™ media with 10 uM forskolin (Sigma), 10 μM dexamethasone (Stemgent), 10 μM TGFβ RI Kinase inhibitor II/Alk5 inhibitor II (Calbiochem or Enzo), 10 μM Nicotinamide (Sigma), 1 μM 3,3′,5-Triiodo-L-thyronine sodium salt (T3) and 1% of B27 supplement for 4-5 days (day15-day21, Pancreatic endocrine progenitors). Media was replaced every day (stage 1) or every other day (stage 2 & stage 3).

Methods 2: Media was changed to 100 ng/ml human Activin (R&D Systems), 25 ng/ml recombinant human Wnt3a (R&D Systems) or 3 μM CHIR99021 (Axon or Selleckchem) in differentiation media (S1) for 1 day and then 100 ng/ml human Activin in differentiation media (S1) for another 2 days (Stage 1, Pancreatic Endoderm). Subsequently, media was replaced with differentiation media (S2) with 50 ng/ml FGF7 (R&D Systems) for 2 days and then differentiation media (S3) with 50 ng/ml FGF7, 0.25 μM SANT-1 (Sigma), 1 μM Retinoic Acid (Sigma), 100 nM LDN193189 and 100 nM α-Amyloid Precursor Protein Modulator TPB for 3 days. Subsequently, media was replaced with differentiation media (S4) with 0.25 μM SANT-1, 50 nM Retinoic Acid, 10 μM Alk5 inhibitor II, 1 μM T3 for 3 days. Subsequently, media was replaced with differentiation media (S5) with 100 nM LDN193189, 100 nM Gamma Secretase inhibitor XX GSiXX (Millipore), 10 μM Alk5 inhibitor II, 1 μM T3 for 7 days. Subsequently, media was replaced with differentiation media (S5) with 10 μM Trolox (Calbiochem), 2 μM R428 (Selleckchem), 1 mM N-acetyl cysteine, 10 μM Alk5 inhibitor II, 1 μM T3 for additional 7 to 20 days.

S1 Media (MCDB131 Media, 8 mM glucose, 2.46 g/L NaHCO3, 2% Fatty acid free BSA, 0.25 mM L-Ascorbic acid 0.002% Insulin-Transferrin-Selenium ITS-X (GIBCO), 2 mM Glutamax, 1% Penicillin-Streptomycin), S2 Media (MCDB131 Media, 8 mM glucose, 1.23 g/L NaHCO3, 2% Fatty acid free BSA, 0.25 mM L-Ascorbic acid, 0.002% Insulin-Transferrin-Selenium ITS-X (GIBCO), 2 mM Glutamax, 1% Penicillin-Streptomycin), S3 Media (MCDB131 Media, 8 mM glucose, 1.23 g/L NaHCO3, 2% Fatty acid free BSA, 0.25 mM L-Ascorbic acid, 0.5% Insulin-Transferrin-Selenium ITS-X (GIBCO), 2 mM Glutamax, 1% Penicillin-Streptomycin), S4 Media (MCDB131 Media, 8 mM glucose, 1.23 g/L NaHCO3, 2% Fatty acid free BSA, 0.25 mM L-Ascorbic acid, 0.002% Insulin-Transferrin-Selenium ITS-X (GIBCO), 2 mM Glutamax, 1% Penicillin-Streptomycin, 10 μg/ml Heparin, 10 μM Zinc Sulfate), S5 Media (MCDB131 Media or BLAR Media, 20 mM glucose, 1.754 g/L NaHCO3, 2% Fatty acid free BSA, 0.25 mM L-Ascorbic acid, 0.002% Insulin-Transferrin-Selenium ITS-X (GIBCO), 2 mM Glutamax, 1% Penicillin-Streptomycin). For 3 dimensional culture, hiPSC or hESC were cultured in 3DKG Stem TeSR™ Base Media with 10 μM Y-27632 for 5 to 7 days and then Media were replaced each Differentiation media with 0.015% Kelcogel and 0.3% Methylcellulose.

### Generation of Three-Dimensional Pancreatic Islet Bud In Vitro: Islet-Like Organoids in Matrigel Through Co-Culture with hADSCs and HUVECs

Primary HUVECs and human Adipose-derived stem cells (hADSC) (Invitrogen or PromoCell) were cultured in 15 cm dish with EBM Medium (Ronza, cc-3121) or MesenProRSTM Medium (GIBCO, 12747-010 or Preadipocyte Growth Medium Kit, C-27417), respectively, at 37 degree Celsius in a humidified 5% CO₂ incubator. For co-culturing experiments, pancreatic endocrine progenitors derived from human iPSC were treated with Accutase, while HUVECs and hADSC were treated with TrypLE (GIBCO, 12604-013) and cells collected into a 50 ml tube respectively. After the cells were counted, 1×10⁶ cells of hiPS-PP, 7×10⁶ cells of HUVEC and 1-2×10⁵ cells of hADSC were co-cultured in 1 well of 24 well with 300 ul of MATRIGEL® matrix. For the purpose of scalable generation of human islets like organoids, 1×10⁶ cells of hiPS-PP (day15-day21), 7×10⁶ cells of HUVEC and 1-2×10⁵ cells of hADSC were co-cultured in 3DKG Custom TeSR® media with 10 uM forskolin (Sigma), 10 uM dexamethasone (Stemgent), 10 uM TGFβ RI Kinase inhibitor II/Alk5 inhibitor II (Calbiochem or Enzo), 10 uM Nicotinamide (Sigma), 1 uM 3,3′,5-Triiodo-L-thyronine sodium salt (T3) and 1% of B27 supplement, R428 (2 μM), Zinc sulfate (1004) and N-Cys (1 mM). (Methods 1) or co-cultured in differentiation media (S5) with 100 nM LDN193189, 100 nM Gamma Secretase inhibitor XX GSiXX (Millipore), 10 μM Alk5 inhibitor II, 1 μM T3 for 7 days. Subsequently, media was replaced with differentiation media (S5) with 10 μM Trolox (Calbiochem), 2 μM R428 (Selleckchem), 1 mM N-acetyl cysteine, 10 μM Alk5 inhibitor II, 1 μM T3 for additional 7 to 20 days (Methods 2). Mixed cells formed spherical, islet-like clusters within a few days. Media was changed every other day.

### Generation of 3D (Three-Dimensional) Pancreatic Islet Buds In Vitro: Islet-Like Organoids in Scalable Gellan Gum Through Co-Culture with hADSCs and HUVECs

Cells were prepared as described above. Briefly, 1×10⁸ cells of hiPS-PP, 2-7×10⁷ cells of HUVECs and 5-7×10⁶ cells of hADSC were co-cultured in 60-100 ml of 3DKG Custom TeSR™ with 10 μM forskolin (Sigma), 10 μM dexamethasone (Stemgent), 10 μM TGFβ RI Kinase inhibitor II/Alk5 inhibitor II (Calbiochem or Enzo), 10 μM Nicotinamide (Sigma), 1 μM 3,3′,5-Triiodo-L-thyronine sodium salt (T3) and 1% of B27 supplement, R428 (2 μM), Zinc sulfate (10 μM) and N-Cys (1 mM) (Methods 1) or co-cultured in differentiation media (S5) with 100 nM LDN193189, 100 nM Gamma Secretase inhibitor XX GSiXX (Millipore), 10 μM Alk5 inhibitor II, 1 μM T3 for 7 days. Subsequently, media was replaced with differentiation media (S5) with 10 μM Trolox (Calbiochem), 2 μM R428 (Selleckchem), 1 mM N-acetyl cysteine, 10 μM Alk5 inhibitor II, 1 μM T3 for additional 7 to 20 days (Methods 2). Mixed cells formed spherical, islet-like clusters within a few days. Media was changed every day or every other day.

### Generation of 3D (Three-Dimensional) Pancreatic Islets Bud In Vitro: Islet-Like Organoids in Scalable Gellan Gum 3D Culture Methods Without (w/o) Using hADSC and HUVECs

Human PSCs, including iPSC or ESC, were initially cultured in matrigel-coated plates (2 dimensional (2D) cultures. Cells were then treated with Accutase (Innovative Cell Technologies, Inc., San Diego, CA) to generate a single cell suspension, washed with PBS and centrifuged at 1000-1300 rpm for 5 minutes to pellet cells. Cells were resuspended with 3DKG Stem TeSR™ Base Medium (Stemcell Technologies, Cambridge, MA) with 10 μM Y-27632 (a RHO/ROCK pathway inhibitor compound) and cultured for an additional for 5 to 7 days until PSC sphere growth reached 50-100 μm diameter. Media was then replaced with differentiation media supplemented with 0.015% Kelcogel and 0.3% Methylcellulose. The culture medium was changed to differentiation medium (S1) containing 100 ng/ml human Activin (R&D Systems), 25 ng/ml recombinant human Wnt3a (R&D Systems) or 3 μM CHIR99021, a glycogen synthase kinase GSK-3 inhibitor (Axon Medchem, Reston, VA; or Selleckchem) for 1 day and then to differentiation medium (S1) containing 100 ng/ml human Activin for another 2 days (Stage 1, Pancreatic Endoderm). Subsequently, the medium was replaced with differentiation medium (S2) containing 50 ng/ml FGF7 (R&D Systems) for 2 days, and then with differentiation medium (S3) containing 50 ng/ml FGF7, 0.25 uM SANT-1 (Sigma), 1 μM Retinoic Acid (Sigma), 100 nM LDN193189 (an ALK2 and ALK3 inhibitor, Sigma) and 100 nM α-Amyloid Precursor Protein Modulator TPB for 3 days. Subsequently, this medium was replaced with differentiation medium (S4) containing 0.25 μM SANT-1, 50 nM Retinoic Acid, 10 μM Alk5 inhibitor II, 1 μM T3 for 3 days. Subsequently, the medium was replaced with differentiation medium (S5) containing 100 nM LDN193189, 100 nM Gamma Secretase inhibitor XX GSiXX (Millipore) 10 μM Alk5 inhibitor II, 1 μM T3 for 7 days. Subsequently, the medium was replaced with differentiation medium (S5) containing 10 uM Trolox (Calbiochem), 2 μM R428 (Selleckchem), 1 mM N-acetyl cysteine, 10 μM Alk5 inhibitor II, 1 μM T3 for an additional 7 to 20 days. After confirmation of the insulin gene expression by either reporter expression or qPCR (typically on day 20-30), the medium was changed to differentiation medium (S5) containing 10 μM Trolox (Calbiochem), 2 μM R428 (Selleckchem), 1 mM N-acetyl cysteine, 10 μM Alk5 inhibitor II, 1 μM T3 and 100 ng/ml recombinant human (rh)Wnt4 (R&D Systems), 400 ng/ml rhWnt5a, or 50% Wnt5a conditioned medium for 1-20 days. Wnt5a conditioned medium was prepared by culturing an L-Wnt5a cell line (ATCC, CRL-2814) in DMEM with 10% FBS, 1% Penicillin-streptomycin for 4 days after cells had reached 70-100% confluence in T175-T225 Frasko cell culture flasks.

### Generation of 3D (Three-Dimensional) Liver Bud In Vitro: Organ Buds

Hepatocyte cells (hiPSC-HEs) from human iPSC were prepared using differentiation methods as previously described. Briefly, hiPSCs were maintained on MATRIGEL® (BD)-coated dishes in complete STEMCELLTM TeSRTM media at 37 degrees Celsius in a humidified 5% CO₂ incubator. For hepatic differentiation, hiPSC (90% confluence in 6 well) were cultured with 100 ng/ml human Activin (Sigma) and 25 ng/ml recombinant human 0Wnt3a (R&D systems) or 3 μM CHIR99021 and 1% B27 supplement minus Insulin in RPMI1640 media for 1 day and then 100 ng/ml human Activin and 1% B27 supplement minus Insulin in RPMI media for another 4 days (Stage 1 Hepatic-Endoderm). Subsequently, media was replaced with differentiation media with 10 ng/ml bFGF, 20 ng/ml BMP4 and 1% of B27 supplement in RPMI1640 media for 3 days (Stage 2). Media was then replaced with differentiation media with 0.1 μM Dexamethasone, 20 ng/ml OncostatinM (R&D Systems) and 10-20 ng/ml Hepatic Growth Factor (HGF, R&D Systems) and 1% of B27 supplement in Hepatocyte Culture Media (Lonza, MD, CC-3198, withdraw EGF and Gentamicin/Amphotericin-B) for 4-22 days (dayl5-dayl9, Pancreatic endocrine progenitors). Media was replaced every day (stage 1) or every other day (stage 2 & stage 3). Primary HUVECs cells and human Adipose-derived stem cells (hADSC) (InVitrogen or PromoCell) were cultured in 15 cm dish with EBM Media (Ronza, cc-3121) or MesenProRS Media (GIBCO, 12747-010 or Preadipocyte Growth Medium Kit, C-27417), respectively, at 37 degree Celsius in a humidified 5% CO₂ incubator. For co-culturing experiments, day 10-hepatocytes derived from human iPSC were treated with Accutase, while HUVECs and hADSC were treated with TrypLE (GIBCO,12604-013) and cells were collected into 50 ml tube, respectively. After the cells were counted, 1×10⁶ cells of hiPS-PP, 7×10⁶ cells of HUVEC and 1-2×10⁵ cells of hADSC were co-cultured in 1 well of 24 well with 300 ul of matrigel. Liver-like organoids were formed within 1 to 2 days. Then, liver-like organoids were taken out from MATRIGEL® matrix and cultured in in 3DKG Custom TeSR™.

### Generation of 3D (Three-Dimensional) Heart Bud In Vitro: Organ Buds

Cardiomyocyte cells (hiPSC-CDs) were prepared from human iPSC using differentiation methods as previously described. Briefly, hiPSCs were maintained on MATRIGEL® (BD)-coated dishes in complete Stemcell™ TeSR™ media at 37 degree Celsius in a humidified 5% CO₂ incubator. For hepatic differentiation, hiPSC (90% confluence in 6 well) were cultured with 100 ng/ml human Activin (R&D Systems) and 10 uM CHIR99021 and 1% B27 supplement minus Insulin in RPMI1640 media for 1 days and then 1% B27 supplement minus Insulin in RPMI media for another 2 days (Stage 1 cardiac-Mesoderm). Subsequently, media was replaced with RPMI1640 with 5 uM IWP-2 and 1% B27 supplement minus Insulin in RPMI media for 1 days (Stage 2). Media was then replaced with 1% B27 supplement minus Insulin in RPMI Media for 6 days or more (Stage 3). Cardiac contraction started around day 13. Media was replaced every day (stage 1) or every other day (stage 2 & stage 3). Primary HUVECs cells and human Adipose-derived stem cells (hADSC) (Invitrogen or PromoCell) were cultured in 15 cm dish with EBM Media (Ronza, cc-3121) or MesenProRS™ Media (GIBCO, 12747-010 or Preadipocyte Growth Medium Kit, C-27417), respectively at 37 degrees Celsius in a humidified 5% CO₂ incubator. For co-culturing experiments, day 13 to day 15 cardiomyocytes derived from human iPSC were treated with Dispase, while HUVECs and hADSC were treated with TrypLE (GIBCO, 12604-013) and cells collected into 50 ml tube respectively. After the cells were counted, 1×10⁶ cells of hiPS-PP, 7×10⁶ cells of HUVEC and 1-2×10⁵ cells of hADSC were co-cultured in 3DKG Custom TeSR™ media. Mini heart like organs capable of contracting were formed within a few days.

### Generation of 3D (Three-Dimensional) Intestine Bud In Vitro: Organ Buds

Intestinal cells (hiPSC-ITs) were prepared from human iPSC using differentiation methods as previously described. Briefly, hiPSCs were maintained on Matrigel® (BD)-coated dishes in complete Stemcell™ TeSR™ Media at 37 degrees Celsius in a humidified 5% CO₂ incubator. For hepatic differentiation, hiPSC (90% confluence in 6 well) were cultured with 100 ng/ml human Activin (R&D Systems), 3 uM CHIR99021, 2 mM Glutamax and 1% B27 supplement minus Insulin in RPMI1640 media for 1 day and then 100 ng/ml human Activin (R&D Systems), 2 mM Glutamax and 1% B27 supplement minus Insulin in RPMI1640 media for another 3 days (Stage 1 Forgut-Endoderm). Subsequently, media was replaced with 500 ng/ml Wnt3a, 500 ng/ml FGF4 and 1% B27 supplement in RPMI 1640 media for 4 days (Stage 2). Cells were transferred to Matrigel® matrix and then a 3D-spheroid Matrigel® dorm was made in the bottom of 24 well. Media was then replaced with 1% B27 supplement, 1% N2 supplement, 500 ng/ml R-spondin, 100 ng/ml Noggin, 50 ng/ml EGF, 2 mM Glutamax™ supplement, 10 uM HEPES in DMEM/F12 Media for 7 days or more (stage3). Intestinal-like organoid spheroids were observed within a week. Media was replaced every day (stage 1) and every other day (stage 2 & stage 3). Primary HUVECs cells and human Adipose-derived stem cells (hADSC) (Invitrogen or PromoCell) were cultured in a 15 cm dish with EBM Media (Ronza, cc-3121) or MesenProRSTM Media (GIBCO®, 12747-010 or Preadipocyte Growth Medium Kit, C-27417), respectively, at 37 degrees Celsius in a humidified 5% CO₂ incubator. For co-culturing experiments, intestinal progenitors (day 7) derived from human iPSC were treated with Accutase, while HUVECs and hADSC were treated with TrypLE (GIBCO®,12604-013) and cells collected into a 50 ml tube respectively. After counting the cells, 1×10⁶ cells of hiPS-PP, 7×10⁶ HUVEC cells and 1-2×10⁵ hADSC cells were co-cultured in 3DKG Custom TeSR™ media.

### Insulin Secretion Assay (Primary Mouse and Human Pancreatic Islets and Human iPSC-Derived Cells)

Insulin release from intact islets was monitored using batch incubation methods (Yoshihara et al., 2010, Nat. Commun. 1:127). Briefly, overnight-cultured isolated pancreatic islets (RPMI-1640 supplemented with 10% (v/v) fetal bovine serum and 1% (v/v) Antibiotic-Antimycotic (Gibco)) were pre-cultured at 37° C. for 30 min (Krebs-Ringer bicarbonate buffer (KRBB) containing 129.4 mM NaCl, 3.7 mM KCl, 2.7 mM CaCl₂, 1.3 mM KH₂PO₄, 1.3 mM MgSO₄, 24.8 mM NaHCO₃ (equilibrated with 5% CO₂, 95% O₂, pH7.4), 10 mM HEPES and 0.2% (v/v) BSA (fraction V, Sigma) (KRBH) with 3 mM glucose). Pancreatic islets were then incubated in KRBH buffer (500 μl/10 islets) with 3 mM or 20 mM glucose to determine insulin secretion levels. After 30 min, islets were pelleted by centrifugation and insulin levels determined by ELISA (Rat/mouse Insulin ELISA KIT (Millipore) and Human Insulin ELISA KIT (Millipore) for mouse and human islets, respectively). For human iPSC derived cells, the cells (1×10⁶ cells/well in 24 well) were pre-cultured in 3 mM glucose KRBH buffer (500 μl/well). The cells were then incubated in KRBB (200 μl/well) with 3 mM or 20 mM glucose to determine c-peptide secretion levels as indicator of insulin secretion levels. After 30 min, the cells were pelleted by centrifugation and c-peptide levels were determined by human c-peptide ELISA KIT (Millipore).

### Quantitative RT-PCR Analysis

Total RNA was extracted using TRIzol reagent (Invitrogen) and RNeasy KIT (Qiagen). Reverse transcription was performed with a SuperScript III First-Strand Synthesis System kit (Invitrogen) or PrimeScript RT reagent kit (TAKARA). Real time quantitative RT-PCR (qPCR) was performed using SYBR Green (Bio-Rad).

### Lentivirus Production for Proinsulin-NanoLuc

Proinsulin-NanoLuc in pLX304 (Addgene, #62057) was obtained from Addgene. Proinsulin-NanoLuc lentivirus was produced using a second-generation viral packaging system. Briefly, 14 μg of Proinsulin-NanoLuc, 6.6 μg of PsPAX2 packaging plasmid (Addgene 12260), 5.4 μg of pMD2.G envelope plasmid (Addgene 12259) and 54 μl Lipofectamin2000 (Invitrogen) were used to transfect a T75 flask of HEK293LTV packaging cells. Twenty-four (24) hours after transfection, media was changed to fresh DMEM with 10% FBS and 1% Penicillin/Streptozocine. Forty-eight (48) hours and 96 hours after transfection, viruses were collected as day 1 and day 3, respectively and passed through 0.2 μm cellulose acetate filters (VWR). Viruses were aliquoted and frozen at −80 degrees Celsius until use.

### Gaussia Luciferase Assay for Insulin Secretion Measurement

Mouse islets, human islets and human islets like organoids were plated in their respective growth media with 10 μg/ml Polybrene® polymer (Santacruz). Viruses were then added. After overnight culture, cells were placed in fresh growth media. Forty-eight (48) to 72 hours after infection, mouse islets, human islets and human islet-like organoids were picked up by hand and then placed into 96 wells with single islet or organoid. Then, insulin secretion assays were performed. Briefly, a single islet or organoid was pre-incubated with 3 mM glucose KRBB at 37° C. for 30 min to 1 hour. The cells were then incubated in KRBB (100 μl/well) with 3 mM for 30 min and then sequentially incubated with 20 mM glucose with or without 100 nM Exendin-4 or 3 mM glucose with 20 mM KCl (100 μl/well). To determine Gaussia Luciferase activity as indicator of insulin secretion levels, 10 μl of samples are used for Luciferase assay using Pierce Gaussia Luciferase Flash Assay Kit (Prod #16159, Thermo Scientific).

INS-1 cells were infected with the virus by spinfection (800 g, 1 hour at 37 degrees Celsius) and then changed to fresh INS-1 growth media. Seventy-two (72) hours after transfection, INS-1 cells were treated with 5 μg/ml Blasticidin (Invitrogen) for 7 days to select for Proinsulin-NanoLuc expressing cells. For insulin secretion assay, the cells (5×10⁴-1×10⁵ cells/well in 96 well) were pre-cultured in 3 mM glucose KRBB (100 μl/well). The cells were then incubated in KRBB (100 μl/well) with 3 mM and then sequentially incubated with 20 mM glucose with or without 100 nM Exendine-4 or 3 mM glucose with 20 mM KCl (100 μl/well). To determine Gaussia Luciferase activity as indicator of insulin secretion levels, 10 μl of samples are used for Luciferase assay using Pierce Gaussia Luciferase Flash Assay Kit (Prod #16159, Thermo Scientific).

### Vascularization Test In Vitro

Human islet-like organoids were embedded in 1 well of 24 well plate with 300 μl of Matrigel® matrix with EBM Media (Ronza, cc-3121). Vascularization was observed within 24-72 hours.

### 3D Culture of hADSCs and WNT Protein Expression

hADSCs undergo changes in the expression of Wnt genes, in particular genes in the Wnt5a pathway, during the spontaneous self-organization that occurs in 3D culture. (FIG. 11A). Wnt5a was found to be the predominant protein expressed among the Wnt proteins in hADSC 3D culture over time. (FIG. 11B).

### Example 5: Wnt Proteins in the Metabolic Maturation of iPSC-Derived Islet Organoids

Fltp and Esrrg genes were found to be expressed in iPSC-derived islet organoids (day 21, generated without co-culture with hADSCs or HUVECs) after treatment with PBS, WNT3a (500 ng/ml), recombinant human (rh)WNT4 (100 ng/ml), or rhWNT5a (400 ng/ml) for 5 days. (FIG. 12A). As shown in FIG. 12B, Esrrg gene expression was induced in hiPSC-derived islet organoids that were generated in the absence of supporting hADSC or HUVECs, in response to increasing doses of rhWNT4 (0, 10, 25, 50, 100, 200 ng/ml) and rhWNT5a (0, 25, 50, 100, 200, 400 ng/ml). In addition, mitochondrial genes involved in oxidative phosphorylation (Cox7a2, Ndufa1, Ndufa7), lactate dehydrogenase (Ldha) and Fltp (a Wnt/planar cell polarity (PCP) effector and reporter gene) were induced in hiPSC-derived islet organoids that were generated in the absence of supporting hADSC or HUVECs, in response to increasing doses of rhWNT4 (0, 10, 25, 50, 100, 200 ng/ml) and rhWNT5a (0, 25, 50, 100, 200, 400 ng/ml), (FIG. 12C). Mitochondrial (Mitotracker; Mito-Red) and insulin (Insulin-GFP) levels were increased in hiPSC-derived islet organoids (day 27) after 8 days treatment with PBS or WNT4 (100 ng/ml). (FIG. 12D). Human iPSC-derived islet organoids (day 27) were generated after 8 days treatment with PBS or WNT4 (100 ng/ml). (FIG. 12E). Insulin production was found in hiPSC-derived islet organoids (day 27) after 8 days treatment with rhWNT4 (100 ng/ml), rhWNT5a (400 ng/ml), or WNTSa secreting fibroblast conditioned media (50%), compared with PBS and control fibroblast conditioned media (50%). (FIGS. 12F-12H). Human iPSC (hiPSC)-derived islet organoids (day 22) treated with rhWnt4 (100 ng/ml) for 12 days showed functional maturation based on their secretion of human c-peptide, as measured in response to low glucose (3 mM, “G3 mM”), high glucose (20 mM, “G20 mM”), or high KCl levels (20 mM, “KCL20 mM”), (FIG. 13).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1.-91. (canceled)

92. A cell culture comprising an induced pluripotent stem cell (iPSC)-derived beta-like cell in a medium comprising a three-dimensional matrix comprising gellan gum.

93. The cell culture of claim 92, further comprising an adipose-derived stem cell and/or an endothelial cell.

94. The cell culture of claim 92, wherein the iPSC-derived beta-like cell is a human iPSC-derived beta-like cell.

95. The cell culture of claim 93, wherein the adipose-derived stem cell is a human adipose-derived stem cell (hADSC).

96. The cell culture of claim 93, wherein the endothelial cell is a human umbilical vein endothelial cell (HUVEC).

97. The cell culture of claim 92, wherein the medium comprises a Wnt4 or a Wnt5a polypeptide, or a fragment thereof.

98. The cell culture of claim 97, wherein the Wnt4 or Wnt5a polypeptide, or a fragment thereof, is a recombinant human Wnt4 or Wnt5a polypeptide, or a fragment thereof.

99. The cell culture of claim 97, wherein the Wnt4 or Wnt5a polypeptide, or a fragment thereof, is present in culture medium of a Wnt4- or Wnt5a-producing cell line.

100. A functional pancreatic islet organoid comprising an iPSC-derived beta-like cell, an iPSC-derived alpha cell, an iPSC-derived delta cell, an iPSC-derived duct cell, an adipose-derived stem cell (ADSC), and an endothelial cell wherein the organoid is vascularized and exhibits glucose-stimulated insulin secretion (GSIS), KCl-stimulated insulin secretion, GLP-1 stimulated insulin secretion, somatostatin secretion, and glucagon secretion.

101. The pancreatic islet organoid of claim 100 surrounded by an iPSC-derived exocrine component.

102. The pancreatic islet organoid of claim 101, wherein the iPSC-derived exocrine component expresses a marker selected from the group consisting of: PDX1, Nkx6-1, and Ptf1.

103. The pancreatic islet organoid of claim 100, wherein the adipose-derived stem cell is a human adipose-derived stem cell (hADSC) and/or the endothelial cell is a human umbilical vein endothelial cell (HUVEC).

104. The pancreatic islet organoid of claim 100, wherein the pancreatic islet organoid expresses a beta cell transcription factor selected from the group consisting of Pdx1, MafA, Pax4, Pax6, NeuroD1, Nkx6-1, Gata6, and Foxa2.

105. The pancreatic islet organoid of claim 100, which is derived from the cell culture of claim 1.

106. The pancreatic islet organoid of claim 100, wherein the iPSC-derived beta-like cell, the iPSC-derived alpha cell, the iPSC-derived delta cell, the iPSC-derived duct cell, the adipose-derived stem cell (ADSC), and the endothelial cell are human.

107. The pancreatic islet organoid of claim 106, wherein the endothelial cell is a human umbilical vein endothelial cell (HUVEC).

108. A method of identifying an agent that modulates pancreatic activity and/or treats a pancreatic disease, the method comprising:
(a) contacting a candidate agent with a pancreatic islet organoid or a pancreatic organoid; and
(b) measuring a pancreatic activity of the organoid contacted with the candidate agent, wherein the candidate agent is identified as an agent that modulates pancreatic activity and/or treats a pancreatic disease if the activity of the organoid is altered relative to a reference; or
(a) administering a candidate agent to a non-human subject transplanted with a pancreatic islet organoid or a pancreatic organoid; and
(b) measuring a pancreatic activity of the non-human subject, wherein the candidate agent is identified as an agent that modulates pancreatic activity and/or treats a pancreatic disease if the pancreatic activity of the non-human subject is altered relative to a reference.

109. The method of claim 108, further comprising inducing or mimicking a pancreatic disease in the pancreatic islet organoid, in the pancreatic organoid, or in the non-human subject.

110. The method of claim 108, wherein the pancreatic disease comprises type 2 diabetes or pancreatic cancer.

111. The method of claim 108, wherein
(i) the pancreatic disease is induced by contacting the pancreatic islet organoid or the pancreatic organoid with any one or more of the following agents: a free fatty acid (FFA), glucose, and a cytokine;
(ii) the pancreatic disease is mimicked by culturing the organoid with pancreatic cancer cells, stellate cells, and immune cells to create a human pancreatic cancer microenvironment; or
(iii) the pancreatic disease is induced or mimicked by administering to the non-human subject any one or more of the following agents: a free fatty acid (FFA), glucose, and a cytokine.

112. The method of claim 108, wherein the pancreatic activity is selected from the group consisting of: insulin secretion, beta cell apoptosis, expression or activity of a NDUFA4, ESRRG, G6PC2, MDH1, LDHA, KCNK3, or MAFA polypeptide or polynucleotide, amylase secretion, apoptosis of an exocrine component, collagen synthesis, and stellate cell activation.

113. The method of claim 108, wherein the candidate agent increases insulin secretion, or wherein the candidate agent identified as an agent that modulates pancreatic activity is further tested for ability to treat a pancreatic disease.

114. A method of enhancing self-organization of adipose-derived stem cells (ADSCs) for generating an induced pluripotent stem cell (iPSC)-derived organoid, the method comprising culturing the ADSCs in a 3-dimensional culture matrix medium comprising a Wnt5a polypeptide or a fragment thereof.

115. The method of claim 114, wherein the 3-dimensional culture matrix medium comprises gellan gum.

116. The method of claim 115, wherein the 3-dimensional culture matrix medium comprises an iPSC-derived beta-like cell and the iPSC-derived organoid is a pancreatic islet organoid or a pancreatic organoid.

117. The method of claim 116, wherein the iPSC-derived beta-like cell and the pancreatic islet organoid or pancreatic organoid is a human iPSC-derived beta-like cell and is a human-derived pancreatic islet organoid or pancreatic organoid.

118. The method of claim 114, wherein the Wnt5a polypeptide or a fragment thereof is a recombinant human Wnt5a polypeptide or a fragment thereof or is present in culture medium of a Wnt5a-producing cell line.
